SEQUENCE LISTING (478 sequences)

SEQ ID NO: 1 - DNA, 401 bp, Homo sapiens
tgggacatacagtgtcctttggaggaccaggagcctcgcccaaacagacttgaacaactgttgaccagacttgacagcatgcagtccacagaagtgcctgaagccccgggggtagggtggtgggaacaaaaaacaaaaccgtttttatcttttgcactctccctttctcctctaggccattgttgaaaagggtagcttttytttttttttaacataaaaagaaatatactatactttccatattttgggcaattttaggatgttttgtggaatctagaatcaaccagatttaccctgaatgtactggaaaatcttttgattgctcccaacttacttgtaaacctctatgtaaaagtgccataaatacaaacctacagatggtcctgaactagaccctctat

SEQ ID NO: 2 - DNA, 401 bp, Homo sapiens
ggttcctgcttctgctttgccttccgccatgattgtaagttttctgaggcctccccagccatgcagaacagtgagtaaattaaatctcttttctttataaattactcagtctcaggtattcctttattgcagcatgagaacagactaatacaacaactgtatacaaattttgggaaaaattaaaaaaaaacaaaatttacwtgtaatcataaaataaccattaatattttactttgttttttatcctaacctttttctaacacataaacgtattcacacacatgtaaatacaaacaaacgtctcctatctatcgcatttctctatgcatgtttatctgtttgcatacatggatatgtatgtgcatgtatatgcattgtattacgaatatagtttattattt

SEQ ID NO: 3 - DNA, 401 bp, Homo sapiens
ctaatacaacaactgtatacaaattttgggaaaaattaaaaaaaaacaaaatttacttgtaatcataaaataaccattaatattttactttgttttttatcctaacctttttctaacacataaacgtattcacacacatgtaaatacaaacaaacgtctcctatctatcgcatttctctatgcatgtttatctgtttgcayacatggatatgtatgtgcatgtatatgcattgtattacgaatatagtttattatttttcttttactcctttaaaaaaatttcaatacagtgtcatgggcgtgtccctatatttattaagtagtcttcaaaaatctaattatatagtctgtgtaatatcatcaaagtattcacgatatttttaacgtatttattatcgaat

SEQ ID NO: 4 - DNA, 401 bp, Homo sapiens
tctgcgtatactatacaagagagaagtaagtggattgacccataaaaggaaaaaagaattagcattttcctttactgttacctggtttatagaacattaaaacatctgccaaaggctatgatagtttagtctattttaatatggaagcagtatatatatatatatatatatggttttgtctttttctgcattagaaaaatsaaatgtggtggttcttttctgggactgttgttggaagtttaatccctccctcccctcaagagaaggaaaacgcaatttctcagtttcttatgatctggtatcaagtttaatgaatcatcaggttacattacacaagggttgggaagacatgggaagatgggagttttacacaccattggaagaatataaattggtacaac

SEQ ID NO: 5 - DNA, 401 bp, Homo sapiens
ttgttggaagtttaatccctccctcccctcaagagaaggaaaacgcaatttctcagtttcttatgatctggtatcaagtttaatgaatcatcaggttacattacacaagggttgggaagacatgggaagatgggagttttacacaccattggaagaatataaattggtacaacagctttggaagttaatgatgtctattcragttaaaggtatacattctttataaccatcaatcctattctcagtcaggtacaaaaggagccaaatatattaatggtgttcatctcagcattgtataataaataatagcaaataataaaaaaattattgcaaataataaataagaaatacaataaaaatagcaaataacaaataacaaaaaattgaaaaaatagcaaaaa

SEQ ID NO: 6 - DNA, 401 bp, Homo sapiens
tgaactgtgatagcaccattgcactttagcctgggtgatggagtgaaaacttgtctcttaaaaaaactagaaggcataatattgaaacatatttatttacacgaggaaaatattcacgatatattttgttcaagaaaaaaatcacaatacaaaatggaatatacataacaatgaaaatttaacaaaatatatttgcatatytatatgtacaaatgacagaggaaatatttcaaaaagttattaccttatgatttttatttattcttcatttatttatattttgcatgtgtcctataataaacatgttttaataaaaaaattcagaagacaattggacactaaaataataaacattttgtagtcctccattcactttcataacagacattttatgtagaaag

SEQ ID NO: 7 - DNA, 401 bp, Homo sapiens
agcatatgaaggcctttgaaatctgcgcttatttcttgttttaggcttgctctccttcaaatccctccattgcagcccagtgggctgccttttccatgaacttcattcacctattccttttcctgcaacccacttccaactatgtaaaaagcacagtctcctctttcacagcactcgcctctggtattgcaatatattttycccatcacatccactagactgtgaactcttgaggttgtttatgttttattctcctttatcccattagtgcctagcacaattcctgacacatcatcagtaaatatttgttgagtgcataagtaaaagagaatatgtattgtaaaagcaatttgcaaactgcaaagtgctgtaaagtaagcattccttatttcatacgtgca

SEQ ID NO: 8 - DNA, 401 bp, Homo sapiens
ttggtgcagtgactcattagaaactgtaatcaccacgtttcaccaagcttaaacctccaagtgattggtgcaacttttttaatgggaatgagacttggactcagcagactacatctcactgggagacaatacagccttgtattaacagcttatactgacgaaaccatgaaaaaacacaatcacatttatttacaatttttwaatttgtttatctgtacttagcagcaacatggttgtttctcggaaggtcagagtagggtgctcatgaacttaagtttaatattgggcattgttactttatttttggccatttacgattgtactatgcctttacttaggaggggcacacagcaagagtgtggacagaacacaggaaactcatggttgcttgtgcgtaacaa

SEQ ID NO: 9 - DNA, 401 bp, Homo sapiens
tggaaggaggactctctcagctgctttaagggttttgcagcttggttggttagtccagaagatggtaattaaaagatcccttcaagggaaagaatagcttcaagaaaagcaagattcatgagtaaattaggagagcttcaggattcaggtgccacatgctgtatgtagctaggagtgcaggctcttctgtgactcaggcayacctgagtgcttcagaggcaaaccagtctcattttatttctggtgcagaactccaagtttcttaaaagttactccactgtcagcgtatgtgtgtaccaccttttgcaagagagcatggagaaagagagagcgtattgtatttccagctctgatgtacttgtcttagctatattgttttaaatctggcatttagactgaat

SEQ ID NO: 10 - DNA, 401 bp, Homo sapiens
aacagagaaaggtatgtttttgattagctaaaaattttaacataaaattctaatattgtgtctataacatgttttacatggattagtatgaattgtgcaaattaccaatgaaggcagtggaagagggcctggggagaggagtgttttagccagagtttgcattctttttatttcctgcactctggatctattcaaatctcrtctcctgccgagggattcagtgcagaactaaaatatgatgacttacttggcaacagcaaaaaaaagagcatttactcaaatgcaataaagataagcaataagtttcagtgatgcatatagtatgttctctgctcgcctgcttgttctttcttctctcagcagaagttcaactgaaaataagctcaaacagcaatttgcca

SEQ ID NO: 11 - DNA, 401 bp, Homo sapiens
taaaaatacaaaaaattaaccaggcgtggtggtgggcacctgtagtcccagctacttgggaggctgaggcaggagaatggtgtgaacccgggaggtggagcttgcagtgagctgagatggcaccactgcactccagcctgggcgacagagcgagactccgcctcaaaaacaaaaacaaaaacaaaaacaaaaaaaacccayagatgacactatcagccaaagctatttcactgcttactcattaaacagacccaacactgaaacaatgccaaggattcatccattcaaatagaaggcacccaacatatcattttagggttaaaaagcacacaattgattaattagaaatcttagttaggaggagtttgtatcctagacttgacaggtaaaacaagctgtta

SEQ ID NO: 12 - DNA, 401 bp, Homo sapiens
tccgagcagaggggaaaacatgggtgaagaccctgatgcattaagataaaaacagttaattttgaaataagtatcaacttatagggaatgacaaaaatagcacatagagtgctgtaaactcttcaccagtttctccaaatggtggcatcttctatgactatggtataatatcaaaaccagaaagttgacattgatacaatmctagtaactagaccacagtcttacacagttttgatcagcttttacatgcaatgtgcttgtgtgtgtgtgtgtgtgtgtgtgtgtgtgtgtgcatgtgcgtgtgtttttatagttctatacagtttgacctcttgtagaggttcatataaccaccactacaatcagggtacagaactgtttcatcactacaaaagatctcc

SEQ ID NO: 13 - DNA, 401 bp, Homo sapiens
tctatataggtatacctactatataggtatttctatataacatagaaagaattggaggtatagcccatctgaatatatacagattccatttgttaggaagtcgatatagaaaaatccagaatttcttactataatttaagagatgattgacctccaaaaagcttcaccacagtatcaactctatgcttcatatattttctytaattcataagcagtacattttattcgtggcataatcacttcttcctatattccaaatgctttcttttctttgtttcctatcattttacatataatatgcaaatactctaaaattgaaatatagcttggcagtttgtaatactgtaaaaccataaagtgtaatatatgactacaaaatattttggtttaatgggcctggt

SEQ ID NO: 14 - DNA, 401 bp, Homo sapiens
ggtcgggagttcgagaccagcctgaccaacatggtaaaactctgtctctactaaaaatacaaaattagctgggcatggtggcacatgcctgtaatctcagctacttgggaggctgaggcaggagaatcgtttgaagctgggaggtgaagattgcggtaagccgagatcgtgacactgcactccagcctgggcaacaagagygaaactctgtctaaaaaaaaaaaaaagactcctcaccacttttgccatgtgtggttacagcaaaaagatggttgtttcaccagacacggaatctgctggtgtcttgagcttggactctccagtctcctgaattgtgatcaatgaatttctgttgtttgtaagctgtccagtgtattgtattttgctatagcagtgtaaac

SEQ ID NO: 15 - DNA, 401 bp, Homo sapiens
ctgtccttcacagttatactccgctctgtctttccattactactgttactagaaatgatttgtaaagtgtaaagcaagtggagtcaaatctggcaatgcctctctcttgctttgaaaacattaataaaggcagaggccaggttcagtggctcactcctataatcccaacattttgggagacggaggcaagaggactgtctrgtctctacaaaaaatttaaaaattagccaggtgtggtggtgcacacctgtagtcccagctattcaggaggctgaggcaggaggacctcttgagcccagtagttcaaggctgtagtgcactatgattgcacttgtgaatagccacgatgctccatcctgcacaacttagcgagactccatctctaaaaagcaaaaaaacaa

SEQ ID NO: 16 - DNA, 401 bp, Homo sapiens
tcaggagacagcaaattaaattgccatttgtaaggaataagatagggcaataagaaaggccagagatattctgttctgtactcaaagagggatagcataacccgaagacaaagtaagggcacggtgctagtattaggatggtgccattactttttttttctttttgagacggagtctcgctctgtcgcccaggctggagckcagtggctcgatctccgctcactaacctccgccttccgggttcacgccgttctcctgcctcagcctcccgagtagctgggactactggcgcccgccaccacgcctggctaatttttgtatttttagtagagacggggtttcaccgtgttagccaggatggtctcgatctcctgacctcatgatctgcccacctcggcctc

SEQ ID NO: 17 - DNA, 401 bp, Homo sapiens
gagagccctgagcaggaagtcacaactctcttcatcaccaggatagcaaagggggagcccacattccatgaagtctatgttccaaggtagaagggggaagtagtagatgccccgtgataggcaggagagctgcattgggtgcacacataaaatgagaacttccccccaaaaattagaggaatttgagggggagtcgagttyttagaattcagcaggtgagggatgaaccaaactctgtttaaacattattatatggtttagattttcctacaaactgtgaatacacaattaattattattttagctttgtgaacagaactatgctagcactgtggctatgacagggagtgaaatatggtttttgctttaaaagacttacagattttgggagaaatgtaagc

SEQ ID NO: 18 - DNA, 401 bp, Homo sapiens
tgaggagtattgtcacaaacgtgggaatacacacagctgaaataaaatgatgatgaggatgatgacaactaatataggtgaatattcaggaagtgccctccctagaccaaatgaggtagattatttcatttatttttcacaacaaccctatattttacattgtaacatgatctgcgttttaaagatgataaaacagacacwgggagcagcatgcccaggattcaaatgtaagatgacctgattctttatactaagctgtttattaaaaaaaaataaattttattgtgtatatttgaggtttacaagatgatgttatggggtacatatagatagtaaagtggttgctatagtgaagaaaattaacatatctatcatctcacatagtttcttttttatgacag

SEQ ID NO: 19 - DNA, 401 bp, Homo sapiens
gtaagatgacctgattctttatactaagctgtttattaaaaaaaaataaattttattgtgtatatttgaggtttacaagatgatgttatggggtacatatagatagtaaagtggttgctatagtgaagaaaattaacatatctatcatctcacatagtttcttttttatgacaggagcagctaaaacttacttacttaacrcaaatccctgatacatttttatttattcatttttttttagacggagcctctctctgttgcccagattggagtgcagtagcgcgatctcggctcactgcaagctccgccccccgggttcacgccattctcctgcctcagcctcccgaatagctgggactacaggcgcccaccaccaacctggctaattttttgcatttttc

SEQ ID NO: 20 - DNA, 401 bp, Homo sapiens
gcatttttcatagagacggggtttcaccgtgtcagccaggatggtctcgatctcctgacctggtgatctgcccgcctgggcttcccaaagtgctgggattataggcgtgagccaccgcgcccggcccctaatacatttttatgaactatagtccgcgtgttgtacattagatctctagaattgtcattctacatatctgcytctttgtgtcctctgatctacaaccccccatttcctccccttcttccctgccccggcaaccactgttttattcgctgtgtatgtgacctcttttcttccttagataccacatgtaaatgatatcatgcaatatttttatttttgtgtctgccttatttcacttagcataatgtcctccaggtcaatctgtgttgtaggaa

SEQ ID NO: 21 - DNA, 401 bp, Homo sapiens
catttttcatagagacggggtttcaccgtgtcagccaggatggtctcgatctcctgacctggtgatctgcccgcctgggcttcccaaagtgctgggattataggcgtgagccaccgcgcccggcccctaatacatttttatgaactatagtccgcgtgttgtacattagatctctagaattgtcattctacatatctgccwctttgtgtcctctgatctacaaccccccatttcctccccttcttccctgccccggcaaccactgttttattcgctgtgtatgtgacctcttttcttccttagataccacatgtaaatgatatcatgcaatatttttatttttgtgtctgccttatttcacttagcataatgtcctccaggtcaatctgtgttgtaggaaa

SEQ ID NO: 22 - DNA, 401 bp, Homo sapiens
tctcgatctcctgacctggtgatctgcccgcctgggcttcccaaagtgctgggattataggcgtgagccaccgcgcccggcccctaatacatttttatgaactatagtccgcgtgttgtacattagatctctagaattgtcattctacatatctgcctctttgtgtcctctgatctacaaccccccatttcctccccttcytccctgccccggcaaccactgttttattcgctgtgtatgtgacctcttttcttccttagataccacatgtaaatgatatcatgcaatatttttatttttgtgtctgccttatttcacttagcataatgtcctccaggtcaatctgtgttgtaggaaatggcaggatcccattctttttaaaacctgaataatattccagt

SEQ ID NO: 23 - DNA, 401 bp, Homo sapiens
tggagttttggtgaattaatgctactttaagaagaaagaaaaggggtcctgaatctaatagtgtttgggaagtatacactagggatccttagggaggcccagtgatctttacatcaaggtttgctctcagccctttgtgataaaatcaattcagacatgtcttcaaatctactcacagacaagagatggaataataataasggtagctgatatttttatagttcttttgtttatcatgtttatttttatggcaattttttagaaacaatttggcaaaactaatgattatcattttaaagtaggcaagaagaaacctagttatagaggacagaaatctagagtcaaggattcctgactcctaatccagtgctctttccactatagcccactgtgaaattttg

SEQ ID NO: 24 - DNA, 401 bp, Homo sapiens
tgctactttaagaagaaagaaaaggggtcctgaatctaatagtgtttgggaagtatacactagggatccttagggaggcccagtgatctttacatcaaggtttgctctcagccctttgtgataaaatcaattcagacatgtcttcaaatctactcacagacaagagatggaataataataagggtagctgatatttttatmgttcttttgtttatcatgtttatttttatggcaattttttagaaacaatttggcaaaactaatgattatcattttaaagtaggcaagaagaaacctagttatagaggacagaaatctagagtcaaggattcctgactcctaatccagtgctctttccactatagcccactgtgaaattttggaaagagtggaaataactg

SEQ ID NO: 25 - DNA, 401 bp, Homo sapiens
cctggcttacaacctgatagaactccccagcttgatgttaacagaataaataaaacaatatatccccaaatccatggggaaggcaagaacatgattggggattttaaatcctgtaattcttggattcagagtatggaaaatacaggtaatttcagtaagagatccccactgttagaatctataacaccctatggtgtcttwaaaaagaagaaaatgcgtcactagtggctcaatcccatactcctttaaaatgtgttaagcttgacaattcaagctgatggcaaataactcctttctcaagtaatgttttaatctgaaagaaatatattatttaaaagggaaatccccattaatgccaagatacggaaacaactacttagaatacacagcaacatgaaaga

SEQ ID NO: 26 - DNA, 401 bp, Homo sapiens
ttctacagggcagtgtagtttttaaattcgtggagcaagatggtgacactggctgtatgtactagcataaagaattgaaaacaatatgacctgaatgatactcaaaaactgatatattaaagactcatgttgagagacagatcatgagcagagccaattttgattttaagcataaaagtggatatgttgttatgaaagttyaagtttgagtatcacacttatgaaagttaagctttgaatgtcacaagatattggggtaaaggaaactgaagagtcatctagtttaactcaaattcttaagtagagaagagggtatgaggcttcacaatgacaagtggtctagtttacaggtaattcttgtccctatttgtaggacttcctgggagggtcaggttaagatc

SEQ ID NO: 27 - DNA, 401 bp, Homo sapiens
tacagggcagtgtagtttttaaattcgtggagcaagatggtgacactggctgtatgtactagcataaagaattgaaaacaatatgacctgaatgatactcaaaaactgatatattaaagactcatgttgagagacagatcatgagcagagccaattttgattttaagcataaaagtggatatgttgttatgaaagtttaastttgagtatcacacttatgaaagttaagctttgaatgtcacaagatattggggtaaaggaaactgaagagtcatctagtttaactcaaattcttaagtagagaagagggtatgaggcttcacaatgacaagtggtctagtttacaggtaattcttgtccctatttgtaggacttcctgggagggtcaggttaagatccca

SEQ ID NO: 28 - DNA, 401 bp, Homo sapiens
ttattttcagatattaaggttaagtactatatcaattttttaaaagtaaaaaattatttctacctgcatagaaggcattacctgggttgattgattgattgatttagagacagggttttactctgtagccccagctaaagtgcagaggcacatcacaccccactgcagcctctgcctcccgggctcaagcaaccctccctyctcagtttcctgagtaggtgaggctacaggcacacaccactacacccagctaatttttgtagtttttgtagagactgccgtgtttcccaggctggtctcaaactcctgggctcaagccatcagcctgccttggcctcccaaagttctaagattatgggcatgagccactgcacctggcctgattgtattttatttttaaa

SEQ ID NO: 29 - DNA, 401 bp, Homo sapiens
caagcaaccctccctcctcagtttcctgagtaggtgaggctacaggcacacaccactacacccagctaatttttgtagtttttgtagagactgccgtgtttcccaggctggtctcaaactcctgggctcaagccatcagcctgccttggcctcccaaagttctaagattatgggcatgagccactgcacctggcctgattktattttatttttaaataagccagttcatgaataattaaatgaaaacattataataataaacagacaaaaagattgctaaataataaatctagtttataactaatatatctatctcacttccttaaattagttttcaatgcccttaatttgtatgtgtgttaacatctctcaattttcaaaaatttttagttttttgaaaa

SEQ ID NO: 30 - DNA, 401 bp, Homo sapiens
ttcatgccaattttacattttgggattcattagataataacctccattccttcttattaaacagaaataaaagcatccaatatacatgcttttctagatgtggaaggctacatttgacaatcaattattaaagtaaaattaaacctggctttgggaggccaaggcaggggcatctcttgagcctagaagctcaagatcatyctgggcaacatagtgagggatccagtccctaaaaaaatatataaaaaaggcgaggtgtggtggcatgtacctgtagtcccagctactcgggaggctgaggtgggagcgaggcttaggtccaggacttagagcctgcagtgagccatgatggcaccactgcactctggcttgagcaatagagtaagagtccatctgaaaaa

SEQ ID NO: 31 - DNA, 401 bp, Homo sapiens
aacctccattccttcttattaaacagaaataaaagcatccaatatacatgcttttctagatgtggaaggctacatttgacaatcaattattaaagtaaaattaaacctggctttgggaggccaaggcaggggcatctcttgagcctagaagctcaagatcatcctgggcaacatagtgagggatccagtccctaaaaaaawatataaaaaaggcgaggtgtggtggcatgtacctgtagtcccagctactcgggaggctgaggtgggagcgaggcttaggtccaggacttagagcctgcagtgagccatgatggcaccactgcactctggcttgagcaatagagtaagagtccatctgaaaaaactaaacaaaacaaaataaacctggtgttgtctgacaa

SEQ ID NO: 32 - DNA, 401 bp, Homo sapiens
ggaggccaaggcgggtggatcacctgaggtcaggagttcaagaccagcctggccaacatagtgaaaccccgtctctactaaaaacacaaaaaattagctgggtgtggtgggggggtgcccgaaatcccagctactcaggaagctgaagcaggagaatcatttaaacccaggaggcggaggttgcagtgagccaagattgcrccattgcacttcagcctgggcaacaggagggaaactctgtctcaaaaaaaaaaaaaaaaaaaaagccaagaggggattagagtggccctaatggttaaaatcatgactaaaagttgtttaaatgaccctttcaatgccattccccttttgcactgtttaatcaagaactggttgcagtggcatatttgaggttaagaaat

SEQ ID NO: 33 - DNA, 401 bp, Homo sapiens
cctcttatgatggctagacttcagacttcctgaaatagcctttctcttggcttctaagccactgcttctcaaactttaatgttcatacaaatcacccagggatcaggtgaaaatgcacattctgattctataagcctggggtgcagcctggtgttctgcatttttaaccagctggtcttcagactcttcttgagtaccagrcttctgtcttacttacattgtaatgtttcttatgctcatggtgacggtacatcctgtactctttaacatagcctaaattttattggtatttcattattttcagcaaagtcaattagctaactaaattgatttaatttttataccttttggaagctgttcatataaaagatttccctcaccagaagtccaatgtcttgact

SEQ ID NO: 34 - DNA, 401 bp, Homo sapiens
atttgcctgggtgtatatgttagtctggtttcttcaagacccaagtaactgttcaatcaaaaactcaatcaattaacaaaatcatggacaagctctggaaaacctgtaacagaggataaaggaagtctttagaacctaaattgaaggagaagtaccataagctttctccagaactttaatacctttgaattaattaattawttttttttttttttgagacagagtctggctctattgcctaggctggagtgcagtggcaccatctcggcaacctctgcctcctgggttcaagcgattctcctgtctcagcctctggagtagctgggattacatgcatgcaccaccacacccggctaatttttgtatttttattagagacggggtttcaccttgttggccag

SEQ ID NO: 35 - DNA, 401 bp, Homo sapiens
agcagtggtttgtagttcttcttgaagaggtccttcacatcccttgtaagttgtattcctaggtattttattctctttgtagcaattgtgaatgggagttcactcatgatttggctctttgtttgtctgttattggtatataggaatgcttgtgatttttgcacattgattttgtgtcctgagactgctgaagttgcttawcagcttaaagagatttgaggctgagatggtggggttttctaaatatacaatcatgttatctgcaaatagagataatttgacttcatctctttctatttgaatatgctttatgtctttctcttgcctggttattgacacttttatatgtttatcgcattatgcactgtgctcagtttagagatgaataaaccgaaactgtc

SEQ ID NO: 36 - DNA, 401 bp, Homo sapiens
aaggcaaaacagatgcaggtatcttcacatgcccggagcaggaggaagagagggagtggggaggtgctacacacttttaaacaaccacatctcagaagaactctagcattagaacagtaccaaaggggaaatctgctcccatgatccaaacacctcccaccaggccccacctccacactggggattacaaatcgacatgasatttgggcagggacacaaatccaaaccatttcactatctaagcacagagcttggttctatgactttcaagctaagtctgctcacaaggaaaatgccactatttcatttattttttaaattcagtaaacacctactgtgcacctactttgctccaggaagtgggaatgtcaaaacaagttttaaaagcccctggaggcgga

SEQ ID NO: 37 - DNA, 401 bp, Homo sapiens
atgcacttagcacagtttctggcacttcgtaattgttaattattatttatacattttgctgtggggaatccagataaggtttgagttgacgtcaatgttcagggacagaaatacttttgcttttcaggtgataaaattggttcaagagatgcattctgccctagtgactagtgctgagacagagatttttaaaaactggarggcagcatggcactgtggagtctctttgattgtagtaatttcccttaccaagtgacttaagtcaaagtcttctcagaatgctagaaaactaacatctggagaaaataaatttctcaagactctctaaaaataacatctccccaacaactacaggctggaattatatttgatagatgcttcattgtacttggcaggcatac

SEQ ID NO: 38 - DNA, 401 bp, Homo sapiens
cagggacagaaatacttttgcttttcaggtgataaaattggttcaagagatgcattctgccctagtgactagtgctgagacagagatttttaaaaactggagggcagcatggcactgtggagtctctttgattgtagtaatttcccttaccaagtgacttaagtcaaagtcttctcagaatgctagaaaactaacatctgragaaaataaatttctcaagactctctaaaaataacatctccccaacaactacaggctggaattatatttgatagatgcttcattgtacttggcaggcatactaaagtgatgttgtggtggttattacaaaatttgctgcaatcatttataaccaagtaagactccttcattaggttacgtgtctattataagtgcttatt

SEQ ID NO: 39 - DNA, 401 bp, Homo sapiens
ctcacactcaacacacccaaaatagaaacacaatcctcctcacacttgtgtggggccacatcattcccccatttgctagaactagcaatcgcatttttatgccaatgacaaattggttctgtgtctgtacagtctcctatagctacaccttccatggctctcctgccaccactaccttgatgcaggcttgtcattaccctycactgccactcttgccatgactttcctgatgggctctgaaactgcagattctccctcgccagtccatcttctgtgctgctaatgggactgcttactaaaacacctgctcaggcccaccaccttacccaaaacctacaattgttccttgctgcctacagaataaattccaaaatggtaagccttggaggcaaagccctttc

SEQ ID NO: 40 - DNA, 401 bp, Homo sapiens
cctcagtgttgctagatttttctttcaattatttttctttaggaaaactagaaatctagatgttttggttaagtttcttaatttttttttacaaccattgcctaagaaataaaagacaaatgaaaaggatctcttttgcagaattggcctgtacatgcaagccctgtgctaaaccacacactcacacatctattatatatrtgtgtgtgtgtgtgtgtgtgtgtgtgtgtgtgtgtgtgtgtttgagacagggtctcactctgtcatccaggctgaagtgcagtggtacaatcatggctcactgcagcctcaatctgctgggttcatgtgatcttcacatctcagcctcctgagtagctgagactacagatgtgtaccaccatgcccagttaatatttcaa

SEQ ID NO: 41 - DNA, 401 bp, Homo sapiens
ttgatacacaaagcacccagtatcaataatgatacactaataacctaacagaaaagccctatacataactttttttccttttcttccatgatgttgaatcagaggtgcattaaatctggctcttgtgacactattccagtttttatgtaaaaaagcaacaaaattctgctcaacaggcagaggaatgaaaaaagaaaataytaaaaatgatttttgcttaggtacatataacttctatgttgtcatctttattttccatctgtatctcattctttgttttcaggctgtaattgtcagcaactgttcttcaggtctataaagccttcaaagctctgaccactggttgttattattattattattattattattattattattattattatttgagatggagt

SEQ ID NO: 42 - DNA, 401 bp, Homo sapiens
cccagaggactcatacaccatatgtataactgttaatagtgaagtttgagattaaaagaaaaacaccataaatgaaagtcacaaactgagggtgttgaagaaaagagatgtgtttttggaggcaatgactttttcaagttcgaggatttgtttatgtgtttgttttgttattactgcctcatatgtcaatacgccacattmaaacaaatgttttagtactcaaaagagtgcttagtgaaagatgaatggtgtatgagaattcataaattcctcattaaatgcaaaaaaagcagcccaacttttgaatgacagtgtttataatactggacttgtataacagtaaatcttttattcctgtcttttgggttttaattcttagtcttggttatgaaaatgtaaac

SEQ ID NO: 43 - DNA, 401 bp, Homo sapiens
ctgtcttttgggttttaattcttagtcttggttatgaaaatgtaaactgggcaatttagaagtcctcaaaggagtagttttgttttttcccatctctgtttttctatatttatataataaaatggaaaatattaatgtgaagtctaatcaaagatacagcacaaaagtcttcctaaactgatagccataaaattcatttcwttagctacagtttaatttgtgaaacattttcgttggtatcttgggccagtcttcttagtatttgatctaatggaagtaagtggacattggatcctgcaatgaccaaaacttttaggctcacggcaagacactgcatcatgtatctaagttttgtaaatgagtgctgttgtttgcaaggtgaagtttggagaatatgcaca

SEQ ID NO: 44 - DNA, 401 bp, Homo sapiens
aggtgagatgcttacccactggcccgtgcatgcttgtagtctgcctcacatgtggttcaaggaatagagagaaccaagagagcagctcaggaacaagcagtgattttgccccagtcttaacaataaggaaagtagtgacaaagacagagagagataagaagtgagcttcttccacagcagaggagctcagctgttttgctrtctacagaaataataggcagctgtgtaacaagcatcacactccctccaaaacaacaggatccgggggatctccattttctcagtcataaaccagttttccaaaaggaagatttctcttgaaaacaaaactaatgctgaatgtttctgagagaatgacatagctctccgaaggacaatttaaaatttcctgcggagagttc

SEQ ID NO: 45 - DNA, 401 bp, Homo sapiens
tatgatttgattataatttaccaagcagttgataagaaattgttatatgtaatccaaagcaagcccttgaaaaccttgtgtctcccaaagataattagaaactaggattatccaggcgacatttgaatttctgttagtaaaatcttgaaagttgccgtactatgtctctatgcgactggactcaggctaagggctactttyagatctcattttagcactctggaccattctcatctgaataacaacagtgaacttcattttagtatgatggcaaaagtctccttttctaaactctaaatcactgaaatagtaatgggaaataaaataacattcaccatctctttgaaagaggacaacagaatttgcctgggtgtatatgttagtctggtttcttcaagacc

SEQ ID NO: 46 - DNA, 401 bp, Homo sapiens; identical to SEQ ID NO: 20

SEQ ID NO: 47 - DNA, 401 bp, Homo sapiens; identical to SEQ ID NO: 21

SEQ ID NO: 48 - DNA, 401 bp, Homo sapiens; identical to SEQ ID NO: 22

SEQ ID NO: 49 - DNA, 401 bp, Homo sapiens
cattgttatcgatacttttattattatttatttatttactatgattattattattattttttgagatggagtctcgctctgtcacctacgctggagtgcagtggcgtaattttggctcactgcaagctccgcctcccgggttcacgccattctcctgcctcagcctcctgagtagctgagtagctgggactgcaggcaccmaccaccatgcccagctaatttttttgtgtttttagtagagacagggtttcaccgtgttagccaggatggtctcgatctcctgacctcgtgatccacacgtctgggcctcccaaagtgctgggattacagtgtgagccactgctcctggtctatttttaattttttttttattgtactttaagtcctgggatacatgtgca

SEQ ID NO: 50 - DNA, 401 bp, Homo sapiens
aagatggagggcatattagtttgctagggctgccataacagtaccctagtctcggtggcttaaataacataaatttattatttcaaaattctggaagctaagagtccaaaatcaagatgtagcaggctggattcctctgaggactgtgagcaacgatcccgttccaggcctctctccttgcttgcagacggctatcttctycctacatcttctcattttcttccctctctgggtatctatgtccaaattcctccccctcgcttttttttaataaggatttcagccatactggattagggccaactgtaaaaacttcactttaacttgactatctctgtaaagaccctatctccaaataaggtcacattctgaggtatgggggattaggacgtcaatatttg

SEQ ID NO: 51 - DNA, 401 bp, Homo sapiens
ggaggccaaggcaggggcatctcttgagcctagaagctcaagatcatcctgggcaacatagtgagggatccagtccctaaaaaaatatataaaaaaggcgaggtgtggtggcatgtacctgtagtcccagctactcgggaggctgaggtgggagcgaggcttaggtccaggacttagagcctgcagtgagccatgatggcrccactgcactctggcttgagcaatagagtaagagtccatctgaaaaaactaaacaaaacaaaataaacctggtgttgtctgacaacctaacttttggaactaacaccagtccatggggagtcctgagtggtatcagaaaaatgccatttacatcagataataaatattcagccctctaccacaaatcctgcacagaataa

SEQ ID NO: 52 - DNA, 401 bp, Homo sapiens
agtggcgtaattttggctcactgcaagctccgcctcccgggttcacgccattctcctgcctcagcctcctgagtagctgagtagctgggactgcaggcacccaccaccatgcccagctaatttttttgtgtttttagtagagacagggtttcaccgtgttagccaggatggtctcgatctcctgacctcgtgatccacacrtctgggcctcccaaagtgctgggattacagtgtgagccactgctcctggtctatttttaattttttttttattgtactttaagtcctgggatacatgtgcagaatgtgcaggtttgttaacataggtatacatgtgccatggtggtttgctgcacccatcaacccgtcatctacattaggtatttctcctaatgctatcc

SEQ ID NO: 53 - DNA, 401 bp, Homo sapiens
gcaactaaatgcctataagctcttgttcaacttcaaaaagaaaatataaatatatggcatcaaatctgagctcttaccaaattcctaagggcttgatattctgattatgatatggtgattatgatttgattataatttaccaagcagttgataagaaattgttatatgtaatccaaagcaagcccttgaaaaccttgtgtytcccaaagataattagaaactaggattatccaggcgacatttgaatttctgttagtaaaatcttgaaagttgccgtactatgtctctatgcgactggactcaggctaagggctactttcagatctcattttagcactctggaccattctcatctgaataacaacagtgaacttcattttagtatgatggcaaaagtctcc

SEQ ID NO: 54 - DNA, 401 bp, Homo sapiens
tatttttagtagagacagggtttcaccatgttggccaggctggtctcgaactcctgacctaaggtgatccacccgcgttggcctcccaaagtgctgggatcacaggcgtgagccaccgcaccaggcccaacacttcttttcatcttggatgtgataatgtgctcatgaagggttgggtaaaatccacaaatccacagattrcaaagcactgtggaattgagttaagcactgtgggaaatcaaaacctaatataatacaattcctattttcaaggatcttcctgattgcaaaaacaattatatggaagcttaacaatttctacctaaataaaatgacgattttggtatttcagagagccagcatggttttctagactgagtgctcacttgccttgcaatcaa

SEQ ID NO: 55 - DNA, 401 bp, Homo sapiens
gcccacaggtgcacagtccagggagtttaatttcaaaaatatgggtgaggacttacaggagaaatcacaagccagtctgagctacaatatttatgcatctaggcagagtccctgagcaacaaatgtggggtatcaaggttaataatgagcatgatggtgagccagagctctgcaatgtgatatgagccctcagaattcctycccaaatcctgtgatgacactaagacaagtcacatttaaagggagcagactacctgattgcagtggggaagtgaagggagcaagtatttctaccaagatgtgtatctgctaccatttaataatatagcctccaatgaagaataacctatatctaaaatcagtgaattagattcctctaaatgccccctttgacttaacca

SEQ ID NO: 56 - DNA, 401 bp, Homo sapiens
agaggccaggttcagtggctcactcctataatcccaacattttgggagacggaggcaagaggactgtctagtctctacaaaaaatttaaaaattagccaggtgtggtggtgcacacctgtagtcccagctattcaggaggctgaggcaggaggacctcttgagcccagtagttcaaggctgtagtgcactatgattgcacytgtgaatagccacgatgctccatcctgcacaacttagcgagactccatctctaaaaagcaaaaaaacaagctctagtaattcctatatctttacaaaaaaagttagcaaactagtgaccatagaccaacattggcccacagatttattttatttggctgacacaagtatggaatcgttgtgttttaaatttgtgttagtt

SEQ ID NO: 57 - DNA, 401 bp, Homo sapiens
gccctcccttaacattccccatcatgaaggcatgcactcaagagtttcatgcaaacctgtctttacatttgcctcttccactttcctggaaagttggataagttacttgaaattttttagtctcatttccctggtctagagtttttcaggattggggaggttgagtcctaacactgggacagtcccaggcatctgttcagkctcattgatgacggagagggaaggagacagcatctcggccacttcatgataacataaataaatcacataactgatgaactatgtgtaacataatcttaatctggcatttattttcagatattaaggttaagtactatatcaattttttaaaagtaaaaaattatttctacctgcatagaaggcattacctgggttgattg

SEQ ID NO: 58 - DNA, 401 bp, Homo sapiens; identical to SEQ ID NO: 28

SEQ ID NO: 59 - DNA, 401 bp, Homo sapiens
gagtcaacattgtctcccagagtgatgataaatcccgagaagatgtacacctccacataacaaagatcctccctggtattgtagagatgagacgtgacatgcatcgacactgtgtcccatgatagactgacaaagaagcatggtgatacctcaattgttcacaacttctgtgggtaaaacatactttgggcaaagttggcycaggtaggggaaaattcctgactcttcattgttttagtggctgctgagaaagctcaaccatttcttttgtaaagccttctgagacccatcaagaagaggtgatcccttcctcctttgcacctttactagcgctttgacatgttcttgtttttgtatcaatttacaatctcctcccttcctagactcttatctcttcgaag

SEQ ID NO: 60 - DNA, 401 bp, Homo sapiens
tttgaatgtggcgtattgacatatgaggcagtaataacaaaacaaacacataaacaaatcctcgaacttgaaaaagtcattgcctccaaaaacacatctcttttcttcaacaccctcagtttgtgactttcatttatggtgtttttcttttaatctcaaacttcactattaacagttatacatatggtgtatgagtcctckggggcctaggtcaacatccaggccaacagattggtccacctgtcccgtgggccacaacaaaaactcagtgctttctcacaacattcagtgcttacacctacttttaaatagatgctttaatttgagttcaagctctttccacgctaaccttttagtgacatgaaaatttctgatttgatttttaaattttgtttatttta

SEQ ID NO: 61 - DNA, 401 bp, Homo sapiens
accttggaaaaatatcccagtgcatatgttatcttgatgtttaagcaagagaagatttacaaatgtttaaaatttccaaatgttttaaactttctacctaaggaatagcagataatatttttcttcagtaaggggtcacaaaatattaatttgatacaagatataacaaaaagtcaattagatttccagcgcatgtagggytatgtcttctgtggacttaaaacattcttcatcaacacttttttttattttggagacagaatttttgctcctattgcccaagctggagtgcaatggtgagatatcacctcactgcaacctccgcctcccaggttcaagtgattctcttgcctcagccccccgagtagctgagattacaggcacgcaccaccatgcttggc

SEQ ID NO: 62 - DNA, 401 bp, Homo sapiens
ccaggcatgtatatgtgcagataagaatgatgtatgtcggagaggcctgtctacactgcctagatgagatttgcccttgatttgcagccacagctgctaatggcaaaacatacagctattttcaaccactccacttagattcacattcttctccgctctggtgctttaaaaagcaccaggatgcttgcttgtctccactgygtcctggcaggccattattctacagactgattttaatttgagcttaggcatgtcatcacaagtttgtgggagagtcatctaaaatcatcagaaacatgatgactgtgttcttgaagcacacatgcaggatgtcacgcaaaagattatagatcactttgaagagtcaacattgtctcccagagtgatgataaatcccgaga

SEQ ID NO: 63 - DNA, 401 bp, Homo sapiens; identical to SEQ ID NO: 36

SEQ ID NO: 64 - DNA, 401 bp, Homo sapiens
cctaatacccaatgtgatagtattaagaagtggtgcctttgggaagtgataaagtcacaagtactccacctttgttaatagaattactgcacttataagaggctccttggagctcccttgccctttctaccctgtgagcatgcagaaagaaggtaccatctttgaagcagagagcaagtcctcaccagataccgaatctgmtggtgtctcgaccttggactccccagccttcagaacagtcagcaatacatttccattatttataaattacccagtctaaggttctttgttatagctgtctgaaatgatgaggttaataattaatgaaataaatcagtttcctccagatatactctatggtcttcagcctgtacctctcttctcatactatccccccagtt

SEQ ID NO: 65 - DNA, 401 bp, Homo sapiens
ttatagctgtctgaaatgatgaggttaataattaatgaaataaatcagtttcctccagatatactctatggtcttcagcctgtacctctcttctcatactatccccccagtttgaaccaccgttaaagggcctaccaaactgttgtattcttcctatggatacaaagcaggtgtcaacatttaggttgtcttgggccaatygaagatgaaaacctagtattcacatctacaatcaagttgtataaacatgtaaaaatgagatactatatacatatgagagtaattggctagatatcaaccaggcaagactgccctaaattgtgatatattttgagaacagaatcaactgtaagtatgatccatgcctcattgcattacagggagaacctcaaaaaattata

SEQ ID NO: 66 - DNA, 401 bp, Homo sapiens
gaggccgaggtgggcagatcatgaggtcaggagatcgagaccatcctggctaacacggtgaaaccccgtctctactaaaaatacaaaaattagccaggcgtggtggcgggcgccagtagtcccagctactcgggaggctgaggcaggagaacggcgtgaacccggaaggcggaggttagtgagcggagatcgagccactgmgctccagcctgggcgacagagcgagactccgtctcaaaaagaaaaaaaaagtaatggcaccatcctaatactagcaccgtgcccttactttgtcttcgggttatgctatccctctttgagtacagaacagaatatctctggcctttcttattgccctatcttattccttacaaatggcaatttaatttgctgtctcctga

SEQ ID NO: 67 - DNA, 401 bp, Homo sapiens
gaaatattctaaaattgatcatgataacaattgtgcaactttgtgaatatactaaaaacctttgaagtgcacattttaaatgaatgaattgtgtggtatgtgaaatattaaataaagcaaaagagagagaaataaaaattaaagcaaaagagagaaagtgatatcttcaggatactgtggaaacacatagaatatactacyagaagccaatgcaaaagtaagttattcctactttaaccttaatttatcctcctacctagagaatgttgaaatgctgctcttacagagatttgctacagatgctaagtgaaagaaaaatctcattcaaaatgagattgttttctcaaatcttcctccttcacattgggggtcatatggcttctgtctgcagactctgtcct

SEQ ID NO: 68 - DNA, 401 bp, Homo sapiens
tagcgtttacaaattagattatagttgaaaagttctttacaattattttttgctattcttcaaaaccatttgagtggctgggcacagtggctcacgcctgtaatcccagcactttgggaggcagaggtgggcggatcacccgaggtctggagttcgagaccagcctgggcaacatggtgaactccatctctactaaacatrtgaatattaacagggcgtggtatctcatggctgtagtcccagctactcgggaggctgaggcaggagaattgcttgaacctaggaggcggaagttgtagcgagctgagatcgtaccactgcactctcgcctgggtgatagagagagactctgtctcaaacaataataacaaccaccacccacaattttgttttccttcctc

SEQ ID NO: 69 - DNA, 401 bp, Homo sapiens
ttactagtaattgtcataattttattatctttccaataggtatatatcatatgtaaaaaaaaacatgtctttgaaataatctcatttctgaaagataatttaaaatgatttctaattgcttttgcaaattcaaatgttttcaataaaagttttcattaaaatacgttaataaaagatttttcttgtataacttaaaaaagragcaataattatgttgtatgtttcaactccttttgacaatggttctcataaactcttccatgtttctctaagtatagaaaataactggcttaataaattaaattttaaatgatcctaatggagcatttacataaccttaaaatgactataacagagccaaaaagtaattaaaacttcataaatgcagagtgacatcatcc

SEQ ID NO: 70 - DNA, 401 bp, Homo sapiens
ttgatttgctcctctgtctatagaaagcagatgtagtcataaagaagagaacattcatgaacttaaaaataaatactataaaaataaacaatggcaatgattcctatcagataaaacccagactccttagtttgttctgtaaaaatatactttttggccgggcacagtggctcatgcctgtaatcctagcactttgagagrccaagacgggcggatcacctgaggtcgggagttcgagaccagcctgaccaacatggtaaaactctgtctctactaaaaatacaaaattagctgggcatggtggcacatgcctgtaatctcagctacttgggaggctgaggcaggagaatcgtttgaagctgggaggtgaagattgcggtaagccgagatcgtgacactgc

SEQ ID NO: 71 - DNA, 401 bp, Homo sapiens
gattactaaagagccaaacgcttctgatacctaaatgaataaatttctactggtaaaaatttaagtcgttgtcgtatctgaatgtggaagataactatggttggagtagaaacttcctaatcataacagcagccctgacttttattaaaacattgaaaaagggaaaagacacatggaaacagctggttttgtaggatgttsaagatccaaagcaagagatactgcaattaatctgtgaggaaggaaaaatatttctatgattctcacaaatgctcacaaatcactttcactttattaattttattttgtaaatcattccctatggtcacctcaaggggtgcattgttcttctctcatttttttcaaggtgaagaaattttggctgaaagagactcactgac

SEQ ID NO: 72 - DNA, 401 bp, Homo sapiens
ctagcataaagaattgaaaacaatatgacctgaatgatactcaaaaactgatatattaaagactcatgttgagagacagatcatgagcagagccaattttgattttaagcataaaagtggatatgttgttatgaaagtttaagtttgagtatcacacttatgaaagttaagctttgaatgtcacaagatattggggtaaargaaactgaagagtcatctagtttaactcaaattcttaagtagagaagagggtatgaggcttcacaatgacaagtggtctagtttacaggtaattcttgtccctatttgtaggacttcctgggagggtcaggttaagatcccaaataacaaaaaaggaagaaatgaaatgagaaattgcttctgcgaaacacaattgtata

SEQ ID NO: 73 - DNA, 401 bp, Homo sapiens
gatggttaccgaaatgttcatgggtttccaagaggtgcaaccttaggtaaatttttttctttttctcacgtttttgttttttgtttttttttttacaatcagcacattatttttacatcagaagaacagagaaaggtatgtttttgattagctaaaaattttaacataaaattctaatattgtgtctataacatgttttahatggattagtatgaattgtgcaaattaccaatgaaggcagtggaagagggcctggggagaggagtgttttagccagagtttgcattctttttatttcctgcactctggatctattcaaatctcgtctcctgccgagggattcagtgcagaactaaaatatgatgacttacttggcaacagcaaaaaaaagagcatttact

SEQ ID NO: 74 - DNA, 401 bp, Homo sapiens
aaacaatatatccccaaatccatggggaaggcaagaacatgattggggattttaaatcctgtaattcttggattcagagtatggaaaatacaggtaatttcagtaagagatccccactgttagaatctataacaccctatggtgtctttaaaaagaagaaaatgcgtcactagtggctcaatcccatactcctttaaaatktgttaagcttgacaattcaagctgatggcaaataactcctttctcaagtaatgttttaatctgaaagaaatatattatttaaaagggaaatccccattaatgccaagatacggaaacaactacttagaatacacagcaacatgaaagagtaagataaaaggagagaaattgaacatcaaacatgttcctctaaaccagaa

SEQ ID NO: 75 - DNA, 401 bp, Homo sapiens
ctcccagtgctactcatgagaactacataaattaagccttcagttcaggatagaacccagacttgtagctgagggcatactcaggacaaagtggcctctgagtatgcttagaactcagaattcaagggcagtcagaagaaggctagacacatcttcaaaatgtgttggaatccatttgtgatagctgtcaatagctttatrtaactaatttacacagctcatgattaactagtagctaaaatgttacagaaatgccctcataaatatatttccaaaatcaatttgggagcattgattatgtctcgtttattgaaagaatgtagtgagttgtaagggtcaaaggcaagatgacattagtgagaaagtgagtaagcattcaaaaaggagggcagatatctaga

SEQ ID NO: 76 - DNA, 401 bp, Homo sapiens
ggggctccagctataaaggcaaacttgtggatcacaaataattatttaaaggagatctgtttgttcacaaaagaagtatagtaatgctataatagctcaggaacagttgagtgtctctgcactactccctgtagacagtggggatgttgtacaaaacaaaggtttataatcagactttttgcagatcaatcaacctagcaytagggtatcttctcctagctagaagatgaaagcaggaacaagccataagcatggtgaaaatttactcatttatttcaacaaaacactgtaatattttaatgagacattgaaaataagacatacaaccttaaaaataaaggcagaaaaatatttttagttgatgctcatgggttgaatttttaggtcacaatagacgagct

SEQ ID NO: 77 - DNA, 401 bp, Homo sapiens
tctgtaaagaccctatctccaaataaggtcacattctgaggtatgggggattaggacgtcaatatttgaattttgggagagatacaattaaatccataagagacagtatgtatatcagtaatttttgagttatggatggcatttattttattctttttccttatctgttatttttttgcaatggactcttggaactaatamaattttaaaaagttattttttgccaggcatggtggctcacgcctgtaatcccagcactttgggaggccgaggtgggcggatcacaaggtcaggagatcgagaccatcctggctaacacagtgaaaccccgtctctactaaaaatatttaaaaattagccaggcgtggtggcgggcgcctgtagtcccagctactcaggag

SEQ ID NO: 78 - DNA, 401 bp, Homo sapiens
tgctgccacttctctaagataatagggatcctgtaaagtctgggaaagattctgggtagtacctctgctgggcactgacataattaggcatttatgtgctgctcaaactgaatggtctcagtcagctgcagtatggcctggctggagagaaggttgccaggtcagaggagcttttaaagaggtgtctgttctactgagccmtgctggtttgtgtgatctggtatcttcttggtgctgactgctcaggactgagaccttctgcaggttgaagcatttgaagaacatagtccatttcacctatgtattctggttggtttggctctcctagtctgcagtgtttagatgccttgtcatcacttgcctgaagacctgactgaaatttggaatcgattgccagaatt

SEQ ID NO: 79 - DNA, 401 bp, Homo sapiens
tcatggaccagattactcattttaattcctgagatctttactggggtggtcagagtgaaaaagtacggcatgaaccactgtacgcagaaaatcatagaaattgttaaacatccttgatcatccttcctttccatgatacttgtgtttctagaaattgcacctcaaaacttagtatgttttattggcattagtgcagagatygtatattcatattatgtttagaactgtcctgatgtttagaattgacgaaatgagctcatgtgtatcaagaagatgtcaacatttatgattctgtttggttcagaatctgtagaatcacctcttgggacacttaatggaattatagtgcattcaaaagtttggaaaactgttgtcaatttgccgtgctctcatatcaaatg

SEQ ID NO: 80 - DNA, 401 bp, Homo sapiens
atttatgattctgtttggttcagaatctgtagaatcacctcttgggacacttaatggaattatagtgcattcaaaagtttggaaaactgttgtcaatttgccgtgctctcatatcaaatgtttagcctgcaaagactcaaaagtcagatggagtcagatgatcccaacctctataaagataatcctggtataggtgacgaycctcaaaagaggatctcataatggtttggcagacatggaaaacaaacagatttttccgctctttttcaaaaggggtaccatggggcctaatgagttttcaatacagaatatgtctctatataatcttgagtacctagaaagtgaagatacttgctgaacaggcacttgtatatgggcaaaagaacctgcacagagttgtc

SEQ ID NO: 81 - DNA, 401 bp, Homo sapiens
ccttatttacaagaaatttttgaagacatggcatttgtgacccagctgagaattctcctggtttatgtagtgagggagggaagagaggagagatgctatttgctcggaagtaattagcaatctgcaaccactcatcagtgaagaaatgaaactaaagccacctgtcaaaattagaactataaaaatacattaactaccacraagtattataaacaaatagccagtttaaaataaagtagttgctatattgaaaaatcaggattgtctattattgttcccaagcatatttaaataggtattacacaagtattcccttgattttctcattatcttcaccctgaaattgattctgtgtctataggtggctactggttcaattacttttagtcaaatctcttttt

SEQ ID NO: 82 - DNA, 401 bp, Homo sapiens
agtgagtaaattaaatctcttttctttataaattactcagtctcaggtattcctttattgcagcatgagaacagactaatacaacaactgtatacaaattttgggaaaaattaaaaaaaaacaaaatttacttgtaatcataaaataaccattaatattttactttgttttttatcctaacctttttctaacacataaacrtattcacacacatgtaaatacaaacaaacgtctcctatctatcgcatttctctatgcatgtttatctgtttgcatacatggatatgtatgtgcatgtatatgcattgtattacgaatatagtttattatttttcttttactcctttaaaaaaatttcaatacagtgtcatgggcgtgtccctatatttattaagtagtct

SEQ ID NO: 83 - DNA, 401 bp, Homo sapiens
gttggcaacagtggtaaaactggcactttgctcactacgagtgggattgcaaattgggacaatttgccaataagtgtcatgaatcttaaaagcacaaaactttctgaccctgtgattccttattctaaagaatcagaaatgtgaaggagttgttcacaacagtactattcataatacagacttagaacagcttaaatgtcygacaatattgaaatgaacaaattattgtcattactgcaatacaatggaacattctacaattgttacaaataatagaatactttgcagccattaataattatagtattgggctgggtgtggtggctcatgcctataatcccagtgctttgggaggaaaatgtaggaggatcacttgagaccaggagcttgagagcagcctg

SEQ ID NO: 84 - DNA, 401 bp, Homo sapiens
ttatttcatacgtgcaaagatctcaacattaagcatgtattagcaatgcaatgcaatatgtaggaattgagtaacaaactcaagattatcatgagcatttgtaaatttgtaaatttgactaaatcttctggaggtgtttaaatgaacaggaaattttcctaggctacactacaaggaaaagtcaaaaaaacaatatttttsagatatggttaaaaagaaacccactaggatccaatatgaatagcatataatatgaagtatttagatagagatgagcaatatcaatactacttagtgagaaaggcttagctggcaccaccgtcatgatatcatggttgttaagcatcctcacagtacaggagtacagattataggaagccacagaatagggtcattggtgc

SEQ ID NO: 85 - DNA, 401 bp, Homo sapiens
atccaggccaacagattggtccacctgtcccgtgggccacaacaaaaactcagtgctttctcacaacattcagtgcttacacctacttttaaatagatgctttaatttgagttcaagctctttccacgctaaccttttagtgacatgaaaatttctgatttgatttttaaattttgtttattttatagttactctttagargcatgtgattcattatttcttattgattacattttaaaataccataacaggctgggcgcggtggctcatgcctgtaatcccagcgctttgggaggccgaggtgggcggatcacgaggtcaggagatcgagaccatcctggctaacacggtgaaaccccgtctctactaaaaatacaaaaaattaaccaggcgtggtggtg

SEQ ID NO: 86 - DNA, 401 bp, Homo sapiens
gtgcaagtttagatttggttttgacagtcaagtccaaagctaatcagttatctcaatattagaccaagcatgcagatataaccggtatggaaaaaaattaactacagtcatgattccaagcagcttactttgatgacacataagtgggcaatattaaaaaaaaaatcacttcaaaggaaattttcctatattaattatcawttgtaaaggtgagaaaattttaattctgtctggtgtttagaaaaatgttaagaaatttaaaaagcatgacagcaatcgtttttgcacacgactctaccactttcttttttcttattgttagtttatttatttatttacttctcttattgtttggggattgtcaaaattacagtggttacaccaccgccgcttctgacaga

SEQ ID NO: 87 - DNA, 401 bp, Homo sapiens
aacaacttaaaggagagcaaaagggacagatgggtggtttatatttgaaaatagattaatttgcttgaatagtatgtgccttcaaattggagtgatgaggtttgcatataatcgaaagcagagaataaaacagaggaatatagtctattcctttctccttaatttgctaccacagtgggtcagtacatggcaatactttcraatgtaatctttgtcacattcagaagaaaaatgccaaggaacaatgaaacataaagggatattttttattccaggaggaaaaagaattttctggaaaatcttaattggcaaatttttatttttattattattttttgttgatagagacggggtttcaccatcttgcgcaggctggtcttgaactcctggtctaaagtaat

SEQ ID NO: 88 - DNA, 401 bp, Homo sapiens
aacaaatcaagaaaaacagcatgagtgtactgggttttctttaaatcagcagacatatagcataacgggctgtgagaattctgatccaagtcacaggtctgccacccaatgcaaggtgtgagttccacgttacgtgaagtgaaagggggaaaccaccaaggcgattttagaataaaatcagccctatccgatttaacccarcttatcaatacagtttttcctacaggatattatactcatttgaacctctgggcctctgcctatgttttcctgttcctagtttgctgtctcagttcctttttgccaatcagaatcctataaatccttcaaggtataatctagagcttattcttctttgaaacttgccctcacgctgatacatttcttgactgatttcctgt

SEQ ID NO: 89 - DNA, 401 bp, Homo sapiens
tcgaactcctgaccttgagcgatctgcctgccttggtcatccaaagtgctgggcttacaggtgtgagccaccgcacccagcctcttaaaagttttatatatccttggaaccaataagcccacttctagggatctctgctaaaatattcactagagatgtagacaaaaagttacatacaggattattctttgctgcattatktatgagatagaaaaatggaaatgatctgaagaggaatttaataaattaataaatttctttgcaggcattaaaattaaatattgagggaatatttaacaacatcagaaaatactcatgttgtaataaaaaatgggaatgcagaagacccgcaacagttcttaccctgtttaaatcagactgtcgaatcctggcttttttct

SEQ ID NO: 90 - DNA, 401 bp, Homo sapiens
tagcttcatctgtactacagagactttgggaaaatttcttgaagacccttttttaatgggaataaccacttccattattttatctctgggtattctgtgagtatctgttgtctcaataagtatttctatatcgtgtatgttatgtatattaaaaaaaacccttgagttcttcagatgaaatctataatgcaaataaatgayattcaaaatttagctattctaaagatattaacttctaaggtaatggtaactttaactaaggttaaacgtaacttctaagcagtgactaccatttttctatttttttcagaataaattcatctcaaaatctacattacaataaattatctcaataatccattttcctatttttgattcaattacataattaaaaaacattt

SEQ ID NO: 91 - DNA, 401 bp, Homo sapiens
agagaaaaaatattgtaataaaccctatgtcttctcaggttttaaaaaatcaatatgaattgttagtattaaaaatgtttggaatgcgtactttgaaataaaatttttgaaaaaagtattgacatgtaatgtttaaaagtcaaactttctgtggaagtaacattaacatccataatcaatcattaatactatgatagtgarccttatttttatttaacaacatttaatcgttgttgtctgccaccaaattctgcattttagctcttttcactaaacattactatgtttttaataaaagcattatagtttggttactagtaattgtcataattttattatctttccaataggtatatatcatatgtaaaaaaaaacatgtctttgaaataatctcatttctg

SEQ ID NO: 92 - DNA, 401 bp, Homo sapiens
aagtaggaaaaagatcaagggaggaagggagggaggaaggaagcggggaagcaggaagaaagggagggaggaaaggtggaaggagagtggaagagaaccacataactcaataaaaggcaatatgtgtactgtctatgtttttgtcttactaaatctcatactgttgatgacaaaccatctttagccatatacacaatctcytagcgggctccagctcaatgacttcaaagtcagcatactcctaaaccagctcctcacccctcctgctccctccttgcccttctctattgtctattgcaagcatcattttccagatctagtaacccaaatacgtaggtgcacttcctgacacccatcttcgcttcaaacaatactttctataggttcaaagtgatgatggc

SEQ ID NO: 93 - DNA, 401 bp, Homo sapiens
ctgtctcttgtacaacaaagcccatgcaatgtgtgcaataaagcccatcaaggcacaaagtggctctccatttgatttgctcctctgtctatagaaagcagatgtagtcataaagaagagaacattcatgaacttaaaaataaatactataaaaataaacaatggcaatgattcctatcagataaaacccagactccttastttgttctgtaaaaatatactttttggccgggcacagtggctcatgcctgtaatcctagcactttgagaggccaagacgggcggatcacctgaggtcgggagttcgagaccagcctgaccaacatggtaaaactctgtctctactaaaaatacaaaattagctgggcatggtggcacatgcctgtaatctcagctacttg

SEQ ID NO: 94 - DNA, 401 bp, Homo sapiens
tcctgttcccactgtagggagggagacacaggagttggctttgcccaaaaatagcctgtgtccatgctgcggaattagcttagtatcaccaagtccaaaacttggagccagaagtcctgctttcagaaaggacttccagcccttgaacattgctgctactcaagtttttaattagttgtgaggtcactggtagccagcacmctgggacagtaccatgatgaacagggtgggtgcctcaggaggaagccaaaaggatgaagacactacctttatgagaatattttctttctaccagtcctgaagggagtctaagtaacaagatcttggatttctactggtaaagggtcatttgccctaaatccctatagtttaccataatttgttctgtcatttccaccttc

SEQ ID NO: 95 - DNA, 401 bp, Homo sapiens
cctcaccagaagccgatgctggtgccatgcttcttgtacaacctgcagaactgtgaactaaataaacctcttttcttgataaatcacccaatctcaggtaatcctttatagcaatacaaaactgactaatacaggtggcaacaaatgacttttgcaatgactaaaagatgatgtaagtgtatatagtgtatatatgttgcrgcccctttctataagattcagatccactgaatgtttcttgggttccctaaatcctctattcaatacctgttggttaggccaggcactattctaactactgaaagtggagtagtggttatgaaagacaaggttcttgttctcatgaagctgacctgatagagaagaagacagtcaagaagcaaataaactggaaatatgac

SEQ ID NO: 96 - DNA, 401 bp, Homo sapiens
aaacggacattgagacaagagaagttacatgtctttcttaggaagatgaacttaaggccaaaacctcaccgtgcagtgtccccatccatttctcttgtgcattactacattttaggtgtgtgttatttttcctaattgcagctgcctcgtgtgggcatagggtgttacaatggtctggatgttccccaggccaagcgtgcrttgttttgcttgcacagaagagtatatggtaaataatacatgttgattgatgggctgattcagtccattgccctttcctgcccccctcccccagaacagatatggtaaaatagaaacttctgatttacaggatgtccgaaaggtatgcaatattttttatcaattgcaaaatactctaaaggctatcctgtcatttgata

SEQ ID NO: 97 - DNA, 401 bp, Homo sapiens
cactccagcctgtgcaacagagcgagaccctgtctcaaaaaaaaaaaaaaaaaaccccaaatgtgataaccagtaactagacattcatgcaagcatcagtactgtgaaccagcaactagatgacattaaaagagtcatgttactcttttcatccagagcaattcgtcttagataatcaaaggcagttaaccttaatttgtrccaaagataaatgcaaaattatcaccaagaagaagttgtgggtggaaattagaaggttttgcagttgattgattatatcgagcaataattcgttgatctgacactgaactctatctaaatattttaatattaggccatatttaccactattctccattcctgttttttttaaatagtaatactataaataagaaatcaaa

SEQ ID NO: 98 - DNA, 401 bp, Homo sapiens
tatcaccaagaagaagttgtgggtggaaattagaaggttttgcagttgattgattatatcgagcaataattcgttgatctgacactgaactctatctaaatattttaatattaggccatatttaccactattctccattcctgttttttttaaatagtaatactataaataagaaatcaaaagtgattagaggaaaggacwtttgaccagaaattgcaggagagattggtgtgatggtccagagaaagctaatcaacagcaactttcttaaagagtctttctgcccctctttatcacctccctactttcagtgtggcaatgcctctgtccttgctattttccttttcttcacttcttttcttcaatttttcactctgggaattaatgactatttcttccaa

SEQ ID NO: 99 - DNA, 401 bp, Homo sapiens
atttatacattttcactcattggtcaggaattaagaatcaaatggggtaatcatgtttgtttcatcgaattcatcttattcgaaaaaagaaattcaacaaaccctgcatttctcatctgtaagcttagattattatagtagatatgacatagttttattcttgtatcttatagcaattagctatgtaagctacagtcacckttgcttgcactttgttaaacctacctggagcaggtggcaagattctgggatcgagaggcaggtggaggagttggaagggtcaattcccgtgttctgcctggggtaactgagactgaatttggaggccatgtctgcagccctggctgtcctgagtgtgctgccaaagcatccgactgcattgctttcagggcaaaggtaat

SEQ ID NO: 100 - DNA, 401 bp, Homo sapiens
catcttgctgtaaaatgaggtggtccctggaaagcccagcacaggaaagctagatatatagagtctgtggatcatccagtgatgacgctgaacataaaagctggagatgtgatgtttccaaataatttatatttgttttaaatttccctccttttcttataaataacccaaatccagatactaggataaatgactggatcrcatccccttgtcacggcaggcttgcaaaaatatattaggaacgagggagggcaatgtgcacagagaagacagacctgctcctgtaccttgtcatttgtactccagtgatgaatttctaaactacgtatttgagcatgttaatgaaaaagaaaagagcaaaggaaagaaaagagaaggggagagagaaaatgacaaaagaa

SEQ ID NO: 101 - DNA, 401 bp, Homo sapiens
ataagccagggactgcttatttaaggcgattattacaaacaactgacagattattttcccagattcttgcacttattcttcatatgcaagtttaaacgagtcccaaactataatttcttgaggatttgtgtgtgtacaaaagtaatcgagataaggatgtgtgaaagactaggtaaaaaaatctatttctaaggaataacyatgctccaagcagacatagaaaaccaaggtcctttgaagtaaggactcgtgagggcaatttcaagacaggtcaaagaattctcgtctctaatgatcaaagatcaaataaataccagatttaccctttaaaatcaagcgaaccctttccttgtttaacagtagatcagtgttaattcatttctgaatagagtctccctctg

SEQ ID NO: 102 - DNA, 401 bp, Homo sapiens
acttgtccaaagccatagaacctataatgaacaaaagtgggtttgaacctatttctccaactgagcttcatgtagacaagcagatgttggaaaaagagtcagaaaagaagagaaaacaaattattagaaaggaaggaagacaatagagattttattgtatcctgaatggcaagatagaagggattctctgggtgatggtasgtgtgatagaagcatgggggccagaaactaaaaagtgaggcctagacgggtggtgaagtagaggaagcatggtggaagctctgaatgtgaactcctaattcttttaatacatggcaatagggagaaggggtcgaacaagatgcattattaaggtcatcttttatgggctccctgttctttggacccatctacaaaaaaga

SEQ ID NO: 103 - DNA, 401 bp, Homo sapiens
ttcttttaatacatggcaatagggagaaggggtcgaacaagatgcattattaaggtcatcttttatgggctccctgttctttggacccatctacaaaaaagactgtacttttctcatgctatgtatatgttcttatattcttggaggatggcagctatcaccttacttaagacacaccctaacctagatctcttgctacartctctctcttcagatattgatttgtgtttccaggtgcccatgcagtgtgtccaactggtcaattcagagacaccaatatctgctcctgcaaacctgttccttccctacatcccctacctcagaccatgtcatcctaccactttagtctcccatgttatcaaccctgacattatctttgatgcctttcttacccccactct

SEQ ID NO: 104 - DNA, 401 bp, Homo sapiens
tgatttgtgtttccaggtgcccatgcagtgtgtccaactggtcaattcagagacaccaatatctgctcctgcaaacctgttccttccctacatcccctacctcagaccatgtcatcctaccactttagtctcccatgttatcaaccctgacattatctttgatgcctttcttacccccactctgtaaattcaacacatctbcagtcttcatagttttaaccctgaaatgtctccaaatacatcacctcctcttaatgcccctgctcatactatgtatttttataccatagtcttatgtcatcgcatatgtgtaagagacatactttcatatgagactttgggtcaagtcttcattatctcttgtagtgataaaagcatggttgatctaatctctgtccttc

SEQ ID NO: 105 - DNA, 401 bp, Homo sapiens
tttaaccctgaaatgtctccaaatacatcacctcctcttaatgcccctgctcatactatgtatttttataccatagtcttatgtcatcgcatatgtgtaagagacatactttcatatgagactttgggtcaagtcttcattatctcttgtagtgataaaagcatggttgatctaatctctgtccttcacagttatactccrctctgtctttccattactactgttactagaaatgatttgtaaagtgtaaagcaagtggagtcaaatctggcaatgcctctctcttgctttgaaaacattaataaaggcagaggccaggttcagtggctcactcctataatcccaacattttgggagacggaggcaagaggactgtctagtctctacaaaaaatttaaaaa

SEQ ID NO: 106 - DNA, 401 bp, Homo sapiens
tgtcatcgcatatgtgtaagagacatactttcatatgagactttgggtcaagtcttcattatctcttgtagtgataaaagcatggttgatctaatctctgtccttcacagttatactccgctctgtctttccattactactgttactagaaatgatttgtaaagtgtaaagcaagtggagtcaaatctggcaatgcctctytcttgctttgaaaacattaataaaggcagaggccaggttcagtggctcactcctataatcccaacattttgggagacggaggcaagaggactgtctagtctctacaaaaaatttaaaaattagccaggtgtggtggtgcacacctgtagtcccagctattcaggaggctgaggcaggaggacctcttgagcccagtagtt

SEQ ID NO: 107 - DNA, 401 bp, Homo sapiens
tcctaatcataacagcagccctgacttttattaaaacattgaaaaagggaaaagacacatggaaacagctggttttgtaggatgttgaagatccaaagcaagagatactgcaattaatctgtgaggaaggaaaaatatttctatgattctcacaaatgctcacaaatcactttcactttattaattttattttgtaaatcwttccctatggtcacctcaaggggtgcattgttcttctctcatttttttcaaggtgaagaaattttggctgaaagagactcactgactttccagaggttatctagggattcattaacagaataaaaattaggctccagctaatgtcactaagccattgctatcctctgcatcttcttgcttccctcaatccaaagaggttc

SEQ ID NO: 108 - DNA, 401 bp, Homo sapiens
gcacttcgtttagtattggtatgtataaccacagcggctgtaatagcacattgcctcagatttagttgagagctgaagaaattctctgcagcctcagaacaagaatttattaaatcttttttagaggggtattaccagacacaaatgtcaagtcaaggaacaaagggactaatccaaattcacaaagaatttgactggctrtgtctcctgaacccgtgacttcctccagcacttcagatgtggcataaagcaggagggatttcctaacgatgcagatgcactagaagccagagggtcagggagacctcttctctcactggtgcttggaagatttccataagtaactcccagtgctactcatgagaactacataaattaagccttcagttcaggatagaacc

SEQ ID NO: 109 - DNA, 401 bp, Homo sapiens
ctacatttgatatctgctcctggctgttcctctaactacacagcctcccaacacaatcagatgatggtttaagagaaacagtctttgacggactaagcccatgccttgcccaagtccacccatatcactgtggtttcctcattcatgtgaatattataggttaaagtcagggttcagggtctagaagcaggacatgaagtrtaaaaagagacatcagaattactggaacactaacatatgaatgagtatgcgcaaatgctattacatggtgagcaaacataagataattcatttcatgaaataagcttagttgccaaaaccatgtttagtcggaatctttttttttttttgcagccatgtgatttttcaaattatcttctgtgtgagtatcaataattgta

SEQ ID NO: 110 - DNA, 401 bp, Homo sapiens
ctgtatttcatcttaacttctgtttctgatgaccaggtaaagacgacataataaaagaactagggactacattgacaatagggcatgaagaaaattcttatactctagccttgctcattttgttgcaataaaaacaaacaaactgaacaatgtgatatgtttctggaataggtggaggaaagacaatggcttttttcagasagaggtgttgcatttatttgagttaatcctgaagttctctgaaatttggcaatggtagttttatgtttcaattcagtgactgctgtttaaactgcaggattgatgcacaaatgatccataacacattttggcatattaaatcatcttcacagttctaatacatcatgaaacccaatttacatataacgtaggcacagaag

SEQ ID NO: 111 - DNA, 401 bp, Homo sapiens
agtttcatgaagtaaataaatcctgtctatgcctcattttaaaaataataagcatgattcttttataatccatgtattttggttagaatgattttaccctacagaataataacacctatttcatgggcagagtgtgagaaatctttcagctgatagatatgaagcatgtgaaacggcacctagttcccagaagtgtacaayaaacagtagctattatcactaacactctagatggtaaataatccccttcttacaaaagaggagcttggatttgagagcattttagtgacttccacaaggtacaaattaaatcaccagattgagagtgctgccaggtcatactgaaattatctgaactgtttgcctactcctaaatctatttccagactcataaattcaaa

SEQ ID NO: 112 - DNA, 401 bp, Homo sapiens
tgcagatctggatctgtgcctttttctctcatgcctaagcacttagcctaagacttagtgtgttcagactgaagctttgcctggcttttcagggctttttccagggatgaggattttgtccttgattgtgattgggtcttggtcagacttctcagatgctaaactccatccagcactcccctgtgccatgcctgtaacatrattggcctttggcgatgccgtatacttagctgaatgctactgctgcctgtccccaccctcctccctgcctatctgtgtctgtgaagatacagcattccaggggtctggttggacttcctcctgccaccgttgtgctgctttcttcctcccaacttcctcactcccacatcacagaggttccagatcatcccagtgacacc

SEQ ID NO: 113 - DNA, 401 bp, Homo sapiens
ccccactgttagaatctataacaccctatggtgtctttaaaaagaagaaaatgcgtcactagtggctcaatcccatactcctttaaaatgtgttaagcttgacaattcaagctgatggcaaataactcctttctcaagtaatgttttaatctgaaagaaatatattatttaaaagggaaatccccattaatgccaagatayggaaacaactacttagaatacacagcaacatgaaagagtaagataaaaggagagaaattgaacatcaaacatgttcctctaaaccagaaacagaagaaagtgttctttagaagattagacaggtcttgaatttttattgttggcaaagaacagtgtttaaaggcaagagctcaacctttctacagggcagtgtagttttt

SEQ ID NO: 114 - DNA, 401 bp, Homo sapiens
aactacttagaatacacagcaacatgaaagagtaagataaaaggagagaaattgaacatcaaacatgttcctctaaaccagaaacagaagaaagtgttctttagaagattagacaggtcttgaatttttattgttggcaaagaacagtgtttaaaggcaagagctcaacctttctacagggcagtgtagtttttaaattcrtggagcaagatggtgacactggctgtatgtactagcataaagaattgaaaacaatatgacctgaatgatactcaaaaactgatatattaaagactcatgttgagagacagatcatgagcagagccaattttgattttaagcataaaagtggatatgttgttatgaaagtttaagtttgagtatcacacttatgaaagtta

SEQ ID NO: 115 - DNA, 401 bp, Homo sapiens
ttcctctaaaccagaaacagaagaaagtgttctttagaagattagacaggtcttgaatttttattgttggcaaagaacagtgtttaaaggcaagagctcaacctttctacagggcagtgtagtttttaaattcgtggagcaagatggtgacactggctgtatgtactagcataaagaattgaaaacaatatgacctgaatsatactcaaaaactgatatattaaagactcatgttgagagacagatcatgagcagagccaattttgattttaagcataaaagtggatatgttgttatgaaagtttaagtttgagtatcacacttatgaaagttaagctttgaatgtcacaagatattggggtaaaggaaactgaagagtcatctagtttaactcaaattct

SEQ ID NO: 116 - DNA, 401 bp, Homo sapiens
gaaccagaaagcctgttgaatcttagtaaaagcactaatactatttccatttttatttctcttccaattttgtataaaaggtacattttcattaggggaattgggtagctaattctcttggaaagattcagctgtcactgagtaacacaatctttccccgccctcctcgcccaggctggcagggactgtgcttcagctaayaacagtggctctgctttgctgggatgcttcgagctgctgtgacagctgacaggagagtagtaaccgctaaggtgtcaagccatcctttccccaattccatttgctttgggaatgtgctaagttgaaagtccataagagccatttaagaacaagtattagtagatggtattataggtttctgttcctctacacagtccatt

SEQ ID NO: 117 - DNA, 401 bp, Homo sapiens
ctaaggtgatccacccgcgttggcctcccaaagtgctgggatcacaggcgtgagccaccgcaccaggcccaacacttcttttcatcttggatgtgataatgtgctcatgaagggttgggtaaaatccacaaatccacagattgcaaagcactgtggaattgagttaagcactgtgggaaatcaaaacctaatataatacarttcctattttcaaggatcttcctgattgcaaaaacaattatatggaagcttaacaatttctacctaaataaaatgacgattttggtatttcagagagccagcatggttttctagactgagtgctcacttgccttgcaatcaaatgcttcaattaacatctacacactgacacttactcactttcctcattcataagaagg

SEQ ID NO: 118 - DNA, 401 bp, Homo sapiens
gttttccaaataggaaacaggaggaggttaatttaaaataaaccactatgaggtttcttacagagttatcatcacctgcctttcaaaataagcatactcattacatgtgagggcaattgttcatctgaacatcttgtttacttgtctgattgttctccccaggtataatgattttcaacaggtcaatcagtggttctaagygcttttcaagcctatttaacaggataatttggtcaaagagcacatgttatataaattgccctcccttaacattccccatcatgaaggcatgcactcaagagtttcatgcaaacctgtctttacatttgcctcttccactttcctggaaagttggataagttacttgaaattttttagtctcatttccctggtctagagtt

SEQ ID NO: 119 - DNA, 401 bp, Homo sapiens
aaaccactatgaggtttcttacagagttatcatcacctgcctttcaaaataagcatactcattacatgtgagggcaattgttcatctgaacatcttgtttacttgtctgattgttctccccaggtataatgattttcaacaggtcaatcagtggttctaagcgcttttcaagcctatttaacaggataatttggtcaaagwgcacatgttatataaattgccctcccttaacattccccatcatgaaggcatgcactcaagagtttcatgcaaacctgtctttacatttgcctcttccactttcctggaaagttggataagttacttgaaattttttagtctcatttccctggtctagagtttttcaggattggggaggttgagtcctaacactgggacag

SEQ ID NO: 120 - DNA, 401 bp, Homo sapiens
ctctggcttgagcaatagagtaagagtccatctgaaaaaactaaacaaaacaaaataaacctggtgttgtctgacaacctaacttttggaactaacaccagtccatggggagtcctgagtggtatcagaaaaatgccatttacatcagataataaatattcagccctctaccacaaatcctgcacagaataactcaataamtattagcagcaatgacaattaacaactgctttcagttcagcagcttatggatccttgatattcttagcttcctaatcgttagtaattccatcaccttttaatgaatgagtcagaaatttaaacagcaaaagggagacaaggcaaaactaaacatctgtgaattttattaattattagcaattccttataaaagccaagag

SEQ ID NO: 121 - DNA, 401 bp, Homo sapiens
catagtaaaagaattaagcagaatatgggaactttgatattggtgtcttaatggggaaacagctttcaatcaagtagccctgtctgtattctatgttgaaaaggagtttttaaaccaaaactacatggattatgctcaatggactctatcgataatttacaactcttctatttactatgttccttttaatcataagctaartttcttaatggaaagaaaatattaaatggtaacaatatggagatatttgtctttggtagaacctctctttacagcctatgaaaggggaaaatcggatactaccaacaccaactttaatctgatatatttctagtcactttggtttctgaaatcagtctgtgcagaaatctttttcatacaatgtcacgtatttaatcttg

SEQ ID NO: 122 - DNA, 401 bp, Homo sapiens
cttctatttactatgttccttttaatcataagctaaatttcttaatggaaagaaaatattaaatggtaacaatatggagatatttgtctttggtagaacctctctttacagcctatgaaaggggaaaatcggatactaccaacaccaactttaatctgatatatttctagtcactttggtttctgaaatcagtctgtgcasaaatctttttcatacaatgtcacgtatttaatcttggtagtaaaacagcattactagttagaaggaatacataaagtgagtttaattattcttttactttccatgaattaataatagaagcattatttttctcattgcatttgtaatgagtgaagactgacaaccctttctgaacacaaattccagtaactcagaattat

SEQ ID NO: 123 - DNA, 401 bp, Homo sapiens
aaaaaaaagagaagagctgctctttcaaaactactgccactgttgaagaaattcaagattgtcataaatgctcgtgtaggttttaaggaaaaagttatctgagaaatataataaatcatctgggtttttcatttgttttaccaaacagttataattttcctcttgttacagtgaactttttattgcccagaatccaattgrtaaaagctctttattaaccaaaacttatttgtactacctcccctgacactatcgtaaaaatgttatagcacaatatttaatagcaaccctaaaatgaaaatgatgacttagaagattctggattattttctgaaacatcaagaaatcaatttactcatagataatctgcctcagttagagtaagtctcagttgactaaga

SEQ ID NO: 124 - DNA, 401 bp, Homo sapiens
tgcctcagttagagtaagtctcagttgactaagatttgagaaaacagaatacccaaatctcaatcgtgcaaacagaatttactctgtatatgactgatacctaaagagtgggaggaacgttcacagaattttagaaaaggtatctaaaaccaaatgagaagataacaatataattaacagtctttgcgttgtatttattgrtttagataattataaattgttccattgcctaaatgaaattaggagacttaaaagctaaaacttagagaagagatccagagaatgaatccaatcccctcatactgcagatgggaaaccaaagcccagaagttaagtgatattgggtttggaccaatatccaagtccaagtttccattcaatgtattttccataacagaacg

SEQ ID NO: 125 - DNA, 401 bp, Homo sapiens
aaccaggagtccacaagtgcagtatttcaactataacatagcatgaagaaagggcaggctgaaagaaaaaaagcccgtggggacgagttttctttacatgtcctacaatagtttttctttcataaaagtaatgaaagaaaaggtggggtggagtggcagtgaggagaagaatgggtgagaagataagacacgcatgccttrtgctgaagaaaagactatttgtacctagtacattgtagaaattgttcaagaacagctcagccatgtaaaaatatgaccctgtaatactgccaaggtcaatcagacccatagtgattgacttagaaagagctggccttacaggataccattgcacaaagcctacgagagtttatacgcctaattgctagcatttggaaggt

SEQ ID NO: 126 - DNA, 401 bp, Homo sapiens
tggccttacaggataccattgcacaaagcctacgagagtttatacgcctaattgctagcatttggaaggtatttttgaatcttgagaatttaattgtacaaaactacagtataaaactcatactttagtttttttcttggtggataagacttaaaagagataccataaaaatgtaaagtagatacacagtcactctttcaygttatgatattttcattattttattagcatttattcctgatatttttgttttaatatttctttggcgtggctttattttccaaatagaatttaagctgcataagaacagatatcatgctttttagtcaccgttttttgaataaactaaattttgactcataaataataatttgtagagagttgcctattaaatgtcaaat

SEQ ID NO: 127 - DNA, 401 bp, Homo sapiens
agctgggtgtagtggtttgtgcctatagtcccaactacttgggaggctgaggcaggaggatagcttcagcctgggtggttgagactgcagtgagctatgattgatcacaacactgcactccagcctgggcagcagagcaagaccctatgttccaaaaaaaaaaaaaaaaaaagtggattttttagttcaattaactaaccragaattccaagtctaagcgttcttgtcagggctcactctttctatgtcttagatccacatgactttgtttctttttaagaattgactgaagcttctcaccaggtgcattggcaggaaaggttgtcaaagctcataccctacagaacatgatccaaaagaaagagagaacagttttactcccagttccaatatatggaatt

SEQ ID NO: 128 - DNA, 401 bp, Homo sapiens
atttctaaggttttatttttttcattggttttcatctgacacataggctggaaaagatgagactatggtaagaggcaggtaaaagtcattgtactagacaacttgaaaaggatatctaaaagataataaaaagacaactggttatttttatttgttttaatttccctaaaaaagatatcaactccaattaccaatctttgyttttaaattttaaaaatcacttattttaaggaatatttaagaaggaacttgtagtgtccagtgaagcaacttcaaaaagaaattagaaaaagagacttgagataactagcaaaagcaatgagtagatcttgttttgaccctagcttgaacaaaacaattataaaatacatgttctgtacaagggagaacttaaatatgac

SEQ ID NO: 129 - DNA, 401 bp, Homo sapiens
tcattgtactagacaacttgaaaaggatatctaaaagataataaaaagacaactggttatttttatttgttttaatttccctaaaaaagatatcaactccaattaccaatctttgtttttaaattttaaaaatcacttattttaaggaatatttaagaaggaacttgtagtgtccagtgaagcaacttcaaaaagaaattmgaaaaagagacttgagataactagcaaaagcaatgagtagatcttgttttgaccctagcttgaacaaaacaattataaaatacatgttctgtacaagggagaacttaaatatgacttgcctatgatatggtactaaggaattatgattttcttagatgtgacaacaataatattgctattatggcaaattgtgggaaata

SEQ ID NO: 130 - DNA, 401 bp, Homo sapiens
attttaaaaatcacttattttaaggaatatttaagaaggaacttgtagtgtccagtgaagcaacttcaaaaagaaattagaaaaagagacttgagataactagcaaaagcaatgagtagatcttgttttgaccctagcttgaacaaaacaattataaaatacatgttctgtacaagggagaacttaaatatgacttgcctrtgatatggtactaaggaattatgattttcttagatgtgacaacaataatattgctattatggcaaattgtgggaaatagttattactttacagtgatgcatacttaaatatttagggttatatgtcatgatatctgtgattcactttcaaatacttgagcaaaataaaatatatataaatcgaatgcggcaaaatgtcag

SEQ ID NO: 131 - DNA, 401 bp, Homo sapiens
agactccttcattaggttacgtgtctattataagtgcttattgaaaacccaggatttggtttctttgtaacatgtaagtttcccgtttatctatgaccttttacatttaagtattgcccaagcacatgaaaaaaaggaatttgtttcaatggagatacacaaatatccttattcttcctggaccttattatgaaaattcartgctactcaagtttctctaataaaactttagagaaactgccaaagacttgggttttattttgttgtcagtctttatttaataatggcatctgtattttacccaaagtaaatcttttccttcttagaaaggtaagcagcggatgacttctctattgaagactcctctttggggccctggtagccatttgccaggccaaata

SEQ ID NO: 132 - DNA, 401 bp, Homo sapiens
gttccaagttagcccttaagtgcagctatctgtccctgtaaggtcacgttctctcaagcatttggtgagcagtgtaagtgccctacaaggagtgcgactgcttgctacactctccagtggtgaagcaggcaaatttggacacagcagctgtctgctcctgaagacagctggaaataaccatgtgtgataggatcagctatstgaggcagggtagcactgttgtctgtaaacagcatgctatctctgagactccaagacagtgacacaagcagcaccaggctcaacctgccctcagaatagcaggggaggcgctttacatccagccttgcataagttgaatccgaatcacaggattttaggactgaaaagtgccttggagaccatgtcacagccctctattt

SEQ ID NO: 133 - DNA, 401 bp, Homo sapiens
ttccttccatatttctctattcatctttaatattacctcattttactgtttaagactaagcatagaacttagaacatttaacattccctgtagaaaggaaaagtttggatactgaaactctgaatattcagattatatatgcaatatgtattgcatgtgctttagaggtcaagattattttcttctggagtttctgtttcwtcgtggattcctaaactgtgttaagataaagcagaatgattacatagcttacaagtattgtcattttggagggatgcttactttcctgccagtttctgagcttgtcaaatctaaagtcagtactgtacatttttcctgaatactacatcacttttaaataggaatcctgcatgttgaaaaatatgtatcagaggtaaaac

SEQ ID NO: 134 - DNA, 401 bp, Homo sapiens
taagccaggctctacccttgtaatcttcattgtcagctacatgaagaggagcttctttcagtaggaagtgcgcatcatccaaaaatatataggtattttattcatttcatgtgattgtttaaaggagactgcaagagtagagaatcaatagagaaggcaatgcgtgaaggagaaaaaattacaggccaaactgctgccacrcagagggtgaagtaatttacccaaggttagtagcagaacttccatttagggacacagtcaaaattagaacccgtgggtcctagctttaggtgcagaatgccttcctcttctccatcctgcctgaggctgtggaaaggcaaacgagctgcccttctcatatggggttagatgactggttaggcactgcacagccacatgtc

SEQ ID NO: 135 - DNA, 401 bp, Homo sapiens
gtgcagaatgccttcctcttctccatcctgcctgaggctgtggaaaggcaaacgagctgcccttctcatatggggttagatgactggttaggcactgcacagccacatgtctgcgtggcatgttgtttctttacctctgccctattcacatttctgtctctctctaccgaagtcagaagttgaaacttctagttgtaaggrcacaaagtggctccatgtcaactttcccccgtaaattatttttatgggcataccaaccaagggcatatctcaaacgcatgattgcttttggcatcgtaccaatgcttctccccccacccctgttttatggataaaaaaggccttttctggttattcttctcagatggggcccttgaaacttctgtaatgcataatgttaa

SEQ ID NO: 136 - DNA, 401 bp, Homo sapiens
gtatgatccatgcctcattgcattacagggagaacctcaaaaaattatattactgcaacctgggatgagaggtaagggaggtggaaagtcggtagagatggtaaattggtggtgaactttattttacaattaacctacggggatgattattaaataatagtatgctttggtactcagaagtcaaaccattggataggacartgagatttagagagaaaactaaaatttccatgctcttatcctttgtgttgtttcttccaaatatatttatcaaaacacattttaagaggggtgggaaacgccagttacacaagaaaagctatcctctgcccgactgaagaaattgttcaaacagagtgcattctttcataagcaatgcttcctaatgtagccagggtcag

SEQ ID NO: 137 - DNA, 401 bp, Homo sapiens
acttctgttttaaattacctggtttgtggtactttgttacagcagccctagtaaacaaatacaccacgttacccgtttggattgcccatttttgtctttatcatgttctctcggggaatctctaaacaaaatgacttgagtatcatctgggtgccagatgctgtctttacacttggcgaagttgttgaggtcctggctctrcatactagttcttatgtccatctgctttgatctggaggtcattattatctacaatgtcacatcaaatcttctgtctacctagaggtttagttaagaggacttcaaattctggttccttgatgtagccacagccacagaatgtcataacagtacatgttggatgtacctctccaggttgctatgaatataatgacatgaaa

SEQ ID NO: 138 - DNA, 401 bp, Homo sapiens
tgggtgccagatgctgtctttacacttggcgaagttgttgaggtcctggctctgcatactagttcttatgtccatctgctttgatctggaggtcattattatctacaatgtcacatcaaatcttctgtctacctagaggtttagttaagaggacttcaaattctggttccttgatgtagccacagccacagaatgtcatarcagtacatgttggatgtacctctccaggttgctatgaatataatgacatgaaaatcattgtaaaattataatatcattggctaatatgtatttaatatgaattatgtgtctaggactgggcagatgctttacatatgttgtctcactcagcagtcacaaagggctctgaagtgggtgctattatcacctatgcccatttt

SEQ ID NO: 139 - DNA, 401 bp, Homo sapiens
actgcaatgctccagccccacccaggggtcagggctcacagccaggttactcaaacagaggtcactgtggctctctcctgctgctcaacccaggctggagccgctagccacagatcttagacacacaccacgggcaaggacacaggggtctggctttattggctgtagggccagtttggctgggcgactaggtggacattstaatgactaagcacctttccttagccactcccagggtcagttaagagacatagattttaaaaccaagagaaacaaaacctagcaaacaagagagatccagagagaaaaatactgtaaaatttacagattagaatagacagttttacctgtacaagaatctgtttttgatttctgctaaaaatattgcatattcattatat

SEQ ID NO: 140 - DNA, 401 bp, Homo sapiens
aatttattaaatcttttttagaggggtattaccagacacaaatgtcaagtcaaggaacaaagggactaatccaaattcacaaagaatttgactggctgtgtctcctgaacccgtgacttcctccagcacttcagatgtggcataaagcaggagggatttcctaacgatgcagatgcactagaagccagagggtcagggagwcctcttctctcactggtgcttggaagatttccataagtaactcccagtgctactcatgagaactacataaattaagccttcagttcaggatagaacccagacttgtagctgagggcatactcaggacaaagtggcctctgagtatgcttagaactcagaattcaagggcagtcagaagaaggctagacacatcttcaaaa

SEQ ID NO: 141 - DNA, 401 bp, Homo sapiens
ccctaaaaattgacaaatgggatctcattaaagtaagatcttctgcaccagacacacacacacacacatgcaaactataatcggagtgaacagacaacctacagaatgggagaaaaaatttgcaatctatccatctgacaaaggtctaatatccagaatttacaaggaacttataaaacattccaaaataacccaagcaamctcattaaaaagttggcaaaggacatgaacagacacttctcaaaagaagacatttatgtggccaacaaacacattaaaaaaagctcaacattgctgatcattagagaactgcaaatcaaaaccacaatgagataccatctcatgccagtcagaatggtgattattaaaaattcaagaaacaacagatactggcgaggctg

SEQ ID NO: 142 - DNA, 401 bp, Homo sapiens
ttaccatattggccaggctggtctcgaactcctgaccttgagcgatctgcctgccttggtcatccaaagtgctgggcttacaggtgtgagccaccgcacccagcctcttaaaagttttatatatccttggaaccaataagcccacttctagggatctctgctaaaatattcactagagatgtagacaaaaagttacatacrggattattctttgctgcattatttatgagatagaaaaatggaaatgatctgaagaggaatttaataaattaataaatttctttgcaggcattaaaattaaatattgagggaatatttaacaacatcagaaaatactcatgttgtaataaaaaatgggaatgcagaagacccgcaacagttcttaccctgtttaaatcaga

SEQ ID NO: 143 - DNA, 401 bp, Homo sapiens
ggcaggaggaccacttgaggcctagactttgagaccatcctggacaacatagagagaccctgtctctacacagggtctaatttaaaaattagccaggtttggtgttacgcacctgtggtcctaggtatttgggaggctgaggcgggaagatcgcttaatcctgggaggttgaggctacaatgagccatgattgcactattktgctccagcctgggcaacagactgagatcctgtctctaaaataataataataataatatacaaacaaaaaacaaaaactaacaaactgcatagaaagtagttggggctccagctataaaggcaaacttgtggatcacaaataattatttaaaggagatctgtttgttcacaaaagaagtatagtaatgctataatagctc

SEQ ID NO: 144 - DNA, 401 bp, Homo sapiens
ttaaaggagatctgtttgttcacaaaagaagtatagtaatgctataatagctcaggaacagttgagtgtctctgcactactccctgtagacagtggggatgttgtacaaaacaaaggtttataatcagactttttgcagatcaatcaacctagcactagggtatcttctcctagctagaagatgaaagcaggaacaagccrtaagcatggtgaaaatttactcatttatttcaacaaaacactgtaatattttaatgagacattgaaaataagacatacaaccttaaaaataaaggcagaaaaatatttttagttgatgctcatgggttgaatttttaggtcacaatagacgagctggtagaaagaattgcttgctttaaacggaatagtttaacaaaaaa

SEQ ID NO: 145 - DNA, 401 bp, Homo sapiens; identical to SEQ ID NO: 35

SEQ ID NO: 146 - DNA, 401 bp, Homo sapiens; identical to SEQ ID NO: 18

SEQ ID NO: 147 - DNA, 401 bp, Homo sapiens; identical to SEQ ID NO: 29

SEQ ID NO: 148 - DNA, 401 bp, Homo sapiens; identical to SEQ ID NO: 15

SEQ ID NO: 149 - DNA, 401 bp, Homo sapiens
ccagttagaaagatcctatcatcctcttaaccatctctataagagctggaatgccttgactttcccaagagaggagacagggagaacaggaacctccatcaccctagggctatttggcaatcttccctcttcttccttcaacacccctttttgaggttagagtcatcctattccttccttctaggttccactggagtcagrgggattaggcaatggggcatggactgcaatgctccagccccacccaggggtcagggctcacagccaggttactcaaacagaggtcactgtggctctctcctgctgctcaacccaggctggagccgctagccacagatcttagacacacaccacgggcaaggacacaggggtctggctttattggctgtagggccagtttg

SEQ ID NO: 150 - DNA, 401 bp, Homo sapiens; identical to SEQ ID NO: 17

SEQ ID NO: 151 - DNA, 401 bp, Homo sapiens; identical to SEQ ID NO: 19

SEQ ID NO: 152 - DNA, 401 bp, Homo sapiens; identical to SEQ ID NO: 23

SEQ ID NO: 153 - DNA, 401 bp, Homo sapiens; identical to SEQ ID NO: 24

SEQ ID NO: 154 - DNA, 401 bp, Homo sapiens; identical to SEQ ID NO: 30

SEQ ID NO: 155 - DNA, 401 bp, Homo sapiens; identical to SEQ ID NO: 32

SEQ ID NO: 156 - DNA, 401 bp, Homo sapiens; identical to SEQ ID NO: 33

SEQ ID NO: 157 - DNA, 401 bp, Homo sapiens
cttgtcatctgtaaaactgagccataatacataccctacctagtgagttaatatgctggattagcaatcagcatatgaaggcctttgaaatctgcgcttatttcttgttttaggcttgctctccttcaaatccctccattgcagcccagtgggctgccttttccatgaacttcattcacctattccttttcctgcaacccwcttccaactatgtaaaaagcacagtctcctctttcacagcactcgcctctggtattgcaatatatttttcccatcacatccactagactgtgaactcttgaggttgtttatgttttattctcctttatcccattagtgcctagcacaattcctgacacatcatcagtaaatatttgttgagtgcataagtaaaagagaat

SEQ ID NO: 158 - DNA, 401 bp, Homo sapiens
tatttaaaaccagttttggaagagggcattgcattgggtctcctttaaactgctactggctgagtttatgaggttccactgagagacatagtccatccagtttgtatgtggcctttatcaggcatgcgggagagcacaaaagactcacaacaaacctttgacctcatgtccataggtttggactgggtttggagagttcayctgcagactagcttgataaaaagtgctgtcacagatgtaaattggaatgtcaggaatagctttattgacttttgtgccagagcataggatctgaaaagtcatttgttctgcaatcttttagaaagacaggggccatgcaatatgatcttcttctactgtgatattactggaaaaaggactggacatttgtgctggctcca

SEQ ID NO: 159 - DNA, 401 bp, Homo sapiens
tctaagttaaagaagaattttaaagagtgagttctattacattctttttgtttgtttgtttgtttttgtttgagggagagtttcactcttgttgcccaggctggagtgcaatggcatgatctcagctcactgcaacctccgcctcctgggttcaagcaattctcctatttcagcctcgcagctagctgggattacaggtgyccaccaccatgcccagctaattttttgtatttttagtagagatggggtttcaatatgttggccaggctggtcttgaactcctgacctcgggtgatccacccacctcagcctcccaaaatgctgggattacaggcatgagccactgtgccaggccctccattttttaatgtatacccaagaagactgaaaacatatgtcca

SEQ ID NO: 160 - DNA, 401 bp, Homo sapiens
ttcacctgcagactagcttgataaaaagtgctgtcacagatgtaaattggaatgtcaggaatagctttattgacttttgtgccagagcataggatctgaaaagtcatttgttctgcaatcttttagaaagacaggggccatgcaatatgatcttcttctactgtgatattactggaaaaaggactggacatttgtgctggytccattttctgctgatatatagttgtgacccaggacaatttatttaacctctttggaacttggttttctaatgtattaagggaggtggttatgccagttgatctccaacatctttgatcattcagccctgaatttctaaggttttatttttttcattggttttcatctgacacataggctggaaaagatgagactatggt

SEQ ID NO: 161 - DNA, 401 bp, Homo sapiens
ttgcataagttgaatccgaatcacaggattttaggactgaaaagtgccttggagaccatgtcacagccctctattttcacagattagaaaggaaacaaaaacaagaatctgagaccgcagaggtatagtgacttgtgatatttgtaattgtcattttgcatacatgcatagacccatcagggatccaagcagatgcatgtrtcattctgtcgtgcttaagctggttcatttgtttcctttcaaatggttaattatgcacatcaacaaaatttcataagatttagccttagatataagagtaaatattttgttctgccctcaattcaaatatgccctcaggagagcgacatcaacagtcatgcggttactgtatggcaggctttgtctggaaccaagcactg

SEQ ID NO: 162 - DNA, 401 bp, Homo sapiens
aaaagttttaggttctaacaatgttttaacaatgattttgaataaaaacaccttgactgtgtgcctcctcactaactaaatatttgtgaatgagcgtattttcattatttttaaataagacaaactgaacttctatacagagctgctatttacacatgaggtgtttgctttttggtttatcttgctgacttgggccaatcycccaagcggttttaactgaagacaaggttatcttgccatccttgatttggggcaatcagccatcacaaaacaattcttggtaataaacattagaaggagaggcctgcaagcagctccagtatgataaataaggatatcgttttccaaataggaaacaggaggaggttaatttaaaataaaccactatgaggtttcttaca

SEQ ID NO: 163 - DNA, 401 bp, Homo sapiens
aaacagggcttgactttgtgatggcctcgtgccaggcacctgttattcatgctgcttctcctggccacaagtgccaagggggcaggattttcagctagttgcccagtgggtcgttcagggtgctgctccttctggagatagcatagattaatattttacctcagaaaggagtatagctcattggtgatacatgagtgcccyacgggcttagaatttagaataatacttgcttatgacgcataaaagcaaactaagatttccttgtctcatattttctctacattggcatagagttgaaaaatcatcagcagttttttttttttttgttgctgttgttgtttcgtttttgagacagggtcacgctatgttgcccaggctggaatgtactggtgtgatgatgg

SEQ ID NO: 164 - DNA, 401 bp, Homo sapiens
taatacaggaacagaaaaccagcaccacatgttctcacttataagtgggagctgaacaatgagagcacatgaacatgcgaggggaacaacacacactgtggcctgttggagggggagggcaagagagagcatcaggataaatggccagatggctaatgcatgtggggcataatacctaggtaatgagttgataggtgcagmaaactaccatggcacacgtttgcatatgtaatgaacctgcacatgtatcctggaacttaaattaaattaaattaaatatttaaaaaaatgaaaaacagaaaagtttgattcaacttgcttaaacaataagaatattaattaccctggtaaccagaaatgcagagattcgacagctctaaggcatggtatagtcaagacct

SEQ ID NO: 165 - DNA, 401 bp, Homo sapiens
tctttacccatgaccatataccctattaagatttttctacccggtactatttctaggctgagccaacttttaggttgtagaaaatagtaaagggaaaaggaataaggacgggataggatttaaaagaacaaagatatttgaggggaggacaggcgagggagaaagtgacagagccctaaaatgatctcttttgcagtctaycctcccccgaaatcagtgcacttaaatgtcttttttttatggtgatttgggcaatatttactagagccagaaataccagagtatcaagtattcctgtctgagctggcattttgcctactagtccatgaattctcccactgggtacatcctgaagccttcttggagattgcaggtcataaatgtgcatgattaattgccta

SEQ ID NO: 166 - DNA, 401 bp, Homo sapiens
agaaaaatccagaatttcttactataatttaagagatgattgacctccaaaaagcttcaccacagtatcaactctatgcttcatatattttctttaattcataagcagtacattttattcgtggcataatcacttcttcctatattccaaatgctttcttttctttgtttcctatcattttacatataatatgcaaatacwctaaaattgaaatatagcttggcagtttgtaatactgtaaaaccataaagtgtaatatatgactacaaaatattttggtttaatgggcctggtgttctactatatatttattttaagtcactcttaaaatgtcaaataaaactcaaaaatctgttttagttccaaatgtagagctattaattttatggcttaatatctga

SEQ ID NO: 167 - DNA, 401 bp, Homo sapiens; identical to SEQ ID NO: 31

SEQ ID NO: 168 - DNA, 401 bp, Homo sapiens; identical to SEQ ID NO: 34

SEQ ID NO: 169 - DNA, 401 bp, Homo sapiens
acagcgctgcagtgtgtgcccttgagagtctcctttacaaacacgccagagcagagatagtgacctcatgcttcccagagaacatcttcgctgggctgtcctgaggtcacctcacactcaacacacccaaaatagaaacacaatcctcctcacacttgtgtggggccacatcattcccccatttgctagaactagcaatckcatttttatgccaatgacaaattggttctgtgtctgtacagtctcctatagctacaccttccatggctctcctgccaccactaccttgatgcaggcttgtcattacccttcactgccactcttgccatgactttcctgatgggctctgaaactgcagattctccctcgccagtccatcttctgtgctgctaatgggactg

SEQ ID NO: 170 - DNA, 401 bp, Homo sapiens
gcaccccttgaggtgaccatagggaatgatttacaaaataaaattaataaagtgaaagtgatttgtgagcatttgtgagaatcatagaaatatttttccttcctcacagattaattgcagtatctcttgctttggatcttcaacatcctacaaaaccagctgtttccatgtgtcttttccctttttcaatgttttaataamagtcagggctgctgttatgattaggaagtttctactccaaccatagttatcttccacattcagatacgacaacgacttaaatttttaccagtagaaatttattcatttaggtatcagaagcgtttggctctttagtaatctccaaatatatacctatacagacgatgactcagtgaaaacacataaagtgtcatctttca

SEQ ID NO: 171 - DNA, 401 bp, Homo sapiens
ctcagtcataaaccagttttccaaaaggaagatttctcttgaaaacaaaactaatgctgaatgtttctgagagaatgacatagctctccgaaggacaatttaaaatttcctgcggagagttctggcaattacaatcataggaccaacattgttcaaagtggttccatggaatgttaacagggatcacatggaaaatagggytctgcggtcaaatccttttgtgaaacacgaggttaaaccaagttaaatcattttctatactaaaggtcataataaaactttaaatattataatatttattgtgaagctccatgttttccagaatctttcttctttttggcagagtatctcatttggcttgtgtttcgtaaaatatactttggaaaaatgtagccttagat

SEQ ID NO: 172 - DNA, 401 bp, Homo sapiens
caggagaatcgtttgaagctgggaggtgaagattgcggtaagccgagatcgtgacactgcactccagcctgggcaacaagagcgaaactctgtctaaaaaaaaaaaaaagactcctcaccacttttgccatgtgtggttacagcaaaaagatggttgtttcaccagacacggaatctgctggtgtcttgagcttggactcyccagtctcctgaattgtgatcaatgaatttctgttgtttgtaagctgtccagtgtattgtattttgctatagcagtgtaaacaggcttacagaccaagatatgtgacataaccccagatttagtttctagaatctcagtctcttgaaaaaaaacattcaattttttattcaaatgttttattctttctgtggaagaatta

SEQ ID NO: 173 - DNA, 401 bp, Homo sapiens
gataataacctccattccttcttattaaacagaaataaaagcatccaatatacatgcttttctagatgtggaaggctacatttgacaatcaattattaaagtaaaattaaacctggctttgggaggccaaggcaggggcatctcttgagcctagaagctcaagatcatcctgggcaacatagtgagggatccagtccctawaaaaatatataaaaaaggcgaggtgtggtggcatgtacctgtagtcccagctactcgggaggctgaggtgggagcgaggcttaggtccaggacttagagcctgcagtgagccatgatggcaccactgcactctggcttgagcaatagagtaagagtccatctgaaaaaactaaacaaaacaaaataaacctggtgttgtc

SEQ ID NO: 174 - DNA, 401 bp, Homo sapiens
tactaggctgcctagtgcaaggtccagccagggttcacagttcagcgtctgaggctctgctgactgccttctttgtttaacttgggcaagcttttgataagccagggactgcttatttaaggcgattattacaaacaactgacagattattttcccagattcttgcacttattcttcatatgcaagtttaaacgagtcccmaactataatttcttgaggatttgtgtgtgtacaaaagtaatcgagataaggatgtgtgaaagactaggtaaaaaaatctatttctaaggaataactatgctccaagcagacatagaaaaccaaggtcctttgaagtaaggactcgtgagggcaatttcaagacaggtcaaagaattctcgtctctaatgatcaaagatca

SEQ ID NO: 175 - DNA, 401 bp, Homo sapiens
tatgtgtaagagacatactttcatatgagactttgggtcaagtcttcattatctcttgtagtgataaaagcatggttgatctaatctctgtccttcacagttatactccgctctgtctttccattactactgttactagaaatgatttgtaaagtgtaaagcaagtggagtcaaatctggcaatgcctctctcttgctttraaaacattaataaaggcagaggccaggttcagtggctcactcctataatcccaacattttgggagacggaggcaagaggactgtctagtctctacaaaaaatttaaaaattagccaggtgtggtggtgcacacctgtagtcccagctattcaggaggctgaggcaggaggacctcttgagcccagtagttcaaggctgta

SEQ ID NO: 176 - DNA, 401 bp, Homo sapiens
gaaagtggaaggatatagaggcaaaacagtgctctgctgcagctgatgcctctcaaagattagtcctacacagttggaacagtgatgactggcccagaatgctgcagttatctcacttcttcagttctaaggttggccaaaggctgtccatgtttgaagtcctagcaattgctgttatacacaaacagtccattctaaacygcaagctactagtaagggaccggagtaaggtcctggacccccgtggtttttcctcctcagtgagctacctaagggagaagtaactgatcttgctgcctttacccacatctctcacatttaccttttgctcacctttctcagggacgcaatcctcagtgatttagtcaaggtgctatatccttttcaggaaggttcccaag

SEQ ID NO: 177 - DNA, 401 bp, Homo sapiens
gagtatctggctcttcatctacaaattaaattcataatatctgctttgcagcatgatcagaggagctctaaggagcatatgttcagtgcctggcacagagagatactcaataaatattttcatttaaaataaccatgtatatgcattaccttatttttagttgtataatgacattttatttcactaaatttgacattaatrttgttggaaattatcctcttggttaattctggtttttctgcagaaaaatagattcatttttctttcaatgaggtaagtctttagtgcctaccatcaggcaaaatggtgtatctagagggcctgtaatattcttaggaaaatgtcaaataaaatggggtgaccataagaaaggtccataccaggatgactgaatcacacag

SEQ ID NO: 178 - DNA, 401 bp, Homo sapiens
tggaatttgcatagtgataatgatcagtaagtcaggtgggatatgtgtagttgtgcatttattcctaactacatagctgtatgttaatacagactgcaagaatgaatggtaaaaagaggccctctatttcttcttctttggttgtgaggccactgatgataaaaggacatatttagataacttgcttttaagcagtaaacrtttattcagcaaactataaatcaggattcttgagttctattacctcttccgtttcacctattctcatgattagaattttgcaaatgatgcaccttctccctgcctcaacttcttttccaactaaagactcccacaaaacatgccatctaccaaattctcaggaggaaagagaagagcgattgcagccataacctcctaac

SEQ ID NO: 179 - DNA, 401 bp, Homo sapiens
gttaggcaaagagagcaatgcctgctggaggcagttctattgcattatttgaattgaatctgatcaacagagaactattctgtgcactcaactacacagtattaagacattataaatatatgggctttttgtcaaaatttcagatctacctatctgattgttctgatttcttgacttgatggtgtggatgacagagaaagkgaaattaacctacaacatgattacttacttcatttttattatttttcctcttgagaaggctaaggatgtcaagaatgttacttgctcttacagtgtttcataaaagaccctgaaaattaaagtagttttagaaactatacatagctccaacccacagcactggaaatatattaccctctagcagtaatagcatataagtg

SEQ ID NO: 180 - DNA, 401 bp, Homo sapiens
taaacacattggaagtggcatcaagagaggtcctgagagccctgagcaggaagtcacaactctcttcatcaccaggatagcaaagggggagcccacattccatgaagtctatgttccaaggtagaagggggaagtagtagatgccccgtgataggcaggagagctgcattgggtgcacacataaaatgagaacttcccccmaaaaattagaggaatttgagggggagtcgagtttttagaattcagcaggtgagggatgaaccaaactctgtttaaacattattatatggtttagattttcctacaaactgtgaatacacaattaattattattttagctttgtgaacagaactatgctagcactgtggctatgacagggagtgaaatatggtttttgctt

SEQ ID NO: 181 - DNA, 401 bp, Homo sapiens
taaagatagcagagaaacctggagctcaggcttgagtctgactgctagacatcatagctgtgtgaattctgggaaagtctgaaaaatcttctgtgcctcagtttgctcatctctaaaatgggaatagtaattgtacatacacttagggttaatgagatgattaaataaattaagatataaaaagcacttaaagagcttggyacctagaaagtgatatatgtgttgttacctattattacggttattaatatgacaaaagaatttgagaaagccactaaatttccactttaaaatgtggataatataatattaatagctttgtatacgagctccctacaatgcaacgaaagcaaaattaattatggatgaattccaatgaagctaagaactccaaaccaatg

SEQ ID NO: 182 - DNA, 401 bp, Homo sapiens
aaacaggttaaagttgcagtctataagagagtggctttggaggcagtaagacttgggttttaatgatagctgtgccatttactagctgtgggacatcagtcaaataacttaaatttcaaaggctctgttttctcatctataatataaaatagaaataatagtagtgcctcagggctgttttaaggattaaatgatagcacrtggagcataacccaggaccaggcatacagtaaacgcttcataaaatgatagctattatatatcttaaatcaagcatcctcaattccttgttgataagaaaaataacaaaccactagcatttggttaaaaccatgagagctaatgtgaaaatggatgtattaaacagcttgccataaaaatgctacattttttcaaacaat

SEQ ID NO: 183 - DNA, 401 bp, Homo sapiens
acattagaaagtctttttagctttctggatctctgtcttcttaactgtgaaatgaacataagtggcttgtcatcctccaatggctatttatgtgtttgttgagttcataagatgtaagtttttaataatatattaaaaatttgaagcatatgacctctaaggacctttcaagatatattttttgactgtgagacttcatgrcatataatttgtctcctgctgtattgcatatccttttgtaattggtgacatgtcttctgctgaactccctccaggttgttttagtgagcaaaaatgcttgtggataaaatgagatcctatcaggctggcaaagttttaagagtttgagagcgttcagtgttgcaaagcatgtgaggaaacaggcattgtcaaatactgtt

SEQ ID NO: 184 - DNA, 401 bp, Homo sapiens
agtgctgagacctggataatggagccagctgccacagaggtaaggggagagagaggagacgctaggagaggggatttaggaattccagaatgtacttgggaaggacagccttgtatcccacttaatttcttctgcagacacaaatggaaaggctggctgtctatgtgcccctgaaataaggccagggtcaaaagtggggcygtggcctggagcagaattgcaggacggcttttcctccccaccaaaagaaagaaaaggaaataattgggtgatagcttactgctccctttcatcatttctttcctctaatagtctgtccttcatggaaaaatagcacatactttccattcatggaaatatcacatgtttatataaaatgattcaaaattaaatttaatttt

SEQ ID NO: 185 - DNA, 401 bp, Homo sapiens
agagaggatgcatggaggtgtcccatgggaccgatgagagccccagggtagagagggtctgttgtgggttgaattgtgtccctgaaaagatgttcaagtcctaattcccagtacctgtgaatgggaccttatctggaaacagggtctttacagagataatcgagttcagatgaagttatattagattagggcggcccagtrtccaatgactagtgtccttatatgaagagggagacttgaagacatagagacaaagggacacaaagagataggagagaaggccatgtgatgacagaggcagagaccaagtgatatgagaacaggccagggaaggccaaggattgccagcagccaacagaagctaggaagaggaaggaaggatccctgcctagcacaaaccc

SEQ ID NO: 186 - DNA, 401 bp, Homo sapiens
aaatcctgctgattattccttccagatatttcttgaatgtacttgtcttgttcctccacagcacttactcttcagccaccatcatctgtcacttggatggctccaacagcctcctacctggcctctcgcctccagcctggccccccttcagtcctccacacagtaggcagggtcctctgtttaagttatagccagctgggsactccctgccaccccccttcagaatctggtatttagccccacatcttcctgctcctggatttggaacacaccagccatcctgagctgctttcaattcacgaaagtagtgtgacccatctcccctttggccctttgcatgtgctgtcgcctctgctgccctctcttttcctcactccctggttttggggctggagggctgt

SEQ ID NO: 187 - DNA, 401 bp, Homo sapiens
gcctcagcctcctcccaaattgctgggattacagatgtgaaccaccgcgcctggccaccattattttttttttatcatccccaacataaattctagccattaaacaatactctcccttctgtcttctccctgtctcctggtaaactttattctgttctgtgtctctatgaatttgactactctagctacctcatgtaagtkgaatcatacaatatttgttcttctgtgtctggtttatttcactaagcataatgttttcaagatcaatccatgttgtggcatatatcaaaattccattctttttaattggtcaatagaattccactgtatatatataccacatttgggttatccattcatctggtgatgagcacttgtattgtttctaccttttggctact

SEQ ID NO: 188 - DNA, 401 bp, Homo sapiens
gggcttccttggatccctgaaaaagaaccagaccagcctggcctgcttctctgcatcccagggtccccagaaaagtgagggctgtttgggggtcctgggtcagatcctcatccaactcctacccccttgaactcccacctccaaaggcaatctaatagctcctgctgggatctcaagctccaagggagaccttcataccaygcacattctgggacaatctttggttctcctctcagcctggacaagcagtgaaatacagcatccagccactgcctggctcagtcagccatgctatcttgggacagaaaccttgacaggaatcccagctctagccaaggaaaacatgagtcaggcctggctaagccaggtggcttccctctctccaccttccatccccacac

SEQ ID NO: 189 - DNA, 401 bp, Homo sapiens
aacctttcctgcctttgtgtggcttctgttttgtttgtttgtttatgtatttatgtatttatttattttgaaacatagtctcactctgtcacccaggctggagtgcagtggcgtgatctcagctcactgtagcctccgcctttgggttgaagccgttctcctgcctcagcctcccgagtagcttgggttacaggtacccgycaccacgcccagctaatttttgtatttatagtagaaatggggtttcaccatgttggccaggctggtcttgaactcctgacctcaagtgatccacccacctcggcctgggtgctgggattacaggcgtgagccaccgtgcctggccagggcttctgttttaaaagcttctccactctctcttctccagaggaagaggtgca

SEQ ID NO: 190 - DNA, 401 bp, Homo sapiens
gggaccccaattcaccctgctaaaagaaaaacatattaagctgaaagctgagtcatagaagaagctgccttctttttttttttcctgagcagatagctacagataaaaagtaaaatatctccacagtagctactctgtgttcaccttatctaatggtgagcttgagaggaatacataattgaccattcccctacctactcsttttctcttgcaacacatggattcagttatgtgacctcttttcccttgagcctgcttttctcctttaaatattgaggccctcagaatcatccttggagaaaggcaccgacctgtctcctaggcatgtgtccttaaccttggcaaaataaacctctgcattgattgatacctgactcaggcactttttggtttacgggaag

SEQ ID NO: 191 - DNA, 401 bp, Homo sapiens
taactttgggaggccaaaacaggcagatcacttgaggccaggagttcgagaccagcttagccaacatggcaaaaccccgtctcctttaaaaacacaaaaattggccgggtacagtggctcacacctataatcccagcactttgggaggctgaagcaggtgaatcacatgaggtcaggagttcaagaccagcctgggaaacrtagtgaaaccctgcctcttctaaaaatacaaaaattagctgggtgtagtagtgcatgcctgtaatcccagctactcaggaggctgagacagaagaatcgcttgaatccaagaggcagatgttgcagtgagccaagattgcaccactgtactccagcctgagcaacagaacgagactctgtctcaaaaacaaaaattagct

SEQ ID NO: 192 - DNA, 401 bp, Homo sapiens
cagcctctcttcgacagggccccaccctgctagcctttgaacttggaagcatcgcccacaaacaccagtgccaccctctagccttgagggaccatatatgtctctgtgaatctccgttgcattggacagcttctctcccaagagctgctcttataaaagagacacaccacattttaatgaggcttcgctctccacagaggyctgcttccatcctaattgagcaagtagactttagtaactttggaactcactcaccccgtgactaatacaccctgtctacccagcgcattccctgtgagatttgttcatgttgttctccccctccttaagcattgtgaacctaggaaaaatatcatctctgtagggcaataaaccatgtgatttgtgaaatcatactttga

SEQ ID NO: 193 - DNA, 401 bp, Homo sapiens
tactgaattaaaagaagtagcaggaatttgagagactgggggctggcagctggaggaggcaggagctgcccagtaggacatccaggcatcgagcaagtgtctgtgaaggagcattcatggaggattcctcacgccagagtcttggaggtgcgggacgttgtgttttagtcatttgctctattatcacataaagtcatgccyctcttgaccttcatgcctttagtaagtgaccagggcagaatctcctggcgagggagagctgaactgggggtggcccccagccccacctcatggaaatcctcagagcagcctggacggctaacatacctgggtgtcaggcagggccagcaggaaacagcaccactcagagtggatgagtgagcagagttacggagggacca

SEQ ID NO: 194 - DNA, 401 bp, Homo sapiens
gagggagagctgaactgggggtggcccccagccccacctcatggaaatcctcagagcagcctggacggctaacatacctgggtgtcaggcagggccagcaggaaacagcaccactcagagtggatgagtgagcagagttacggagggaccattttacaaaggtatgggtgggcttgggggaaaccagacgcagtgcagtcytcagaggtgggaaagctcagcaccgttaattccacctggctgggggcaggtggggaaagagagaaagtggtaggggcaagggagctgggatgctgcctgacaaagctgtgaccttctgtcataggacacagccggtcctcagtgaccccagaaggaggaaggagggaggtgatgttatggggctgaattgttcccccaaa

SEQ ID NO: 195 - DNA, 401 bp, Homo sapiens
gctgttacatgagcccacccatcttgccatcaaagggatgtatttgcaccctgagtggtagcagggacagcttgctatgtgaaagctataagacaatgacacctcagagaccaagccacccagctatgggatcgtctagtgtgggagagagaacatacttttcctacccctttttcagtgaggcaagaagcagtagggatktttttaactgccattaacagaaaacccaactcaaaggggcttaaccaataagaaatttattatctcctataacaagaaaccgcaaggtaggctagctccagaaaaggttaatttagcagctcaatggcatcatcaaggacccagatgctatcctcagtgctgctgtcagcttcaggctggcagctggatgcctatagtgg

SEQ ID NO: 196 - DNA, 401 bp, Homo sapiens
caaggaggttgacagtttttcctctgtccgcatttgatctatagctgcaattacaagctttccttcacaatgcctgcatgccaacagatgtcttttgcaagtcagcaatgctgccatcacagctgctggctcaaactcccagcatgtgtgctactgaagacctttccctccacattctgagggatgccaggctctgccccyaccccctcccacactagctgggtctgagctgcctgggatatatcagagttgggtgagaggttaatgatctatttcaacagagaccccaaaagacaggagagactactccctccacccaaaacccacctgggatggctcagaaagcaaggtctgcaaacacgggggataggggtgggctacctgcccagagcttcctccca

SEQ ID NO: 197 - DNA, 401 bp, Homo sapiens
tatagtccatgtgcccatgtcaccaatgaggcttagagaggctgtgaactacccaaggtccctcacaggcaggagcagggccagacctgaaccccccgtttctggcctcctaggccaagctccctgtgtgcctcctggacagtgtccctgcagactcctgagtcctcactgaaggtcagagcctgcccgagtttgaggagrtccttctctcccccttccagcgtccccaactccaggcagcccatgggctaggggattttcttcttctttggaacttagcacatgttctgagtctctgacactgtggcccagcaaggcgtctgccaggagcagctccgaagcccaccctcttccagcctcactcagggcatgtggcgacttgttctgtctccaggacattg

SEQ ID NO: 198 - DNA, 401 bp, Homo sapiens
gctgtctggagagtccccctcccttcttccctcctactctttccttgcccctactccatgcctgtgcccatatcttgcccctccctctgcaccctgaagccctgctcagctgacctgcctgcctcagacccccagggcccctttgcctggctggcctcccccaactcctaagcccaggactccagggcactgatcccagasccccagcagcgcccagccccgagtttggcagtatcttctgagtgaaggacttgctggctgtaacggaaaccctccagcagatcagtccatgtccagttggagcacaggcccacaagctgggctgtctgggttggaatccagctgctccccttcctgccctgggacctggggcaaattccttcacctctctgtgcccagtt

SEQ ID NO: 199 - DNA, 401 bp, Homo sapiens
ctgagtgaaggacttgctggctgtaacggaaaccctccagcagatcagtccatgtccagttggagcacaggcccacaagctgggctgtctgggttggaatccagctgctccccttcctgccctgggacctggggcaaattccttcacctctctgtgcccagttccccatctataaaacataggtggcaatagcagggttgyggtgggattccagctgcaaagtcctttgagcagtgccaagcacaagataagaatgtgttgctattattattattatcattattattatatgcctttgtgctcacttttgaagtctgagcactagagtcctgatctttggccacctaggggtccatgcaggggtgaagaccacgccctctcctggctggatcccaggtgag

SEQ ID NO: 200 - DNA, 401 bp, Homo sapiens
agccgcagagatttgcaggatgagggtggcctcattaatccaacactctagttaactctcatatctatgtcaacaattttcaaaggattattataaagcaaggaccatctttgataatcgtgatcaaagatttctaggatcttagggcaatgtttcttaaagtctattccatggctcttgagctttgtgagatgttaattsgtgttttatatacattaaaactaataagtttgggaaatattggattttaaaagtgaatatatttaactttgggaggccaaaacaggcagatcacttgaggccaggagttcgagaccagcttagccaacatggcaaaaccccgtctcctttaaaaacacaaaaattggccgggtacagtggctcacacctataatcccagc

SEQ ID NO: 201 - DNA, 401 bp, Homo sapiens
aggaagaaggtaaactccagctgagaagaccaaccccaggagctaacagatagcgggaactcaatacatagagttattttctttaagtgacttgccaaagggcacagagctataagtggcagaaccagaactcaaatccagaaagagaacaaggatgctaaagataaactgggcttagttcacagctgctcccatgacagyccccatggtggaggggcctcttcctctgggagtccggctgttttgtcaccctgaaagtgaagggcacagcaatgctttctgaggtaggggacccctcctccaccatggccccaggaatccctcctctgccaacatccagccagcccagctatcacatcacagctgcatttttctgctcttctctacaaaataactccaag

SEQ ID NO: 202 - DNA, 401 bp, Homo sapiens
agctgggattgcagggacctacattcttgttccggttgagtcactaactacctaggtgtgctagggcagggcctgtcacttctttgggactcagattccttgcctgtaaagaaggttggccctcagagcccccttgcaattttaaggttctagaattccacttttgggaccagctggtgccccatgaacagcttgttgtgkttcctacattagtttagattggggtgtcctacaagtccctgtggtgagaaagggcagtcttcctcccgcagcgagtcagagcctaccttggcagaggcgtcggccttcctaatgggctgaatctccctctgcctctgaattatgttcaactcgacaaatactgactgggccgatcagcatgcctggctctgtgctgagtg

SEQ ID NO: 203 - DNA, 401 bp, Homo sapiens; identical to SEQ ID NO: 197

SEQ ID NO: 204 - DNA, 401 bp, Homo sapiens
ctggagagtccccctcccttcttccctcctactctttccttgcccctactccatgcctgtgcccatatcttgcccctccctctgcaccctgaagccctgctcagctgacctgcctgcctcagacccccagggcccctttgcctggctggcctcccccaactcctaagcccaggactccagggcactgatcccagagccccrgcagcgcccagccccgagtttggcagtatcttctgagtgaaggacttgctggctgtaacggaaaccctccagcagatcagtccatgtccagttggagcacaggcccacaagctgggctgtctgggttggaatccagctgctccccttcctgccctgggacctggggcaaattccttcacctctctgtgcccagttcccca

SEQ ID NO: 205 - DNA, 401 bp, Homo sapiens; identical to SEQ ID NO: 198

SEQ ID NO: 206 - DNA, 401 bp, Homo sapiens; identical to SEQ ID NO: 196

SEQ ID NO: 207 - DNA, 401 bp, Homo sapiens
aattcctcagatgattccaatgtacagccaatgatgagcagctgctgggctgaagggactggggccattttataccaggtgagtaattcaaggtgttgatgtcaccctgctccttagaggactattagccaatcccttgagtttcagatacaaccagttatccctctccagggagcttgaaatttttttctcccttttctkttattggcaaaaagcaaatccctgcatggaagcctggtgtctacatagagctgacaagggggcagcttggggaaggtgggctaggctttgggagcgagtgggcccctccaaggagaaggcccttggattcatctacaggaagcccagagttctctgacaccaacccaactctcccacaccatgccctgcctagaggccca

SEQ ID NO: 208 - DNA, 401 bp, Homo sapiens
aatgaggtggggatcagatcttagagggcctcaggtgccaggataaagatcttggatggcaatgtatagaaagcagaaaaccactgaaggtcagattcatgtaacttaaaaagaaaaaattaaatggagcacaaccaggtaagaggttcctggaatggtgtatgcaaaaaaacaaagataatctgaatgccctgttggcayctccaacccagcttgtccacgatggaagccggcctcatttttccccatccctacctgtctgcctgtttcccctgtcctagtgagcagcatgggcatccccaagagtcacccaggactcttccctctcccgtatcccccactgcaagctcatctccaaatcctgctgattattccttccagatatttcttgaatgtacttg

SEQ ID NO: 209 - DNA, 401 bp, Homo sapiens
ccacacccccaactccacagccaatgagctgtttgggtaaccccaagaccccactcccacacagcctacggccagcaaaggcatgcaggcaggattctagaaccttggcccggctttgggttgctgcctttgaaagtgtagcaggtgtggggctctagctccggccctcctcaaaggagacaggagcattccctgtgtctktccggccttccacctaccctggccttctctgcacagggttcccttttggggcactgtgattgttctcaaaggcagagcccaccctggccaggagtggcacaagattcttatcccacttgtagtagaagacttttcctgattcctcttcccagcctatccaggggcttccgttctcctgccggtctcacctcaggctgaaa

SEQ ID NO: 210 - DNA, 401 bp, Homo sapiens
tggttcctacattagtttagattggggtgtcctacaagtccctgtggtgagaaagggcagtcttcctcccgcagcgagtcagagcctaccttggcagaggcgtcggccttcctaatgggctgaatctccctctgcctctgaattatgttcaactcgacaaatactgactgggccgatcagcatgcctggctctgtgctgastgtcactgaactcgaggaacttccctccaggaattctgagtctagtagaatagacaggcaatgaaaccttactgagagaagcgcagggctgggcatgatggctcatgcctgtaatcccagcactttgagatgctgaggcgggaggattgctcgaggccaggagttcgagagagaagggcaagagacaaggtccatgaagg

SEQ ID NO: 211 - DNA, 401 bp, Homo sapiens
acaggcccacaagctgggctgtctgggttggaatccagctgctccccttcctgccctgggacctggggcaaattccttcacctctctgtgcccagttccccatctataaaacataggtggcaatagcagggttgtggtgggattccagctgcaaagtcctttgagcagtgccaagcacaagataagaatgtgttgctattrttattattatcattattattatatgcctttgtgctcacttttgaagtctgagcactagagtcctgatctttggccacctaggggtccatgcaggggtgaagaccacgccctctcctggctggatcccaggtgagcatcactgaactcctccgcccaaggtgcatccagtgctgactggatgagagaaggaagggggacag

SEQ ID NO: 212 - DNA, 401 bp, Homo sapiens
cccacacccccaactccacagccaatgagctgtttgggtaaccccaagaccccactcccacacagcctacggccagcaaaggcatgcaggcaggattctagaaccttggcccggctttgggttgctgcctttgaaagtgtagcaggtgtggggctctagctccggccctcctcaaaggagacaggagcattccctgtgtcyttccggccttccacctaccctggccttctctgcacagggttcccttttggggcactgtgattgttctcaaaggcagagcccaccctggccaggagtggcacaagattcttatcccacttgtagtagaagacttttcctgattcctcttcccagcctatccaggggcttccgttctcctgccggtctcacctcaggctgaa

SEQ ID NO: 213 - DNA, 401 bp, Homo sapiens
ttccccagctacgtggaactgtaagtccattaaacctctttcctttgtgaatttcctttgtgaatacccattcttgggtatttctttatcagcagtgtgaaaacggactaatacagtggctttgttcattcacaggaaaagtggtgagcagctggctgctctctcgtccctacatgcagtccctccctttcctgtaggcayaatgcttaaggagccaggcctgggcttctgggctcagaaccagagcctttccttactgagtgcaaatgattgaaaatctcaggctacaaaagtgggaggcatgcacctccccaacagagacatgccattgccagtggggacactggaatccctttccccaggcagtgtgggatgggtgtggccgtgtgcctaggactggc

SEQ ID NO: 214 - DNA, 401 bp, Homo sapiens
ctaaaagcatttaattttgtaagggtgacaagtccaagaaagataagaagctggaggttggagggcagattatggggaggaggagcacagagcaggagagacagctatgtcttgtgccccatggagctagcaaagcacctagactcagatccaatcctttcatgttctgtagacaagtctagtgatcccagggtggcaggsaagggctagaagcagctcctgacaaaccacccatgtgtcccatagcagaccagagcctggtttcaggccttcggtctttagggtgaggatgtgatagccaaggccaaagtccagctggccggggggaatgtgggccgggtcctcgggagggaggttgtggctggggctgcaaatttgagagcacacattgaacatacagt

SEQ ID NO: 215 - DNA, 401 bp, Homo sapiens
caaggaaaagaggtttatttggctcttggttctgcaggctgggaagttcaataagcatggggctagcatctagcatttgctcagctccttgtaagggattttgtgctgcatcacaacatggtggaaggtcaaaggggaggtggacatatctgaaaaggcaaagcctgagggggatcctggttttgtaatagcccactcttscaggaactaatctgctctcatcagagtgagaactcacttattgccttgggaacagcaccaagccattcttgagagctctatcctcaagacccaaacatctcccactaggccctacctcccaacaccacatgggggatcaaatttcaacataagatttggtgagcacaaatcacatcccaactatagcaggcctctaggat

SEQ ID NO: 216 - DNA, 401 bp, Homo sapiens
tcaggtgccaggataaagatcttggatggcaatgtatagaaagcagaaaaccactgaaggtcagattcatgtaacttaaaaagaaaaaattaaatggagcacaaccaggtaagaggttcctggaatggtgtatgcaaaaaaacaaagataatctgaatgccctgttggcacctccaacccagcttgtccacgatggaagcyggcctcatttttccccatccctacctgtctgcctgtttcccctgtcctagtgagcagcatgggcatccccaagagtcacccaggactcttccctctcccgtatcccccactgcaagctcatctccaaatcctgctgattattccttccagatatttcttgaatgtacttgtcttgttcctccacagcacttactcttcag

SEQ ID NO: 217 - DNA, 401 bp, Homo sapiens
gagaaagcccttctctgaattggcctgggcagatgcgtggtagacggtggctttcaaacaagctcctgcacgctgtctgctttctgctctggtgctgcaggcaattgaaatgatctccatgccttctctgcaatcctggcacttcctgattctgtggggtctgacagtgacttccctgatcttccctaatgcagcccttcygtccattatgaatgcctctaaatcccttgtattaaaaccccgtgtatttggaatatctagagcagcttccagtattataaccaaaccctgaccgacacgctggcatagataaagaagctacgtaaaatgatgtgtggggaaacatcctgttcaatcttcccggcagtgctgaggttaggctgtcattttcccagtttcat

SEQ ID NO: 218 - DNA, 401 bp, Homo sapiens
acctgcaagccaagagggcttgaacaccagtctcctcttcagtgtgcgcatgcacaactctttcgagaggtttggaagtaaaaagaaggagtgggatggagcagcagttgaagagagggttataagatcccctctgtcttataaggaggtgttgctttgtttgattgtttgtcttgaaaggatgcgaagacctgagtactwgtagaggctgagagaagaagccaggggagagggagaggggacatgaaacatcacatctggatgccaagcacgtcttctttctgtctcactaatcggagtcataggagcttagccctgaaagggaccttagagtttagctccttcattttgcagatagagagagagcaggcacagaagaggacctgcccggggtacttccg

SEQ ID NO: 219 - DNA, 401 bp, Homo sapiens
ctctgtcagcctctcttcgacagggccccaccctgctagcctttgaacttggaagcatcgcccacaaacaccagtgccaccctctagccttgagggaccatatatgtctctgtgaatctccgttgcattggacagcttctctcccaagagctgctcttataaaagagacacaccacattttaatgaggcttcgctctccasagaggcctgcttccatcctaattgagcaagtagactttagtaactttggaactcactcaccccgtgactaatacaccctgtctacccagcgcattccctgtgagatttgttcatgttgttctccccctccttaagcattgtgaacctaggaaaaatatcatctctgtagggcaataaaccatgtgatttgtgaaatcata

SEQ ID NO: 220 - DNA, 401 bp, Homo sapiens; identical to SEQ ID NO: 192

SEQ ID NO: 221 - DNA, 401 bp, Homo sapiens; identical to SEQ ID NO: 199

SEQ ID NO: 222 - DNA, 401 bp, Homo sapiens
aagacatgcaggtgtgtctgacagacggggtgggggcagataaacctcaaagcaaccaggtcaccaagtcctgggagagagcccagctttgtgatacccacatagtagctcacaactatgcgccagatccaaatgggctcctgtgccactttctttaacactcctggccactccatgagggacgctgtgatctccacattktacagacaagaagccgagttgcagagtgatcgcataaagcccagggacacgcagatagtcatggtgcagccaggatttgaacctaggcagattccagaggctgctgtctgagttgctggtctgagtttccttctagctaaaggtagcagggaaagaacttaccgcctataggaaaacaagatctattttcataagcaaca

SEQ ID NO: 223 - DNA, 401 bp, Homo sapiens
cagggctgggcatgatggctcatgcctgtaatcccagcactttgagatgctgaggcgggaggattgctcgaggccaggagttcgagagagaagggcaagagacaaggtccatgaagggaaaaattcactccatcagggaggtcagggatggtttcatagaggagatggttcctaggttggattttgaagaatgaataagarttcgacagattgacaaggcacgtaaacttctctaggcagaggaaatagaatgtgcaaaggcacgtcgttacgaaacagcatagtacagaggaaaactgcaagggtctgaactgaaatgaagacaagaggagcaccaggaatgaggcggagcggcacgtgggagcaaagcacacaggtgtccctgaccacacgcctccttc

SEQ ID NO: 224 - DNA, 401 bp, Homo sapiens
caggcagcacccttgaattgcttaagctccatcagagaagagatatgcgtgtctcctgtgcccttcgcccctgggttctctcatgataaaggatagaccaagagcgtgaccttctgggtatgatgggtgcttcccccagccccggactccgtctcaacatgggtccaggcacccaaggcacagctctgagaggaacaaaarcttggggagctgtcctaagtatccctcacccctcgcttcttccccagagtcctggggagacatcatttgtgacatctgttgcctggtgttcttgccttaggttcacaggcatgtgagtttcctctctactggatgagaccggtggggctgtgggctgcctgccccagcccttctccctctccatctctgtggctggagcc

SEQ ID NO: 225 - DNA, 401 bp, Homo sapiens
ctgattatatttcatccgtgagaggtggtctcacggtgatgaagggaggaattcatgcaacattagctcacactgtatcaaaccagaaggatgatacggtttggctgtgtccccacccaaatctcatctttaattgtaacttccacaattcccgtgtgttgcgggaggaacctggtaggaggtgattgaattatgggggcsgttctttcctgccctgttctcatgatagtgaatgaatctcacaagatctgatggtttttaaaatgggagtttccctgcacaagctctctttgcctgctgcaatccacataagatgtgacttgctcctccttgccttccaccatgattgtgaggcttccccagctacgtggaactgtaagtccattaaacctctttccttt

SEQ ID NO: 226 - DNA, 401 bp, Homo sapiens; identical to SEQ ID NO: 190

SEQ ID NO: 227 - DNA, 401 bp, Homo sapiens
ctcttttcctcactccctggttttggggctggagggctgttcctggtagaaaggttttttaggaggctggagaacaagagaaagaagttgggagaaagcagggaactccctgtggtggacactcatctctcatctgccctgcactttccttctcctgggagcagtcaccaccctcttgtggagctgctcctacacacagakgtgtggtccaatgggcgcctccatctttttttttaaatggagacaggttctcactatgttgcccaggctggtcttgaactcctggggagactccatctttcgtgatccttccttcctgtcccagtgatgggttgtgaggctggcaatctgacctgacccagactagtcacattcttcccaaaaattgttctaaattgagc

SEQ ID NO: 228 - DNA, 401 bp, Homo sapiens
gcactttccttctcctgggagcagtcaccaccctcttgtggagctgctcctacacacagaggtgtggtccaatgggcgcctccatctttttttttaaatggagacaggttctcactatgttgcccaggctggtcttgaactcctggggagactccatctttcgtgatccttccttcctgtcccagtgatgggttgtgaggytggcaatctgacctgacccagactagtcacattcttcccaaaaattgttctaaattgagctaaggagagaaggcctctttccttttcgggtgtaaggctgaaaggacagactgtagacctccatgctgctagacatcctgccttgtggaaaaaggtggtatacagaaaggtggtatactgaaaatgacaggccaaaagac

SEQ ID NO: 229 - DNA, 401 bp, Homo sapiens
caaaactacaaatctccaattctctaaggtgaggtgggggtcggggagggaacaggcaggtgtctggcaggagtggctggcctccaagggtccctccagctctgactagctttgtgctctaagccaggccctggtaggacccctgttcccttgctgcaagcctctccttgctcccacggctgttcacggccctgactgctsaagatgacattttctaaacactggagacagagataagaagcagacggggagggagaaagcccgagaactggagctgagcccagagaggaaagtgggctccttgctgagagcaggcggaggcatgcagaccctgccgccaatgaacacgcaggcagggacctctcgccattgctttgacacagtgctgagtcagggtccaa

SEQ ID NO: 230 - DNA, 401 bp, Homo sapiens
cagacagtggtcctgagccgggacagcaggatgacaaaggctcacatttgcactctggagcaaaaacatcaccggatggaaattgaagtagagagaaaaagaatgagtataggggatgaggaatctctgatgttaactcaggggtaccatcagtcccctaaagctgcaaataatgtgaatcaatccctctctctctctctstctctcactcacacacacacacacacacacacacacacacacacacacacacacacacacttatcttgagagccttccaatttttttctgcaatgcacggacctcacaaaaaaatgactcagccttacataagagacgggaatttgagggggaccaggagtgtgatgactcattgccttgaaatttccagtgggatacac

SEQ ID NO: 231 - DNA, 401 bp, Homo sapiens
ggtccctagatgaggtctgacacgtggctttgtggcactaggtaaatatctatagaaaaaataacttgctgtattagtcttcctggaatgagctggcatttgagctgggaccagtttcctggagaagggatttcaggaaaagcaaccaggtatccctcaaagcaaacacagagaggcttgattaggtacagatggcttgcrtgggggaatttgggtgagctgtgctcttaaacctgtctgggtgttttcggggaagccatataacagtaacaacaacaataaaaccagcgctacctttatgcagagagggaggggctcttctgacagggcctcctgagagtggtctcaaggtcacttccgggcttccttggatccctgaaaaagaaccagaccagcctggc

SEQ ID NO: 232 - DNA, 401 bp, Homo sapiens; identical to SEQ ID NO: 188

SEQ ID NO: 233 - DNA, 401 bp, Homo sapiens
ggatggcctgacagtggggccctggggcccatctgctggaggaaaaagtcagtgagggacagaacagcacagctgccagtctggctagacctcctctctgaactggaactcctccggagatgagtatgggtgtgatgggcctgcggccgaggatgttgaggggtctccaccaactctcctttcctgatcaggttaatactygaaaacaactttcatctctgcttggcctcagcccagtcttttccttctgccctcccagcatgagtcctggagcctgaaagagaccacggttgggagaagatgtaagtaaggaactactatggcctccagacctggtctccttccaaatctactgtctcaggctgttccctctggcccatcttccttatctaataggtcta

SEQ ID NO: 234 - DNA, 401 bp, Homo sapiens
cgcccacaaacaccagtgccaccctctagccttgagggaccatatatgtctctgtgaatctccgttgcattggacagcttctctcccaagagctgctcttataaaagagacacaccacattttaatgaggcttcgctctccacagaggcctgcttccatcctaattgagcaagtagactttagtaactttggaactcactyaccccgtgactaatacaccctgtctacccagcgcattccctgtgagatttgttcatgttgttctccccctccttaagcattgtgaacctaggaaaaatatcatctctgtagggcaataaaccatgtgatttgtgaaatcatactttgagcatctcctaattttaaccatacacaatgcctgagaactgggaaatgaagaa

SEQ ID NO: 235 - DNA, 401 bp, Homo sapiens; identical to SEQ ID NO: 193

SEQ ID NO: 236 - DNA, 401 bp, Homo sapiens; identical to SEQ ID NO: 194

SEQ ID NO: 237 - DNA, 401 bp, Homo sapiens
gcaagcaagaaagaatgaacaaacttgggaccccaattcactctgccaaagaaaagaaaaacttgggaccccaattcaccctgctaaaagaaaaacatattaagctgaaagctgagtcatagaagaagctgccttctttttttttttcctgagcagatagctacagataaaaagtaaaatatctccacagtagctactctrtgttcaccttatctaatggtgagcttgagaggaatacataattgaccattcccctacctactccttttctcttgcaacacatggattcagttatgtgacctcttttcccttgagcctgcttttctcctttaaatattgaggccctcagaatcatccttggagaaaggcaccgacctgtctcctaggcatgtgtccttaac

SEQ ID NO: 238 - DNA, 401 bp, Homo sapiens
taacatggtgaaaccccgtctctactaaaaatacaaaaattagccgggcatggtggtgggtgcctgtaatcccagctacttgggaggctgaggcaggagaatcgcttgaacccgggaggcggaggttgcagtgagcggagatagcgccattgcactccagcatgggcaatagagtaagactctgtctgaaaaaaaaaaaaragaataaaaagagaaagaaggaaagcccttacttccaccccctaaacacacatgcgcacattccacgcacacatatatgtgtgtacatgctcggaagtgaagtggtttttgtaggatttcgtggctataacaccgtcgtttatgttctgggaggctggggtgagtcagcaagtattctgaaagggaaagacatggtgggg

SEQ ID NO: 239 - DNA, 401 bp, Homo sapiens
tgcgtctatttgattcttttcttctttattagtcttgctagcggtctatcaattttgttgatcttttcaaaaaaccagctcctggattcattaatcttttgaagggttttttgtgtctctatttccttcagttctgctctgattttagttatttcttgccttctgctagcttttgaatgtgtttgctcttgcttttctagytcttttaattgtgatgttagggtgtcaattttagatctttcctgctttctcttgtgggcatttagtgctataaatttccctctacacactgctttgaatgtgtcccagagattctggtatgttatgtctttgttctcgttggtttcaaagaacatctttatttctgccttcatttcattatttacccagtagtcattcag

SEQ ID NO: 240 - DNA, 401 bp, Homo sapiens
aatttttcacaatgtttgaataaaaactaatatggctaaatctctaaacatttccaaattcttgtcttagggcaaattgccagaaacagaattgctgggttacaaggtatgcgtgcttcttaaagatttaatgtctattgctaaattcacataaaatttaaattatgtgttacgcttttttgtctctggagataattaatyacattcagagtcaggaatcctgagtgtgatactcccttataataagatatggctgggtgcatttatacacatacacttttatccgtgaactgttttgatattgtgctgttttgtgataactcagaagaattgcagtacacatttatgtggctatctttctgctttcctcccagggaatgttgttagagaaagaaaacaaa

SEQ ID NO: 241 - DNA, 401 bp, Homo sapiens
agcagagcaatataagcatgctgtgaaaagtctaaaatggtgatgaaagcaaaggagtaatgtttctgtgccattatgtctgagagtactgtggtggttccttgaaaagttgctgcttatattcataattgcaaaaatgtggaaccagcccaaatgtccgccaatcaatgagtggataaagaaaatgtgatggccaggtgyggtggctcacgcctgtaatctcagcactttgggaggccaaggcgggcagatcacgaggtcaggagttcgagaacagcctgaccaacatggtgaaacctcatctctgctaaaaatacaaaaattagccaagcgtggtggtgtgcacctgtaatcccagctactgaggaggctgaagcaggagaattgcttgaatccaggag

SEQ ID NO: 242 - DNA, 401 bp, Homo sapiens
gtcaggaaacaacagatgctggagaggctatggagaaataggaacacttttaccctgttggtgagagtgtaaatagttcaaccattatggaagacagtgtggcaattcctcaaggatctagaaccagaaataccatttgacccagctgtcccatgactgggtacatacccaaagggttataaatcattctgctataaagayccatgcacatgtttgtttattgcagcactatttacaatagcaaagacttggaaccagcccaaatgcccatcaatgatagactggataaagaaaatgtgacacatatacactatggaatactatgcagccataaaaaagaatgagttcatgtcctttgtagggacatggatgaagctggaagctatcattctcagcaaact

SEQ ID NO: 243 - DNA, 401 bp, Homo sapiens
ctacaaagaatctgttcttggctgcgtttcaggccaagattcagtgctcacacctgtaatcccaacactttgggaggctgaggtgggtggattgcttgagtccaggagtttgagagcagcttgggcaccatggcaaaaccttgtccctacaaaaaatacaaaaaaattagctgggcatgatggtgtgcacctgtagtcccrgatacttgggaggctgaggtgggaggatcatctgagactgggaagttgaggttgcagcgagccctgatgatgccattgtacaccagcctgggtgacagagtgagatcctgtctccaaaacaaacaaacaaacaacaaagaaacaaatgcctgttctattttaatgttaatgctggtcagctgtgtctaaactctgaaagg

SEQ ID NO: 244 - DNA, 401 bp, Homo sapiens
gcggtaatcagtaaggccagcaatcatccttgaaaacagagcaacacagggcagtgcagacatcacatcatgagtattaaaaataatctctgtatgctttggggaccaaaaaatacacgaggagcagttgatcactttgacagatgtaacagtcctctctctctcctctctctcttttttttttttttttttgagacagartctcactctgtcactcaagctggagtacagtggtgcaatgttggctcactgcaacctctgcctcctgagttcaagcgattctcgttgcctcggcctcctgagtagctgggattacaggtgcgtgccaccacattaggctaattttagtatttttagtagagacgggattttgccatgttggccaggctgatctcaaactc

SEQ ID NO: 245 - DNA, 401 bp, Homo sapiens
tgtcttcatttgttcccggaatccccccttctgaatagatgctgctgactgtcctcgttccacccttctttcacatagttagctgtggaagtcttactcatctgcaaggactaggtcaagcctgattcattgaagaccactcttctcaacttctttgtgcagaagggccaatttccttttctctcttccttccttttcccytttttttttttttttttgaaaatttccaatctgctcacagacatacaaagcacaaagcacaagttgtaattttttattatgagattcagcaggtagaatttctactttgtcaattgctgtaagagtttctaaataaatactctcaatttctgaacttatttcatatggagtaataatgagagctggctcttggatcatat

SEQ ID NO: 246 - DNA, 401 bp, Homo sapiens
aattgaggggttcaattaagggatccccttaatttccatggccatatgattcagaagtgtcaacaatggaataatctactatgtatatagtttgtagaaaatagttagaatgcacatattttttcatcttttttttcccaacctgcctaggtgaaaatctaacttggacatacttctttatagcaacatctctatagatayaaatgacaatttagatccaatttatcacttactcatgttaactaatgttcactccagtcagctatattgaaaatattaaaataattaaacaaatttctgggagtttgtgataatgggatttgactggttcttcctttggtattttctgtgatgtttctccttcattttattgccaaagtcagagggattgttatagtata

SEQ ID NO: 247 - DNA, 401 bp, Homo sapiens
ctatgtgaaaattacttgaataatgatagtgaatacttttatgatactatggtttacatgctgttcttacatggcattaatttattttgcctctatgaatactatcggttatcacctcctgcagatgaggaaacaaaggtatggttgtctttgacttggttttagcgatggaattataggtttcaaaaaatagcatgggartgcagagggaaatctaggaggcaggatctgagttgtgaacattaggaagttgctagacaaatgctacggtttgaacgtgtcccccaaaagttcatgtgttggaaatgtaatctcaatgcaacagtcttggaaggtggggcctcacaggaggtaattaggtcaggagggctctatcctcatgaatggcttaatattgttat

SEQ ID NO: 248 - DNA, 401 bp, Homo sapiens
gtgtgtacagctctattatcatacactgcattacaggtgggtgtgagttccacctccagcctgtgagatgcctgcagggagccatcctgagtcctccgaattgacacagtgtctttggggtaggaatttcccagtgaatgcttacttaaatgaattcagtaagtgcgttttgatttttccatagattgacacagtggaacrgtggctaagagatgattctttactataagcacccttcataggcaaagtgggaatgtgcttattgatgaggtcaaataattcagactgcggttcccacattggtcttgagtagtggatggctccactaaaacactaggtttcatgggtttcacctgttttctggtagttaatttaaggagatggtgcactaagacaactta

SEQ ID NO: 249 - DNA, 401 bp, Homo sapiens
gagtcctccgaattgacacagtgtctttggggtaggaatttcccagtgaatgcttacttaaatgaattcagtaagtgcgttttgatttttccatagattgacacagtggaacagtggctaagagatgattctttactataagcacccttcataggcaaagtgggaatgtgcttattgatgaggtcaaataattcagactgyggttcccacattggtcttgagtagtggatggctccactaaaacactaggtttcatgggtttcacctgttttctggtagttaatttaaggagatggtgcactaagacaacttagatggaaaaaataaatagcagcataaaaagcaaactgtgattgtatagaactgtgagataatattcaacttcgtttcatttatttatt

SEQ ID NO: 250 - DNA, 401 bp, Homo sapiens
taatttaaggagatggtgcactaagacaacttagatggaaaaaataaatagcagcataaaaagcaaactgtgattgtatagaactgtgagataatattcaacttcgtttcatttatttatttatttatttagtgacagagcgtcgctctattgcccaggctggagtgcagtggcacgatctcactgcaacctccgcctccyaggttcaagtgatcctcctgcctcatcctcctgagtagttgggattacaggcacctgccaccactccctgctaatttttgtatttttaatagagatggggtttcaccatgttggccaggctgctcttgaactcctgacctcaagtgatctgcccaccttgtcctcccaaagtgctgggattacaggtatgagccactgca

SEQ ID NO: 251 - DNA, 401 bp, Homo sapiens
tttatttagtgacagagcgtcgctctattgcccaggctggagtgcagtggcacgatctcactgcaacctccgcctcctaggttcaagtgatcctcctgcctcatcctcctgagtagttgggattacaggcacctgccaccactccctgctaatttttgtatttttaatagagatggggtttcaccatgttggccaggctgstcttgaactcctgacctcaagtgatctgcccaccttgtcctcccaaagtgctgggattacaggtatgagccactgcacccggcctcaacttgattttaatgtgtgggtgagaaatatatgttctcatgtactcagttttggtttaagtgtataaatggacttacacgtgacacaggttggaaagaagcagaacactttag

SEQ ID NO: 252 - DNA, 401 bp, Homo sapiens
ccgggcatggtggtgggtgcctgtaatcccagctacttgggaggctgaggcaggagaatcgcttgaacccgggaggcggaggttgcagtgagcggagatagcgccattgcactccagcatgggcaatagagtaagactctgtctgaaaaaaaaaaaagagaataaaaagagaaagaaggaaagcccttacttccacccccyaaacacacatgcgcacattccacgcacacatatatgtgtgtacatgctcggaagtgaagtggtttttgtaggatttcgtggctataacaccgtcgtttatgttctgggaggctggggtgagtcagcaagtattctgaaagggaaagacatggtggggaggataaaacggctcttcattttaagaaatccctcttatcaag

SEQ ID NO: 253 - DNA, 401 bp, Homo sapiens
gctctatagaattttgaattccagttttatttctgagagacagaggcacagaagcaagagtcactcagttactacacgggaaacgtggattttaatctgggttaagtagcattctagttgaagagtatacagtggcaagtgtgagacaacctgagcatttaaaaatgactatgaatatataaaactccgcaagtacataaytagaaaagagaatgtcttccaaaacaaaactcatttgccatcactggaggtgattattacatgaattggttattctgagaattgagaattaaaaataattaaagatttaatgtctttctagaagatactgaatttcagcataactaaatagttctacttgatgaaggaaaacttttctttattgaagaatgtcagctgtg

SEQ ID NO: 254 - DNA, 401 bp, Homo sapiens
tcaatagatttttattcaaggaattccatgttgtaagttcttccactgtccatcgaggtcactttagatcctctaaagagctggagtcaaaatttatcttcaaaagatttatcttcaagttagccctttttaatgaaactgatgcttattttaatgcagttgtcctgtcaggccataattctttgatttttggcatctgtygtcatctccttttaatatggctatactgatgaagatttcaaaatttacctttggaaaaatcaagaatctttaagatctaatttcttcaaccaatttactttagggtcatttttagtgtaggtggatctgcctagttctcaatgtgacaccctctctaaacctgagtgagttcaaatcatattcattcctaagcgatcaca

SEQ ID NO: 255 - DNA, 401 bp, Homo sapiens
ctaaagagctggagtcaaaatttatcttcaaaagatttatcttcaagttagccctttttaatgaaactgatgcttattttaatgcagttgtcctgtcaggccataattctttgatttttggcatctgtcgtcatctccttttaatatggctatactgatgaagatttcaaaatttacctttggaaaaatcaagaatctttragatctaatttcttcaaccaatttactttagggtcatttttagtgtaggtggatctgcctagttctcaatgtgacaccctctctaaacctgagtgagttcaaatcatattcattcctaagcgatcacactcaagaatagtgcagagtgtggaatatgccaatacataaggtaaaaaagtaaattatcaggtctttcatat

SEQ ID NO: 256 - DNA, 401 bp, Homo sapiens
cattagtttcatgggaaattgtcaggtggcagagctttccttttgcactactcctaggactataaaaagcacaccacaagcatatttcagcaaagctaaaattaaaagtttaattaacaaaaatgctacttaataatacatttggatcgatttggctatgtcacatagtaactgccagtcatatctgaaaaaacagaaacracaaatgacaaaatgatataaacctctcaaatcatcttgtggatggatcactttgtggtccaggagtcacagttccacagctctcactgtacttgcttgagtgtgttgatctaagacccctttggcacctctgcatcaaagtgcctgttccaccccttcctgcagccttcacatgcctccgatgctggagggcatgcagg

SEQ ID NO: 257 - DNA, 401 bp, Homo sapiens
tggatgacaagagtgaaactccgtctcaaaaaaaaaaaaaaagaattaacgacattttaagagatgaaaatggatgctaagaagaaagattaattcatcctttctagttaggagagaagaaggctgagctgtaacatataatgaagctttatgtaaggcatttgatacaggtttattggataaataaatgaatttaatagycatctaattatgttaccaaaaattgaaaaatagataaggctacgtaaatcttattttgcctagaacacattaacatctttatgtctgtctacattgttttcaatgacaaagtcaaagtacaataatataggaccataacagttcaaaccctcatgcattttaaaaggaaaattaatccatacgatctaagtttatttgca

SEQ ID NO: 258 - DNA, 401 bp, Homo sapiens
tctggaagggtttttctgatgttatcttctagaatttttatagtttcaggtcttagatttaaatctctgatccatcttgaattgacttttgaataaggtcagagacgaagatccagtttcattcttctacatgtggcttgccaattatctcagcaccatttgttgaatatggtatcctttccccacttttatgtttttgtytgctttgtcaaagatcagttggctgtgagtattttgctttatttctgggttctctattctgttccattggcttatgtgcctgtttttataccagtaccatgctgttttggtgactatggcattgtagtgtagtttgaagtcaggtaatgtgatgcctccagatttgttctttttgcttagtcttgctttggctatgtggg

SEQ ID NO: 259 - DNA, 401 bp, Homo sapiens
aaggtcagagacgaagatccagtttcattcttctacatgtggcttgccaattatctcagcaccatttgttgaatatggtatcctttccccacttttatgtttttgtttgctttgtcaaagatcagttggctgtgagtattttgctttatttctgggttctctattctgttccattggcttatgtgcctgtttttataccastaccatgctgttttggtgactatggcattgtagtgtagtttgaagtcaggtaatgtgatgcctccagatttgttctttttgcttagtcttgctttggctatgtgggctcttttttggttccctatgaattttaggattttttttttctctagttctgtgaagaatgatggtggtattttaatgaaaattgcattgaattt

SEQ ID NO: 260 - DNA, 401 bp, Homo sapiens
ctatattttcctagtttaatctaggaagattgtatatttccaggaatttatccatcttctctaggttttctagtttaggcacttataggtgttcatagtagccttgaatcgtgtatttctgtgctattagttgtgatatctcctgtttcatttctaatttagcttatttggatctttttctcttcttggttaatctcgcamatggtctatcaattttatttatctttttaaaaaactaggtttttgtttaatttaccttttgtctttttttgtgtgtttgtttcaatttcatttagttctgctgtgatcttcgttatttcctttattctgctggtttgggtttggattgttcttgtttctccagttctgtgaggtgtgaccttagattgtctatttgtgct

SEQ ID NO: 261 - DNA, 401 bp, Homo sapiens
ctttgtcttttttaactgctgttgctttaaagtttgttttgtctgatgtgaaactaactactcccgcttgctcttggtgtccatttgcatggcatatctttttacacccctttaccttaagtttatgtgaatctttaggtgttaggtgagtcttctgaagacagcagaaacttggttggtgaatccttatccattctgccrttctgtatcttttaagtggagcatttaggccacttacattcaacgtttagtactgggatgtgaggtactattctatttatcgtgttatttgttgcctgaataccttgttgctttttcattgtgttattgttatataggtcctataaaatttatgctttaaggaggttctattttggtgtatttgaggatctgtttcaaga

SEQ ID NO: 262 - DNA, 401 bp, Homo sapiens
cttgatactttccttcagcttgactttagataacctgatgactacgtgcctgggtgatgatctttttgcaataaatttcccaggtgttctttgggcttcttgtatttggatgtctagatttctagcaaggccggggaagttttccttgattattccctcaaatatgttttccaaatttttggatttctcttcttctttggsaacatcaattattcttaggtttggtcatttaacataatcccaaacttcttggaggctttgctcattttttgaaagtctttttaatttgtctttgacagactgggttaatttgagagccttgtcttcaagctttgaagttcttttttctggttttttgattccattgctgagactttccagtgcattttgcatttctctaa

SEQ ID NO: 263 - DNA, 401 bp, Homo sapiens
gcttgactttagataacctgatgactacgtgcctgggtgatgatctttttgcaataaatttcccaggtgttctttgggcttcttgtatttggatgtctagatttctagcaaggccggggaagttttccttgattattccctcaaatatgttttccaaatttttggatttctcttcttctttgggaacatcaattattcttrggtttggtcatttaacataatcccaaacttcttggaggctttgctcattttttgaaagtctttttaatttgtctttgacagactgggttaatttgagagccttgtcttcaagctttgaagttcttttttctggttttttgattccattgctgagactttccagtgcattttgcatttctctaagtgtgtccttgatttcc

SEQ ID NO: 264 - DNA, 401 bp, Homo sapiens
caaggatgctaggatattgtgaggttagctacaccaagccccgcaaacacacacacacacgtacacacacacacaatgggagtgatggatccagacaaggatgctaggatattgtgaggttagctacaccaagccccgcaaacacacacacacacgtacacacacacacaatgggaatgatggatccagacaaggatgctrggatattgtgaggttagctacaccaagccccgcaaacacacacacacacgtacacacacacacaatgggaatgatggatccagacaaggatgctaggatattgtgaggttagctacaccaagccccacaaacacacacacacacgtacacacacacacaatgggaatgatggatccagacaatgttatgaggaaaggaaa

SEQ ID NO: 265 - DNA, 401 bp, Homo sapiens
tgttatgtaggtaaactcctgtcacacgggtttgttgtatggtcccaatgctaagatactaagcctagtactcaatagttatttttttctgcttttctccctcctcccgtcctccaccctcaaataggcctcagtgtctgctgttctcttctttgtgtctatgtgttctcatcatttagcttctgcttataagtgagaacwtgcagtatttggttttctgttcctgtgttagtttgctaaggataatggcctccagctccacccatgttcctgcaaaggacgtgatcttgtgctttcttatggctgggtagtagtccatgttgtatatgtaccacactttctttatctggtctatcattggtgggcatttaggttgatttcatttcttttaatttgcccaa

SEQ ID NO: 266 - DNA, 401 bp, Homo sapiens
ttcggctgtgaatccatctggtccgggacttttttggttggtaagctattgattattgcctcaatttcagagcctgttattggtctattcagagattcaacttttttctggtttagtcttgggaggatgtatgtgtcgaggaatttatccatttcttctagattttctagtttatttgcgtagagctgtttatagtattcrctgatgttaatttgtatttctgtaggatcggtggtgatatctcctttatcattttttattgcgtctatttgattcttttcttctttattagtcttgctagcggtctatcaattttgttgatcttttcaaaaaaccagctcctggattcattaatcttttgaagggttttttgtgtctctatttccttcagttctgctctg

SEQ ID NO: 267 - DNA, 401 bp, Homo sapiens
tcctggacaatatcctgcagagtgtttaccaacttggttccattctcctcgtcactttcaggtacactaatcagacgtagattttgtcttttcacatagtcccatatttcttggaggcttttttcgtttctttttattctttttttctctaaacttctctgcttgcttcatttcattcatttcatcttccatcactgatamcctttcttccagttgatcgcattggctactgaggcttctgcattcgtcacatagctctcgtgccttggttttcagctccatcaggtcctttaaggacttctctgcattggttattctagttatccatttgtctaattttttttcaaagcttttaacttctttgctattggttcgaatttcctcctgtagctaggagtagt

SEQ ID NO: 268 - DNA, 401 bp, Homo sapiens
tgaagtaagcgctttctcctgttctctagtgtattcccagcaaaactttgagcatactttaaaaatgaatttattgcacatacttccatatccagggagagctctgattcctcctctcatacactccaagaggaagggtcaaggaagtattgaactcaggaactttttttagggtggtggtgttggtgagaatctcaatawgaagaaggaagatttcatgtacgtccgtgtgaagagaccaccaaacaggatttgtgtgagcaacatggctgtttgtttcacctgggtgcaggcgggctgagtccgaaaagagagtgagcgaagggagatggggtggggccgttttataagatttgggcaggtaaaggaaaattacagtcaaagggggtttgtcctctggc

SEQ ID NO: 269 - DNA, 401 bp, Homo sapiens
gggctaggctaaaacagtaaggtcaagttgtttggacagaaaggttacagggtgtggtcctggctcttgtgtaagaattctgactgcactaaccatgcctaggaaggaaaggagttgttgttttgtaagggattgaggtttgggagattaattggacacgatcagcagggaaagcatgtgtgtttttatgagaattatgcygagataggtaacagatgaggatgaaatttgggcctcactgaagtaatgggggctgtctgtgaagccttgtggcagtacagcccaggtaatttgctgagcttgatgggtgtcagggtagtctaagtgaaagcaaagagaggctgggatgaagggtgcaaaggaatagtaaagaaagcatgtttgagatccagaacagaata

SEQ ID NO: 270 - DNA, 401 bp, Homo sapiens
aggacaggtaaaatgggggaattgtaaggagagtttataggctttaaaaggccatgccgtagcaggcgagtgataacaggctttaatccttttaaagcgtgctgtgggatgggatcttggctttgagcagggtgagggtgattaggttttaatgagatggtaaggggtgcatgatgggtcaccaaggagggagtagaggtrtcttatacttgtgggttaaggtcggggggataaaagaggacacaaaggaggctttgggttggggagaagggtggcaatgagatgcagctgtagtccaggaacagtcagggaagcagataatttagttaaagtgtctcagcctaataagggaactgggcaggtggggataattaaaaaggagtgcttaaaagagtgttgtc

SEQ ID NO: 271 - DNA, 401 bp, Homo sapiens
agcaggcgagtgataacaggctttaatccttttaaagcgtgctgtgggatgggatcttggctttgagcagggtgagggtgattaggttttaatgagatggtaaggggtgcatgatgggtcaccaaggagggagtagaggtgtcttatacttgtgggttaaggtcggggggataaaagaggacacaaaggaggctttgggtwggggagaagggtggcaatgagatgcagctgtagtccaggaacagtcagggaagcagataatttagttaaagtgtctcagcctaataagggaactgggcaggtggggataattaaaaaggagtgcttaaaagagtgttgtctaagttggcacgagagttggggagttttaagaggtttagaagcctggctgtcaatacgca

SEQ ID NO: 272 - DNA, 401 bp, Homo sapiens
gtgattaggttttaatgagatggtaaggggtgcatgatgggtcaccaaggagggagtagaggtgtcttatacttgtgggttaaggtcggggggataaaagaggacacaaaggaggctttgggttggggagaagggtggcaatgagatgcagctgtagtccaggaacagtcagggaagcagataatttagttaaagtgtctyagcctaataagggaactgggcaggtggggataattaaaaaggagtgcttaaaagagtgttgtctaagttggcacgagagttggggagttttaagaggtttagaagcctggctgtcaatacgcacaacagttatggaggcaagggaaagagacccttgaaaagaaggtaatgtggagtgggtagcctccatattgattaag

SEQ ID NO: 273 - DNA, 401 bp, Homo sapiens
tgattaggttttaatgagatggtaaggggtgcatgatgggtcaccaaggagggagtagaggtgtcttatacttgtgggttaaggtcggggggataaaagaggacacaaaggaggctttgggttggggagaagggtggcaatgagatgcagctgtagtccaggaacagtcagggaagcagataatttagttaaagtgtctcrgcctaataagggaactgggcaggtggggataattaaaaaggagtgcttaaaagagtgttgtctaagttggcacgagagttggggagttttaagaggtttagaagcctggctgtcaatacgcacaacagttatggaggcaagggaaagagacccttgaaaagaaggtaatgtggagtgggtagcctccatattgattaaga

SEQ ID NO: 274 - DNA, 401 bp, Homo sapiens
cagctgtagtccaggaacagtcagggaagcagataatttagttaaagtgtctcagcctaataagggaactgggcaggtggggataattaaaaaggagtgcttaaaagagtgttgtctaagttggcacgagagttggggagttttaagaggtttagaagcctggctgtcaatacgcacaacagttatggaggcaagggaaasagacccttgaaaagaaggtaatgtggagtgggtagcctccatattgattaagaaggggatgggcttaccttccactgtgagagttacctaaagcttggcatccgtgatggtctacagggcttctgaggtgatcgggcagcatcagtcttcagccgctaagccgagaaggagtcagagagccttgggccagagttccaggg

SEQ ID NO: 275 - DNA, 401 bp, Homo sapiens
ctgtagtccaggaacagtcagggaagcagataatttagttaaagtgtctcagcctaataagggaactgggcaggtggggataattaaaaaggagtgcttaaaagagtgttgtctaagttggcacgagagttggggagttttaagaggtttagaagcctggctgtcaatacgcacaacagttatggaggcaagggaaagagrcccttgaaaagaaggtaatgtggagtgggtagcctccatattgattaagaaggggatgggcttaccttccactgtgagagttacctaaagcttggcatccgtgatggtctacagggcttctgaggtgatcgggcagcatcagtcttcagccgctaagccgagaaggagtcagagagccttgggccagagttccaggggct

SEQ ID NO: 276 - DNA, 401 bp, Homo sapiens
accttccactgtgagagttacctaaagcttggcatccgtgatggtctacagggcttctgaggtgatcgggcagcatcagtcttcagccgctaagccgagaaggagtcagagagccttgggccagagttccaggggctctgggagtggctgccaggtgagttgaacagtccgatttccagtggggtcctacacagatgggaygcggcttaggaggaatcctgggctgagggcattccttggcctggtggccagatttctggcacttgtagcaagctcctgggggaggaggttctggaggaacgcctggccagtgcggttcaggcgtttggaaattcttgtgtgctggagatgtggctggggtttgtctcacagtggaggcaaggaattgcaacttttttcta

SEQ ID NO: 277 - DNA, 401 bp, Homo sapiens
gcgtggaaataagggattgggggttcttgtcccctagaaaagcgggacttgccgctaagggtgaaggagaaggggttgaggggtagtgagggaagttggagaagagagtaaaaagaggccacttaccggatttgaaattggtgagacgtttcttgggctggtcggtctgaggacttgaggtcgtaggtggatctttctcayggagcaaagagcaggaggacaggggattgatctcccaagggaggttccctgacccgagtcatggcaccaaatttcatgcgcgtccatgtgaagagaccaccaaacaggctttgtgtgagcaacatggctgtttatttcacctgggtgcaggcgggctgagtccgaaaagagagtcagcaaagggagataggggtggggcc

SEQ ID NO: 278 - DNA, 401 bp, Homo sapiens
catcagttaaggtggggcagggcattttcacttcttttgtgattcttcagttacttcaggccatctgggcggtatatgtgcaagtcacaggggatgcagtggcttggcttgggctcagaggccctgacataaggtaaaagaattgcttcagtttttgtggcctctggtctatgtttgggcttattgttttaggttctttcytcattcattcattcattcattcattcgtttcaacaaatttttctggaaggcttactttgtgtcaagtattgtctaggccctgggaatagcatagagataattcagacagtgctccatgtccctatcttatatttcagtagactgtctctcttaggcctagatggtacaatgagattaccttgggcttgcctacctcttgg

SEQ ID NO: 279 - DNA, 401 bp, Homo sapiens
tttttttttcccccatcacaatagaggctactgatttacttatgggaatggggaaaggtgagattccaggatttggagttgtttgttaagaggcacgtgtgtcctaatcttcatgattacgaaaatgttggtcagattcaggggacataccacggggtgagagtcaaagggcaaatccagggccttatttttctctctctkccacatctcaccggatctcgtctcacctgccatactcagctgggaagttgttagaactcagaacactccaaagggaacataaggtcttaacttgttttctgttatctcgtcccccaccttattctcaacctgaaaagaacctattttttattccccaagttttaggatgcctgcattttgtgttgtttttgtgtacattt

SEQ ID NO: 280 - DNA, 401 bp, Homo sapiens
tcagatgtgtgtgcacgtgtatttatcaaccaaaggatacttttttggtgaggagtcggaaatactctccagttagaggcagactcaacttgattatgtttccagttcttgagcagggctgccgcacacagttgtgcaggttgcttactgcatgaggcttcctggccaggggacaggccagggctgtaacccaaccccccytctactcactaagctgcacactttggttccagactatgtccacaggaagaaggggcacctttttctaatttcctccagtatgttagcagtgactgctgatgcaaacaacaagttagccacctaggaaagaggctaggtaacttccctttcatcctggcatcctggggtattcctcttttcatttttccccttctctacca

SEQ ID NO: 281 - DNA, 401 bp, Homo sapiens
tggtctccaacttcttacctcaagtgatctacccaccttggcctcccaaagtgctgggattacaggcctgagccaaggcacctggcccagcatctgcttgcttcttaaaatgcattaaatgcaggaacagatataaaaattaagacacaagaatggatttagtttaaaaagatgtgaaaaccagtagtgtaatataactcrtatataatctaagtgttggaaatagggaccccaagactcagatttgttccccaactctgactcttaatgtcccctaccatcttagcaagttggttgaccactggctttcattttccttgtctgtgagtccactctccgtaataccttcctccaaggatattgtgtgcctggaatgagataatggatgtgaaatactttaa

SEQ ID NO: 282 - DNA, 401 bp, Homo sapiens
agcttaaaatatagtaattttcttcttatcaactccgtgctttcaaattgtatcctgtatcaggagttgagtaactcaaataatctccagtaagatattcaaataagaatatcctaagtggaatgttaatataggcaaatgttaaaaactctggctgctttccagtaccgttgtctcttattaagtggttgatttgctcaygagagaatgtaagcaaagtgttaaatacacagggggagttctaaagtatccagacagtgtaggggttgcagaaaaaatgattacttggaaatttaactctgttgcaccttgggacttttctttgtatcactgcttttcttatttcaagccacactgggagtttcaaatcatatacacttaaaaaaaatgaaaaaaaccct

SEQ ID NO: 283 - DNA, 401 bp, Homo sapiens
tggatctatatgtatatatgtgtgagcatacatacatacacacacacacacacacacacacacacactaaacgatgcttactccttttgtcattgctctcataataagtcttgtgtttacagctctaacatggcatttattttatttcagtgactgtctacttggttcttcatggctagattctgagtccaggtgatcatrgatgggtttgtattgatctgaatatttcttgaggctagaactgcttagcacaattgccacttcatataaacttagttgatttagaatttacaataatttgagtgacaaacaaagaatttttagactctaagcaaaagtaaaagcaaaatcatattgaggctgggatgccatttggatagacaaaaaaccttttcaccact

SEQ ID NO: 284 - DNA, 401 bp, Homo sapiens
cttttcaccactgtatctcctgaccaagaagtgaagaagtgtccctggcatacttggataaatgactgaaatagatatatagaagcttttcagaaggattattcttcaactctcaatatgttaatttgagaacatgcatatctaattattagttattaaaacaggtcctttaaaattaaaaatactccaagtacttgaaartaacaaaactggagtaaaaaataatagagtagacatttgacagtttctccctctattgttctcagttcttgaacatggttaactaacatactcacacttcatctattggttgacattcaggaataaagtacttgcaatgtttagattcttaaaaatacattttatgtatcaaacctttctgaaaaccattgttaatggat

SEQ ID NO: 285 - DNA, 401 bp, Homo sapiens
attcttaaaaatacattttatgtatcaaacctttctgaaaaccattgttaatggataattatctaggatgcattgctcatcttcctgtcttattaaacagtctatcagatatgacctaaccttttcattaagactcagcaccatcactttattatttgtaaactgttgggtcacagaatggtatcctcaggtgtctgagcytataaaaggctgtggctgtttgcctgatttctcttttgacttttcctctgccatttcctatcatttgactggcttccctgggtctgcttcctacatacagtccattgtatgtttaatttcatgtcctccgttggctaaagacattctgttggtttcatgaatccttgaatatgtcaattagttctggcctttatggctcc

SEQ ID NO: 286 - DNA, 401 bp, Homo sapiens
cacagcactaatgaccacaactttacaagtaaagtatacatcttcaattttgaaattcaatttaaaattgtgctacaaatctggtagataaaatagtcacccagatatacatgaggttgtagtttacacataattactatattttcatttatttggcttgttagccaaaatgcaagtgaacaatcatagtctccactgtgyaaggggccattatttcagtagactccatgtgagaagggcctgctcatatattaataaatcaacaggttcaggtcaaggaggaatggccaaaccattttctcgctagattgcactttcattgaaaacctgctcattaacttatttataaaactgatatctctatatttcatattctgatatattagcatgatttaatgtta

SEQ ID NO: 287 - DNA, 401 bp, Homo sapiens
cactgatgctaaaactgggcctagacaggttacccaccttgtaagctgggaagctaaaatttgaatccaaagagtgtaactccaaaggctacttactattatgctagactgattctctaggcagagctctgttgtgatcaggggaatagcatctctttccattctagtttcaagcctaggaacacactgaataaatcaaaygtgttcaagaggacactgtgtccttcaggatgacctggggaaaaggagaatgtggtcaggtacatcgatggttgtccctttggagggacagaataggggtagagttagagagctggttttatttggcaattctacccatgaccacaaatgaagcctcaaagttcagttatagtttctcctattcagatcaagataaggac

SEQ ID NO: 288 - DNA, 401 bp, Homo sapiens
tcattcaaactctttgcttaaagatagcccaaggggagtctcagaagccacacaaaaattttctgtgacataccaggtttgatttttggtttagaaaatatatgcttagagtagagaaatagccttcacacattcttcttaagaatgatatcttcagtgttagttatgagaacacatttcattgcagcttcagatccagaracctctggccactcagttctcctgcagtcatggtacctggcaagcaggttgcagataattggcctgtctccctagggaatatgtgctttatttattggtaatatgaactcaaaataaaataaagaacaaagtttcaatggagccattcctctccccccccatgatgttgttaataataaatgaggcccagaatttggttc

SEQ ID NO: 289 - DNA, 401 bp, Homo sapiens
taccaggtttgatttttggtttagaaaatatatgcttagagtagagaaatagccttcacacattcttcttaagaatgatatcttcagtgttagttatgagaacacatttcattgcagcttcagatccagaaacctctggccactcagttctcctgcagtcatggtacctggcaagcaggttgcagataattggcctgtctscctagggaatatgtgctttatttattggtaatatgaactcaaaataaaataaagaacaaagtttcaatggagccattcctctccccccccatgatgttgttaataataaatgaggcccagaatttggttcaaaacagggtttctggaacctacactcctacagtcagtaagtggaccaggttctttttcagtgccagcat

SEQ ID NO: 290 - DNA, 401 bp, Homo sapiens
gcttccttccctcttttcctttctgtggacctcatctctcataactcctctctgttctccccaccttggcctcagagacaagaacacttattgctttgttttacggccatcatgaaaatagacattggctccagcatagtgtagtatacagaattttaatgagtacagagcaagcagttcagtccttcactccgatatgaraggggtgatgtcaacctatccaggaaatattgaattgattttaaaactgctgtaagggtgaataaatcggtggtgctgtgcattagcatgagactgcctgcacccacccatgtctccccgtttatttgtcttaatgctaggattattagaaaccagctgaataggtgcccttcaaaatacagatcgagggatcagagaat

SEQ ID NO: 291 - DNA, 401 bp, Homo sapiens
ttaagcatctttgtggtgtcaatccagttcatgtccaggccaattttcaaggttagttcctgacactgccctgattctactgagcatctgcctccttgtttgggaagaagtgggaaagtgatttgacacagtggcctaccagcgaatgtgcttgtcagtgtaacaggtggctgccctgtgaggttgctaatcagaacctgsacttttgtattttgtttagaaaagctaagtccttctagaggtccgtgtaatgcaaattcatccaaagaaggcaggctcaggaaggagggggggaagccaggtgcttgcttcattcaagtaacccaaccacagaatccaagttctgcaactctgatttgggcttagtaaaccagacccaccaatccaagagaataaagaat

SEQ ID NO: 292 - DNA, 401 bp, Homo sapiens
ctgagttgacttttcttttctttttttttttgagatggagcttcactctgtcacccaggctggagtgcagtggcgcaatctcagctcactgcagcctccgcctccggggttcaagagattctcttgcttcagcctcccaagtagctgggattacaagtgtccactaccatgcctggctaatttttgtatttttagcagagrcggggttttgccatgttggccaggctggtcttgaactcctgacctcaggtgatctgcccacctcggcctcccaaagtgctgggattataggtgtgagccactgtgcccagctgagttgacttttgttagtttgagttgctaagaaatacttagtatatacattcttactacatttttcctgatttgaaaaggaaaaaaaa

SEQ ID NO: 293 - DNA, 401 bp, Homo sapiens
tttttttttttttttgagatgaagtctcgctcttgttccgcaggctggaggactgcaatggtgcaatggtgcaatctcggctcactgcaacctccacctcctgggctcaagcaattctcttgcctcagcctcctgagtagctgggattacaggcatgcgccaccacgcccagctaattttgttacttttagcagagacgargtttcaccatgttgaccaggctggcctcgaactcctgacctcaggtgatccacccgcctcgacctcccaaagtgctgggattacaggcgtgagccaccgtgcccggctgcttaatctgttttatagaaacacactggagctcagataagtgaagtcatagtgcattgggaggaactgagaaatttcccaaggagtaagat

SEQ ID NO: 294 - DNA, 401 bp, Homo sapiens
aagcaattctcttgcctcagcctcctgagtagctgggattacaggcatgcgccaccacgcccagctaattttgttacttttagcagagacgaagtttcaccatgttgaccaggctggcctcgaactcctgacctcaggtgatccacccgcctcgacctcccaaagtgctgggattacaggcgtgagccaccgtgcccggcygcttaatctgttttatagaaacacactggagctcagataagtgaagtcatagtgcattgggaggaactgagaaatttcccaaggagtaagatgacaaatttctgacaggattatttcagtgtggcctcattcataccataagaagccattcctctgaattgtatgcaaaaagaaactagaaggcctagtttgttattaaa

SEQ ID NO: 295 - DNA, 401 bp, Homo sapiens
ctttttacctttttttctggctgaaaatataattatctgcattggcatcattcttcaaagcattagtttcatatggatttcagcacaagcttaaaagccaccaactattctctcctttgcaactatttcttggttagcagctgtagtgaaaaagttacaattcatgggggaattaagaagaaacttctatttggaaatacrcgagagcagaaagtcttgaagaaaactaattggtaatccagccaaagagggtaattcagagatagatctttaaagatatatatatgtatacacacacacacacacacacacacacacacatacacacacacacacacacagaattgagggaaatgactattcttatctgctctagtgttttttttttttaaggataacat

SEQ ID NO: 296 - DNA, 401 bp, Homo sapiens
gaagagaatagtttcagcatcattccgtttccccctgaatgtatctccagattcattttttccacaagagtaagaacaaggagctttgctagaatacaaggtacttttgccagggtaccagagtaaagacaacatggtctctgcccaccagaatttatgtagggacacaagaaattgagaaaatcattgcaacaatgtagyaatgggtagttttgtgtaccagggtgcaatcatggctcactgcagccttgaactcctgggctcaagcaatcctcccacctcagcctcctgagtagctgggaccaggggcacaaaccaccatgtccagctaattttatttatttttttttttgtagacacaaggtctcactacgttgtccaggctgctctagaactcttgg

SEQ ID NO: 297 - DNA, 401 bp, Homo sapiens
aagggctttggtgatacatggacatatgtatatttacataggaagaaactcatgacctttgctttaattctcaaatgggaactaacactaaaaatcgatgatttactgacataatgtgtgtagtgaagctgaggccacacctgggcccagggtaaccctttccaggtactgacataggaaggtttctgcccaaagccattrgtgactctcttctcatcttttaactgaggtagattgtactcttctgggagctaaaaggacttgactgagtcttttgaagaaagtaaattattccattgccttgcagaaatgttttatagagctgtccctacaactcagtgatttccaccagtgtgttgtgagcagttatatcaaagcccttcttgcatgtgttaaattca

SEQ ID NO: 298 - DNA, 401 bp, Homo sapiens
cagtgagcggagatagcgccattgcactccagcatgggcaatagagtaagactctgtctgaaaaaaaaaaaagagaataaaaagagaaagaaggaaagcccttacttccaccccctaaacacacatgcgcacattccacgcacacatatatgtgtgtacatgctcggaagtgaagtggtttttgtaggatttcgtggctayaacaccgtcgtttatgttctgggaggctggggtgagtcagcaagtattctgaaagggaaagacatggtggggaggataaaacggctcttcattttaagaaatccctcttatcaagcctactattaacttgtttctttggcaactactatgcatttgctacggaaatatatccccagaataaaatttccatggaaggaaaa

SEQ ID NO: 299 - DNA, 401 bp, Homo sapiens; identical to SEQ ID NO: 258

SEQ ID NO: 300 - DNA, 401 bp, Homo sapiens; identical to SEQ ID NO: 259

SEQ ID NO: 301 - DNA, 401 bp, Homo sapiens
aagcctttgggatttacatgccaaactttgaggtagcaagattatgggataatttaattctgagatgtctgcttcagaataagttctgctcctgaatattctgaactccttccataaagaggaagcaataccaaagaagaactgtttaactcagtcgccctagggaacaaatcaactaacgaatttggaagagaaaggaaraaaattttatgtggcttttaacaaatttgctcattatctttttaatattttctgggctgtgaatagtgattagtatatcaaattaatccttggatggtttgcttgacactgagttaggatatttctaagaacagtatgtgaagatcatcctatccaattggatcagtttcttctctcccacactctgcatatcattttct

SEQ ID NO: 302 - DNA, 401 bp, Homo sapiens
atgggataatttaattctgagatgtctgcttcagaataagttctgctcctgaatattctgaactccttccataaagaggaagcaataccaaagaagaactgtttaactcagtcgccctagggaacaaatcaactaacgaatttggaagagaaaggaagaaaattttatgtggcttttaacaaatttgctcattatcttttyaatattttctgggctgtgaatagtgattagtatatcaaattaatccttggatggtttgcttgacactgagttaggatatttctaagaacagtatgtgaagatcatcctatccaattggatcagtttcttctctcccacactctgcatatcattttctcccctacacccaactatagcaatggaacaatccagtgtcttgg

SEQ ID NO: 303 - DNA, 401 bp, Homo sapiens
tctcaatgcctattgaacttaaagctgccatcacttgtttaatattgaatttttctctgtaggtgttggcctctgatatccttgcggctcagctgcaatgtatttcaatcctttttaataattcaaatatgtaccatgatgttacatcccattctagtattctctcatctctgggtcttttcttccatttcccactcctcyagcagtaaaagctcttgtggcatttgcatccatgtacaaccaacagatcattagtttcatgggaaattgtcaggtggcagagctttccttttgcactactcctaggactataaaaagcacaccacaagcatatttcagcaaagctaaaattaaaagtttaattaacaaaaatgctacttaataatacatttggatcgatt

SEQ ID NO: 304 - DNA, 401 bp, Homo sapiens
cagaactgtctatacttttaccatggcaaattgtattagagctgtttaggttgctctgcctgttccattttaaactttaggaggacaagagcttttttgagtctgtctccctcatcttggcccctctgggcctatttgccaatgaaaggggtttagtcaatgttggattttcagcactgttcccaggaggtgtcaggagcrggaggattcagaatcattttgccatagtggtagccttcaaggagtttatagtaatgttaaatagaagattaatgttaaaagagaaactttagcagattaaacctttaaagagtttatttgggcagacagcaattcatgaattggacagctctagactggaagtggttcagggcttcagcagaggtggttgatggaaaagc

SEQ ID NO: 305 - DNA, 401 bp, Homo sapiens
tcaaaacatcttgctctggtcaccttttatagatgacctttccccatcttaatttctctgttctgtgttttgtggcataccgattctgggtatgcagtatgtgctcagccagcaaatctggtaatcagtatcttaattggcttggtatttcccagtgttcctttttccaaggcataatttattctttctgtttttgaatgyagcacatagaattgtattacctgaatttcaccattctgtaaatccagtcattgctgtatacttttctagccctgaccaggaaatctattctttatttatcatgctaacaacaacaacaacagagagagaagcaaagtcttgcttaaatgcttttaattgctatgctgtccacatgtgctcatgatgagaaatgcacctct

SEQ ID NO: 306 - DNA, 401 bp, Homo sapiens
aagcaaaaactttgggttatttcaggtattagttacattttcaaaaagtttttctaatgcagtcttttatttctcttattggtttacacatatttgtcatgaactaccacccaaagaacagttttatagccatactcagcaccaggtttcattacatcgattgtgcagatcaagaaacccgtagtaataaggacatggcarcacagataagcagctccaaatccttacacattggccaagcagtaaggggcattctgatttgctttcaaatgtctattcaatttgttcaaatgggcaagaattccctcacaactcagaagacatttctatgaaatattttcatgcctttggataatttttcagcagtaaaaatggaggagagaagaggctgaaaggaaaaa

SEQ ID NO: 307 - DNA, 401 bp, Homo sapiens
tggtttacacatatttgtcatgaactaccacccaaagaacagttttatagccatactcagcaccaggtttcattacatcgattgtgcagatcaagaaacccgtagtaataaggacatggcaacacagataagcagctccaaatccttacacattggccaagcagtaaggggcattctgatttgctttcaaatgtctattcmatttgttcaaatgggcaagaattccctcacaactcagaagacatttctatgaaatattttcatgcctttggataatttttcagcagtaaaaatggaggagagaagaggctgaaaggaaaaaaatatatctctaattttcttctgccctagatataaatattttcttatatatgttattttctaagatgacagaaataagg

SEQ ID NO: 308 - DNA, 401 bp, Homo sapiens
cctgtgacttctggtgtggatgctaccaaggtcctatgagagatggcattgtcagtgtgccaagccacttctcaggtcctcacagcatgcaagctcttacaattattatttcttctccaagtggtaggaaagctgctctttgcacagctagtttcctctgaaatgtttcccactccatgaatgctttgttctgaattttcyagaatttatcttttgtaattcaaagccaaatcttcagaatctccaatagttttgttttttcttgttctcaagtgcctgcctctgctaatgtcatgaatcagttgtggaaatgcaaagcccaaagtgtactctattttccccaccatcagcctcatgtacccctctatagactgcagagaaaaaaaaatggctaagaagga

SEQ ID NO: 309 - DNA, 401 bp, Homo sapiens
agggagtgtggggaagcctctgtggtctccactcatgtcaagacaaacatcatgccatataggtggctcctcactctgtgtgtttggcagaggccttgctttaaggatggttggagacagcacatagggcaggtacaggagaagatctgggctttttcagggtacagaatgtggataagacccagaagaaaggaaaagagwtggggaactagggagtctggctcatgtttgtttccaccttaacttctcttggaagtttgaaatagtccatgaaagtgaaagcacttttcatcacagtcatcaactgctataggcatttgcagcctgcaaatagattaatttcactggatgttaatttcactgtgaggctttagccagcactactgtttctcaacacatag

SEQ ID NO: 310 - DNA, 401 bp, Homo sapiens
tatgagatattggaaaacaggatgagagaagagaaaaggcttttggcaaattgactacaagacagggagacaagaagtcttgatttaacagttgaacactttttattttttctctcatcccttgaatgctgataagacataacaaaacctacttggatgtgcttgcttgcacatgtttccttggtattcattttgcagacsccctctgttttgattcaaaaatgcttttctttttctcaaacacaataagctagaagttctgcctcttatgaaacattcaagagccaatgaatgcctgctatctagaaaagtgaaatgccctcatagaattcttgcgacctttatgcattgtcaacagattctgtgaataaaacaaaacaaaacatgaattttaacactga

SEQ ID NO: 311 - DNA, 401 bp, Homo sapiens
taataataacaaaagaacagtgagatttggataagaagggaaaaaataaacttaaaattcctgctttatatttatgagacaccatcttgctaaaaggtatgttgatgaaaagagttgatgaaaagagtcaaactctgtaaaatctttgaagagggttattctaagccaaatgtgagtgaccgtcacctaaggcacagtctyacgaggtgctgagaacatatgtgcccaaaatggttgggcttcagcttgattttacacattttagggggatataagacatcaatgaatataatgtgagggatgcatgggttcggtctggaaaggcgggacaactcaaagtggaagtgaggtgggggtggcatagagggtggtgagggatggcttggggtgtggtggagtgg

SEQ ID NO: 312 - DNA, 401 bp, Homo sapiens
actctattttccccaccatcagcctcatgtacccctctatagactgcagagaaaaaaaaatggctaagaaggaaaaatacaaatgtttccattatattgggcaggagaggaggtggtaaggttaacattggaattggagtgggagtgggaggccgttaggtgggaggggtgaaaactattgttgggcttgtggagcttggraaatctccttctgtggttctgatataattcttgcctctccctagaacctcactttgtgaggttcagtgtttaccagtgtctttcattgtgtgacacattcttcaataagccatcgtgtattgagaagcgggctgtgggtcattgttcactgcacttcagggcactcatcctttcctctgtccctaaggaacacaagaacc

SEQ ID NO: 313 - DNA, 401 bp, Homo sapiens
atgatgcagctctcattccacctccttcctgaaatctctcctgacctcttcattctaattaacatacctttctccggacatccagagcatctcatcttgcctttcctaaggtactttttcccaacaccttccacctggtttaatacctatctgtatatatcttctcctcctccccaccactatatattatgccccacatgsgcagagtctgtgtatctacatctttgtctctttacatggtgtctggcacagcaccttcttcagagtgggttttcaatatgtgattattgaaaagtaaatgagcaagatggccatgacaaagcaaaggagtaataaacaacttcttcctaagagacgaaagatatacaatgttttgctttgtgaatacacagccatgcagg

SEQ ID NO: 314 - DNA, 401 bp, Homo sapiens
acaaggatgctaggatattgtgaggttagctacaccaagccccacaaacacacacacacacgtacacacacacacaatgggaatgatggatccagacaatgttatgaggaaaggaaaagtgcaaatcttcctagactaagataaccttcatttgagtttcctgattagctaagcaatagaaccaaagactttgcgttgtaygggaccttagaggtcacctattcccacttccttgtcaaagcaggagtttccagaaaagtctttttctgtcatctttgtaaaatgtaaatctgatcatgtcactctcttgttttaatatcagtcaatgattccccattgtcctcaggataaagtccatattccctaacatgacttacaaggtctggcccctcttatctctc

SEQ ID NO: 315 - DNA, 401 bp, Homo sapiens; identical to SEQ ID NO: 275

SEQ ID NO: 316 - DNA, 401 bp, Homo sapiens; identical to SEQ ID NO: 274

SEQ ID NO: 317 - DNA, 401 bp, Homo sapiens
ttttacttaatattctttttctcttccagaatcccattcaggatgccacattacattcagtatgtcttcttagactcctatgggctgtgacagtatctcagaatttccttgcttttgaatatattgagttttggtaaggtattttgtagactattcctcaagtggaatgtatctgatgtttttctcatgagtagactgggkttatggatttggagaggaagaccaccatatcatatcaagggtgcatgctatcaatgtgacttctcactgttgatgtttgatggaggtaacctgttggtaacctgactgaggtagtgtttgccagatttctccactatgaagttacttttcccctcgcgttcacactgtgctctttgaaaatgagtttctatacacacgcc

SEQ ID NO: 318 - DNA, 401 bp, Homo sapiens
ttggaaagaagcagaacactttaggcaacaaggcacttgatgaagcaccatggtactctaagaccttctccactttgtccacatcccaccttcctttcatgatgcagctctcattccacctccttcctgaaatctctcctgacctcttcattctaattaacatacctttctccggacatccagagcatctcatcttgcctktcctaaggtactttttcccaacaccttccacctggtttaatacctatctgtatatatcttctcctcctccccaccactatatattatgccccacatgcgcagagtctgtgtatctacatctttgtctctttacatggtgtctggcacagcaccttcttcagagtgggttttcaatatgtgattattgaaaagtaaatgag

SEQ ID NO: 319 - DNA, 401 bp, Homo sapiens
ctgtagatcggagctgttcctattcggccatcttgtgcaaccctcttttcttattcttgaactgttgaataagctaccagcattttaaataagttcaggattatgttgccaggaaatagtctttgatatctgtgacaattttgttgttgctgtttgttttttctttctttttttttttttatggggaaaagcatgaagttrtctttctcattgcctttctcatcatgttttaagtacagagttcttctgaagggcacactttgtggttctcatggaatgtaaatagctcaaatggctccacttccaagaaaccttctttaaccttaaggctgaatgaagtaagcgctttctcctgttctctagtgtattcccagcaaaactttgagcatactttaaaaatg

SEQ ID NO: 320 - DNA, 401 bp, Homo sapiens; identical to SEQ ID NO: 265

SEQ ID NO: 321 - DNA, 401 bp, Homo sapiens
tgaaatattttcatgcctttggataatttttcagcagtaaaaatggaggagagaagaggctgaaaggaaaaaaatatatctctaattttcttctgccctagatataaatattttcttatatatgttattttctaagatgacagaaataaggatgcaccagtcttcccaggtctgctctacttgataacggcatcaaacaawcaaacaaacaaaacaaaaccaaacaaaaaaagaaaaaaagaaaggaaggagaggaaagacccactgtgaaaaatatgagcctgccacgttcagaccttaaaactgagcaggaggacagggtacagtggagaataacactaagttatttagtgctaagaatgtcaaggtataattttcaaaaagttaaaacagggctctca

SEQ ID NO: 322 - DNA, 401 bp, Homo sapiens; identical to SEQ ID NO: 254

SEQ ID NO: 323 - DNA, 401 bp, Homo sapiens; identical to SEQ ID NO: 266

SEQ ID NO: 324 - DNA, 401 bp, Homo sapiens
ctctttaagatcattgaagcttaaaatataagcttgaagttttacatattgataagttgaaatattttggcaaaaaaattgtattaagcaaccaaaaatttgagaaagtacggctaaaatatctttcctcattgtaataaaatagagatgtgaagttagtaacattagtataatatggagtttaattcaatagaaatcackcactcaaaccttttcaacaaccatttattgaatattgacaacgagcaaggccaaagtcagtagagaattaaataatgaaccaaatccatattctgtcctcaatgaatgtataagataaagtataatcaaaagcaaggtggggagtggtgaataccataaggaatttggaaaaggcatgtactgctcttagcaagagacta

SEQ ID NO: 325 - DNA, 401 bp, Homo sapiens
aaatgtaagggacaccctcatagtgacttctttcctagcagtctgcctctgtttgcggtgttgtctggggtcattgttgaaagttattgacatatttctgcctctttgtgagttatctgtgaagctacgccacagtactctgttgttggctattctgcatgctcccagtgctcaggtgaattccttggaagatactgagcraaaacctagagcttatatgaccataatatctagaccattagcatgaactggtaaaaaccaaagtagaggaaaaagattgaaccaacaactcagtagaaattggtaggtttatatgagctttgttttgtggctcgaacattttagatacccaaagtccaagcattactcagttgggactggaaatcagatgaagtcatcta

SEQ ID NO: 326 - DNA, 401 bp, Homo sapiens
tccactcctgcaataacaatattaagccattcatgaggatagagccctcctgacctaattacctcctgtgaggccccaccttccaagactgttgcattgagattacatttccaacacatgaacttttgggggacacgttcaaaccgtagcatttgtctagcaacttcctaatgttcacaactcagatcctgcctcctagayttccctctgcactcccatgctattttttgaaacctataattccatcgctaaaaccaagtcaaagacaaccatacctttgtttcctcatctgcaggaggtgataaccgatagtattcatagaggcaaaataaattaatgccatgtaagaacagcatgtaaaccatagtatcataaaagtattcactatcattattcaagta

SEQ ID NO: 327 - DNA, 401 bp, Homo sapiens
gttaaacgttacacacgtagcttttgttttactttcttgtctggtagccaagtatttttcaagtcactcaacattctactgaggcatgctagtccactgaatatgattataaaggaaagcctaacatgacctaaaaagagggaggacggagagctttggctgttgtgagcaccacagctaatgaagaagcaggataatttrcatgagcgatatcccttcagttatttctgtaaatctcagagccagttcctgtcaagttgtgaggaaacaaacattttttattaaggaataaaaagataggccaggcccagtggctcacacctgtaatcccagcactttgagaggctgaggcgggcagatagcgaggtcaggagtttgagaccagcctggctaacatggtg

SEQ ID NO: 328 - DNA, 401 bp, Homo sapiens
ctagggcagacggcctactactgtaaaatgcatatggtgtaaggatagtggtaggggaggggatagagtttgttcctatgtgggaaacaggaaggttttgggatttgtggcaagaaagagttagattttaaaaattaccccagcaacagcagttttatctaaatagtttcagtattagcaagggaaaatggcagtgaaccracattttatggattgattagtgctaggtattgtgcgtgacatatataatcttattcaatcattacagtaatccatgagagtatccatttttccttttaactagagaaataactcatttacctctggactctaaaaatcacctcttttcctctgcaatacatcagatgtgtgtgcacgtgtatttatcaaccaaaggatac

SEQ ID NO: 329 - DNA, 401 bp, Homo sapiens
ttgtggagcttggaaaatctccttctgtggttctgatataattcttgcctctccctagaacctcactttgtgaggttcagtgtttaccagtgtctttcattgtgtgacacattcttcaataagccatcgtgtattgagaagcgggctgtgggtcattgttcactgcacttcagggcactcatcctttcctctgtccctaakgaacacaagaaccacacctgtcttttcagcatcctgtcttgcaagcccagtcacacggtgggtgctctggaaatatttattgtatgaatgattgaagacggtatgaatgactgaagacggtatgaatgacagccaggtttagttttgggaaaaaattatattcaagacaattgaggaggtgatgctattctggattagga

SEQ ID NO: 330 - DNA, 401 bp, Homo sapiens
ctattccaaaagatagagaaagagggaatcccccctaaatcatctatgaagccagcatcatcctaataccaaaaccagggaaggatataacaaaaaaaaagaaaatacagaccaatattcctgatgaacatagatgcaaaaatcctcaacaaaatactagcaaactgaatccaacagcatatcaaaaagataatccaacaygatcaaatggatttcgtaccaggattgcagggatgctttaatatatgtaagtcaataaatgtgatataccatataaacacaattaaaaacaaaaatcacatgatcatctcaatagatgcagaaaaagcatctgacagaatccagcattcctttatgagtaaaaccctcagcaaaattggcatagaagggacataccttaa

SEQ ID NO: 331 - DNA, 401 bp, Homo sapiens
aaagtagtaaaatctgttgaattattggctactttaccatcattacctttactaaatggttttcagtatttcttctagttttaattttctattctgctgagttcttaaattgattcaacaaatgtttactgagaaattctataagccaagcatagtgctgtgcactaaaagagtcattacaatcaatatgagggccaggtrcagtggctgatgcctgtaatcccagcacatagggaggctgaggtgggcagattgcttgagctcaggagttcaagaccagcctgggcaacatggcaaaaccccatacctacaaaaaatacaaaaattagccaggcgtggtggcacgcacctgtagtcccagctacttgggaggctgaggttggagaatcacttgagcccgg

SEQ ID NO: 332 - DNA, 401 bp, Homo sapiens
ccaagcatagtgctgtgcactaaaagagtcattacaatcaatatgagggccaggtgcagtggctgatgcctgtaatcccagcacatagggaggctgaggtgggcagattgcttgagctcaggagttcaagaccagcctgggcaacatggcaaaaccccatacctacaaaaaatacaaaaattagccaggcgtggtggcacrcacctgtagtcccagctacttgggaggctgaggttggagaatcacttgagcccgggaggtggaggttgcagtgagccgagattgtcccactgaactccagcttgggtgacagagccacgccctgtctcaataaaataaaataaataaaataaaaatgaggatgaaacattaaaggatttttacgaagtcttttaataaca

SEQ ID NO: 333 - DNA, 401 bp, Homo sapiens
tctgctatccttggcagctgcaaagaaagtcagtcaaaaggaatatattatttcgatgtagtctaacttctataacaaacaactccccagttgcagcagcttgacatagtgaaactgttatttcttgctcatgtcacagcgcagtgtgggaggatggagggactctgttccacattcattcatgaacccaggcaccttcartcttgtggctccaccctcatattggcccttaaagttctttcaaggtagctaatgagaaaaaaggcagaaaagagaacgtgatgaccacacaaaggaagcgtatgtaggttatttctgcaagtggcacgtgttgctccagctcatatccaattggccagaatgccactgcatagccttacctggaacacgtggtgtctccc

SEQ ID NO: 334 - DNA, 401 bp, Homo sapiens
acatagtgaaactgttatttcttgctcatgtcacagcgcagtgtgggaggatggagggactctgttccacattcattcatgaacccaggcaccttcaatcttgtggctccaccctcatattggcccttaaagttctttcaaggtagctaatgagaaaaaaggcagaaaagagaacgtgatgaccacacaaaggaagcgtaygtaggttatttctgcaagtggcacgtgttgctccagctcatatccaattggccagaatgccactgcatagccttacctggaacacgtggtgtctccctgtgcccagggagacaaaaacaacaatgttggtgaacttgggcatacattcatctcaagagccataaaggcctgttagattagatttgtttgttcttaactgt

SEQ ID NO: 335 - DNA, 401 bp, Homo sapiens
agggactttgttctccctattttacagatgaaaacagagtttggttgcaaaaccaaacttaattctataacagaaaagcaaatacttcagatgggcaaatacagatatttcagatgggcaaaaccaaactcaattctgtaacagaaaagcaaataattatagtaaatacttatgtaccaattgttctatgtcagatacacrtaaatatattaacttatttaatatttacaataatcttatgcagaggggactttgttcgccctattttacaaatgaaaaaaaccagacacaaagaggtcaagtgacttgcccaaggttacacagctaataagtgtgctcttagccattatatgctgattcgtatcccatataagctacctgccaccagacaagagcataca

SEQ ID NO: 336 - DNA, 401 bp, Homo sapiens
tttaagtatgcctgtaggtttattgtgagagaatgagagatactttccctaggtccatctcaaggagaaagcaaggctctgtgacctagatgtctgagacttgactcactccaaggaagagagttttcagaagcttaggctcccaaaggagatagttgtattattcaagaggaaagctcagaagtttctaaaagatatgayctttttatcccagtggaccttaaggccccatcctatgcagggctactaatgaggtgatggaggcctaggtctaaatcctgaaacctgtggtccaggaaggataaaaaatccccagggccaagttagatgtgcagggtgtggcaccagatacaccctctttccagcgagccccttcagcagcaagaccagaggcccttcct

SEQ ID NO: 337 - DNA, 401 bp, Homo sapiens; identical to SEQ ID NO: 292

SEQ ID NO: 338 - DNA, 401 bp, Homo sapiens
aactatatacatagtagattattccattgttgacacttctgaatcatatggccatggaaattaaggggatcccttaattgaacccctcaattgaaagaggagcaaactaaagcacagagtacattgtccatataaacaaccacttagggctgccatcttcaatgggaaggtctctctcaagttatagactgaaatctaagyctggcccagcattcactgggcaccttattttctacctacattttcataagatgtatcacaaacttcagcaattatttgttgtgtactatctgctatatatttatgcaagttaacattgaaataataaaatgagaagccattagacaggaaccctgggttcctacctaaggaaactaaaacctagctcagcctcatttcat

SEQ ID NO: 339 - DNA, 401 bp, Homo sapiens
tggtcatatgaaatactaaagatagtttcctgtaaaaacctcccacagaaacgaaaagtttttaaataaaacatgtcatccaaaattgtaaagtgagaaagattgttttcatctcatgtctttgaattgtattgtgagaggtattggtcttcttccaccaatccaaagttaaggacaaataaatttcattgtatcggaaamtttggaggaaaaatttgatgctattattttccttccatggaaattttattctggggatatatttccgtagcaaatgcatagtagttgccaaagaaacaagttaatagtaggcttgataagagggatttcttaaaatgaagagccgttttatcctccccaccatgtctttccctttcagaatacttgctgactcaccccag

SEQ ID NO: 340 - DNA, 401 bp, Homo sapiens
ccacaagtataagacacctctactccctccttggtgacccatcatgcaccccttaccatctcattaaaacctaatcaccctcaccctgctcaaagccaagatcccatcccacagcacgctttaaaaggattaaagcctgttatcactcgcctgctacggcatggccttttaaagcctataaactctccttacaattccccyattttacctgtcctaaaaccagacaaggcttacaagttagttcagaatctgtgccttatcaaccaaattgttttgtctatccacctcgtggtgccaaacccatatactctcctatcctcaatacctccctctactacccattattctgttctggatctcaaacatgctttctttactattcctttgcacccttcatccca

SEQ ID NO: 341 - DNA, 401 bp, Homo sapiens
tttattggtgctcaaagtgtagcaaggttgggagatgtagcaaagcacattctaaataaatcaacggaagcaagcaaactaaatcacaggggggaattacagtgtccaaatcatttatacactggtgattaccatttcacctgggtccaactgatgtcacactagagtttccaaagctctaggaatcattcatcaatccarcgaaacctgctcatccatggttccacattgaaactggccaacatggtaagaattacaatagtccaggacaggaaggttaattagtcacaagaaagaacatgtggagaagaaaatgtactgagtgggagaggttctagtatcattcctggtattagttagcaatgcgatcttagggtagttgctttctatgtctgcctcag

SEQ ID NO: 342 - DNA, 401 bp, Homo sapiens
gttgtgcgtattgacagccaggcttctaaacctcttaaaactccccaactctcgtgccaacttagacaacactcttttaagcactcctttttaattatccccacctgcccagttcccttattaggctgagacactttaactaaattatctgcttccctgactgttcctggactacagctgcatctcattgccacccttctycccaacccaaagcctcctttgtgtcctcttttatccccccgaccttaacccacaagtataagacacctctactccctccttggtgacccatcatgcaccccttaccatctcattaaaacctaatcaccctcaccctgctcaaagccaagatcccatcccacagcacgctttaaaaggattaaagcctgttatcactcgcc

SEQ ID NO: 343 - DNA, 401 bp, Homo sapiens
tgcgtattgacagccaggcttctaaacctcttaaaactccccaactctcgtgccaacttagacaacactcttttaagcactcctttttaattatccccacctgcccagttcccttattaggctgagacactttaactaaattatctgcttccctgactgttcctggactacagctgcatctcattgccacccttctccccwacccaaagcctcctttgtgtcctcttttatccccccgaccttaacccacaagtataagacacctctactccctccttggtgacccatcatgcaccccttaccatctcattaaaacctaatcaccctcaccctgctcaaagccaagatcccatcccacagcacgctttaaaaggattaaagcctgttatcactcgcctgct

SEQ ID NO: 344 - DNA, 401 bp, Homo sapiens
accttaacccacaagtataagacacctctactccctccttggtgacccatcatgcaccccttaccatctcattaaaacctaatcaccctcaccctgctcaaagccaagatcccatcccacagcacgctttaaaaggattaaagcctgttatcactcgcctgctacggcatggccttttaaagcctataaactctccttacmattcccccattttacctgtcctaaaaccagacaaggcttacaagttagttcagaatctgtgccttatcaaccaaattgttttgtctatccacctcgtggtgccaaacccatatactctcctatcctcaatacctccctctactacccattattctgttctggatctcaaacatgctttctttactattcctttgcaccct

SEQ ID NO: 345 - DNA, 401 bp, Homo sapiens
ctgatatattagcatgatttaatgttaggaaagatgctctatattttattcttctcaaagtccctaggagatgactagaagagagttaattgcttatattgtaaagctgatagttaccacatttattttttcttttctagcaacaaacttcacatccatcaaattcttaacatgaaccaggttctttgcttacattcgcayaggcatatcggtgagtgaggtgctgtttattatttccatttcactgatgctaaaactgggcctagacaggttacccaccttgtaagctgggaagctaaaatttgaatccaaagagtgtaactccaaaggctacttactattatgctagactgattctctaggcagagctctgttgtgatcaggggaatagcatctctttc

SEQ ID NO: 346 - DNA, 401 bp, Homo sapiens
gtagcaggcgagtgataacaggctttaatccttttaaagcgtgctgtgggatgggatcttggctttgagcagggtgagggtgattaggttttaatgagatggtaaggggtgcatgatgggtcaccaaggagggagtagaggtgtcttatacttgtgggttaaggtcggggggataaaagaggacacaaaggaggctttggrttggggagaagggtggcaatgagatgcagctgtagtccaggaacagtcagggaagcagataatttagttaaagtgtctcagcctaataagggaactgggcaggtggggataattaaaaaggagtgcttaaaagagtgttgtctaagttggcacgagagttggggagttttaagaggtttagaagcctggctgtcaatacg

SEQ ID NO: 347 - DNA, 401 bp, Homo sapiens; identical to SEQ ID NO: 268

SEQ ID NO: 348 - DNA, 401 bp, Homo sapiens
accaagccccgcaaacacacacacacacgtacacacacacacaatgggaatgatggatccagacaaggatgctaggatattgtgaggttagctacaccaagccccgcaaacacacacacacacgtacacacacacacaatgggaatgatggatccagacaaggatgctaggatattgtgaggttagctacaccaagccccrcaaacacacacacacacgtacacacacacacaatgggaatgatggatccagacaatgttatgaggaaaggaaaagtgcaaatcttcctagactaagataaccttcatttgagtttcctgattagctaagcaatagaaccaaagactttgcgttgtacgggaccttagaggtcacctattcccacttccttgtcaaagcag

SEQ ID NO: 349 - DNA, 401 bp, Homo sapiens
ttaattgtgatgttagggtgtcaattttagatctttcctgctttctcttgtgggcatttagtgctataaatttccctctacacactgctttgaatgtgtcccagagattctggtatgttatgtctttgttctcgttggtttcaaagaacatctttatttctgccttcatttcattatttacccagtagtcattcaggagcrggttgttcagtttccatgtagttgagcggttttgagtgagtttcttaatcctgagttctagtttgattgcactgtggtgtgagagacagtttgttataatgtctgttcttttacatttgctgaggagtgctttacttccaactatgtggtcaattttggagtaggtgtggtgtggtgctgaaaaaaatgtatattctgtt

SEQ ID NO: 350 - DNA, 401 bp, Homo sapiens; identical to SEQ ID NO: 261

SEQ ID NO: 351 - DNA, 401 bp, Homo sapiens
cagggttgctgccgaacgagccatgaactgggctgggtttttcatatttgatgaaaaagcctaaatgctcagtgatttgggagaggtctgataaagaaaagtagcattaaccttgactatgcctttagctccagccacctttttaagagtaaattgctgggcaggtgggggagggctagtcacgaaacgtaactgtaagcyggaccagttgtgaggatgggaggtgataaaaggattatagggtggaggagcagaggctgaggaagaattgggacctagctcagcctggcgaggaggggagaggtcagacgggtctgtagaaaaggaagattagaaagactcagtgatgcttggggttgggactgaggggacaggcaggaggtaaagaaggaagatttggg

SEQ ID NO: 352 - DNA, 401 bp, Homo sapiens
caaggatgctaggatattgtgaggttagctacaccaagccccgcaaacacacacacacacgtacacacacacacaatgggaatgatggatccagacaaggatgctaggatattgtgaggttagctacaccaagccccgcaaacacacacacacacgtacacacacacacaatgggaatgatggatccagacaaggatgctrggatattgtgaggttagctacaccaagccccacaaacacacacacacacgtacacacacacacaatgggaatgatggatccagacaatgttatgaggaaaggaaaagtgcaaatcttcctagactaagataaccttcatttgagtttcctgattagctaagcaatagaaccaaagactttgcgttgtacgggaccttaga

SEQ ID NO: 353 - DNA, 401 bp, Homo sapiens
agttgttcctttccatgtttagtgcttccttcaggagctcttttagggcaggcctggtggtggcaaaatctctcagcatttgcttgtctgtaaagtattttatttctccttcacttatgaagcttagtttggctggatatgagattctgggttgaaaattcttttctttaagaatgttgaatattggctcccactctcttytggcttgtagagtttctgcagagatatcagctgttagtctgatgggcttccctttgtgggtaacctgacctttctctctggctgcccttaacattttttccttcatttcaactttggtgaatctgacaattatgtgtcttggagttgctcttctcaagagtatctttgtggcgttctctgtatttcctgaatttgaatgt

SEQ ID NO: 354 - DNA, 401 bp, Homo sapiens; identical to SEQ ID NO: 269

SEQ ID NO: 355 - DNA, 401 bp, Homo sapiens; identical to SEQ ID NO: 270

SEQ ID NO: 356 - DNA, 401 bp, Homo sapiens
tttatttctgagagacagaggcacagaagcaagagtcactcagttactacacgggaaacgtggattttaatctgggttaagtagcattctagttgaagagtatacagtggcaagtgtgagacaacctgagcatttaaaaatgactatgaatatataaaactccgcaagtacataactagaaaagagaatgtcttccaaaamaaaactcatttgccatcactggaggtgattattacatgaattggttattctgagaattgagaattaaaaataattaaagatttaatgtctttctagaagatactgaatttcagcataactaaatagttctacttgatgaaggaaaacttttctttattgaagaatgtcagctgtgaatgtagatgcaataatacggtcag

SEQ ID NO: 357 - DNA, 401 bp, Homo sapiens
cacttttatttgggataaaacacattttgagacttggcctagggacttaaccaaagataaatccattctagccacaaaatccccttggacattttctccctttctggccctaggatatttatgtttgctgctccctctacctggagtgctttctccctgaatacctgttggttttctccctcagctctttcaggatatatracattcaaaatttcagctgtgatttctgtctctctttcctgctttgtatccaccatacgtattactgtctaatatactacatatgatgcttatttattttatttgtctgttgtccctcactagaatgtatgcttcaggaaggcaaggatatttaatgttttgtatccccaaatcttataatagtgcatggtgcacagtat

SEQ ID NO: 358 - DNA, 401 bp, Homo sapiens
atttgatcctgtcattatgatgttatctggttattttgctcaatagttgatgcagtttcttcctagccttgatggtctttacaatttggcatgtttttgcagtggctggtaccagttgttcctttccatgtttagtgcttccttcaggagctcttttagggcaggcctggtggtggcaaaatctctcagcatttgcttgtstgtaaagtattttatttctccttcacttatgaagcttagtttggctggatatgagattctgggttgaaaattcttttctttaagaatgttgaatattggctcccactctcttttggcttgtagagtttctgcagagatatcagctgttagtctgatgggcttccctttgtgggtaacctgacctttctctctggctgccc

SEQ ID NO: 359 - DNA, 401 bp, Homo sapiens; identical to SEQ ID NO: 267

SEQ ID NO: 360 - DNA, 401 bp, Homo sapiens
agtcccggatacttgggaggctgaggtgggaggatcatctgagactgggaagttgaggttgcagcgagccctgatgatgccattgtacaccagcctgggtgacagagtgagatcctgtctccaaaacaaacaaacaaacaacaaagaaacaaatgcctgttctattttaatgttaatgctggtcagctgtgtctaaactcygaaagggagagagcataatgattccttcctgtcacaacctgaactagttcttcagatttctttggaatttccttggtggagagggaatccctgcattcacttggaggggcttacagttttatttttggcttacaggtggtcaaaaacatactaagtgcctcacaaactccgggcacacaaagacagccaataattaatac

SEQ ID NO: 361 - DNA, 401 bp, Homo sapiens; identical to SEQ ID NO: 253

SEQ ID NO: 362 - DNA, 401 bp, Homo sapiens
atacagtggcaagtgtgagacaacctgagcatttaaaaatgactatgaatatataaaactccgcaagtacataactagaaaagagaatgtcttccaaaacaaaactcatttgccatcactggaggtgattattacatgaattggttattctgagaattgagaattaaaaataattaaagatttaatgtctttctagaagayactgaatttcagcataactaaatagttctacttgatgaaggaaaacttttctttattgaagaatgtcagctgtgaatgtagatgcaataatacggtcagaaagtcaccattttgcagcccttgataggatggaaattcaaataagaacctttgacagatgctgaaaccactaaaagaaaggtggacagggaccattgtga

SEQ ID NO: 363 - DNA, 401 bp, Homo sapiens
gcacaatgataggttggggtaggagtggggaagaagatttggatcaaatagttggtgagtctagttgggagtaagttccacttgagtgcccagggcaacatgacttgcaaaaaaggttaggttccttggccgagttaataaaagctattatttatatcaaagagggttttaagtgtctttcttgtctggcttgcccaactygatgggagcctgcatgtggatttccggtattgtatctcagcatgctggccgactgcagtgttggagagacacacacagcctggagagggacaggaagctctgtggtggaaggttggttgaagacatctgcattgtttagcttggaaactattacttagcttataacattaattgctaaggattatttatttgtaacaaaa

SEQ ID NO: 364 - DNA, 401 bp, Homo sapiens
attcaacagattttactactttcccccttccctttaatgattcagaggagagaagtaacttgcccaaggtcacattgctgctaattttgaagaatccagctctctctcaactgctagtgatttttttaaaaccatgatttcaactactctgattaaatggaattaaatctatggctaagccatatcttgtgaatggaggaygtttggaacctcaagatatgacatctaatttggaaatttggaaagcttttactgagacagaaagaatggtctatttcttgaaagtgctgttaaaaagcgacagaaatgcaattattttggatttgttttagtgcttcagtatttttagaatttccccataaacacctcccttgaggagcatacagcacttggcatgctaa

SEQ ID NO: 365 - DNA, 401 bp, Homo sapiens
gagagaagtaacttgcccaaggtcacattgctgctaattttgaagaatccagctctctctcaactgctagtgatttttttaaaaccatgatttcaactactctgattaaatggaattaaatctatggctaagccatatcttgtgaatggaggatgtttggaacctcaagatatgacatctaatttggaaatttggaaagcwtttactgagacagaaagaatggtctatttcttgaaagtgctgttaaaaagcgacagaaatgcaattattttggatttgttttagtgcttcagtatttttagaatttccccataaacacctcccttgaggagcatacagcacttggcatgctaaagtaactcaggatgctttcagcaaatgcagttgacagcttgaccaga

SEQ ID NO: 366 - DNA, 401 bp, Homo sapiens
ctcattagctaccttgaaagaactttaagggccaatatgagggtggagccacaagattgaaggtgcctgggttcatgaatgaatgtggaacagagtccctccatcctcccacactgcgctgtgacatgagcaagaaataacagtttcactatgtcaagctgctgcaactggggagttgtttgttatagaagttagactacrtcgaaataatatattccttttgactgactttctttgcagctgccaaggatagcagagatgtcctctgctttctaaattacatgtttgttgatatttttacgatcagggactttttaagtaaagaaaccaagaagcttgttcactaggcttcagtcacctcacatggaagtcagtaccttcctgtataaacactagggtct

SEQ ID NO: 367 - DNA, 401 bp, Homo sapiens; identical to SEQ ID NO: 256

SEQ ID NO: 368 - DNA, 401 bp, Homo sapiens
ccttccactgtgagagttacctaaagcttggcatccgtgatggtctacagggcttctgaggtgatcgggcagcatcagtcttcagccgctaagccgagaaggagtcagagagccttgggccagagttccaggggctctgggagtggctgccaggtgagttgaacagtccgatttccagtggggtcctacacagatgggacrcggcttaggaggaatcctgggctgagggcattccttggcctggtggccagatttctggcacttgtagcaagctcctgggggaggaggttctggaggaacgcctggccagtgcggttcaggcgtttggaaattcttgtgtgctggagatgtggctggggtttgtctcacagtggaggcaaggaattgcaacttttttctat

SEQ ID NO: 369 - DNA, 401 bp, Homo sapiens
gggtatgtcttgttcagatcccctatattgtaaattgaaaaaaacaaaaacaaaaaacaaaaaaacaaaccctaaagtctgtatagcaaaagtgaaaaaaattcagaaagcagagttcacagcactaatgaccacaactttacaagtaaagtatacatcttcaattttgaaattcaatttaaaattgtgctacaaatctgrtagataaaatagtcacccagatatacatgaggttgtagtttacacataattactatattttcatttatttggcttgttagccaaaatgcaagtgaacaatcatagtctccactgtgcaaggggccattatttcagtagactccatgtgagaagggcctgctcatatattaataaatcaacaggttcaggtcaaggaggaa

SEQ ID NO: 370 - DNA, 401 bp, Homo sapiens
tggtgtataagaatgcttgtgattttttacattgattttgtatcctgagactttgctgaagttgcttatcagcttaaggagattttgtgctgagacaatggggttttctagatatacaatcatgtcatctgcaaacagggacaatttgacttcctcttttcctaattgaatgccctttatttccttctcctgcctgatcgycctggccagaacttccaacactatgttgaataggagtggtgagagagggcatccctgtcttgtgcgagttttcaaagggaatgcttccagtttttgtccattcagtatgatattggctgtgggtttgtcatagatagctcttattattttgagatatgtcccatcaatacctaatttattgagagtgtttagcatgaagg

SEQ ID NO: 371 - DNA, 401 bp, Homo sapiens
ggtgtcagggtagtctaagtgaaagcaaagagaggctgggatgaagggtgcaaaggaatagtaaagaaagcatgtttgagatccagaacagaataatgggtagtagagggaggtattgaggataggagagtatatgggtttggcaccacgaggtggatagacaaaacaatttggttgataaggcacagattctgaactaayttgtaagccttgtctggttttaggacaggtaaaatgggggaattgtaaggagagtttataggctttaaaaggccatgccgtagcaggcgagtgataacaggctttaatccttttaaagcgtgctgtgggatgggatcttggctttgagcagggtgagggtgattaggttttaatgagatggtaaggggtgcatgatgggt

SEQ ID NO: 372 - DNA, 401 bp, Homo sapiens
ggggatgggcttaccttccactgtgagagttacctaaagcttggcatccgtgatggtctacagggcttctgaggtgatcgggcagcatcagtcttcagccgctaagccgagaaggagtcagagagccttgggccagagttccaggggctctgggagtggctgccaggtgagttgaacagtccgatttccagtggggtcctrcacagatgggacgcggcttaggaggaatcctgggctgagggcattccttggcctggtggccagatttctggcacttgtagcaagctcctgggggaggaggttctggaggaacgcctggccagtgcggttcaggcgtttggaaattcttgtgtgctggagatgtggctggggtttgtctcacagtggaggcaaggaattgc

SEQ ID NO: 373 - DNA, 401 bp, Homo sapiens
tattgaggataggagagtatatgggtttggcaccacgaggtggatagacaaaacaatttggttgataaggcacagattctgaactaacttgtaagccttgtctggttttaggacaggtaaaatgggggaattgtaaggagagtttataggctttaaaaggccatgccgtagcaggcgagtgataacaggctttaatccttytaaagcgtgctgtgggatgggatcttggctttgagcagggtgagggtgattaggttttaatgagatggtaaggggtgcatgatgggtcaccaaggagggagtagaggtgtcttatacttgtgggttaaggtcggggggataaaagaggacacaaaggaggctttgggttggggagaagggtggcaatgagatgcagctgt

SEQ ID NO: 374 - DNA, 401 bp, Homo sapiens
cttggcatccgtgatggtctacagggcttctgaggtgatcgggcagcatcagtcttcagccgctaagccgagaaggagtcagagagccttgggccagagttccaggggctctgggagtggctgccaggtgagttgaacagtccgatttccagtggggtcctacacagatgggacgcggcttaggaggaatcctgggctgarggcattccttggcctggtggccagatttctggcacttgtagcaagctcctgggggaggaggttctggaggaacgcctggccagtgcggttcaggcgtttggaaattcttgtgtgctggagatgtggctggggtttgtctcacagtggaggcaaggaattgcaacttttttctattattgtacaccttgaaggtgaggtta

SEQ ID NO: 375 - DNA, 401 bp, Homo sapiens
cagcagtggctgcagaacagtggatattggtgaaccacagatgctgctgcctaatcgttcctctggaagttttgtctcagaggagtacctggccatgtgaggtgtcagtccgccccctactggggggtgcctcccagttaggctacttgggggtcagggacccacttgaggaggcagtctgcccgttctcagatctgaagytgcgtgctgggagaaccactactctcttcaaagctgtcagagagggacatttaagtctgcagaggttattgctgtctttttgtctgtgccctgcccccagaggtggagcctacagaggcaggcaggcctccttgagctgtggtgggctccacccagtttgagcttcccggccgctttgtttacctaatcaaacaactaac

SEQ ID NO: 376 - DNA, 401 bp, Homo sapiens
ctgtcaatacgcacaacagttatggaggcaagggaaagagacccttgaaaagaaggtaatgtggagtgggtagcctccatattgattaagaaggggatgggcttaccttccactgtgagagttacctaaagcttggcatccgtgatggtctacagggcttctgaggtgatcgggcagcatcagtcttcagccgctaagccragaaggagtcagagagccttgggccagagttccaggggctctgggagtggctgccaggtgagttgaacagtccgatttccagtggggtcctacacagatgggacgcggcttaggaggaatcctgggctgagggcattccttggcctggtggccagatttctggcacttgtagcaagctcctgggggaggaggttctggag

SEQ ID NO: 377 - DNA, 401 bp, Homo sapiens
ttgaacagtccgatttccagtggggtcctacacagatgggacgcggcttaggaggaatcctgggctgagggcattccttggcctggtggccagatttctggcacttgtagcaagctcctgggggaggaggttctggaggaacgcctggccagtgcggttcaggcgtttggaaattcttgtgtgctggagatgtggctgggrtttgtctcacagtggaggcaaggaattgcaacttttttctattattgtacaccttgaaggtgaggttaattaagtcctgttgtggggtttgagggccagattctaatttttggagttttatttaatgttgggagcagattgggtaataaaatgtatattgagaataagatggccttttgaccttttagggtctagggctg

SEQ ID NO: 378 - DNA, 401 bp, Homo sapiens; identical to SEQ ID NO: 257

SEQ ID NO: 379 - DNA, 401 bp, Homo sapiens
tcttgcaatgatcatttattgtgttttttttataaatcatgtccccccaaatttgaaaatgatgggcttatagttggcattttttgggtgggcccatatatatggatgctgtggaatcgaagtgtggtatggagtacacgcatgtacagctgtttggttggtggcagaggtagcactaggaaaatgggaatggtggttgcraaaggggccacaggtatggtgtaagatcttgacatatgggaagctgtcagtacctaacatacgggaaaaacattattttagagctacatgtgcctgctctctaaatcatagaagcattgaagaaagactttggatttacctgaagtcctgagttctagtcctgtttttgtctccaactggttgtgtagcctacaataagt

SEQ ID NO: 380 - DNA, 401 bp, Homo sapiens
gctaaagtaactcaggatgctttcagcaaatgcagttgacagcttgaccagacatctggatttgtgtatgtctgagagagtccagatacttctccaagatatttttgcttttcagtcttcagttgaactaatacttcccctctttcttgagcctgaacaccttcctagggctgaacctagacaactgtgaagatggggttscttctgtccaaacacactgctaaaactagactgctaactgttcagcacggaccctgtgttccagatgtatgagggtatgtttgaagaaggtctgcagcagcaagggctctggagtggcatataggctgaaaaatctgatgaaatcaggatgccagattacttggtctagtgcagtgatattgttctgagaagtgaggtgg

SEQ ID NO: 381 - DNA, 401 bp, Homo sapiens
tatttcattccttaggtataatagtgattgttttataaatttgggagctccagttgtatatatatagaggcatatatatttaagaatgtgatatttttctgttggactagtccttttatcattatataatgtccctctttgtcttttttaactgctgttgctttaaagtttgttttgtctgatgtgaaactaactactccygcttgctcttggtgtccatttgcatggcatatctttttacacccctttaccttaagtttatgtgaatctttaggtgttaggtgagtcttctgaagacagcagaaacttggttggtgaatccttatccattctgccgttctgtatcttttaagtggagcatttaggccacttacattcaacgtttagtactgggatgtgag

SEQ ID NO: 382 - DNA, 401 bp, Homo sapiens; identical to SEQ ID NO: 262

SEQ ID NO: 383 - DNA, 401 bp, Homo sapiens
accctgtatcctctgacctggaactgtgagtggtaaccccttttctcaaccacttctgtgactttttttgtatgcgcattgggattgtgtgccttcttaggtactcttggctcttttccacttaccaccctgcagaaatcacagctttgggaactgcacctatattcctccttcggccctttgacctacagcttggcatcrtgtcaaactcagggctttgctgtgagctgcgcagtatctgaaccgcattgtggaaagccaggaaatgggctctttttctctgaaatccccagttctcacctgtgacttctggtgtggatgctaccaaggtcctatgagagatggcattgtcagtgtgccaagccacttctcaggtcctcacagcatgcaagctcttacaa

SEQ ID NO: 384 - DNA, 401 bp, Homo sapiens
ttataagataaccagaacaaactattctagttgttgttatttcaactttcatctgcttattttctgcacaactgatgacagtagtggcaagaagtgaagactttggaattaaactgattggattagaaacccagctttgtcatttactacatgtgtgatgaaagcaagttgtttaagctctccaaacttcagtttcgtcaygagtaaaatggcaataattgtacctatatctcaggattcttataaagattaagtgagatagtacacagtgcttggctccacaattggtaattcactggtagtcactgtgctaaaaatgaagataaacaaaaattcaaggtttgttctactgaactgttgttaccttttgtctgcctcttgaagcttagtgactttgtatt

SEQ ID NO: 385 - DNA, 401 bp, Homo sapiens
ctacagtgctggtttagacagattttccctcttgcactctctctcagtgctgtagaagaatttttggaataagctaaatcatcgagactgaattcttcatatcaggaaagtgagacctagagagaccaagtaaattgtcccacatgttagtgccacagccagaactccaactcagttctcccgactctcaatacgtggtgwttttccagttaaacatctgagttatctgggttttttttttttttttttttttttttttttgagatgaagtctcgctcttgttccgcaggctggaggactgcaatggtgcaatggtgcaatctcggctcactgcaacctccacctcctgggctcaagcaattctcttgcctcagcctcctgagtagctgggattacaggca

SEQ ID NO: 386 - DNA, 401 bp, Homo sapiens
cgacctcccaaagtgctgggattacaggcgtgagccaccgtgcccggctgcttaatctgttttatagaaacacactggagctcagataagtgaagtcatagtgcattgggaggaactgagaaatttcccaaggagtaagatgacaaatttctgacaggattatttcagtgtggcctcattcataccataagaagccattcytctgaattgtatgcaaaaagaaactagaaggcctagtttgttattaaaagacttcgtaaaaatcctttaatgtttcatcctcatttttattttatttattttattttattgagacagggcgtggctctgtcacccaagctggagttcagtgggacaatctcggctcactgcaacctccacctcccgggctcaagtgattc

SEQ ID NO: 387 - DNA, 401 bp, Homo sapiens
gggggcgattttcgttttctttagtaacagcccctggaatgctttctacaagcacggaattaggcaatggtaagcacttcttgaagattactcttctgtttcatcctggtatcttgaggtggatctccttggtatctctgatccacgatgtttgcaaagccagaccactcccttatgactccaaggggcttcctggtttaytgcttctctggcctagcccttccaatgttcagatgtggtatatgacacagttacctgctctgcggtcttgcaatgatcatttattgtgttttttttataaatcatgtccccccaaatttgaaaatgatgggcttatagttggcattttttgggtgggcccatatatatggatgctgtggaatcgaagtgtggtatggagt

SEQ ID NO: 388 - DNA, 401 bp, Homo sapiens
tttactgcttctctggcctagcccttccaatgttcagatgtggtatatgacacagttacctgctctgcggtcttgcaatgatcatttattgtgttttttttataaatcatgtccccccaaatttgaaaatgatgggcttatagttggcattttttgggtgggcccatatatatggatgctgtggaatcgaagtgtggtatrgagtacacgcatgtacagctgtttggttggtggcagaggtagcactaggaaaatgggaatggtggttgcaaaaggggccacaggtatggtgtaagatcttgacatatgggaagctgtcagtacctaacatacgggaaaaacattattttagagctacatgtgcctgctctctaaatcatagaagcattgaagaaagactt

SEQ ID NO: 389 - DNA, 401 bp, Homo sapiens
ccattagttataaaaagggaacaatatcaccaagaaaaccccggagatgtaatcaaatctcggtgaagaggaaatgtggaagtttgattctagcactaatccattaataactggggtgtgtccccaccaacaaacactattctagatgattccctgatggatattataaatttcttgtggttataagataaccagaacaamctattctagttgttgttatttcaactttcatctgcttattttctgcacaactgatgacagtagtggcaagaagtgaagactttggaattaaactgattggattagaaacccagctttgtcatttactacatgtgtgatgaaagcaagttgtttaagctctccaaacttcagtttcgtcatgagtaaaatggcaataattg

SEQ ID NO: 390 - DNA, 401 bp, Homo sapiens
ttgacttgaaatcgttacatactacaatttagcttagcttgctgaaggatggggacctaataaaagtgaagaatatgtaaataaagtgctattttaatgaaagtcaaggaaatatgcttcctgtgtctactatatatatgtactggctcctatgtcatttcccctccatacttctgggcacagtgaatgtctcctttgagsaatggtctcttctctcttcttgtctgggtgttacctgccttttaatgccagctcaaatctcacctcctataaaacctcctcctaacttgtccccggatagggttgtcttccttctctaaacacagctcttgttatctgtagttccctagcacttggcttatatgtcatcatgttatttaattgattatctatatatctag

SEQ ID NO: 391 - DNA, 401 bp, Homo sapiens
gtgagcccagctaagtgttctagaacagcagaggagcggagacaggcagccatattttagggttaatggcacagcttggccatatttgtgcatgtgtgcagtgatttgtttttcagacacagactcaatgacttcccaggctaaaagaccaagatattttgtgaaaaaaatataaaaccaaccaggcttaaaaaaaaaaayctcagctagcaatccacaatgtttatcctttgctggaatcaattcaaagtctgagcagttatatggatttctgagtggatacatttacaagataaatgaacaaaaaagtccacctaaactaagtaaatctgaagatttagtgatttttacaatgtctgttgtgggaatttatgatgtcagtgtctacggacatttaaccc

SEQ ID NO: 392 - DNA, 401 bp, Homo sapiens
taaggcccacccacattatggagaacaatctgctttattcaaagtctactgatttaaatgttaatctcatctaaaaaatatgttcacagcaacatccagactggcttttgatgaaatatctgggtatcatagcctagtcaagttgacacataaaaatagccatcctaggttattaattacaaacatgttcaaatctaggcrgaccctaatgaatgtctctggttactggtttggtggtactcctcctgaatgggactgcttcactcccgtctgtctggtacggagaaaaagaatggctattgaaacccacaaatacatgcttaatcaagcattgaagcaaaattccaaacactaaagtcaaagaaattttaaatatcatccagcaggtgagcaaaaaatgt

SEQ ID NO: 393 - DNA, 401 bp, Homo sapiens
gcatggtggctcacgcgtgtaatcccagcactttgggagtccgaggagggcggatcacctgaggtcaggagttctagaccagcctggccaacgtggcgaaacctcgtctctactaaaaatacaaaaattagctgggcgtggtggcaggctcctgtaatcccagctacttggaaggctgaggcaggagaatcacttgaaccygggaggtggaggttgcggtgagctgaaatcgtgccattgcactccagcctgggcaatgagagtgaaactctgtctcaaaaaaaaaaaaaaaaaaaaaaaaaaaagaatattagcctggaaagagagccacaggattaaacaatccaacctactcctttggagtagaggtaatacacatggagaaaagtttcttgacttgc

SEQ ID NO: 394 - DNA, 401 bp, Homo sapiens; identical to SEQ ID NO: 260

SEQ ID NO: 395 - DNA, 401 bp, Homo sapiens
ctgaggaagaccaatgattaacagaagaaacgatagctaacatcatagacatctcaattggttcagcttacacaatcctgactgaaaaattaaacttgagcaaacttccaactcgatgggtctgtcacaaagccattgcattcagatcagctgaagaaatcagcagagctgtcaatggaaattttaaacaagtgagatccwaatcctgaagcatttcttcaaagcattgtaataggagatggaacctagctttaccagtgtgattttgaagacaaatcacaatccaagcaatggctaccaagaggtggaagtggtcagtcaaaacaaaagcagactagtcaagagcaaaagtcatagcaacagttttttggggacgctgaagatattttgcttgttgactt

SEQ ID NO: 396 - DNA, 401 bp, Homo sapiens
tctctggctaaagctataccaggttagaaaaatctggctatatgacatttaataaaatataaacaccatggtgatcattgtttacttgtgagatcccaggtttcagaggttctgagagggctgcagctgccttagcaattggataaaggctattttgaaaacagtgtcatcacatgccagcctagtaatcaatgccaagtytgggaccactaacagagcatggctgtcttcttaccactgccttttaacttagccaaaggatggcaggtggattctctaggcatttataattttttaactaaaaagcaataggaaaagagaaaacttattggctcaccagaggccataataaacattcagaaacagctcatctgcctatggagttcatttaagacagaacc

SEQ ID NO: 397 - DNA, 401 bp, Homo sapiens
aagaaacaatatggaggaaatgaccctcggcccctcagggagagtttaaaaaacaaaaatccaaataacctaattccacagctttcaccattcccagtggcacttcctatggttccaagaaacttaaattcgacttgatagggttaaatgtccgtagacactgacatcataaattcccacaacagacattgtaaaaatcastaaatcttcagatttacttagtttaggtggacttttttgttcatttatcttgtaaatgtatccactcagaaatccatataactgctcagactttgaattgattccagcaaaggataaacattgtggattgctagctgaggtttttttttttaagcctggttggttttatatttttttcacaaaatatcttggtcttttag

SEQ ID NO: 398 - DNA, 401 bp, Homo sapiens
agaagaaaaaaaaatcgggcacttcactgagaatatcttaataatatttgaagttgggcctgtttgttgtctgagtataaagcatacatgatgtagaatgatcaaaacaaaaactgctgtcctattctgaatttgaggtcattttttctctgtcagtaggtctggttttacttgtcaggagggactgaagaaagcacaggrcgtaagttcctccaagagcattcttggagcagcatctcatcgctgtccttgggtgtagcctcccttttgtgaggttcactccctcagctggaggatgaaccacagtgctgctgcagacctagctgatgtcacagttgttcaaaaggtacattttcttaccctggggggtagagcaacgtggctccttcagttctatgaat

SEQ ID NO: 399 - DNA, 401 bp, Homo sapiens
gatccagaacagaataatgggtagtagagggaggtattgaggataggagagtatatgggtttggcaccacgaggtggatagacaaaacaatttggttgataaggcacagattctgaactaacttgtaagccttgtctggttttaggacaggtaaaatgggggaattgtaaggagagtttataggctttaaaaggccatgcygtagcaggcgagtgataacaggctttaatccttttaaagcgtgctgtgggatgggatcttggctttgagcagggtgagggtgattaggttttaatgagatggtaaggggtgcatgatgggtcaccaaggagggagtagaggtgtcttatacttgtgggttaaggtcggggggataaaagaggacacaaaggaggctttgg

SEQ ID NO: 400 - DNA, 401 bp, Homo sapiens; identical to SEQ ID NO: 255

SEQ ID NO: 401 - DNA, 401 bp, Homo sapiens
cacctcccacctggcacacctccgacattaaggatgacacgtgagttttacagggacacacagccaacccatattattctgaccctgataccccagaacctcatgtccttctcacagagcaaaacacaatcatgccttttcaaaagtttccaaaagtcttaactcattccaaatgtaaaaatttcaaagtctcatctgagwcaaggttacaatcccttctgccaatgagtccctgaatttaaaagggatttcttttccttcaaggtacaataggcattgggtaagttttctcaatccaaagggaagaaattccccagaaaaataacacaaatgcaagtccaaaccccagcaggacagtattcattcaatacagtatctcacagctctgtaatcatcaagag

SEQ ID NO: 402 - DNA, 401 bp, Homo sapiens
aaagttgtaaaagaaaaaatgaagttacagaaacaaaggtggctccatcgtgcaatgaatagtgcattggacttccaaaattacagaaacaaaaaatacaattgcagatacaaaactcacatgatatactcaatagtgagtagagataataaaagaagaaatcagtgaacttgagggccagtcaatcttaatgacagagaraaaaagatggagaaaaataaatgaacaaagcctcagggatctgtgggactataataaaagatgtaatatttctgctattagagtgtcagaaagagaagagagtgtggggctgtgacatgatgcctggaaactttctaaatttggtgaaaaaaataaacacagattgaaaaagctaagtgaacctgaaatataataaactt

SEQ ID NO: 403 - DNA, 401 bp, Homo sapiens
aagagattctgtacgagctgttccttcaggtaccgtctctgggaaggggcggcggaggggcggagcacgtcgggccggaaacatagcgcgcacctggaccacccggagcccggcccccttccgtcccatcctgagcaggggttttaatttcgtttcctcttggcgcacctgtctccaggtgagcggctggggagtgaaatragggcgcgttccccatccccatccgcgctgcagagtcctggggttgggcagggacgtggttccttcaacccccgtttctgattaagggcttcccagaggctgcctgggcctcgggtgtgcgcgggcagaaccgaaaacagcctggtcactgttctccgtcaagtagtaagcacgcctgacatcccccaaaaggagagggg

SEQ ID NO: 404 - DNA, 401 bp, Homo sapiens
cttcaggtaccgtctctgggaaggggcggcggaggggcggagcacgtcgggccggaaacatagcgcgcacctggaccacccggagcccggcccccttccgtcccatcctgagcaggggttttaatttcgtttcctcttggcgcacctgtctccaggtgagcggctggggagtgaaatgagggcgcgttccccatccccatycgcgctgcagagtcctggggttgggcagggacgtggttccttcaacccccgtttctgattaagggcttcccagaggctgcctgggcctcgggtgtgcgcgggcagaaccgaaaacagcctggtcactgttctccgtcaagtagtaagcacgcctgacatcccccaaaaggagaggggtcgcttttataatgtttcctttt

SEQ ID NO: 405 - DNA, 401 bp, Homo sapiens
aaaaaaaaaaaaacaaacaaaaactatttccattggatagcactaatctatattgtagtgagctatacacagcctcaactctttatattgaccctttaatgaaacacctacctttctactatgagaggtcactgtctttcttataccaaaacacagtttagtttactcatggctattgtataatacatgtaaactgttaaytttttttccttaaccttttggatttgttttggtagatgaatgtcttgttttcacagcagtggcatgtgtataatccagtgctgcagcttccttactatgaaatgacagctccacttcctaatagtgcatccgtgtcttcctcactgaatcatgttccagatcttgaggctggacccagctcatataaatggactcaccaa

SEQ ID NO: 406 - DNA, 401 bp, Homo sapiens
tttaagagcacccaagtcacctcttagaatgctttactgcttagaaatttcttccaccagatactataagtcatctctctcaagttcaaaattccacagatctctatctatagaggacatggacaaaacgtcaccagtctctttgctacagcacaagaagagtgacctttactccagtttccaacaagttcctcatctccrcctgagaccacctcagcctgcacttcattgtccatatcactatcagcattgtgatcaaagctatgccacaagtctctatgaagctccaaaccttcccacatcttcctgtctcctgagccctccaagtctctaaggagttccaaactttcccacattttcctgtcttcttctgagcctttcaaactgtttcaacctctgcc

SEQ ID NO: 407 - DNA, 401 bp, Homo sapiens
cacattaaactcgaatttagaagaattagtggtattttgaaacatccagagtgagaagactatttaaatggccagtttagaggcagaaaaataaaaggcataatacaaacatggaaaataatattgggggaatttaaagaatttaagtaagacatggttaaagacattttgaaatgtgaaagccaactaaatagaatttargcaagaaaaatctgaagaaatatgacacactttaaaataatgtaagaaatttggctgctatcattaatttttttaatagatgagtgagtctctaaggaaagtcaactgtcagcgttttgcaaggagcaagactttctgaaaatgacccaggttatctgctcttgacaaagaaatagctgaaatgaggattttctcccact

SEQ ID NO: 408 - DNA, 401 bp, Homo sapiens
attaaacttacttcaggaaaatgaaatggaggctttcctataatattctgtgcaagagaaccattttatgataaaactgcttttttattctttgaccacttaaattgtcctactttagctttttatttctccaagtttcaataatattcactatttagaaaatacgttttcttgacaaaatatgcactatttagaaaatayaggtaccagaaattttttaaaaaaactaatgaagtgagtgtgtgcttaagaaaagaagtatcgtacccaaacaaaacaatgcaagtaaattttgaatgcagaaaaacctaccttttacaaagatgaatgacaatgaaatgtcaccttcttaactatttttatgtacttcacttttttgtttttgcttcattcaattttta

SEQ ID NO: 409 - DNA, 401 bp, Homo sapiens
tttaaaaaaactaatgaagtgagtgtgtgcttaagaaaagaagtatcgtacccaaacaaaacaatgcaagtaaattttgaatgcagaaaaacctaccttttacaaagatgaatgacaatgaaatgtcaccttcttaactatttttatgtacttcacttttttgtttttgcttcattcaatttttattgaatcaaactttawgcaaacttgcttaacgaaatctgaaattggggagtgactgtattgaaatttgaaagaaagctacaatgtaaggcagggttgttgcccagagagcctagcagctgcctgacaagacacctgacaagctggaattgttgaattcagcagggttcagggtctagtctttcattttgaagttcaggagcctgattatatctgca

SEQ ID NO: 410 - DNA, 401 bp, Homo sapiens
tgactgtattgaaatttgaaagaaagctacaatgtaaggcagggttgttgcccagagagcctagcagctgcctgacaagacacctgacaagctggaattgttgaattcagcagggttcagggtctagtctttcattttgaagttcaggagcctgattatatctgcacatctattatggattgcttcacattaaataatctmatttacaaaccaatccctctctcaccatgattgacattataacaaccattttgattaaaggatacaacttctttaggtcacatatttctaaagtagctaagcatacatgacccacaacaggctagtcttccttgtgatcttcctcttctagccaaaagtgataagtcagtactggtgctcaatccaaagtaggccaatca

SEQ ID NO: 411 - DNA, 401 bp, Homo sapiens
aaactaataaagttttgttgcaattaaaatgtgtttttctgcttgttttatttgattgatgtaaaatactttaaattcaataacttcttaccaataattttataaagaacctgacaaaataatcactttaaagagttaactttgctttgaattcctgtctcctcttttctaatgtaaggtgaaagatctacagtactggcrattcattttaaaaatatatatgaaaagactgcccttctgtgacaatataattcacacttttaccaattttactagttttcagtagacttcagggtgaagaggataaaacagcatccatacttttgaacatttatgtaaatatatcattagaaataaataataaagggaaatttatttccaaatcactctccttttttctt

SEQ ID NO: 412 - DNA, 401 bp, Homo sapiens
atgaaggcaggaatgttttgtaggtctgctatagactgaatgtttgtgcccccacaaaattcatatgttgaagccaaatactcagtgtcatggtatttggaggtgaggcctttggaaagtaattaggtcatgaggatggagcttccatgaatgagattactatccttataagaagagacataagagagcttatttcctctytctttctgatctccaccacgtgaaaatataaggctgtcatctgcaaaccaggaagtaggccctcaccagataccagatctatcagcaccttgatcttggatttcccagccttcagaactgtaagaagtaaatgtttactgtttaagccaggcagattgtggtatatttgttatagcagactgaactgactaagataaggc

SEQ ID NO: 413 - DNA, 401 bp, Homo sapiens
gtaggtctgctatagactgaatgtttgtgcccccacaaaattcatatgttgaagccaaatactcagtgtcatggtatttggaggtgaggcctttggaaagtaattaggtcatgaggatggagcttccatgaatgagattactatccttataagaagagacataagagagcttatttcctctctctttctgatctccaccaygtgaaaatataaggctgtcatctgcaaaccaggaagtaggccctcaccagataccagatctatcagcaccttgatcttggatttcccagccttcagaactgtaagaagtaaatgtttactgtttaagccaggcagattgtggtatatttgttatagcagactgaactgactaagataaggcctaagtaaatctataacaa

SEQ ID NO: 414 - DNA, 401 bp, Homo sapiens
cccccacaaaattcatatgttgaagccaaatactcagtgtcatggtatttggaggtgaggcctttggaaagtaattaggtcatgaggatggagcttccatgaatgagattactatccttataagaagagacataagagagcttatttcctctctctttctgatctccaccacgtgaaaatataaggctgtcatctgcaaamcaggaagtaggccctcaccagataccagatctatcagcaccttgatcttggatttcccagccttcagaactgtaagaagtaaatgtttactgtttaagccaggcagattgtggtatatttgttatagcagactgaactgactaagataaggcctaagtaaatctataacaaagcgtgtaggcagcaggctaaaaatgcct

SEQ ID NO: 415 - DNA, 401 bp, Homo sapiens
tgtcatggtatttggaggtgaggcctttggaaagtaattaggtcatgaggatggagcttccatgaatgagattactatccttataagaagagacataagagagcttatttcctctctctttctgatctccaccacgtgaaaatataaggctgtcatctgcaaaccaggaagtaggccctcaccagataccagatctatcarcaccttgatcttggatttcccagccttcagaactgtaagaagtaaatgtttactgtttaagccaggcagattgtggtatatttgttatagcagactgaactgactaagataaggcctaagtaaatctataacaaagcgtgtaggcagcaggctaaaaatgcctttgcatttatggaagtaacattccttgacaaagctcc

SEQ ID NO: 416 - DNA, 401 bp, Homo sapiens
aatataaggctgtcatctgcaaaccaggaagtaggccctcaccagataccagatctatcagcaccttgatcttggatttcccagccttcagaactgtaagaagtaaatgtttactgtttaagccaggcagattgtggtatatttgttatagcagactgaactgactaagataaggcctaagtaaatctataacaaagcgtrtaggcagcaggctaaaaatgcctttgcatttatggaagtaacattccttgacaaagctcccctttagtgcagcaatgaaatttcagaaaatgaaaattaagagcattttccggattgcaaagatagacatccacctttgcaaatgtctagtctttgtaagatagtagcagtgtttcaaatgacacttgcagaaaaagaag

SEQ ID NO: 417 - DNA, 401 bp, Homo sapiens
cttatattcaaattggaacttttgcttatctcataacagctatatactgaataaaatggaggataatcacaaagatttaagatctttgatgacacaaataaaagagacagatgaaataaccgttctggtgacagaaagaaacaccccataattgatacggcaaaataaagaagggcttctttgttttgaattctcctcagmaggcctaggtatatgaatcggattcaataaatttgctattcattttacactgcctctatagttaatcttaaaaacatttaatttttaaacaaaattaagatgaaaaagttggagattttaagttaaaaatcaaagatctagacaaaggagctagagaaagagctaaactgccaaacaatttctatttcaaaaacacaata

SEQ ID NO: 418 - DNA, 401 bp, Homo sapiens
ttcactctgtcttgaaaatactgacactggaaaattgttaaataaagttaattttacctacaatttttaaagctccagaaaactattacatgacagacagtacatacctatttatgattagtactaatgtctattcactaccttctactgttatcaaagaaacaatagtttttcttaatgaacacaaaataatcattaacmaattattttggcagcacataagtataagaaaagagtgtaaatacaaaattaacagagagtgtttgcttctcacctttctcaacagctctttggagtaactggtgctttctatcagttttcaagagcataatattttccaatctctatcaaaataagaaatgtaatggaaaatggatctttcttcttctggaattgcttga

SEQ ID NO: 419 - DNA, 401 bp, Homo sapiens
atattgcattggatcaagaataagaggagaaaccagtatgtttgttttttttaatagcgacggggttttaccttcttggccaggcttgtcttgaactcctgacctcgtgatccacctgccttggcctcccaaagtgctgggattacaggtgtgagccactgcacccggccagaattgtgaatttttatgtgtttgtgtgcmtgtatatatatatgcttgcacttttatttgtttgcttgttataactgatcattgccatgtgacaattttaaaattaacttaacttgcagttccctgaatagataatgctctctcataattccaagcttttgacccatctgaaactgaaattagtagtatgctctgcaatgccttgggtcacttgttcccaaaaacacaga

SEQ ID NO: 420 - DNA, 401 bp, Homo sapiens
cacccggccagaattgtgaatttttatgtgtttgtgtgcctgtatatatatatgcttgcacttttatttgtttgcttgttataactgatcattgccatgtgacaattttaaaattaacttaacttgcagttccctgaatagataatgctctctcataattccaagcttttgacccatctgaaactgaaattagtagtatgytctgcaatgccttgggtcacttgttcccaaaaacacagacttaacttgtaagtcactgcttcaacaaaagaagttttataatttatccatagtcttccacaacaattttcaaatcacagggatcaaaagaaactttagttatcaacaatgacaaaaacaaaaagtgattcttaaaggaatatattattaaatatattagt

SEQ ID NO: 421 - DNA, 401 bp, Homo sapiens
tggaagagaaatatcactacagacacaattcatgagcacccagacatggtggtgatacctgtgaagagtcaataaatctgactaggtcccatcctacaggattaaggaatcaaaaccaaaaatactacatgcacaatgtacctgcagaaattctatgtctttaaattttcacaggtaacattaaagtatgtctgtcaactyggaaaatgaagctaccagcacaccatcagcaggccacttacttactttaaatggttttatttatttttattgaagaaactgcggtgcgtgctttaccctgatctcttatttgcccatctatccatacaaccaagtgtttgaagtactaatctcaggaacaatagccagtaaatgctaatctgagaaccagttctgctgtt

SEQ ID NO: 422 - DNA, 401 bp, Homo sapiens
ctacagacacaattcatgagcacccagacatggtggtgatacctgtgaagagtcaataaatctgactaggtcccatcctacaggattaaggaatcaaaaccaaaaatactacatgcacaatgtacctgcagaaattctatgtctttaaattttcacaggtaacattaaagtatgtctgtcaacttggaaaatgaagctacyagcacaccatcagcaggccacttacttactttaaatggttttatttatttttattgaagaaactgcggtgcgtgctttaccctgatctcttatttgcccatctatccatacaaccaagtgtttgaagtactaatctcaggaacaatagccagtaaatgctaatctgagaaccagttctgctgttgtccaggtttagggca

SEQ ID NO: 423 - DNA, 401 bp, Homo sapiens
caaccaagtgtttgaagtactaatctcaggaacaatagccagtaaatgctaatctgagaaccagttctgctgttgtccaggtttagggcacgttctgcctgtgcagcagggacagtgacacatgctccttggcatggcacctgccactcatagtcagatacagacccaggttggggagactcaggaggctttagaggatgrctccggtgtgctgaggagacagaggcagattagcggtctagggaatgtgggtcagggcttttgtgatttcaattgttggaccaaaaatgtttcagaacaacttttctcttaacaataaagaacattaaaaacaactacttacttcctactattagcatcatttaataaaattaaatcactttaacactgaaaactaaaat

SEQ ID NO: 424 - DNA, 401 bp, Homo sapiens
tttttctgttgctttttgtcttgagatagaatgatgtgtggaatagtacatatatactatattgatctcttcactatttagtagttcatttgatttttttttttttttttgagacagagtcttgctctgtcacccaggttggaatgcagcagcgccatctcagctcagcacaacctccgactccccaggatcaagcgatcstcctacctcagcatcctgagcagctgggaccacaggcacacaccaccatgcctgactaacttttgatttttgtagggacgggttttaccatattgcccagggtggtctcaagctcctgggctcaagagatcctcctgccttggcctcccaaagtgttgagattacaggcatgagccacgatgctcggtcctagttcattc

SEQ ID NO: 425 - DNA, 401 bp, Homo sapiens
cagacaaagcttcagcccagagaactttctcaaacaaaattttttttgacttactagtcaccctatataaagctccttgaggcttaggattgtcttttgtctttagatagatagatagatagatagatagatagatagacagacagatagatagataatatagatagatagtatatatatatgtgtgtgtgtgtgtgtgtrtatatatatatatatcctgaaaaactaaagaaaaagcatttggatctgtaaaataatttaataatctaggtaattaattctttcactcataattttgtgtgacaggtttacttaaagcaaaaagtaatttcatatacaaaagatgcatataatttcttccttacctaaacctttttcttaagaacctatacccaaaccac

SEQ ID NO: 426 - DNA, 401 bp, Homo sapiens
gacaaagcttcagcccagagaactttctcaaacaaaattttttttgacttactagtcaccctatataaagctccttgaggcttaggattgtcttttgtctttagatagatagatagatagatagatagatagatagacagacagatagatagataatatagatagatagtatatatatatgtgtgtgtgtgtgtgtgtatrtatatatatatatcctgaaaaactaaagaaaaagcatttggatctgtaaaataatttaataatctaggtaattaattctttcactcataattttgtgtgacaggtttacttaaagcaaaaagtaatttcatatacaaaagatgcatataatttcttccttacctaaacctttttcttaagaacctatacccaaaccacag

SEQ ID NO: 427 - DNA, 401 bp, Homo sapiens
caaagcttcagcccagagaactttctcaaacaaaattttttttgacttactagtcaccctatataaagctccttgaggcttaggattgtcttttgtctttagatagatagatagatagatagatagatagatagacagacagatagatagataatatagatagatagtatatatatatgtgtgtgtgtgtgtgtgtatatrtatatatatatcctgaaaaactaaagaaaaagcatttggatctgtaaaataatttaataatctaggtaattaattctttcactcataattttgtgtgacaggtttacttaaagcaaaaagtaatttcatatacaaaagatgcatataatttcttccttacctaaacctttttcttaagaacctatacccaaaccacagac

SEQ ID NO: 428 - DNA, 401 bp, Homo sapiens
gtggaggtgaggaggaggggagcctgaaatttttggatatcaagaaacacgtttcatattcaaaaagaacaattcatttagactagggccactagaaaataaacaaaaacttgtcattttcacatgaaaatgaaactaaaatactggtcttattaatcatgaaaaaaaaagaaacatgaaagagaataggtagcttatttmcttgcccattttccccacttctcctactttgtaaatttgtaaatatgacaataaatctagactattaagtggaagagtatttcctaatgaatactctagaatcagatgtgcatccgtcaagtgggttcagtcacgattctagcgccactagcctaagagctgctccagaaggtgagcatgtactctttccctctcaacaa

SEQ ID NO: 429 - DNA, 401 bp, Homo sapiens
catattccttatgctgggaactgtgtactagggagggccggggtgggttacttttaagcaatgcaaatactaatgcccaagtttgggtcagactagggctgtccaagccctagatgatgagccttgagcaaggttgaagagacttgagtaaggatgagaattaatgaggcagtgtagagggaatgtcttttcccaatacamaatttaggtactatgactattaactagaagaaggtaagagccagagggcaacagtaagaaacagccaagaggtaatgaaatcttaaaagagatgctggtgttcaagctatatgtgtgaacagacatggaaaatgaattcctaaagtgaaactctaatgcaaagactgggtggatggttatgttgaagttggccagcaaac

SEQ ID NO: 430 - DNA, 401 bp, Homo sapiens
ttttgttttgtatgctttcagacaggtctcactctgttgcccaggcctgagtgtggtggtgtgaccatgactcactgcagcctgaaactcctgggctcaagcaatcctcccacctcagcctccggaatagacggggactatagttgcgtgccaccatgcttggctaattttacatgtttgtttgtttgtttatagagacgsggtttcgaaatgttgcccaggttggtctagaactcccggactcaagtgatcccccgaccaggcctcccaaagtgctgggattatcggtgtgagccaccaacccctgcagagtctacatctgaaataaaccacagaagcaaggatatcacttaaacaatgtaacttaccaattcagtctgagtgagttttgtagtttataa

SEQ ID NO: 431 - DNA, 401 bp, Homo sapiens
caccaatacaactttaaattttgtagtagccacactttttcaaagttgaaaaaaagtaaaattaatttgagtatattttatttaacaaaacataccatggtatcaatatgaaatcactataaaattcttaagatacttcacattctttttgtagtactaagtccttcctccaagttggtgtgtattttacacctacagacyacctccattccggaaagctgcatgtggcttgtggctcgtagcttcttcattggacagtacagctctccagctacacagacttctctggatcccagaacaccaacagtgctctccggtctttgggcgtctgtgtgagtggttctctcttttgagaaagttctttctggctttcttatggctgcctcctttgcctccttccc

SEQ ID NO: 432 - DNA, 401 bp, Homo sapiens
ccttccccaaactcagcttaaatatcacccgctaagatacgtctttcccaatgactttaggcaaacagtctccccattttattccctagcagagcacctcggagatcacagttgtcttaggacttctctcattctgtcattttttgtgcttgctttccttgcttttcaaatatttgtttatgtcctgttttccaccactaractataactattgctccatgagaacaaggaccacttttattctgttcaccatgggcacatctgagataccgaatttttgctaaatataataaatgcacgtcattactcttagttgctccctggccacttctaacataatattcagaaacaactgggagcataatatacgcacaagaaaacaataaataaatatatataat

SEQ ID NO: 433 - DNA, 401 bp, Homo sapiens
actcttgggctcaagcaatcctctcgcctcaacctcccaagtagctagttaattttttaatttttagtagcgatagggtcttactatggtgcccagactgatcttgaactcctgggctcaagcaatcctcctacctcggcctcccaaactgctgggcttacaggggtgaaccactatgcctggccactgtaattttatgtraagttttcttctcaaattttaatacctttcttcataatactaaatatgttgtcacccattcttggcatggaagtctactgagtccacatccatagggagcaattgaaaattctgtgtgtatgagtttgctttgatttatgttctatttggttataaactttactgcagctacaggaataaaatttggctaatataaacgt

SEQ ID NO: 434 - DNA, 401 bp, Homo sapiens
tacaaagtttcaaaaaagtagtaattcttacaggggtgctttaaagaggcagaatcagatcctgtttgaagtctgaattttgtactttgaactatcaaccttcattaagtattaggtaagggttttgctaagacagtgacatgtaccttttcagaacagtgtgagagcgtatttcccttggctataagacagagatgtccracttaaattataatgtggttcttggagctaagagttctatccaacgctaatgagaaaaaaaattaactcagcaaacatttcttgagtcccgactacgtttaggacacagaattaaaaaataaaaactacccaacattctatcagcttcatgtttttagatgatcactcataatgatatcaagtcctcaaattccagcttg

SEQ ID NO: 435 - DNA, 401 bp, Homo sapiens
tgtcccgctgagacctcagaaagaatttaacagagtatcagccaaggtaagagagtagggagtaaaacagaacaggagtcaccagggccaagtaaatatctgccagagagcagactgtactctcagaaaaggaccgccagcatcagggatcaggttgaggcaagtggaggggaaggtcacacaaaggagagaaggaaagayttgaggggaagcctactcaatcctgcaaaataacacatcaacttcattttgtgaaacttttttaaacgaccaaacctctagagttgggcgaatcaccgtagaagaagtattagaaggtacacaaatcataaagaggacttgaaagccggggtagggggaaaattgacagggcaaaactaggaatgaccttactgagtatc

SEQ ID NO: 436 - DNA, 401 bp, Homo sapiens
agcttcaagctagaagcttacatagactgtactatgaacatttttttaaataaataaatgcaaaaaggaagcaagcgaggtttgaaaattactcatatacctctaatccagaatgaagaaattctagttatctaaataaaatgcataagctctgacgttagaatcagaaaattttcagatatgcaaaccacaggccacatkgttgaagggcaaatgtagcagagaatcaaggagactatcaggttcacaacagttaatttcagtgaaaaaagaccagggtctgtgtaagaagagaaagtgtattgacagtttgatcaacacttttaaaattattttgattccttcatttgaaacctttcatggaaaaggaaaaaaaaaaaaacctgagttgatatggaggc

SEQ ID NO: 437 - DNA, 401 bp, Homo sapiens
aaataaaaaaaaatatgactttgaggagtgttttatgaagttggacaggcattccatgtatacaattagcctactttaccaacctcgtaacccagctcatcagtactggcactttttttttgagatggagtcttgctctgtcgcccaggctggagtgcagtggcgcgatctcagctcactgcaagctccacctcccgggtwcacgccattctcctgcctcagcctcccgagtagctgggactacaggcgcccgccatggcgcccggctaattttttgtatttttagtacagacagggtttcaccgtgtcagccaggatggtctcgatctcctgacctcgtgatccgcccgcctcggcctctcaaagtgctgggattgcaagcgtgagccaccacgcccaga

SEQ ID NO: 438 - DNA, 401 bp, Homo sapiens
ggaaacaatcacttgccctgattatatatacctagaaggatgtgattcacacatcaacttaacaataaccatatgtgtaattacatgagtactcctattagaatgtggtagggacctcgggaatttctgaagtctccaatccccagtctctgaagtaatactcaaggcacgcccatcttcccgttatcaacattatctacsaactcactgaaattccagtagaatttaactgttcagttttcaattaccttttttccttcttgaaaaaagattataaccttttattttaaagttggaacaagaaagtacccataactatatttgagaatgctggccgggcgcagtggctcacacctgtaatcccagcactttgggaagccgaaatgggcagatcatgaggt

SEQ ID NO: 439 - DNA, 401 bp, Homo sapiens
catgagaaatgaaggcttatttctaccaaaagacccaaaaaagttcacagcagccttatttgcagacacaaactcgaaagaaaactaaatgtccacttacagtggaatggataaatggtggtataatcatagaatagaacatagacaatttagggggaaaaaacaaataacatacacaacatggatgactcttacagacayaatgttgataaaaaaagacactgatgagttcatacgacatggttccaactttatatagtttaaaacaggcaaaactaatctatagtaaaagacgccaaagactattcctgttaggtagaggcaggatcgattagaaaatacacaaattacaataagccttctggagtagggtgctagttactcagactatacttacataa

SEQ ID NO: 440 - DNA, 401 bp, Homo sapiens
gccttggatatagatcaagaagactatgttcaccactgacattagtcaaattggctggcatatccatatgtgatctatgaggtatgctatggcctgaatgttgtgtctccccaaaattcataaattgaaaccatatttgcaatgtgagggtaatttggaggtggggcctttggtgagtgattagatcatgagggcaatgcyctcatgaacaggattagtacccttataaaagaggtcccaataattctctggccacctccacccatgtgaggttacagtgagaagtcagccatctatgaggaacaggcccttactaaacattaaatctgctggcaccttggtcttggacttcccagcctccagaactgtgagaaataaacctctatggtgtataagcctcc

SEQ ID NO: 441 - DNA, 401 bp, Homo sapiens
tattttaattttctttctataagaaacaattcagagtcagacatgaaatatatgtgtgtaactaggtaacaggcaatatttggttcctattctttgacaaggataagctattgagaccaatcagaaccttccaatgtgtttccaatgttcttggctacatttctacttaaactcaaacctcaggaagaatataatctgttmagataacttttgtttttctcaaacattctctctctgcctatcccaaaagaatcaatcaatgctttatgttactaagttcactgattatgaggacaatgtctgaagatttcatacacataatattgggatcttccctttccttgaatttcagaatatgttggagccaatttctataaccaaaacgtagtatttgtgtatct

SEQ ID NO: 442 - DNA, 401 bp, Homo sapiens
ttccgtggcaaagcctcactaaaacctatctaatccctagctccaattctttaatctccatggtgcttctgattaatagttaaaccacccggaagtatgttaaggatactaacattttcaccccgaagtattatccttcgccatcctctagaatatgctatgcaaagaggcctaaaacaaaataaactgcgacttcagcasctgagaagtaatgattcctttcccctcccacacatcaaaacttcctatcctatcccctcactactttttttcctttttaaaagtaacaatggtcattgagaactactaaatcctgcaacactcacaaggtagtatgtacaaccacagttttcccccgcgatacttcactgaagtattcaatacagccaatgacttaaact

SEQ ID NO: 443 - DNA, 401 bp, Homo sapiens
aacaaaaggaaagttctgttttaaataaactccaaccttcagaatcctatacattcagtatccatcaacctgaacactaaatttcaagaaaaaacaacagatgggcatccatataatagtttgcaaagaaaccctgataagacttcaggagtttctgtggtctctattttccccctaccatgaatcataggtaatgtgctyaaataaagcattatttctgcagttatttacatgtttatctttatcagagatcctataaaagaattatccagttcaccacccaaactttagcactcagaacacggttttccctgttgatgaagacccttgtaaaattccagatcttaaaagattaaagaaaatgtgatctaatattaattcattttcctccaaagtataac

SEQ ID NO: 444 - DNA, 401 bp, Homo sapiens
ctagccttgctcattttgttgcaataaaaacaaacaaactgaacaatgtgatatgtttctggaataggtggaggaaagacaatggcttttttcagagagaggtgttgcatttatttgagttaatcctgaagttctctgaaatttggcaatggtagttttatgtttcaattcagtgactgctgtttaaactgcaggattgaygcacaaatgatccataacacattttggcatattaaatcatcttcacagttctaatacatcatgaaacccaatttacatataacgtaggcacagaagatgaaaagctgagaacagacccctgtttcaaaaggcatcaggcacactactttattttcttgaagctccagtaacttctataaatccatcgctggaaaaggtta

SEQ ID NO: 445 - DNA, 401 bp, Homo sapiens
tcacctcatatgcctgctttttttttttttttttttttttttttgagactgagtctcgctctgccgcccaggctggagtgcagtggcgcgatctcggctcactgcaagctccacctcccgggttcacgccattctcctgcctcagcctcccgagtagctgggactacaggtgcccaccaccaggcacagttaatttttttktgtgtttttagtggagacggggtttcaccatattagccaggatggtctcgatctcctgacctcgtgatccgcccgcctcggcctcccaaggtgctgggattacaggcgtgagccaccgcacccagcatgcctgcctttttaactgggagtggttattttgaccctaggggaactaggctgcttatagcacaatcataaga

SEQ ID NO: 446 - DNA, 401 bp, Homo sapiens
caacataccagaatctctgggacacatttaaagcagttgtgtagagggaaatttatagcactaaatgcccacaagagaaaggaggaaagatctaaaactgacaccctaacatcacaattaaaagaactagagaagcaagagcaaacacattcaaaagctagcagaaggcaagaaataactaaaatcagagcagaactgaargaaatagagacacaaaaaccccttcaaaaaattaatgaatccaggagctggttttttaaaggatcaacaaaattgatagaccgcaagcaagactaataaagaagaaaagagagaagaatcaaacagacgcaataaaaaatgataaaggggatatcaccaccaatcccacagaaaaacaaactaccatcagagaatactac

SEQ ID NO: 447 - DNA, 401 bp, Homo sapiens
cccgctctgaacacctgtggccctctgggcacagctgtgatctgaactcataaccccaagccattgtcttaaaaatctatactaaaatgaaaattttgtaacattgtagctacctccctgatatggtttgtctctgtgtccccaccaaatctcatctcaaattataatccccaactattaagggaaggacctggtgggagrtgattgtatcatgggggtagatttcctccatgctgatcttgtgattgtgagtcctcacaagatctgatggtttaaaagtgcttggcagtccccatccaccctgcctcctgccaccatgtaagacatgccttgcttccacttgaccttctgccatgattgtttaagtttcctgaggcctcctaagccatgtggaactgtga

SEQ ID NO: 448 - DNA, 401 bp, Homo sapiens
gaattcaggaccctgtctctctaaaaataaaataaaataaaataaaattagctgttcgtggtagagtctgtctgaagtcctagctactcaggaggctgagacagaaggatcacttgagcccaggagttcaaagttacagtaagctatgatcatgccactgagctctagaataggctacagagcaagatgaaaagaaaaaaraagaagaagaagaaaagaaaacagtagattaaggaaggtgaagtggcccactgctctgaaagcaaccattctcaatcctataaaactgggtgtttttgacatccagaaaatgccaggcactgtgttagactctagagacagagatttaaaaagaaaaaaaaaaagacttccataccctcaaagcacttctagtgacaaga

SEQ ID NO: 449 - DNA, 401 bp, Homo sapiens
gagtctcagttttgcaggatgaaaagcgttctgtggatagatgctggtgatgttgtgcaacaatgtaaatattcttaatgtcactgaattatacacttagaatggtaaaaatggtaattttacaagtatcctatcacaattttaaaaagaaaaaaagactgcacagtttatcactcagctctgcactatctcacttttacrtatttctcgcaggttagatttttcttcccacaggtttcacgtgatctaggtctggaatacaccttgttctggaaggcagtggcaccaggctgagtatcagtttatgcttagtccatagatagagattgacaggagttgggaaaggtcagagtgcagaaaggaaaacttaccacttgtgtccatttgggggaaactattaa

SEQ ID NO: 450 - DNA, 401 bp, Homo sapiens
cccatctgactcttcttgacaaactaaaaaggctgccttgcaggggagggcagccacccacggtgtcaccctctggctgctcaccggccacccttgggcctctcctctgggccgcccttccttctcgagtgttctccgtgtgagggccctggcccagctggccccaaacgggatttgggcacccaacccagaccggcaggkttgcaggtggttgatgaactcagggccctgccgttctgtttccaggatcccctggcctggggtctaggggatgaggtaatgggactgcccctgcctcccacacatctcctagcggagcgaggggctcacctggtgcacaggcccactttggtggggtggaacagaccacacagagggattgcgggtaaatagaaacgtgt

SEQ ID NO: 451 - DNA, 401 bp, Homo sapiens
gcagtggcaccatcacggctcattgcagcctcaacctcctgggctcaagtgatcctcccacctcagcctctcgagtagctgggactataggcatgtaccacgatgcctggctaatgtttttgtttttgtttttttgtagaaatggggtttcaccatgttgcccaggttagtctcaaactcctgagctcaagcaatccaccygccttggcctcctaaagtgctgggattacaggtgtgagccaccacgcctggcccaaacacagttttaaaatcccattttttttcctactgttatagtcaatagacctaatattagtttgtcaatttgttatgaaagtttgtaaacacttcttcttgatttctgtactcacctgctcattgactgtgtcgagtagctagtc

SEQ ID NO: 452 - DNA, 401 bp, Homo sapiens
gaaggtacacgtggccctcccatgtacctgaagggctgccagcaaccaaatccatccctgggagcagcctgtctctctggtggtcttcaagatctcccttgcggcgtctctttcctcgctgtgtgtctcctcactctcctctctgctcattggtttcttctatttacatccagcacctgctccctcgcaatttctgcttcyactggacacacacggaaagcacctttcaccttctggccctggtgcaaactggctcaggctcttcatgtttcccagctcctgtttcagaaaggatgaccctgatcaacccagctcctacgctcagtctacaccacatcacaggaggctggccaggctatggatgagccaccctgagcttagaggctcacagcccgtccagt

SEQ ID NO: 453 - DNA, 401 bp, Homo sapiens
agagccagtgatttgggtcctcctggaatgttagggaaagcaggttgggggtctctgtctgtcaccccagggcacctagtctttaaaacctgtgctgccccaagaaagggcaagtctcagagccagggctgcccaaaggtcttctgtccccagcaggagtggactgaatagcgtgcccctgggaggtttgtcttcctaagyagatccaatcggtcttcttgttctgatgaagtaaaacagagtggatatcccttgagttagtgctggtgggaggaagtttggggatggagaaagagtctcattctctcccccacctgtcaaaactacaaatctccaattctctaaggtgaggtgggggtcggggagggaacaggcaggtgtctggcaggagtggctggcct

SEQ ID NO: 454 - DNA, 401 bp, Homo sapiens
cgcataaagcccagggacacgcagatagtcatggtgcagccaggatttgaacctaggcagattccagaggctgctgtctgagttgctggtctgagtttccttctagctaaaggtagcagggaaagaacttaccgcctataggaaaacaagatctattttcataagcaacaagaagccctcatgaggacgacaggactggtwgtgtgaagtccaaaagagctccctgccttctcctggcttaggtccacctctgtctgcaagctgatgagtctggccctgaagcgagcagatctttcttaccccactggggcttagctggactcggagctcctgcagccctgacctccccagggcatctgcgactcccagattcagcctaggacttacagagctccctagaa

SEQ ID NO: 455 - DNA, 401 bp, Homo sapiens
actctctgcctggcactctgctggatgttgctccccctggcctggggagcttctggccttacccacttcagagaagatagtagggagtgtgggagagtttccaggtctcagcctgtgtggtatgcgacccacgttgatccctcctgcctctctagtgatgttggtgacttcagctctgtctttgggcctgaacccacagcmccagagcctgcctggagtgaggctgtcccattctccctgccagtccttgtgatgctggccctctacctcactgggccctgtgatgctagccctctaccttaccggaccctggccctcttttgccttccagaggcctagaaaaggatggcagcttcggtgagggcctcaccatgaagaagcagagtggcatgcacctgact

SEQ ID NO: 456 - DNA, 401 bp, Homo sapiens
aacgttcaatgagcaaactttgaggaaggagggctttaacctcatttataaccaaatacattctaatttaaaaaacaaacttcctttttttcccacggggtgaggggaagggggttctaaaaattaaaacaggttaagaaagaacagagagaggaaggaagaaagggagggagaaaaggaaggaaagaaggaagggaaggragggagggagggagggaggggaaggaaggagagagggaaagaaggaagccaaagctttaacaacaagcacaagcactccttatgatgctggcagaagcataaattcgctcagcctttccagagtgcaatctggcaagatatgaaacagcttaaaacatatatgcctatccagagagggagaaagaagaaaggagagtggt

SEQ ID NO: 457 - DNA, 401 bp, Homo sapiens
cgtatgttggaacctaatggcccctgtgatagtgttaagatgtggggcctttgggaggtgattatgtcattagggcttcttcctcagggatgggactcctgcgcttataaaagaggcctgaggaagcttgtctgccttcctcagacaggaaggatgctgctttctagctgcatcctcttatggtggaagcttctgcccttygccacgtgcagatacagctagaagcaccatctatgaggaaggggccctcatcagacaccaaccttgatcttggacttcctggcctccagaactgtgaacaatacatttctatggtttataaattacccagtctaaggtattttgttacagcagcaggaatggactaagtggaccaagaaacatgtggtgtgtattcacag

SEQ ID NO: 458 - DNA, 401 bp, Homo sapiens
tttcaagttcccacattagccagacacacaccttgtgaatcacagactctcatttaatatgtataagtttgtgtgtgcttgcgtgtatacacattggttggataactgaccttagactatccctgccctgttgaagcacaaagagagcattgataaaatgtctcacttgcctttgtatcaacaatgcctacttggcatagsgtgtcacacacataagcatccaaaatgatttctggaacagatgaaagaacttgcatatgaactcattctactgcaaaggggtcaagtggggaaacctggcctcatgatcaagcctcagttatcagggggaagttgtcactttagagatagtacaacatatatatatatatattttttttttaatagagatgaggtcttgc

SEQ ID NO: 459 - DNA, 401 bp, Homo sapiens
acaatatagtaggaaattacatgatcctaaactaaaagacagcaagaagccaaggaggtaagtggcttaattttgtctagagaatgtttgctggtccagactaaattcaacagaacacagctgagtttaaaggaacttcagagccctggaagaatccaggggtcaaagtctgggcctattcatgataaagggaggaaggartctaatggaagatacccactggaatttgagaccctagaaaggtacttcctgaaagtaaactctcccacctcactccactgccaaggggactgcagagagttgcctaagcaagaactgagagggagaagaaacccgtccctgagagattataaccatgggctgtccctttcatgaattccagcccaaattcacaatgctag

SEQ ID NO: 460 - DNA, 401 bp, Homo sapiens
tattatacatagacatagtacatacatatatataaatgtacatatatattaggcatatttcataaattattattataccaaaaattctaattattatgccccaaactgtaacgaatactgagtaaaatgaggcattaatttccacggtcaaggaacccacaaactggtgaaagagacaagattataggatcacttttctaygtaaagtattaaaaaaaataatacttaacaattattgattatccactagataccagccacttttgcacattacatgtgtggagttattaaattgacacaaaaaccctgcaaggtaaatgctattattaaattcatcttataaataaggtaaatgaggtatagagagattaggtaaatcacatacagcacataagggtcag

SEQ ID NO: 461 - DNA, 401 bp, Homo sapiens
tagtaaacagaacacagagaacataattgaatatttgatgattgaaaatcttgcagtgtctgtgagttctcatactgaacatcagttctcatcacacaagcctgtaggtgtacttgtgggatggcaacagattacacagaaactttaatggccttgagaagcactcataaggcttctttttctttatctttatcctttaakaaataaataaatatttgatatcagttgggtaaggtttttgtttgtaaattttttccatctgattaagtttaggggaattttgtttgtctactgctctctgaataagatgagctctctctgcctctctgcacactgagtcggggaagttgagggggtcctttatcaccttcctgagcactgtgggactagatactttcctc

SEQ ID NO: 462 - DNA, 401 bp, Homo sapiens
tgctattaaaagtctggtagttccatgaagtttataattggatttagcctgtacattgtgctctacagtagaaaaattttcatccatgtttaacaaaatcttcatcacatcattagtatttagtcattaaatgctaaagagcataggcttaaaaatttagttgttttctgctaacccataacccagattgttacttacagrtctagattactctctttcctgtactacactgagtaaatatacaaagcactcttcaatctcccttctacaaattcttaaatggctccttttgaaatcataccttgttagtgccagaaagtggcttggaggcaaagttttttttttttttttttttttttttttgagacagtctcgctctatcacccaggctggagtgcaat

SEQ ID NO: 463 - DNA, 401 bp, Homo sapiens
gcttcaaaacagggcgtgggcgtgagcccccaaggtttgttgcctagaggcacttatgagctactcggtaggtcctttctagcacagactgaggaaatgttctttacacatcatgactagataccactaggctctgtttacctcaaggtcatttgttatgaatgtcaattatttcagtataaagtttttgtcactttttgrgctaaagttatatagggctacttgttatctaaaaatcaatggagcatttttgtcacaaaactttttggcatacatgcattcaatttatttataaataattaacatgtactactaaataatgtgtattttaaaacacatacaagaagtcaggcacagagactcatgcctgtaatcccagcacttcgggaagccgaggcggg

SEQ ID NO: 464 - DNA, 401 bp, Homo sapiens
tccccacccaccccgtgtcagtgtgtgggtgtgtgtgtgcatgtgtgtgattccaatggggactgagaaccactgggtgggggtgggaagggaagggacaggatggagaattttactagtcaggtctaattattaacctaaggcaggagagtcgcttctttcaaatactaaattgcaaagtgagaggtgttcagaactagmgggagaaatatgttaatactacatcggactaaattaagtggtttattaatctaattttaagattaatgatgacaaacaaaagataaactcaatctgtctcccaaaaatgaacagaaaagtaaggacaatgataatgaagacaccataaacagaaaggggcctaaaaagacttcaaaacataataacaaaaaaaagaagaa

SEQ ID NO: 465 - DNA, 401 bp, Homo sapiens
ataatgaacaccttctaaatgaagcagttcttcaaacactcttcacaactaaacagcttcaatttttactttgtaatagaatgcaataggcctctttaaaaaaaaaattaagaatatgtcagcggataattactaaactgaaaactgtcattctgaagtgctttggacacacaaaaaatatacacacttacattaaactasatattaggttcagaattctgaaatgttatgtttctatttatccactactgatgaactcaaagagtcgctcttatgtgtccttacagaaaacatttccttacagattcacgtgaaaaaaagttttcaaattggtttctttaatttcatattttgaaagttggcataatctagacagtatcagcaaatgcttttccttcagt

SEQ ID NO: 466 - DNA, 401 bp, Homo sapiens
tcaaagcacagaagatcacataagtgcagaaaagactcatgattaaatattagtcgtttcaggctcatccacaaataccaaacaatgagcatcaacagtatctttatcttccaactccaacagtgttttaataatgtggcttattacagttgaatgtggaggtactttttagcacaatgaatccctctctaaattcaagtwatagaactgttaaacattatgaaaaaagtcagccacagaaaaccaaacatcatgagcaatattctccccacattttgtcttgtgtcgcacacatgtgtcttgtatcttttaattgagaggaggggaaatagaagtcttttgctaactcctataagaaatctgcagagaaacctgtgaacagcagcaatctttaagtggtc

SEQ ID NO: 467 - DNA, 401 bp, Homo sapiens
tttaaatacggaatacatctgtaatttccctaccctgactgcctcctctaagttacaatacctcttacatactctactaacatccacctcaccctgtatggcagttatatctatacgtctctagtaataacatgttgtctctctaaaagcaagaataaagttgaatctcagagcctacttaataaactaacgggtatacartaaaaataatgatggataataaaagccttccaattattaaatatctgtatcactcctggcactgcaaggctgtgtgtgtgtgtgtgtgtgtctgtgtgtgtgtctgtgtgtgtgtgttttgagacggcgtcttgctctgtcactcaggctggagtgcagtgctgtcactcaggctggagtgcaatgatgccatctctgct

SEQ ID NO: 468 - DNA, 401 bp, Homo sapiens
gtttgacagcaattttagtggaagcagcagcagaagtactgctccttgcagagcagggctcccccattagcagtatgaccagagggccagctcaggagcagttctgtagtcacatttatacctactcttaattacacctactcttaattacacgcaaactaaggaacaggttctgcagaaatttctagaagtggggtaacrtctaggttgttgtcacggaaagggacagaaactttcaggtgttgccatggcaatgataaactaacatgacactggtgggcatgtctttatggagaagtgttttcatctcttccctgcttcagctagtcttcacctggtgcaaagtccaagccccaactctggagtcaagtcccgtctcctacctcagtaggaatttactt

SEQ ID NO: 469 - DNA, 401 bp, Homo sapiens
ttttttgaaatgtggagtcttgcctgtcgcccaggctggagtgcaatggcgcaatgttggctcactacaacctccatctcccgggttgaagcgattctcctgccttagcctcccgagtagctgggattacaagcgcctgccaccatacctggctaatttttgtattttcagtagagatggggtttccccatcttggccacrctggtctcaaactcctaacctcatgatccacccgcctcggccttccaaagtgctgggattacaggcgtgagccactgcgcccagcctacttctaaatttttgaagaaacaaattgaaaaagattatacaaataaatggaaagatgtctcatgttcacgaactgaaagaatattgttaaaatgtccatactacccaaagtt

SEQ ID NO: 470 - DNA, 401 bp, Homo sapiens
tgttgtggcatcatttctcaaacttttctgtaccaattagaatagctctctgaggtctgacaggcagaactggctgaagagcctaaaggcattgctaagataagtagtcaggcaagtttttgatacctaggagcttgtgatttcttgttattgggtcctcttcttaattaacaatcattgttcctctattgcagctagcayacacccctgtcagcaagctgcttttcagaatgaacctgcctggacaatagcaatgtaagaggaaacacttccacacagctgcaaaacctaaccactcccagatagacagggaatccatcaatcaactctgagaataaatcctgctgatacaatcctctggtggtgtccctttgagagtattattattattatttttaaac

SEQ ID NO: 471 - DNA, 401 bp, Homo sapiens
tcacttcccaccaggttcctcccagggcacatgggaattgtgggagttacaattcaagatgagatttgggtgggcacacagccaaaccatattagaagaagtactgaagagtcactcaaagcaagtaaatgtcatgtgtctgagttactgttagatactctttgaagaagtctttgaataaagagtacatttctggaacaygtggttagtcaggtggtgactaaagtggttgatcatggacacaactgactgcaggctccacagagcagcttttggagtccaagatcactgccaaaaaagaacagagggatgggagggtcctttctgattataactgtaaaagcaacttatgcctcaagacttttaaacgttttgcccataatttcatgaaaagttactgg

SEQ ID NO: 472 - DNA, 401 bp, Homo sapiens
ggcacatattttttcccaacaaacatgaagttcaaacattactcaccatctcttagcttctatgtgaagatggacacaccctcaaggcttctgggccccagagctgttgttgcttttttttttttgaggcagagtctcgctctgttgcccaggctggagtgcagtggtgcaatctcaggtcactgtagtcccgagtagctrggactacaggcgcctgccaccacactcagctaatttttgtatttttaatagagatggggtttcatcatgttggccaggatggtcttgatctattgacctcacgatccacccgcctcagcctcccaaagtgctgggattacaggcgtgagccaccgcacccagcctgttgttgctttttaatgattggagtagatagtgcc

SEQ ID NO: 473 - DNA, 401 bp, Homo sapiens
gcttttttttttttgaggcagagtctcgctctgttgcccaggctggagtgcagtggtgcaatctcaggtcactgtagtcccgagtagctgggactacaggcgcctgccaccacactcagctaatttttgtatttttaatagagatggggtttcatcatgttggccaggatggtcttgatctattgacctcacgatccaccygcctcagcctcccaaagtgctgggattacaggcgtgagccaccgcacccagcctgttgttgctttttaatgattggagtagatagtgccagagcaggtgcctatgcggtatagtccagtctcctcattcctagtaagatcatgtgcaaatttctctcaggactcagtgccaggcaagtctacagttcttccagtaagtaa

SEQ ID NO: 474 - DNA, 401 bp, Homo sapiens
atcctgatttctttctttacttttatgctttatgtatctctgaaagcagcttgaaattctttcgagaatgaggctacatgtaaataatcaatactactactaataatatttgagcagctttatcctttatcatctctacatcccaaataattatactgttaacagttcaatgaaatattctgttgtctagggggaagagcsagggctctaccagggcccgccatggtaaaaatctgacttcagtgatctttataaaatttttacaggttaattttgcatctgagaaagagaacatacgcctatattgacatttttatagttcttaatatccactgtctctttccaaagaaattaatcaattataactgccaccaaccatgagtgagattgcctgaatttgt

SEQ ID NO: 475 - DNA, 401 bp, Homo sapiens; identical to SEQ ID NO: 370

SEQ ID NO: 476 - DNA, 401 bp, Homo sapiens; identical to SEQ ID NO: 266

SEQ ID NO: 477 - DNA, 401 bp, Homo sapiens; identical to SEQ ID NO: 371

SEQ ID NO: 478 - DNA, 401 bp, Homo sapiens; identical to SEQ ID NO: 373

(19) United States
(12) Patent Application Publication
(10) Pub. No.: US 2014/0329719 A1
(43) Pub. Date: Nov. 6, 2014

(54) GENETIC VARIANTS FOR PREDICTING RISK OF BREAST CANCER

(71) Applicants: Patrick Sulem, 105 Reykjavik (IS); Simon Stacey, 201 Kopavogur (IS)

(72) Inventors: Patrick Sulem, 105 Reykjavik (IS); Simon Stacey, 201 Kopavogur (IS)

(73) Assignees: ILLUMINA, INC., San Diego, CA (US); DECODE GENETICS EHF, 101 Reykjavik (IS)

(21) Appl. No.: 14/126,828

(22) PCT Filed: Jun. 14, 2012
(86) PCT No.: PCT/IS2012/050009

§ 371 Date: Dec. 16, 2013

(30) Foreign Application Priority Data
Jun. 16, 2011 (IS) 050016

(51) Int. Cl. C12Q 1/68; G06F 19/10

(52) U.S. Cl. CPC C12Q 1/6886; G06F 19/10; C12Q 2600/156; C12Q 2600/118
USPC 506/16; 506/38; 702/19

(57) ABSTRACT

The invention pertains to certain genetic variants that have been determined to be susceptibility variants of breast cancer. Methods of disease management, including diagnosing increased susceptibility to breast cancer, methods of predicting response to therapy and methods of predicting prognosis using such variants are described. The invention further relates to kit, medium and apparatus useful for assessing risk of breast cancer.

### BACKGROUND OF THE INVENTION

Breast cancer is by far the most common cancer in women worldwide. Current global incidence is in excess of 1,151,000 new cases diagnosed each year [Parkin, et al., (2005), CA Cancer J Clin, 55, 74-108]. Breast cancer incidence is highest in developed countries, particularly amongst populations of Northern European ethnic origin, and is increasing. In the United States the annual age-standardized incidence rate is approximately 122 cases per 100,000 populations, more than three times the world average. Rates in Northern European countries are similarly high. In the year 2010 it is estimated that 209,060 new cases of invasive breast cancer will be diagnosed in the U.S.A. and 40,230 people will die from the disease [Jemal, et al., (2010) CA Cancer J Clin, 60, 277-300]. To this figure must be added a further 54,010 ductal and lobular carcinoma in-situ diagnoses expected in 2010. From an individual perspective, the lifetime probability of developing breast cancer is 12.1% in U.S. women (i.e., 1 in 8 women will develop breast cancer during their lives). As with most cancers, early detection and appropriate treatment are important factors. Overall, the 5-year survival rate for breast cancer is 89%. However, in individuals presenting with regionally invasive or metastatic disease, the rate declines to 84% and 23%, respectively [Jemal, et al., (2010) CA Cancer J Clin, 60, 277-300].

Increasingly, emphasis is falling on the identification individuals who are at high risk for primary or recurrent breast cancer. Such individuals can be managed by more intensive screening, preventative chemotherapies, hormonal therapies and, in cases of individuals at extremely high risk, prophylactic surgery. Mass screening programs constitute a huge economic burden on health services, while preventative therapies have associated risks and quality of life consequences.

### Genetic Predisposition to Breast Cancer

The two primary classes of known risk factors for breast cancer are endocrine factors and genetics. Regarding the latter, approximately 12% of breast cancer patients have one or more first degree relatives with breast cancer [Anon (2001), Lancet, 358, 1389-99]. The well known, dominant breast cancer predisposition genes BRCA1 and BRCA2 confer greatly increased breast cancer risk to carriers, with lifetime penetrance estimates ranging up to 90%. The presence of BRCA1 and BRCA2 mutations can account for the majority of families with 6 or more cases of breast cancer and for a large proportion of families comprising breast and ovarian or male breast cancer. However such families are very rare indeed. BRCA1 and BRCA2 mutations are found much less frequently in families with fewer cases or in families characterized by breast cancer cases only. Together, mutations in BRCA1 and BRCA2 can account for 15-20% of the risk for familial breast cancer. In non-founder populations, if all common BRCA mutations could be detected, between 2-3% of incident breast cancer patients would be expected to harbor a mutation [Gorski, et al., (2005), Breast Cancer Res Treat, 92, 19-24; (2000), Br J Cancer, 83, 1301-8]. This low “chance to find” statistic precludes the responsible use of BRCA mutation testing outside families with an obvious hereditary predisposition (Anon[(2003), J Clin Oncol, 21, 2397-406]). Rare, high penetrance mutations are known to occur in the TP53 and PTEN genes, however, these together account for no more than 5% of the total genetic risk for breast cancer [Easton, (1999), Breast Cancer Res, 1, 14-7]. Linkage studies have been largely unsuccessful in identifying any more, widespread mutations conferring high risk for breast cancer [Smith, et al., (2006), Genes Chromosomes Cancer, 45, 646-55].

Epidemiological studies have indicated that the majority of breast cancer cases arise in a predisposed, susceptible minority of the population [Antoniou, et al., (2002), Br J Cancer, 86, 76-83; Pharoah, et al., (2002), Nat Genet, 31, 33-6]. Data from twin studies and observations of the constant, high incidence of cancer in the contralateral breast of patients surviving primary breast cancer indicate that a substantial portion of the uncharacterized risk for breast cancer is related to endogenous factors, most probably genetic [Lichtenstein, et al., (2000), N Engl J Med, 343, 78-85; Peto and Mack, (2000), Nat Genet, 26, 411-4]. Knowledge of the genetic factors that underpin this widespread risk is very limited. Segregation analyses predict that the uncharacterized genetic risk for breast cancer is most likely to be polygenic in nature, with risk alleles that confer low to moderate risk and which may interact with each other and with hormonal risk factors. Nevertheless, these studies predict as much as 40-fold differences in relative risk between the highest and lowest quintiles of a distribution that could be defined by genetic profiling that captures these low to moderate risk alleles [Antoniou, et al., (2002), Br J Cancer, 86, 76-83; Pharoah, et al., (2002), Nat Genet, 31, 33-6]. 88% of all breast cancer cases are expected to arise amongst a predisposed 50% of the population and the 12% of the population at highest risk accounts for 50% of all breast cancer cases [Pharoah, et al., (2002), Nat Genet, 31, 33-6; Pharoah, (2003), Recent Results Cancer Res, 163, 7-18; discussion 264-6]. Much focus is therefore directed towards the identification of such genetically predisposed individuals and developing personalized medical management strategies for them.

Understanding of the genetic factors contributing to the uncharacterized genetic risk for breast cancer is limited. Variants in several genes have been confirmed as moderate penetrance breast cancer risk genes; CHEK2, ATM, PALB2, BRIP1, NBS1 and RAD51C [Renwick, et al., (2006), Nat Genet, 38, 873-5; (2004), Am J Hum Genet, 74, 1175-82; Meijers-Heijboer et al., (2002) Nat Genet, 31, 55-9; Vahteristo et al., (2002) Am J Hum Genet, 71, 432-8; Rahman et al., (2007) Nat Genet, 39, 142-3; Erkko et al., (2007) Nature, 446, 316-9; Seal et al., 2006 Nat Genet, 38, 1239-41; Steffen et al., (2006) Int J Cancer, 119, 472-5; Meindl et al., (2010) Nat Genet, 42, 410-4]. Furthermore, a recent genome-wide association studies have identified low penetrance associations between breast cancer and common genetic variants at the following loci: 1p11 (NOTCH1), 2q33 (CASP8), 2q35 (IGFBP2, IGFBP5), 3p24 (NEK10), 5p12 (MRPS30), 5q11 (MAP3K1), 8q24 (MYC), 9p21 (CDKN2A/B), 10p15 (ANKRD16, FBXO18), 10q21 (ZNF365), 10q22 (ZMIZ1), 10q26 (FGFR2), 11p15 (LSP1, IGF2), 11q13 (CCND1), 14q24 (RAD51L1), 16q12 (TOX3), 17q23 (COX11) [Stacey et al., (2007) Nat Genet, 39, 865-9; Stacey et al., (2008) Nat Genet, 40, 703-6; Easton et al., (2007) Nature, 447, 1087-93; Ahmed et al., (2009) Nat Genet 41, 585-90; Turnbull et al., (2010) Nat Genet, 42, 504-7; Thomas et al., (2009) Nat Genet, 41, 579-84; Hunter et al., (2007) Nat Genet, 39, 870-4.]

No universally successful method for the prevention or treatment of breast cancer is currently available. Management of breast cancer currently relies on a combination of primary prevention, early diagnosis, appropriate treatments and secondary prevention. There are clear clinical imperatives for integrating genetic testing into all aspects of these management areas. Identification of cancer susceptibility genes may also reveal key molecular pathways that may be manipulated (e.g., using small or large molecular weight drugs) and may lead to more effective treatments. The present invention provides additional genetic variants for breast cancer than can be integrated in prevention programs for breast cancer.

### SUMMARY OF THE INVENTION

The present invention is based on the finding by the present inventors that certain genetic variants on chromosomes 2, 3 and 21 are associated with risk of breast cancer. The invention provides various diagnostic applications based on this surprising finding, including methods, kits, media and apparati useful for determining breast cancer risk.

In one aspect, the invention provides a method of determining a susceptibility to breast cancer in a human individual, the method comprising steps of (i) obtaining sequence data about a human individual identifying at least one allele of at least one polymorphic marker, wherein different alleles of the at least one polymorphic marker are associated with different susceptibilities to breast cancer in humans, and (ii) determining a susceptibility to breast cancer from the sequence data.

In another aspect, the invention provides a method of determining a susceptibility to breast cancer in a human individual, the method comprising steps of (i) analyzing sequence data from a human individual identifying at least one allele of at least one polymorphic marker, wherein different alleles of the at least one polymorphic marker are associated with different susceptibilities to breast cancer in humans, and (ii) determining a susceptibility to breast cancer from the sequence data.

The at least one polymorphic marker is suitably selected from the group consisting of rs1556283, rs7586009 and rs1983011, and markers correlated therewith. The correlated markers are markers that are in linkage disequilibrium (LD) and correlated with the respective anchor marker (rs1556283, rs7586009 and rs1983011), as described in more detail in the detailed description of the invention.

In another aspect, the invention provides a method of assessing a susceptibility to breast cancer in a human individual, comprising (i) obtaining sequence information about the individual for at least one polymorphic marker selected from the group consisting of rs1556283, rs7586009 and rs1983011, and markers correlated therewith, wherein different alleles of the at least one polymorphic marker are associated with different susceptibilities to breast cancer in humans; (ii) identifying the presence or absence of at least one allele in the at least one polymorphic marker that correlates with increased occurrence of breast cancer in humans; wherein determination of the presence of the at least one allele identifies the individual as having elevated susceptibility to breast cancer, and wherein determination of the absence of the at least one allele identifies the individual as not having the elevated susceptibility.

Further provided is a method of determining a susceptibility to breast cancer in a human individual, the method comprising screening nucleic acid from a sample from the individual for the presence or absence of at least one allele selected from the group consisting of rs1556283 allele C, rs7586009 allele C and rs1983011 allele C, and an allele of at least one polymorphic marker that is in linkage disequilibrium and correlated with rs1556283, rs7586009 or rs1983011 by a value of r² greater than 0.2, and determining a susceptibility to breast cancer from the presence or absence of the at least one allele, wherein determination of the presence of the at least one allele is indicative that the individual is at increased susceptibility to breast cancer, and wherein determination of the absence of the at least one allele is indicative that the individual is at decreased susceptibility to breast cancer.

Also provided is a method of determining a susceptibility to breast cancer in a human individual, the method comprising analyzing nucleic acid from the individual for evidence of the presence of an allele selected from the group consisting of rs1556283 allele C, rs7586009 allele C and rs1983011 allele C, and determining an increased susceptibility to breast cancer in the individual from evidence that the allele is present in the individual, or determining a decreased susceptibility to breast cancer from evidence that the allele is absent in the individual. In certain embodiments, the evidence is provided by providing marker alleles that are correlated with rs1556283 allele C, rs7586009 allele C and/or rs1983011 allele C.

The invention also provides a method of determining a susceptibility to breast cancer, the method comprising obtaining sequence data about a human individual identifying at least one allele of at least one polymorphic marker, wherein different alleles of the at least one polymorphic marker are associated with different susceptibilities to breast cancer in humans, and determining a susceptibility to breast cancer from the sequence data, wherein the at least one polymorphic marker is a marker within a gene selected from the group consisting of the NRIP1 (Nuclear receptor-interacting protein 1) gene, the LOC100134259 gene, the TTC7A (Tetratricopeptide repeat domain 7A) gene, the SOCS5 (Suppressor of cytokine signaling 5) gene, the CRIPT (Cysteine-rich PDZ-binding protein) gene, the RHOQ (Ras homolog gene family, member Q) gene and the TBL1XR1 (Transducin (beta)-like 1 X-linked receptor 1) gene. Such a marker is suitably a marker that is in linkage disequilibrium with any one of the above named genes, i.e. the human NRIP1gene, the LOC100134259 gene, the TTC7A gene, the SOCS5 gene, the CRIPT gene, the RHOQ gene and the TBL1XR1 gene.

The invention further provides a method of identification of a marker for use in assessing susceptibility to breast cancer in human individuals, the method comprising (a) identifying at least one polymorphic marker correlated with a marker selected from the group consisting of rs1556283, rs7586009 and rs1983011; (b) obtaining sequence information about the at least one correlated marker in a group of individuals diagnosed with breast cancer, identifying the presence or absence of at least one allele of the at least one marker; and (c) obtaining sequence information about the at least one correlated marker in a group of control individuals; wherein determination of a significant difference in frequency of at the least one allele in the at least one correlated marker in individuals diagnosed with breast cancer as compared with the frequency of the at least one allele in the control group is indicative of the at least one correlated marker being useful for assessing susceptibility to breast cancer. In a suitable embodiment, an increase in frequency of the at least one allele in the at least one correlated marker in individuals diagnosed with breast cancer, as compared with the frequency of the at least one allele in the control group, is indicative of the at least one allele being useful for assessing increased susceptibility to breast cancer, and wherein a decrease in frequency of at least one allele in the at least one correlated marker in individuals diagnosed with breast cancer, as compared with the frequency of the at least one allele in the control group, is indicative of the at least one allele being useful for assessing decreased susceptibility to, or protection against, breast cancer.

It is known that individuals who have presented with a first primary tumor, may be at increased risk of later developing a second primary tumor. It is useful to be able to identify those individuals who are at increased risk of developing such second primary tumors. Thus, in another aspect of the invention, a method of determining risk of developing at least a second primary tumor in an individual previously diagnosed with breast cancer is provided, the method comprising obtaining sequence data about the individual identifying at least one allele of at least one polymorphic marker, wherein different alleles of the at least one polymorphic marker are associated with different risk of developing a second primary tumor in humans previously diagnosed with breast cancer, and determining the risk of developing at least a second primary tumor in the individual from the sequence data, wherein the at least one polymorphic marker is selected from rs1556283, rs7586009 and rs1983011, and markers in linkage disequilibrium therewith.

Further provided is a method of predicting prognosis of an individual diagnosed with breast cancer, the method comprising obtaining sequence data about a human individual identifying at least one allele of at least one polymorphic marker selected from the group consisting of rs1556283, rs7586009 and rs1983011, and markers correlated therewith, wherein different alleles of the at least one polymorphic marker are associated with different susceptibilities to breast cancers in humans, and predicting prognosis of breast cancer from the sequence data.

The markers shown herein to be associated with risk of breast cancer may also be useful for determining whether individuals are more or less likely to respond to a particular therapeutic agent for treating breast cancer. Thus, in another aspect the invention relates to a method of assessing probability of response of a human individual to a breast cancer therapeutic agent, comprising obtaining sequence data about a human individual identifying at least one allele of at least one polymorphic marker selected from the group consisting of rs1556283, rs7586009 and rs1983011, and markers correlated therewith, wherein different alleles of the at least one polymorphic marker are associated with different probabilities of response to the therapeutic agent in humans, and determining the probability of a positive response to the therapeutic agent from the sequence data.

Yet another aspect of the invention relates to methods of monitoring the progress of treatment of individuals undergoing treatment for breast cancer. Such a method suitably comprises steps of obtaining sequence data about a human individual identifying at least one allele of at least one polymorphic marker selected from the group consisting of rs1556283, rs7586009 and rs1983011, and markers correlated therewith, wherein different alleles of the at least one polymorphic marker are associated with different outcome of breast cancer treatment in humans, and determining the probability of a positive treatment outcome from the sequence data.

In another aspect of the invention, a method of establishing a diagnosis is provided, by combining use of diagnostic risk markers of breast cancer as identified by the present inventors, in combination with other diagnostic and clinical methods that are useful for making a diagnosis of breast cancer in an individual. Thus, in one aspect the invention provides a method of diagnosing breast cancer in a human individual, the method comprising (A) obtaining sequence data from the individual, identifying the presence or absence of at least one at-risk allele of breast cancer selected from the group consisting of the C allele of rs1556283, the C allele of rs7586009 and the C allele of rs1983011, and marker alleles correlated therewith; and (B) performing at least one of, or a combination of (i) considering symptoms experienced by the human individual and/or the family history of breast cancer for the human individual; (ii) clinical or self-exam screening of a breast for lumps or other abnormalities; (iii) mammographic screening of a breast for breast cancer; (iv) fine needle aspiration cytology; (v) biopsy of breast tissue; and (vi) determination of the presence or absence of at least one additional genetic risk factor of breast cancer in the individual; whereupon a diagnosis of the presence or absence of breast cancer for the individual is made.

Also provided is a method of assessing a subject's risk for breast cancer, the method comprising (a) obtaining sequence information about the individual identifying the presence or absence of at least one allele of at least one polymorphic marker in the genome of the individual; (b) representing the sequence information as digital genetic profile data; (c) transforming the digital genetic profile data on a computer processor to generate breast cancer risk assessment report for the subject; and (d) displaying the risk assessment report on an output device; wherein the at least one polymorphic marker is selected from the group consisting of rs1556283, rs7586009 and rs1983011, and markers correlated therewith. In one suitable embodiment, the digital genetic profile data comprises data indicating the presence or absence of at least one allele of the at least one polymorphic marker.

Further provided is a method of determining a susceptibility to breast cancer in a human individual. Such a method comprises in one embodiment screening nucleic acid from a sample from the individual for the presence or absence of at least one allele selected from the group consisting of rs1556283 allele C, rs7586009 allele C and rs1983011 allele C, and an allele of at least one polymorphic marker that is in linkage disequilibrium and correlated with rs1556283, rs7586009 or rs1983011 by a value of r² greater than 0.2, and determining a susceptibility to breast cancer from the presence or absence of the at least one allele, wherein determination of the presence of the at least one allele is indicative that the individual is at increased susceptibility to breast cancer, and wherein determination of the absence of the at least one allele is indicative that the individual is at decreased susceptibility to breast cancer.

Another method of the invention relates to a method of determining a susceptibility to breast cancer in a human individual, the method comprising analyzing nucleic acid from the individual for evidence of the presence of an allele selected from the group consisting of rs1556283 allele C, rs7586009 allele C and rs1983011 allele C, and determining an increased susceptibility to breast cancer in the individual from evidence that the allele is present in the individual, or determining a decreased susceptibility to breast cancer from evidence that the allele is absent in the individual.

Kits are also provided. In one embodiment, a kit for assessing susceptibility to breast cancer in humans is provided, the kit comprising reagents for selectively detecting at least one allele of at least one polymorphic marker in the genome of the individual, and a collection of data comprising correlation data between the at least one polymorphism and susceptibility to breast cancer. The at least one marker is suitably selected from the group consisting of the markers rs1556283, rs7586009 and rs1983011, and markers in linkage disequilibrium therewith.

The present invention also provides diagnostic reagents. In one such aspect, the invention relates to the use of an oligonucleotide probe in the manufacture of a diagnostic reagent for diagnosing and/or assessing a susceptibility to breast cancer in humans, wherein the probe is capable of hybridizing to a segment of a nucleic acid whose nucleotide sequence is given by any of SEQ ID NO:1-478, and wherein the segment is 15-400 nucleotides in length. In a suitable embodiment, the segment of the nucleic acid to which the probe is capable of hybridizing comprises a polymorphic site. The polymorphic site is suitably selected from the group consisting of the markers rs1556283, rs7586009 and rs1983011, and markers correlated therewith.

The invention also provides computer-implemented aspects. As is known in the art, sequence data can conveniently be stored and analyzed in digital format, and either such sequence data (e.g., genotype data) or results derived therefrom (e.g., disease-risk estimates) can be provided in digital format to an end-user.

One such aspect relates to a computer-readable medium having computer executable instructions for determining susceptibility to breast cancer in humans, the computer readable medium comprising (i) data indicative of at least one polymorphic marker; and (ii) a routine stored on the computer readable medium and adapted to be executed by a processor to determine risk of developing breast cancer for the at least one polymorphic marker; wherein the at least one polymorphic marker is selected from the group consisting of rs1556283, rs7586009 and rs1983011, and markers correlated therewith.

Another computer-implemented aspect relates to an apparatus for determining a genetic indicator for breast cancer in a human individual, comprising (i) a processor; and (ii) a computer readable memory having computer executable instructions adapted to be executed on the processor to analyze marker information for at least one human individual with respect to at least one polymorphic marker selected from the group consisting of rs1556283, rs7586009 and rs1983011, and markers correlated therewith, and generate an output based on the marker information, wherein the output comprises a measure of susceptibility of the at least one marker or haplotype as a genetic indicator of breast cancer for the human individual.

In one embodiment, the computer readable memory further comprises data indicative of the risk of developing breast cancer associated with at least one allele of at least one polymorphic marker, and wherein a risk measure for the human individual is based on a comparison of the marker information for the human individual to the risk of breast cancer associated with the at least one allele of the at least one polymorphic marker.

The invention also provides risk assessment reports. One such aspect relates to a risk assessment report of breast cancer for a human individual, comprising (i) at least one personal identifier, and (ii) representation of at least one risk assessment measure of breast cancer for the human subject for at least one polymorphic marker selected from the group consisting of rs1556283, rs7586009 and rs1983011, and markers correlated therewith. Such reports may be provided in any suitable format, including electronic format (e.g., on a computer-readable medium) or a paper format (e.g., a reported printed or written on paper).

A further aspect of the invention is to provide use of variants for selecting individuals for administration of therapeutic agents for treating breast cancer. One such aspect provides use of an agent for treating breast cancer in a human individual that has been tested for the presence of at least one allele of at least one polymorphic marker selected from the group consisting of rs1556283, rs7586009 and rs1983011, and markers correlated therewith.

Preferably, an at-risk variant for breast cancer is used for selecting individuals who would benefit from administration of the therapeutic agents. Thus, in certain embodiments, the at least one allele is selected from the group consisting of the C allele of rs1556283, the C allele of rs7586009 and the C allele of rs1983011.

It should be understood that all combinations of features described herein are contemplated, even if the combination of feature is not specifically found in the same sentence or paragraph herein. This includes for example embodiments that relate to any one or a combination of the markers disclosed herein, for analysis individually or in haplotypes, in all aspects of the invention as described herein.

### BRIEF DESCRIPTION OF THE DRAWINGS

FIG. 1 provides a diagram illustrating a computer-implemented system utilizing risk variants as described herein.

FIG. 2 provides a diagram illustrating a system comprising computer implemented methods utilizing risk variants as described herein.

FIG. 3 shows an exemplary system for determining risk of breast cancer as described further herein.

FIG. 4 shows a system for selecting a treatment protocol for a subject diagnosed with breast cancer.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. Sequence codes for nucleotides used herein are: A=1, C=2, G=3, T=4. For microsatellite alleles, the CEPH sample (Centre d′Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 by longer than the shorter allele in the CEPH sample, allele 2 is 2 by longer than the shorter allele in the CEPH sample, allele 3 is 3 by longer than the lower allele in the CEPH sample, etc., and allele −1 is 1 by shorter than the shorter allele in the CEPH sample, allele −2 is 2 by shorter than the shorter allele in the CEPH sample, etc.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

The sequence listing presented herein provides flanking sequence for the polymorphic markers described herein, with the polymorphic site indicated in the sequence using the sequence conucleotide ambiguity code as shown above.

A nucleotide position at which more than one sequence is possible in a population (either a natural population or a synthetic population, e.g., a library of synthetic molecules) is referred to herein as a “polymorphic site”.

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “2 rs1556283” refers to the 2 allele of marker rs1556283 being in the haplotype, and is equivalent to “rs1556283 allele 2”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., breast cancer), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of breast cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of breast cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa, placenta, gastrointestinal tract or other organs.

The term “breast cancer therapeutic agent” refers to an agent that can be used to ameliorate or prevent symptoms associated with breast cancer.

The term “breast cancer-associated nucleic acid”, as described herein, refers to a nucleic acid that has been found to be associated to breast cancer. This includes, but is not limited to, the markers and haplotypes described herein and markers and haplotypes in strong linkage disequilibrium (LD) therewith.

The term “Breast Cancer”, as described herein, refers to any clinical diagnosis of breast cancer, and includes any and all particular sub-phenotypes of breast cancer. For example, breast cancer is sometimes categorized as estrogen receptor (ER) positive breast or estrogen receptor negative breast cancer; breast cancer is sometimes also categorized as progesterone receptor (PR) positive or negative. Breast cancer is furthermore sometimes diagnosed as invasive ductal, as invasive lobular, as tubular, as medullary, or as otherwise invasive or mixed invasive. Breast cancer can also be categorized as DCIS (Ductal Carcinoma In-Situ) or LCIS (Lobular Carcinoma In-Situ), or otherwise non-invasive. Invasive breast cancer can also be defined as stage 0, stage 1, stage 2 (including stage 2a and stage 2b), stage 3 (including stage 3a, stage 3b and stage 3c) or stage 4 breast cancer. In the present context, “breast cancer” can include any of these sub-phenotypes of breast cancer, and also includes any other clinically applicable sub-phenotypes of breast cancer.

The term “estrogen receptor positive breast cancer”, or “ER-positive breast cancer”, as described herein, refers to tumors determined to be positive for estrogen receptor. In the present context, ER levels of greater than or equal to 10 fmol/mg and/or an immunohistochemical observation of greater than or equal to 10% positive nuclei is considered to be ER positive. Breast cancer that does not fulfill the criteria of being ER positive is defined herein as “ER negative” or “estrogen receptor negative”.

The term “estrogen receptor negative breast cancer”, or “ER negative breast cancer”, as described herein, refers to tumors determined to be negative for estrogen receptor.

The term “progesterone receptor positive breast cancer”, or “PR-positive breast cancer”, as described herein, refers to tumors determined to be positive for progesterone receptor. In the present context, PR levels of greater than or equal to 10 fmol/mg and/or an immunohistochemical observation of greater than or equal to 10% positive nuclei is considered to be PR positive. Breast cancer that does not fulfill the criteria of being PR positive is defined herein as “PR negative” or “progesterone receptor negative”.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine an pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “NRIP1” or “NRIP1 gene”, also known as; “RIP140 or F1177253”, as described herein, refers to the Nuclear receptor-interacting protein 1 gene on human chromosome 21q11.2.

The term “LOC100134259” or “LOC100134259 gene”, as described herein, refers to a gene of yet unknown function located on human chromosome 2p21.

The term “TTC7A” or “TTC7A gene”, also known as; “TTC7; MGC131720 or MGC134830”, as described herein, refers to the Tetratricopeptide repeat domain 7A gene on human chromosome 2p21.

The term “SOCS5” or “SOCS5 gene”, also known as; “CIS6; CISH6; Cish5; SOCS-5 or KIAA0671”, as described herein, refers to the Suppressor of cytokine signaling 5 gene on human chromosome 2p21.

The term “CRIPT” or “CRIPT gene”, also known as “HSPC139” as described herein, refers to the Cysteine-rich PDZ-binding protein gene on human chromosome 2p21.

The term “RHOQ” or “RHOQ gene”, also known as “ARHQ; TC10; TC10A or RASL7A”, as described herein, refers to the Ras homolog gene family, member Q gene on human chromosome 2p21.

Also, the term “TBL1XR1” or “TBL1XR1 gene”, also known as “C21; DC42; IRA1; TBLR1 or F1112894”, as described herein, refers to Transducing(beta)-like 1 X-linked receptor 1 gene on human chromosome 3q26.32.

### Identification of Variants as Diagnostic Markers of Breast Cancer

Through association analysis of a population of individuals diagnosed with breast cancer, the present inventors have discovered that certain alleles at certain polymorphic markers at human chromosome locations 21q21.1, 2p21 and 3q26.32 are associated with breast cancer. Particular markers within these regions were found to be associated with an increased risk of breast cancer.

Through genotyping followed by “genealogy-based imputation” (see Example 1) of 2601 samples from Icelandic breast cancer patients and 36219 controls we selected approximately 60 SNPs for further investigation, based on their association P values, after excluding signals originating from previously known loci. These SNPs were then genotyped in breast cancer case:control foreign cohorts from USA, Spain, Holland, Sweden, Germany and Belarus. After replication genotyping, SNPs from three loci on chromosomes 21, 2 and 3 showed combined P values <5×10⁻⁷.

### Methods of Determining Susceptibility to Breast Cancer

Accordingly, in a first aspect, the present invention provides a method of determining a susceptibility to breast cancer in a human individual, the method comprising (i) analyzing sequence data from a human individual identifying at least one allele of at least one polymorphic marker, wherein different alleles of the at least one polymorphic marker are associated with different susceptibilities to breast cancer in humans, and (ii) determining a susceptibility to breast cancer from the sequence data. In one embodiment, the at least one polymorphic marker is selected from the group consisting of rs1556283, rs7586009 and rs1983011, and markers in linkage disequilibrium therewith. In a further embodiment, the at least one polymorphic marker is rs1556283, and markers in linkage disequilibrium therewith. In certain embodiments, the at least one polymorphic marker is a marker that is in linkage disequilibrium and correlated with at least one anchor marker, e.g., rs1556283, rs7586009 and/or rs1983011, as described in more detail herein.

In another aspect, the invention provides a method of using a polymorphic marker selected from the group consisting of rs1556283, rs7586009 and rs1983011, and markers in linkage disequilibrium therewith, the method comprising analyzing sequence data from a sample comprising nucleic acid from a human individual identifying at least one allele of at least one polymorphic marker, wherein different alleles of the at least one polymorphic marker are associated with different susceptibilities to breast cancer in humans, and wherein a determination of the presence of the at least one allele is indicative of a susceptibility to breast cancer for the human individual.

In certain embodiments, the sequence data is nucleic acid sequence data. Nucleic acid sequence data identifying particular alleles of polymorphic markers is sometimes also referred to as genotype data. Nucleic acid sequence data can be obtained for example by analyzing sequence of the at least one polymorphic marker in a biological sample from the individual. Alternatively, nucleic acid sequence data can be obtained in a genotype dataset from the human individual and analyzing sequence of the at least one polymorphic marker in the dataset. Such analysis in certain embodiments comprises determining the presence or absence of a particular allele of specific polymorphic markers.

In certain embodiments, nucleic acid sequence data is obtained using a method that comprises at least one procedure selected from (i) amplification of nucleic acid from the biological sample, (ii) hybridization assay using a nucleic acid probe and nucleic acid from the biological sample, (iii) hybridization assay using a nucleic acid probe and nucleic acid obtained by amplification of the biological sample, and (iv) high-throughput sequencing.

Estrogen receptor (ER) negative breast cancer is a subtype of breast cancer arising in approximately 25% of cases in individuals of European ancestry. ER negative breast cancer presents challenges in terms of both prevention and treatment, primarily because it is not usually responsive to hormonal therapies. It is therefore of significant diagnostic value to detect increased risk of ER negative breast cancer in individuals. Accordingly, in certain embodiments, the breast cancer phenotype detected by the variants described herein is estrogen receptor negative breast cancer. Particularly, in certain embodiments, rs7586009, and markers that are correlated and in linkage disequilibrium therewith, are useful for detecting susceptibility to ER negative breast cancer. In certain alternative embodiments, rs1983011, and markers that are correlated and in linkage disequilibrium therewith, are useful for detecting susceptibility to ER negative breast cancer.

Identification of particular alleles in general terms should be taken to mean that determination of the presence or absence of the allele(s) is made. Usually, determination of both allelic copies in the genome of an individual is performed, by determining the occurrence of all possible alleles of the particular polymorphism in a particular individual (for SNPs, each of the two possible nucleotides possible for the allelic site). It is also possible to determine whether only particular alleles are present or not. For example, in certain embodiments, determination of the presence or absence of certain alleles that have been shown to associate with risk of breast cancer is made, but not necessarily other alleles of the particular marker, and a determination of susceptibility is made based on such determination. In certain embodiments, sequence data about at least two polymorphic markers is obtained.

In certain embodiments, the at least one polymorphic marker is a marker associated with the human NRIP1 gene, the LOC100134259 gene, the TTC7A gene, the SOCS5 gene, the CRIPT gene, the RHOQ gene or the TBL1XR1 gene. In certain embodiments, the marker is selected from the group consisting of rs1556283, rs1983011 and rs7586009, and markers in linkage disequilibrium therewith.

In certain embodiments, markers correlated with rs1556283 are selected from the group consisting of the markers set forth in Table 3 (A and B). In certain embodiments, markers correlated with rs1983011 are selected from the group consisting of the markers set forth in Table 5 (A and B). In certain embodiments, markers correlated with rs7586009 are selected from the group consisting of the markers set forth in Table 4 (A and B). In some preferred embodiments, correlated markers with rs1983011 are selected from the group consisting of the markers set forth in Table 8. In some other preferred embodiments, correlated markers with rs1556283 are selected from the group consisting of the markers set forth in Table 6. In some other preferred embodiments, correlated markers with rs7586009 are selected from the group consisting of the markers set forth in Table 7. These correlated markers are thus particularly useful in the methods of the invention, as described further herein.

Surrogate markers correlated with particular key markers can in general be selected based on suitable numerical values of the linkage disequilibrium measure r², as described further herein. For example, markers that are in linkage disequilibrium with rs1556283, rs1983011 and rs7586009 are exemplified by the markers listed in Tables 3, 5 and 4 herein, but the skilled person will appreciate that other markers in linkage disequilibrium with these markers may also be used in the diagnostic applications described herein. Further, as also described in more detail herein, the skilled person will appreciate that since linkage disequilibrium is a continuous measure, certain values of the LD measures D′ and r² may be suitably chosen to define markers that are useful as surrogate markers in LD with the markers described herein. Numeric values of D′ and r² may thus in certain embodiments be used to define marker subsets that fulfill certain numerical cutoff values of D′ and/or r². In one embodiment, markers in linkage disequilibrium with a particular anchor marker (e.g., rs1556283) are in LD with the anchor marker characterized by numerical values of D′ of greater than 0.8 and/or numerical values of r² of greater than 0.2. In one embodiment, markers in linkage disequilibrium with a particular anchor marker are in LD with the anchor marker characterized by numerical values of r² of greater than 0.2. The markers provided in Tables 3, 4 and 5 provides exemplary markers that fulfill this criterion. In other embodiments, markers in linkage disequilibrium with a particular anchor marker are in LD with the anchor marker characterized by numerical values of r² of greater than 0.3, greater than 0.4, greater than 0.5, greater than 0.6, greater than 0.7, greater than 0.8, greater than 0.9, and greater than 0.95. Other numerical values of r² and/or D′ may also be suitably selected to select markers that are in LD with the anchor marker. The stronger the LD, the more similar the association signal and/or the predictive risk by the surrogate marker will be to that of the anchor marker. Markers with values of r²=1 to the anchor marker are perfect surrogates of the anchor marker and will provide identical association and risk prediction data. In one preferred embodiment, surrogate markers of rs1556283, rs1983011 and rs7586009 are those markers that have values of r² to one of these markers of greater than 0.8.

Further, as described in more detail in the following, LD may be determined in samples from any particular population. In one embodiment, LD is determined in Caucasian samples. In another embodiment, LD is determined in European samples. In another embodiment, LD is determined in Icelandic samples. In certain other embodiments, LD is determined in African American samples, in Asian samples, or the LD may be suitably determined in samples of any other population.

The sequence data that is obtained may in certain embodiments be amino acid sequence data. Polymorphic markers can result in alterations in the amino acid sequence of encoded polypeptide or protein sequence. In certain embodiments, the analysis of amino acid sequence data comprises determining the presence or absence of an amino acid substitution in the amino acid encoded by the at least one polymorphic marker. Sequence data can in certain embodiments be obtained by analyzing the amino acid sequence encoded by the at least one polymorphic marker in a biological sample obtained from the individual. In certain embodiments, the at least one polymorphic marker that is assessed is an amino acid substitution in a polypeptide encoded by the human NRIP1gene, the LOC100134259 gene, the TTC7A gene, the SOCS5 gene, the CRIPT gene, the RHOQ gene or the TBL1XR1 gene. In other words, the marker may be an amino acid substitution in any of the above human polypeptide.

In certain embodiments of the invention, determination of the presence of particular marker alleles or particular haplotypes is predictive of an increased susceptibility of breast cancer in humans. In certain embodiments, determination of the presence of a marker allele selected from the group consisting of the C allele of rs1556283, the C allele of rs7586009, and the C allele of rs1983011 is indicative of increased risk of breast cancer in the individual. These marker alleles confer increased risk of breast cancer with relative risk or odds ratio of greater than unity, and are sometimes also referred to as at-risk alleles or at-risk variants. Individuals who are homozygous for at-risk alleles are at particularly high risk of developing breast cancer, since their genome includes two copies of the at-risk variant.

Measures of susceptibility or risk include measures such as relative risk (RR), odds ratio (OR), and absolute risk (AR), as described in more detail herein.

In certain embodiments, increased susceptibility refers to a risk with values of RR or OR of at least 1.10, at least 1.11, at least 1.12, at least 1.13, at least 1.14, at least 1.15, at least 1.16, at least 1.17, at least 1.18, at least 1.19, at least 1.20, at least 1.25, at least 1.30, at least 1.35, at least 1.40, at least 1.45, at least 1.50, at least 1.55, at least 1.60, at least 1.65, at least 1.70, at least 1.75, and at least 1.80. Other numerical non-integer values greater than unity are also possible to characterize the risk, and such numerical values are also within scope of the invention. Certain embodiments relate to homozygous individuals for a particular markers, i.e. individuals who carry two copies of the same allele in their genome. One preferred embodiment relates to individuals who are homozygous carriers of the C allele of rs1556283, homozygous carriers of the C allele of rs1983011 or homozygous carriers of the C allele of rs7586009.

In certain other embodiments, determination of the presence of particular marker alleles or particular haplotypes is predictive of a decreased susceptibility of breast cancer in humans. For SNP markers with two alleles, the alternate allele to an at-risk allele will be in decreased frequency in patients compared with controls. Thus, determination of the presence of the alternate allele is indicative of a decreased susceptibility of breast cancer. Individuals who are homozygous for the alternate (protective) allele are at particularly decreased susceptibility or risk.

To identify markers that are useful for assessing susceptibility to breast cancer, it may be useful to compare the frequency of markers alleles in individuals with breast cancer to control individuals. The control individuals may be a random sample from the general population, i.e. a population cohort. The control individuals may also be a sample from individuals that are disease-free, e.g. individuals who have been confirmed not to have breast cancer. In one embodiment, an increase in frequency of at least one allele in at least one polymorphism in individuals diagnosed with breast cancer, as compared with the frequency of the at least one allele in the control group is indicative of the at least one allele being useful for assessing increased susceptibility to breast cancer. In another embodiment, a decrease in frequency of at least one allele in at least one polymorphism in individuals diagnosed with breast cancer, as compared with the frequency of the at least one allele in the control sample is indicative of the at least one allele being useful for assessing decreased susceptibility to, or protection against, breast cancer.

In general, sequence data can be obtained by analyzing a sample from an individual, or by analyzing information about specific markers in a database or other data collection, for example a genotype database or a sequence database. The sample is in certain embodiments a nucleic acid sample, or a sample that contains nucleic acid material. Analyzing a sample from an individual may in certain embodiments include steps of isolating genomic nucleic acid from the sample, amplifying a segment of the genomic nucleic acid that contains at least one polymorphic marker, and determine sequence information about the at least one polymorphic marker. Amplification is preferably performed by Polymerase Chain Reaction (PCR) techniques. In certain embodiments, sequence data can be obtained through nucleic acid sequence information or amino acid sequence information from a preexisting record. Such a preexisting record can be any documentation, database or other form of data storage containing such information.

Determination of a susceptibility or risk of a particular individual in general comprises comparison of the genotype information (sequence information) to a record or database providing a correlation about particular polymorphic marker(s) and susceptibility to disease, such as breast cancer. Thus, in specific embodiments, determining a susceptibility comprises comparing the sequence data to a database containing correlation data between the at least one polymorphic marker and susceptibility to breast cancer. In certain embodiments, the database comprises at least one measure of susceptibility to breast cancer for the at least one polymorphic marker. In certain embodiments, the database comprises a look-up table comprising at least one measure of susceptibility to breast cancer for the at least one polymorphic marker. The measure of susceptibility may in the form of relative risk (RR), absolute risk (AR), percentage (%) or other convenient measure for describing genetic susceptibility of individuals.

Certain embodiments of the invention relate to markers associated with the human NRIP1 gene, the LOC100134259 gene, the TTC7A gene, the SOCS5 gene, the CRIPT gene, the RHOQ gene or the TBL1XR1 gene. Markers that are associated with these genes are in certain embodiments markers that are correlated (in linkage disequilibrium (LD)) with at least one genetic marker within the genes. In certain embodiments, markers associated with the NRIP1 gene are selected from the markers within the human NRIP1 gene. In certain other embodiments, markers associated with the LOC100134259 gene are selected from the markers within the human LOC100134259 gene and so forth for the human TTC7A gene, the SOCS5 gene, the CRIPT gene, the RHOQ gene and the TBL1XR1 gene. In certain embodiments of the invention, more than one polymorphic marker is analyzed. In certain embodiments, at least two polymorphic markers are analyzed. Thus, in certain embodiments, nucleic acid data about at least two polymorphic markers is obtained.

In certain embodiments, a further step of analyzing at least one haplotype comprising two or more polymorphic markers is included. Any convenient method for haplotype analysis known to the skilled person may be employed in such embodiments.

One aspect of the invention relates to a method for determining a susceptibility to breast cancer in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of rs1556283, rs1983011 and rs7586009, and markers correlated therewith, and wherein determination of the presence of the at least one allele is indicative of a susceptibility to breast cancer. Determination of the presence of an allele that correlates with breast cancer is indicative of an increased susceptibility to breast cancer. Individuals who are homozygous for such alleles are particularly susceptible to breast cancer. On the other hand, individuals who do not carry such at-risk alleles are at a decreased susceptibility of developing breast cancer. For SNPs, such individuals will be homozygous for the alternate (protective) allele of the polymorphism.

Determination of susceptibility is in some embodiments reported by a comparison with non-carriers of the at-risk allele(s) of polymorphic markers. In certain embodiments, susceptibility is reported based on a comparison with the general population, e.g. compared with a random selection of individuals from the population.

In certain embodiments, polymorphic markers are detected by sequencing technologies.

Obtaining sequence information about an individual identifies particular nucleotides in the context of a nucleic acid sequence. For SNPs, sequence information about a single unique sequence site is sufficient to identify alleles at that particular SNP. For markers comprising more than one nucleotide, sequence information about the genomic region of the individual that contains the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the invention. Sanger sequencing is a well-known method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al. Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLiD) technology (Applied Biosystems, http://www.appliedbiosystems.com); Strausberg, R L, et al Drug Disc Today 13:569-577 (2008).

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucelotide Polymorphisms (“SNPs”). These SNPs are believed to have arisen by a single mutational event, and therefore there are usually two possible alleles possible at each SNPsite; the original allele and the mutated (alternate) allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions, inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. All sequence variants can be referred to as polymorphisms, occurring at specific polymorphic sites characteristic of the sequence variant in question. In general, polymorphisms can comprise any number of specific alleles within the population, although each human individual has two alleles at each polymorphic site—one maternal and one paternal allele. Thus in one embodiment of the invention, the polymorphism is characterized by the presence of two or more alleles in a population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 9 million human SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007). http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the disease-associated markers described herein. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 21,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site. The allele codes for SNPs used herein are as follows: 1=A, 2=C, 3=G, 4=T. Since human DNA is double-stranded, the person skilled in the art will realize that by assaying or reading the opposite DNA strand, the complementary allele can in each case be measured. Thus, for a polymorphic site (polymorphic marker) characterized by an A/G polymorphism, the methodology employed to detect the marker may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+ strand or − strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a single-stranded segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In certain embodiments, polymorphic markers are detected by sequencing technologies. Obtaining sequence information about an individual identifies particular nucleotides in the context of a sequence. For SNPs, sequence information about a single unique sequence site is sufficient to identify alleles at that particular SNP. For markers comprising more than one nucleotide, sequence information about the nucleotides of the individual that contain the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the invention. Sanger sequencing is a well-known method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al. Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLiD) technology (Applied Biosystems, http://www.appliedbiosystems.com); Strausberg, R L, et al Drug Disc Today 13:569-577 (2008).

It is possible to impute or predict genotypes for un-genotyped relatives of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

In the present context, an individual who is at an increased susceptibility (i.e., increased risk) for a disease, is an individual in whom at least one specific allele at one or more polymorphic marker or haplotype conferring increased susceptibility (increased risk) for the disease is identified (i.e., at-risk marker alleles or haplotypes). The at-risk marker or haplotype is one that confers an increased risk (increased susceptibility) of the disease. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.10, including but not limited to: at least 1.11, at least 1.12, at least 1.13, at least 1.14, at least 1.15, at least 1.16, at least 1.17, at least 1.18, at least 1.19, at least 1.20, at least 1.30, at least 1.40, at least 1.50, at least 1.60, at least 1.70, at least 1.80, at least 1.90, and at least 2.0. In a particular embodiment, a risk (relative risk and/or odds ratio) of at least 1.15 is significant. In another particular embodiment, a risk of at least 1.20 is significant. In yet another embodiment, a risk of at least 1.25 is significant. In a further embodiment, a relative risk of at least 1.30 is significant. In another further embodiment, a significant increase in risk is at least 1.40 is significant. However, other cutoffs are also contemplated, e.g., at least 1.16, 1.17, 1.18, and so on, and such cutoffs are also within scope of the present invention. In other embodiments, a significant increase in risk is at least about 10%, including but not limited to about 20%, 25%, 30%, 35%, 40%, 45%, 50%, 55%, 60%, 65%, 70%, 75%, 80%, 85%, 90%, 95%, and 100%, 150. In one particular embodiment, a significant increase in risk is at least 10%. In other embodiments, a significant increase in risk is at least 20%, at least 25%, at least 30%, at least 35%, at least 40%, at least 50%, at least 60% and at least 70%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype of the present invention is one where at least one allele of at least one marker or haplotype is more frequently present in an individual at risk for the disease or trait (affected), or diagnosed with the disease or trait, compared to the frequency of its presence in a comparison group (control), such that the presence of the marker or haplotype is indicative of susceptibility to the disease or trait (e.g., breast cancer). The control group may in one embodiment be a population sample, i.e. a random sample from the general population. In another embodiment, the control group is represented by a group of individuals who are disease-free, i.e. individuals who have not been diagnosed with breast cancer. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus is other embodiments of the invention, an individual who is at a decreased susceptibility (i.e., at a decreased risk) for a disease or trait such as breast cancer is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk of less than 0.90, including but not limited to less than 0.85, less than 0.80, less than 0.75, less than 0.7, less than 0.6, less than 0.5, and less than 0.4. In one particular embodiment, significant decreased risk is less than 0.90. In another embodiment, significant decreased risk is less than 0.85. In yet another embodiment, significant decreased risk is less than 0.80. In another embodiment, the decrease in risk (or susceptibility) is at least 10%, including but not limited to at least 15%, at least 20%, at least 25%, at least 30%, at least 35%, at least 40%, at least 45%, and at least 50%. In one particular embodiment, a significant decrease in risk is at least about 15%. In another embodiment, a significant decrease in risk at least about 20%. In another embodiment, the decrease in risk is at least about 25%. Other cutoffs or ranges as deemed suitable by the person skilled in the art to characterize the invention are however also contemplated, and those are also within scope of the present invention.

A genetic variant associated with a disease or a trait (e.g. breast cancer) can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk is the product of the locus specific risk values and also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider the case were a total of sixteen variants that have been associated with breast cancer. One such example is provided by the markers rs1045485, rs3817198, rs4973768, rs889312, rs11249433, rs999737, rs6504950, rs4415084, rs1011970, rs13387042, rs9397435, rs3803662, rs13281615, rs10995190, rs2981582 and rs614367, all of which are used in the marketed deCODE BreastCancer test for breast cancer susceptibility (http://www.decodediagnostics.com). The total number of theoretical genotypic combinations is then 3¹⁶=43,046,721. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with any plurality of these and other variants associated with breast cancer may be assessed. This includes the variants that are shown and claimed herein to be predictive of breast cancer risk.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randombly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275(2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.125, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appi. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes for two markers are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and particular SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value between markers indicative of the markers being in linkage disequilibrium can be at least 0.1, such as at least 0.15, 0.20, 0.25, 0.30, 0.35, 0.40, 0.45, 0.50, 0.55, 0.60, 0.65, 0.70, 0.75, 0.80, 0.85, 0.90, 0.91, 0.92, 0.93, 0.94, 0.95, 0.96, 0.97, 0.98, or at least 0.99. In one preferred embodiment, the significant r² value can be at least 0.2. In another preferred embodiment, the significant r² value is at least 0.4. In yet another preferred embodiment, the significant r² value is at least 0.8. Alternatively, markers in linkage disequilibrium are characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. In certain embodiments, linkage disequilibrium is defined in terms of values for both the r² and |D′| measures. In one such embodiment, a significant linkage disequilibrium is defined as r²>0.1 and |D′|>0.8, and markers fulfilling these criteria are said to be in linkage disequilibrium. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (Caucasian, African (Yoruba), Japanese, Chinese), as defined (http://www.hapmap.org). In one such embodiment, LD is determined in the CEU population of the HapMap samples (Utah residents with ancestry from northern and western Europe). In another embodiment, LD is determined in the YRI population of the HapMap samples (Yoruba in Ibadan, Nigeria). In another embodiment, LD is determined in the CHB population of the HapMap samples (Han Chinese from Beijing, China). In another embodiment, LD is determined in the JPT population of the HapMap samples (Japanese from Tokyo, Japan). In another embodiment, LD is determined in a European population. In yet another embodiment, LD is determined in samples from the Icelandic population.

If all polymorphisms in the genome were independent at the population level (i.e., no LD), then every single one of them would need to be investigated in association studies. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Nati Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Nati. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. Markers shown herein to be associated with breast cancer are such tagging markers. If desired, neighboring haplotype blocks can be assessed concurrently, as there may also exist linkage disequilibrium among the haplotype blocks.

It has thus become apparent that for any given observed association to a polymorphic marker in the genome, that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers originally used to detect an association may be used as surrogate markers. The surrogate markers have in one embodiment relative risk (RR) and/or odds ratio (OR) values smaller than originally detected. In other embodiments, the surrogate markers have RR or OR values greater than those initially determined for the markers initially found to be associating with the disease. An example of such an embodiment would be a rare, or relatively rare (<10% allelic population frequency) variant in LD with a more common variant (>10% population frequency) initially found to be associating with the disease. Identifying and using such surrogate markers for detecting the association can be performed by routine methods well known to the person skilled in the art, and are therefore within the scope of the present invention.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessarily from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The Risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

As an example, for marker rs9397435, allele G has an allelic OR for breast cancer of 1.15 and a frequency (p) around 0.063 in Caucasian populations. The genotype relative risk compared to genotype AA are estimated based on the multiplicative model.

For GG it is 1.15×1.15=1.32; for AG it is simply the OR 1.15, and for AA it is 1.0 by definition.

The frequency of allele A is q=1−p=1−0.063=0.937. Population frequency of each of the three possible genotypes at this marker is:

Pr(GG)=p²=0.00397,Pr(AG)=2pq=0.118, and Pr(AA)=q²=0.878

The average population risk relative to genotype AA (which is defined to have a risk of one) is:

R=0.00397×1.32+0.118×1.15+0.878×1=1.019

Therefore, the risk relative to the general population (RR) for individuals who have one of the following genotypes at this marker is:

RR(GG)=1.32/1.019=1.30,RR(AG)=1.15/1.019=1.13,RR(AA)=1/1.019=0.98.

### Combining the Risk from Multiple Markers

When genotypes of many SNP variants are used to estimate the risk for an individual a multiplicative model for risk can generally be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A1g1,g2)=Pr(A1g1)Pr(A1g2)/Pr(A) and Pr(g1,g2)=Pr(g1)Pr(g2)

Obvious violations to this assumption are markers that are closely spaced on the genome, i.e. in linkage disequilibrium, such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 hypothetical markers associated with a particular disease along with the risk relative to the population at each marker:

| Marker | Genotype | Calculated risk |
|---|---|---|
| M1 | CC | 1.03 |
| M2 | GG | 1.30 |
| M3 | AG | 0.88 |
| M4 | TT | 1.54 |

Combined, the overall risk relative to the population for this individual is: 1.03×1.30×0.88×1.54=1.81. In an analogous fashion, overall risk for any plurality of markers (or haplotypes) may be assessed.

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, for a particular disease, if the overall genetic risk relative to the population is 1.8 for an individual, and if the average life-time risk of the disease for individuals of his demographic is 20%, then the adjusted lifetime risk for him is 20%×1.8=36%.

Note that since the average RR for a population is one, this multiplication model provides the same average adjusted life-time risk of the disease. Furthermore, since the actual life-time risk cannot exceed 100%, there must be an upper limit to the genetic RR.

### Risk Assessment for Breast Cancer

As described herein, certain polymorphic markers and haplotypes comprising such markers are found to be useful for risk assessment of breast cancer. Risk assessment can involve the use of the markers for diagnosing a susceptibility to breast cancer. Particular alleles of certain polymorphic markers are found more frequently in individuals with breast cancer, than in individuals without diagnosis of breast cancer. Therefore, these marker alleles have predictive value for detecting breast cancer, or a susceptibility to breast cancer, in an individual. Tagging markers in linkage disequilibrium with at-risk variants (or protective variants) described herein can be used as surrogates for these markers (and/or haplotypes). Such surrogate markers can be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that confers decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants (anchor variants), i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. In certain preferred embodiments, markers with particular values of r² (e.g., values greater than 0.2) to the at-risk anchor variant are useful surrogate markers. The at-risk variant identified may not be the functional variant itself, but is in this instance in linkage disequilibrium with the true functional variant. The functional variant may be a SNP, but may also for example be a tandem repeat, such as a minisatellite or a microsatellite, a transposable element (e.g., an A/u element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers and/or haplotypes as described herein. The tagging or surrogate markers in LD with the at-risk variants detected also have predictive value.

The present invention can in certain embodiments be practiced by assessing a sample comprising genomic DNA from an individual for the presence of certain variants described herein to be associated with breast cancer. Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of breast cancer. Alternatively, the invention can be practiced utilizing a dataset comprising information about the genotype status of at least one polymorphic marker described herein to be associated with breast cancer (or markers in linkage disequilibrium with at least one marker shown herein to be associated with breast cancer). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with breast cancer. A positive result for a variant (e.g., marker allele) associated with increased risk of breast cancer, as shown herein, is indicative of the individual from which the dataset is derived is at increased susceptibility (increased risk) of breast cancer.

In certain embodiments of the invention, a polymorphic marker is correlated to breast cancer by referencing genotype data for the polymorphic marker to a database, such as a look-up table that comprises correlation data between at least one allele of the polymorphism and breast cancer. The correlation data may for example be a value of Relative Risk (RR) or odds ratio (OR). In some embodiments, the table comprises a correlation for one polymorphism. In other embodiments, the table comprises a correlation for a plurality of polymorphisms. In both scenarios, by referencing to a look-up table that gives an indication of a correlation between a marker (particular genotype status at the marker) and breast cancer, a risk for breast cancer, or a susceptibility to breast cancer, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

Risk markers may be useful for risk assessment and diagnostic purposes, either alone or in combination. Results of disease risk assessment based on the markers described herein can also be combined with data for other genetic markers or risk factors for the disease, to establish overall risk. Thus, even in cases where the increase in risk by individual markers is relatively modest, e.g. on the order of 10-30%, the association may have significant implications when combined with other risk markers. Thus, relatively common variants may have significant contribution to the overall risk (Population Attributable Risk is high), or combination of markers can be used to define groups of individual who, based on the combined risk of the markers, is at significant combined risk of developing the disease. Combined risk can be assessed based on genotype results for any combination of markers found to be associated with risk of breast cancer. Such combinations can for example include marker rs13387042 on chromosome 2q35 (Stacey, S N et al. Nat Genet 39:865-9 (2007)), marker rs4415084 on chromosome 5p12 (Stacey, S N et al. Nat Genet 40:703-6 (2008)) marker rs1219648 on chromosome 10q26 (Easton, D F et al Nature 447:1087-93 (2007); Hunter, D J et al. Nat Genet 39:870-4 (2007); Stacey, S N et al. Nat Genet 40:703-6 (2008)), marker rs3803662 on chromosome 16q12 (Stacey, S N et al. Nat Genet 39:865-9 (2007); Stacey, S N et al. Nat Genet 40:703-6 (2008)), marker rs13281615 on chromosome 8q24, marker rs3817198 on chromosome 11p15, and marker rs889312 on chromosome 5q11 (Stacey, S N et al. Nat Genet 40:703-6 (2008)). Other markers that have or will be found to be associated with risk of breast cancer can also be suitably selected. Alternatively, markers in LD with any one of these markers could be assessed.

Thus, in certain embodiment of the invention, a plurality of variants (markers and/or haplotypes) is used for overall risk assessment. These variants are in one embodiment selected from the variants as disclosed herein. Other embodiments include the use of the variants of the present invention in combination with other variants known to be useful for diagnosing a susceptibility to breast cancer In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, such as those described herein, or other methods known to the skilled person, may subsequently be used to determine the overall risk conferred based on the genotype status at the multiple loci. Assessment of risk based on such analysis may subsequently be used in the methods, uses and kits of the invention, as described herein.

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived there from. The present invention also provides for assessing markers in individuals who are members of a target population. Such a target population is in one embodiment a population or group of individuals at risk of developing the disease, based on other genetic factors, biomarkers, biophysical parameters (e.g., weight, BMD, blood pressure), or general health and/or lifestyle parameters (e.g., history of breast cancer, history of breast cancer, previous diagnosis of breast cancer or other cancer, family history of cancer, family history of breast cancer).

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17 2009 (Epub ahead of print); Rafnar, T., et al. Nat Genet 41:221-7 (2009); Gretarsdottir, S., et al. Ann Neurol 64:402-9 (2008); Stacey, S. N., et al. Nat Genet 40:1313-18 (2008); Gudbjartsson, D. F., et al. Nat Genet 40:886-91 (2008); Styrkarsdottir, U., et al. N Engl J Med 358:2355-65 (2008); Thorgeirsson, T., et al. Nature 452:638-42 (2008); Gudmundsson, J., et al. Nat Genet. 40:281-3 (2008); Stacey, S. N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Frayling, T M, Nature Reviews Genet 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers described herein to be associated with risk of breast cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations. In one embodiment, the invention relates to individuals of Caucasian origin.

In certain embodiments, racial contribution is determined by self-reporting of an individual. The racial contribution in individual subjects may also be determined by genetic analysis. Genetic analysis of ancestry may be carried out using any suitable set of polymorphic markers, as well understood by the skilled person. One embodiment of such unlinked microsatellite markers is described in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population. Correlated markers that are in linkage disequilibrium are therefore always suitable for practicing the invention in the particular population in which the correlated markers are identified.

### Models to Predict Inherited Risk for Breast Cancer

The goal of breast cancer risk assessment is to provide a rational framework for the development of personalized medical management strategies for all women with the aim of increasing survival and quality of life in high-risk women while minimizing costs, unnecessary interventions and anxiety in women at lower risk. Risk prediction models attempt to estimate the risk for breast cancer in an individual who has a given set of risk characteristics (e.g., family history, prior benign breast lesion, previous breast tumor). The breast cancer risk assessment models most commonly employed in clinical practice estimate inherited risk factors by considering family history. The risk estimates are based on the observations of increased risk to individuals with one or more close relatives previously diagnosed with breast cancer. They do not take into account complex pedigree structures. These models have the further disadvantage of not being able to differentiate between carriers and non-carriers of genes with breast cancer predisposing mutations.

More sophisticated risk models have better mechanisms to deal with specific family histories and have an ability to take into account carrier status for BRCA1 and BRCA2 mutations. For example, the Breast and Ovarian Analysis of Disease Incidence and Carrier Estimation Algorithm (BOADICEA) (Antoniou et al., 2004) takes into account family history based on individual pedigree structures through the pedigree analysis program MENDEL. Information on known BRCA1 and BRCA2 status is also taken into account. The main limitations of the BOADICEA and all other breast cancer risk models currently in use are that they do not incorporate genotypic information from other predisposition genes. Current models depend strongly on family history to act as a surrogate to compensate for the lack of knowledge of non-BRCA genetic determinants of risk. Therefore the available models are limited to situations where there is a known family history of disease. Lower penetrance breast cancer predisposition genes may be relatively common in the population and may not show such strong tendencies to drive familial clustering as do the BRCA1 and BRCA2 genes. Patients with a relatively high genetic load of predisposition alleles may show little or no family history of disease. There is a need therefore to construct models which incorporate inherited susceptibility data obtained directly through gene-based testing. In addition to making the models more precise, this will reduce the dependency on family history parameters and assist in the extension of the risk profiling into the wider at-risk population where family history is not such a key factor.

### Integration of Improved Genetic Risk Models into Clinical Management of Breast Cancer Primary Prevention

Clinical primary prevention options currently can be classified as chemopreventative (or hormonal) treatments and prophylactic surgery. Patients identified as high risk can be prescribed long-term courses of chemopreventative therapies. This concept is well accepted in the field of cardiovascular medicine, but is only now beginning to make an impact in clinical oncology. The most widely used oncology chemopreventative is Tamoxifen, a Selective Estrogen Receptor Modulator (SERM). Initially used as an adjuvant therapy directed against breast cancer recurrence, Tamoxifen now has proven efficacy as a breast cancer preventative agent [Cuzick, et al., (2003), Lancet, 361, 296-300][Martino, et al., (2004), Oncologist, 9, 116-25]. The FDA has approved the use of Tamoxifen as a chemopreventative agent in certain high risk women.

Unfortunately, long term Tamoxifen use increases risks for endometrial cancer approximately 2.5-fold, the risk of venous thrombosis approximately 2.0-fold. Risks for pulmonary embolism, stroke, and cataracts are also increased [Cuzick, et al., (2003), Lancet, 361, 296-300]. Accordingly, the benefits in Tamoxifen use for reducing breast cancer incidence may not be easily translated into corresponding decreases in overall mortality. Another SERM called Raloxifene may be more efficacious in a preventative mode, and does not carry the same risks for endometrial cancer. However risk for thrombosis is still elevated in patients treated long-term with Raloxifene[Cuzick, et al., (2003), Lancet, 361, 296-300; Martino, et al., (2004), Oncologist, 9, 116-25]. Moreover, both Tamoxifen and Raloxifene have quality of life issues associated with them. To make a rational risk:benefit analysis of SERM therapy in a chemopreventative mode, there is a clinical need to identify individuals who are most at risk for breast cancer. Given that a substantial proportion of risk for breast cancer is genetic, there is a clear clinical need for genetic tests to quantify individuals' risks in this context. One can anticipate similar issues arising from any future cancer chemo-preventative therapies that may become available, such as the aromatase inhibitors. Moreover, as chemopreventative therapies become safer, there is an increased need to identify patients who are genetically predisposed, but do not have massively elevated risks associated with BRCA1 & 2 mutation carriers.

Patients who are identified as being at high risk for breast cancer are considered for prophylactic surgery; either bilateral mastectomy or oophorectomy or both. Clearly such drastic treatments are recommended only for patients who are perceived to be at extremely high risk. In practice, such risks can currently be identified only in individuals who carry mutations in BRCA1, BRCA2 or genes known to be involved in rare breast cancer predisposition syndromes like p53 in Li-Fraumeni Syndrome, PTEN in Cowden's Syndrome.

Estimates of the penetrance of BRCA1 and BRCA2 mutations tend to be higher when they are derived from multiple-case families than when they are derived from population-based estimates. This is because different mutation-carrying families exhibit different penetrances for breast cancer (see [Thorlacius, et al., (1997), Am J Hum Genet, 60, 1079-84] for example). One of the major factors contributing to this variation is the action of as yet unknown predisposition genes whose effects modify the penetrance of BRCA1 and BRCA2 mutations. Therefore the absolute risk to an individual who carries a mutation in the BRCA1 or BRCA2 genes cannot be accurately quantified in the absence of knowledge of the existence and action of modifying genes. Since the treatment options for BRCA1 and BRCA2 carriers can be severe, it is important in this context to quantify the risks to individual BRCA carriers with the greatest accuracy possible. There is a need, therefore, to identify predisposition genes whose effects modify the penetrance of breast cancer in BRCA1 and BRCA2 carriers and to develop improved risk assessment models based on these genes.

Furthermore, there are individuals who are perceived to be at very high risk for breast cancer, perhaps because of a strong family history of breast cancer, but in whom no mutations in known predisposition genes can be identified. Consideration of prophylactic surgery is difficult in such cases because one cannot test the individual to discover whether or not she has inherited a high penetrance predisposition gene. Accordingly, the individual's risk cannot be assessed accurately. There is a clear clinical need, therefore, to identify any high penetrance predisposition genes that remain undiscovered and to develop associated genetic tests for use in primary prevention strategies. Such genes may for example be the genes disclosed herein to be associated with risk of breast cancer. Although the variants shown herein to be associated with risk of breast cancer are fairly common, and conferring a relatively low risk of breast cancer, it is quite possible that higher risk variants exist within one or more of the genes which they are associated with.

### Early Diagnosis

Clinical screening for breast cancer in most western countries consists of periodic clinical breast examination (CBE) and X-ray mammography. There is good evidence to indicate that CBE has little added benefit when used in the context of a good mammographic screening program. In the United Kingdom, women between the ages of 50 and 70 are invited to undergo screening mammography every three years. The situation in the United States varies depending on healthcare provider, however the American Cancer Society recommends annual mammographic screening from age 40. Mammographic screening has proven effectiveness in reducing mortality amongst screened women.

It is unlikely that genetic testing would ever be employed as a means of reducing access to existing mammographic screening programs. However, mammographic screening is not without shortcomings and it is conceivable that genetic testing should be used to select people for augmented screening programs. Mammography is less effective in women under 50 possibly because the density of breast tissue is higher in younger women, making mammographic detection of tumors more difficult. However, breast cancers in predisposed individuals tend to occur at early ages groups and there is a clear association between high breast density and breast cancer risk. Therefore there is a problem with simple increases in mammographic screening for individuals with high predisposition because they would be managed by a technique that performs sub-optimally in the group at highest risk. Recent studies have shown that contrast-enhanced magnetic resonance imaging (CE-MRI) is more sensitive and detects tumors at an earlier stage in this high-risk group than mammographic screening does [Warner, et al., (2004), Jama, 292, 1317-25; Leach, et al., (2005), Lancet, 365, 1769-78]. CE-MRI strategies work particularly well when used in combination with routine X-ray mammography[Leach, et al., (2005), Lancet, 365, 1769-78]. Because CE-MRI requires specialist centers that incur high costs, screening of under-50's must be restricted to those individuals at the highest risk. Present CE-MRI trials restrict entry to those individuals with BRCA1, BRCA2 or p53 mutations or very strong family histories of disease. The extension of this screening modality to a wider range of high-risk patients would be greatly assisted by the provision of gene-based risk profiling tools. Breast imaging using ultrasound methodologies may also be useful for augmented screening of high risk individuals.

There is good evidence to support the notion that early-onset breast cancers and cancers occurring in genetically predisposed women grow faster than cancers in older, less strongly predisposed women. This comes from observations of higher rates of interval cancers in younger women, that is, cancers that arise in the intervals between screening visits in a well-screened population are higher amongst younger women. Therefore there are suggestions that screening intervals, by whatever method, should be reduced for younger women. There is a paradox here in that more frequent screening using more expensive methodologies seems to be required for an age group in which the overall rates of breast cancer are comparatively low. There is a clear clinical need here to identify those young individuals who are most strongly predisposed to develop the disease early, and channel them into more expensive and extensive screening regimes. The variants disclosed herein to confer risk of breast cancer can be useful for identification of individuals who are at particularly high risk of developing breast cancer. Such individuals are likely to most benefit from early and aggressive screening programs, so as to maximizing the likelihood of early identification of the cancer.

### Treatment

Currently, primary breast cancer is treated by surgery, adjuvant chemotherapy, radiotherapy, followed by long term hormonal therapy. Often combinations of three or four therapies are used.

Breast cancer patients with the same stage of disease can have very different responses to adjuvant chemotherapy resulting in a broad variation in overall treatment outcomes. Consensus guidelines (the St Galen and NIH criteria) have been developed for determining the eligibility of breast cancer patients for adjuvant chemotherapy treatment. However, even the strongest clinical and histological predictors of metastasis fail to predict accurately the clinical responses of breast tumors [Goldhirsch, et al., (1998), J Natl Cancer Inst, 90, 1601-8; Eifel, et al., (2001), J Natl Cancer Inst, 93, 979-89]. Chemotherapy or hormonal therapy reduces the risk of metastasis only by approximately ⅓, however 70-80% of patients receiving this treatment would have survived without it. Therefore the majority of breast cancer patients are currently offered treatment that is either ineffective or unnecessary. There is a clear clinical need for improvements in the development of prognostic measures which will allow clinicians to tailor treatments more appropriately to those who will best benefit. It is reasonable to expect that profiling individuals for genetic predisposition may reveal information relevant to their treatment outcome and thereby aid in rational treatment planning. The markers of the present invention, conferring risk of breast cancer, are contemplated to be useful in this context.

Several previous studies exemplify this concept: Breast cancer patients who are BRCA mutation carriers appear to show better clinical response rates and survival when treated with adjuvant chemotherapies [Chappuis, et al., (2002), J Med Genet, 39, 608-10; Goffin, et al., (2003), Cancer, 97, 527-36]. BRCA mutation carriers demonstrate improved responses to platinum chemotherapy for ovarian cancer than non-carriers [Cass, et al., (2003), Cancer, 97, 2187-95]. Similar considerations may apply to predisposed patients in whom the genes involved are not known. For example, infiltrating lobular breast carcinoma (ILBC) is known to have a strong familial component but the genetic variants involved have not yet been identified. Patients with ILBC demonstrate poorer responses to common chemotherapy regimens [Mathieu, et al., (2004), Eur J Cancer, 40, 342-51].

Genetic predisposition models may not only aid in the individualization of treatment strategies, but may play an integral role in the design of these strategies. For example, BRCA1 and BRCA2 mutant tumor cells have been found to be profoundly sensitive to poly (ADP-ribose) polymerase (PARP) inhibitors as a result of their defective DNA repair pathway [Farmer, et al., (2005), Nature, 434, 917-21]. This has stimulated development of small molecule drugs targeted on PARP with a view to their use specifically in BRCA carrier patients. From this example it is clear that knowledge of genetic predisposition may identify drug targets that lead to the development of personalized chemotherapy regimens to be used in combination with genetic risk profiling.

Cancer chemotherapy has well known, dose-limiting side effects on normal tissues particularly the highly proliferative hemopoetic and gut epithelial cell compartments. It can be anticipated that genetically-based individual differences exist in sensitivities of normal tissues to cytotoxic drugs. An understanding of these factors might aid in rational treatment planning and in the development of drugs designed to protect normal tissues from the adverse effects of chemotherapy.

Genetic profiling may also contribute to improved radiotherapy approaches: Within groups of breast cancer patients undergoing standard radiotherapy regimes, a proportion of patients will experience adverse reactions to doses of radiation that are normally tolerated. Acute reactions include erythema, moist desquamation, edema and radiation pneumatitis. Long term reactions including telangiectasia, edema, pulmonary fibrosis and breast fibrosis may arise many years after radiotherapy. Both acute and long-term reactions are considerable sources of morbidity and can be fatal. In one study, 87% of patients were found to have some adverse side effects to radiotherapy while 11% had serious adverse reactions (LENT/SOMA Grade 3-4); [Hoeller, et al., (2003), Int J Radiat Oncol Biol Phys, 55, 1013-8]. The probability of experiencing an adverse reaction to radiotherapy is due primarily to constitutive individual differences in normal tissue reactions and there is a suspicion that these have a strong genetic component. Several of the known breast cancer predisposition genes (e.g. BRCA1, BRCA2, ATM) affect pathways of DNA double strand break repair. DNA double strand breaks are the primary cytotoxic lesion induced by radiotherapy. This has led to concern that individuals who are genetically predisposed to breast cancer through carriage of variants in genes belonging to these pathways might also be at higher risk of suffering excessive normal tissue damage from radiotherapy. It is contemplated that the genetic variants described herein to confer risk of breast cancer may be useful for identifying individuals at particular risk of adverse reaction to radiotherapy.

The existence of constitutively radiosensitive individuals in the population means that radiotherapy dose rates for the majority of the patient population must be restricted, in order to keep the frequency of adverse reactions to an acceptable level. There is a clinical need, therefore, for reliable tests that can identify individuals who are at elevated risk for adverse reactions to radiotherapy. Such tests would indicate conservative or alternative treatments for individuals who are radiosensitive, while permitting escalation of radiotherapeutic doses for the majority of patients who are relatively radioresistant. It has been estimated that the dose escalations made possible by a test to triage breast cancer patients simply into radiosensitive, intermediate and radioresistant categories would result in an approximately 35% increase in local tumor control and consequent improvements in survival rates [Burnet, et al., (1996), Clin Oncol (R Coll Radiol), 8, 25-34].

Exposure to ionizing radiation is a proven factor contributing to oncogenesis in the breast [Dumitrescu and Cotarla, (2005), J Cell Mol Med, 9, 208-21]. Known breast cancer predisposition genes encode pathway components of the cellular response to radiation-induced DNA damage [Narod and Foulkes, (2004), Nat Rev Cancer, 4, 665-76]. Accordingly, there is concern that the risk for second primary breast tumors may be increased by irradiation of normal tissues within the radiotherapy field. There does not appear to be any measurable increased risk for BRCA carriers from radiotherapy, however their risk for second primary tumors is already exceptionally high. There is evidence to suggest that risk for second primary tumors is increased in carriers in breast cancer predisposing alleles of the ATM and CHEK2 genes who are treated with radiotherapy [Bernstein, et al., (2004), Breast Cancer Res, 6, R199-214; Broeks, et al., (2004), Breast Cancer Res Treat, 83, 91-3]. It is expected that the risk of second primary tumors from radiotherapy (and, possibly, from intensive mammographic screening) will be better defined by obtaining accurate genetic risk profiles from patients during the treatment planning stage.

### Secondary Prevention

Approximately 30% of patients who are diagnosed with a stage 1 or 2 breast cancer will experience either a loco-regional or distant metastatic recurrence of their original tumor. Patients who have had a primary breast cancer are also at greatly increased risk for being diagnosed with a second primary tumor, either in the contralateral breast or in the ipsilateral breast when breast-conserving surgery has been carried out. Secondary prevention refers to methods used to prevent recurrences or second primary tumors from developing. Methods currently in use comprise; long-term treatment with Tamoxifen or another SERM either alone or alternated with an aromatase inhibitor, risk-reducing mastectomy of the contralateral breast, and risk-reducing oophorectomy (in patients who are at risk for familial breast-ovarian cancer). Considerations regarding the use of Tamoxifen have been discussed above. With risk-reducing surgical options, it is clear that the risk needs to be quantified as well as possible in order to make an informed cost versus benefit analysis.

There are some indications that patients with known genetic predispositions to breast cancer fare worse than the majority of patients. Patients carrying the CHEK2 gene 1100delC variant have an estimated 2.8-fold increased risk of distant metastasis and a 3.9-fold increased risk of disease recurrence compared to non-carriers [de Bock, et al., (2004), J Med Genet, 41, 731-5]. Patients with BRCA1 node-negative tumors have a greater risk of metastasis than similar patients who do not carry a BRCA1 mutation[Goffin, et al., (2003), Cancer, 97, 527-36; Moller, et al., (2002), Int J Cancer, 101, 555-9; Eerola, et al., (2001), Int J Cancer, 93, 368-72]. Genetic profiling can therefore be used to help assess the risk of local recurrence and metastasis, thereby guiding the choice of secondary preventative treatment. Genetic profiling based on the variants described herein may be useful in this context. In certain embodiments, such profiling may be based on one or more of the variants described herein. In other embodiments, such profiling may include one or several other known genetic risk factors for breast cancer. Such risk factors may be well established high-penetrant risk factors, or they may be one or more of the common, lower penetrance risk factors that have been previously described).

In general, patients with a primary tumor diagnosis are at risk for second primary tumors at a constant annual incidence of 0.7% [Peto and Mack, (2000), Nat Genet, 26, 411-4]. Patients with BRCA mutations are at significantly greater risks for second primary tumors than most breast cancer patients, with absolute risks in the range 40-60%[Easton, (1999), Breast Cancer Res, 1, 14-7]. Carriers of BRCA mutations have a greatly increased risk for second primary tumors [Stacey, et al., (2006), PLoS Med, 3, e217; Metcalfe, et al., (2004), J Clin Oncol, 22, 2328-35]. Patients with mutations in the CHEK2 gene have an estimated 5.7-fold increased risk of contralateral breast cancer [de Bock, et al., (2004), J Med Genet, 41, 731-5]. Carriers of the BARD1 Cys557Ser variant are 2.7 fold more likely to be diagnosed with a second primary tumor [Stacey, et al., (2006), PLoS Med, 3, e217]. Genetic risk profiling can be used to assess the risk of second primary tumors in patients and will inform decisions on how aggressive the preventative measures should be.

### Diagnostic and Screening Methods

In certain embodiments, the present invention pertains to methods of diagnosing, or aiding in the diagnosis of, breast cancer or a susceptibility to breast cancer, by detecting particular alleles at genetic markers that appear more frequently in breast cancer subjects or subjects who are susceptible to breast cancer. In particular embodiments, the invention is a method of determining a susceptibility to breast cancer by detecting at least one allele of at least one polymorphic marker (e.g., the markers described herein). In other embodiments, the invention relates to a method of diagnosing a susceptibility to breast cancer by detecting at least one allele of at least one polymorphic marker. The present invention describes methods whereby detection of particular alleles of particular markers or haplotypes is indicative of a susceptibility to breast cancer. Such prognostic or predictive assays can also be used to determine prophylactic treatment of a subject prior to the onset of symptoms associated with breast cancer.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). The layman may also be a service provider who provides a service that comprises analyzing sequence data (e.g., genotype data for particular markers) so as to provide risk assessment measures of particular diseases or traits associated with such markers. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may thus also be a service provider who interprets genotype information, which may be for example provided by the customer, to provide risk assessment service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

A certain aspect of the invention relates to a method of assessing a subject's risk of breast cancer, comprising steps of (a) obtaining sequence information about the individual identifying at least one allele of at least one polymorphic marker in the genome of the individual; (b) representing the sequence information as digital genetic profile data; (c) transforming the digital genetic profile data on a computer processor to generate breast cancer risk assessment report for the subject; and (d) displaying the risk assessment report on an output device. The sequence information may be obtained by any method, as described in the foregoing. The sequence information is suitably represented as digital genetic profile data, which may for example be in the form of actual genotypes, genotype counts at particular markers, or other indications of the particular genotype status of an individual at one or a plurality of markers (or haplotypes comprising two or more markers). Transformation of the digital genetic profile data is the risk assessment, whereby the genotype information from the individual is transformed into a risk estimate, based on the known correlation between particular alleles at one or more markers and risk or susceptibility of breast cancer. The output device may be any suitable device for displaying the report, for example a website accessible via the internet, a data carrier, or a printed report.

The invention in a related aspect provides a risk assessment report of breast cancer for a human individual. Such a report comprises (a) at least one personal identifier; and (b) representation of at least one risk assessment measure of breast for the human individual for at least one polymorphic marker—which may be suitably be selected from any of the markers described herein. The personal identifier is any convenient identifier that can be used to identify the individual. The identifier may for example be a name, pseudoname, alias, or other numerical, alphanumerical or other codes that is associated with a unique individual. The identifier may also be an encrypted form of a personal identifier, for example a social security number or the like.

In addition, in certain other embodiments, the present invention pertains to methods of diagnosing, or aiding in the diagnosis of, a decreased susceptibility to breast cancer, by detecting particular genetic marker alleles or haplotypes that appear less frequently in breast cancer patients than in individual not diagnosed with breast cancer or in the general population.

In one embodiment, determination of a susceptibility to breast cancer can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to breast cancer, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. In certain embodiments, the oligonucleotide is from about 15 to about 100 nucleotides in length. In certain other embodiments, the oligonucleotide is from about 20 to about 50 nucleotides in length. In a particular embodiment, the nucleic acid probe is a portion of a nucleotide sequence as set forth in any one of SEQ ID NO:1-478, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to breast cancer.

Sequence analysis can also be used to detect specific alleles or haplotypes. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid associated with breast cancer, and the presence of a specific allele can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site associated with breast cancer. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment, determination of a susceptibility to breast cancer is made by detecting at least one marker or haplotype of the present invention, in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, means for amplification of nucleic acids, means for analyzing the nucleic acid sequence of a nucleic acid, means for analyzing the amino acid sequence of a polypeptide encoded by a nucleic acid associated with breast cancer, etc. The kits can for example include necessary buffers, nucleic acid primers for amplifying nucleic acids (e.g., nucleic acids comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with breast cancer diagnostic assays.

In one embodiment, the invention pertains to a kit for assaying a sample from a subject to detect a susceptibility to breast cancer in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with breast cancer risk. In one such embodiment, the polymorphism is selected from the group consisting of rs1556283, rs1983011 and rs7586009, and polymorphic markers in linkage disequilibrium therewith. In one embodiment, the fragment is at least 20 base pairs in size. Such oligonucleotides or nucleic acids (e.g., oligonucleotide primers) can be designed using portions of the nucleic acid sequence flanking the polymorphisms (e.g., SNPs or microsatellites). In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the kit comprises reagents for detecting one or more markers, two or more markers, three or more markers, four or more markers or five or more markers. In certain embodiments, the kit comprises reagents for detecting no more than 1000 markers. In certain other embodiments, the kit comprises reagents for detecting no more than 100 markers, no more than 50 markers, no more than 20 markers or no more than 10 markers.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, the kit further comprises a set of instructions for using the reagents comprising the kit. In certain embodiments, the kit further comprises a collection of data comprising correlation data between the polymorphic markers assessed by the kit and susceptibility to breast cancer. The collection of data may be provided on any suitable format. In one embodiment, the collection of data is provided on a computer-readable format.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

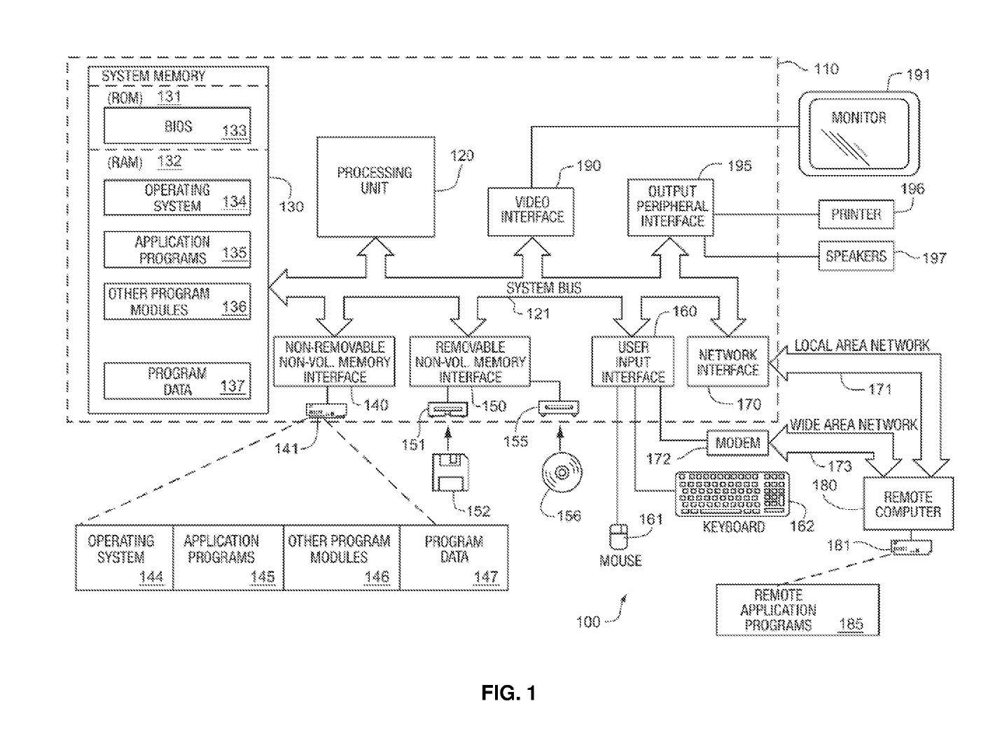

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well-known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that performs particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications of the polymorphic markers and haplotypes described herein to be associated with breast cancer (e.g., rs1556283, rs7586009 and rs1983011, and markers correlated therewith). Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider, etc.), or for deriving information from the genotype data, e.g., by comparing the genotype data to information about genetic risk factors contributing to increased susceptibility to breast cancer, and reporting results based on such comparison.

In certain embodiments, computer-readable media suitably comprise capabilities of storing (i) identifier information for at least one polymorphic marker or a haplotype, as described herein (e.g., rs1556283, rs7586009 and rs1983011, and markers correlated therewith); (ii) an indicator of the identity (e.g., presence or absence) of at least one allele of said at least one marker, or a haplotype, in individuals with the disease; and (iii) an indicator of the risk associated with the marker allele or haplotype.

The markers described herein to be associated with increased susceptibility of breast cancer, are in certain embodiments useful for interpretation and/or analysis of genotype data (including sequence data identifying particular marker alleles). Thus in certain embodiments, determination of the presence of an at-risk allele for breast cancer, as shown herein, or determination of the presence of an allele at a polymorphic marker in LD with any such risk allele, is indicative of the individual from whom the genotype data originates is at increased risk of breast cancer. In one such embodiment, genotype data is generated for at least one polymorphic marker shown herein to be associated with breast cancer, or a marker in linkage disequilibrium therewith. The genotype data can subsequently be made available to a third party, such as the individual from whom the data originates, his/her guardian or representative, a physician or health care worker, genetic counsellor, or insurance agent, for example via a user interface accessible over the internet, together with an interpretation or analysis of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk variants in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

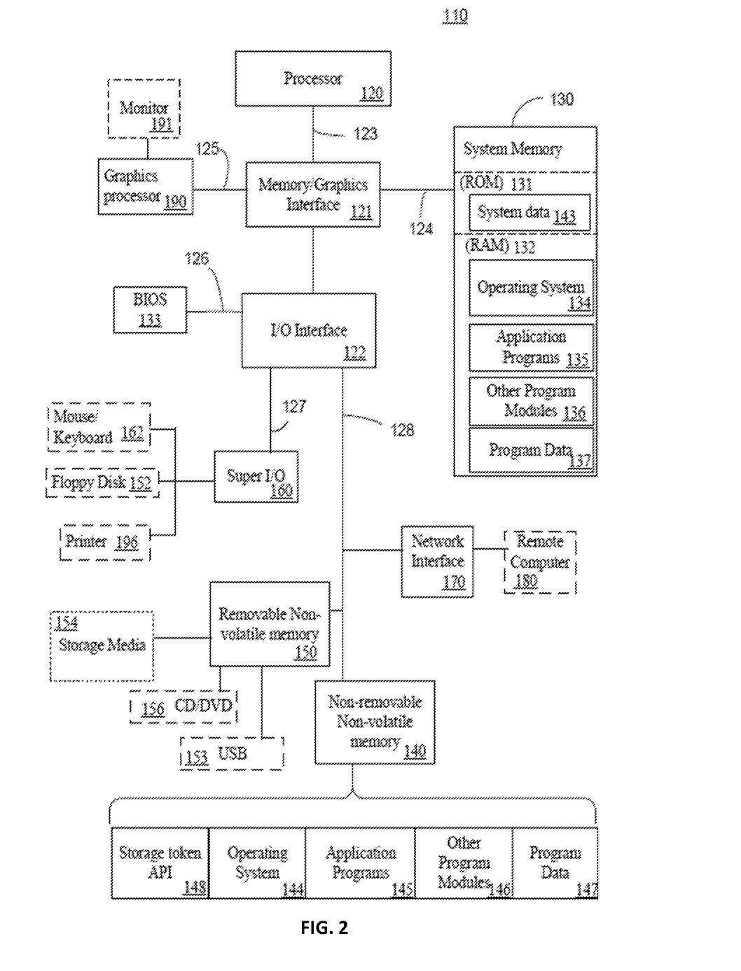

With reference to FIG. 2, a second exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 2. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (ISA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 2 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-μ) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 2 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 2, provide storage of computer readable instructions, data structures, program modules and other data for the computer 110. In FIG. 2, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 2 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to breast cancer in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, a system of the invention is a system for identifying susceptibility to breast cancer in a human subject, and comprises:
(a) at least one processor;
(b) at least one computer-readable medium;
(c) a susceptibility database operatively coupled to a computer-readable medium of the system and containing population information correlating the presence or absence of at least one allele of at least one marker selected from the group consisting of rs1556283, rs7586009 and rs1983011, and markers correlated therewith, and susceptibility to breast cancer in a population of humans;
(d) a measurement tool that receives an input about the human subject and generates information from the input about the presence or absence of the at least one allele in the human subject; and
(e) an analysis tool or routine that:
   (i) is operatively coupled to the susceptibility database and the information generated by the measurement tool,
   (ii) is stored on a computer-readable medium of the system,
   (iii) is adapted to be executed on a processor of the system, to compare the information about the human subject with the population information in the susceptibility database and generate a conclusion with respect to susceptibility to breast cancer for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

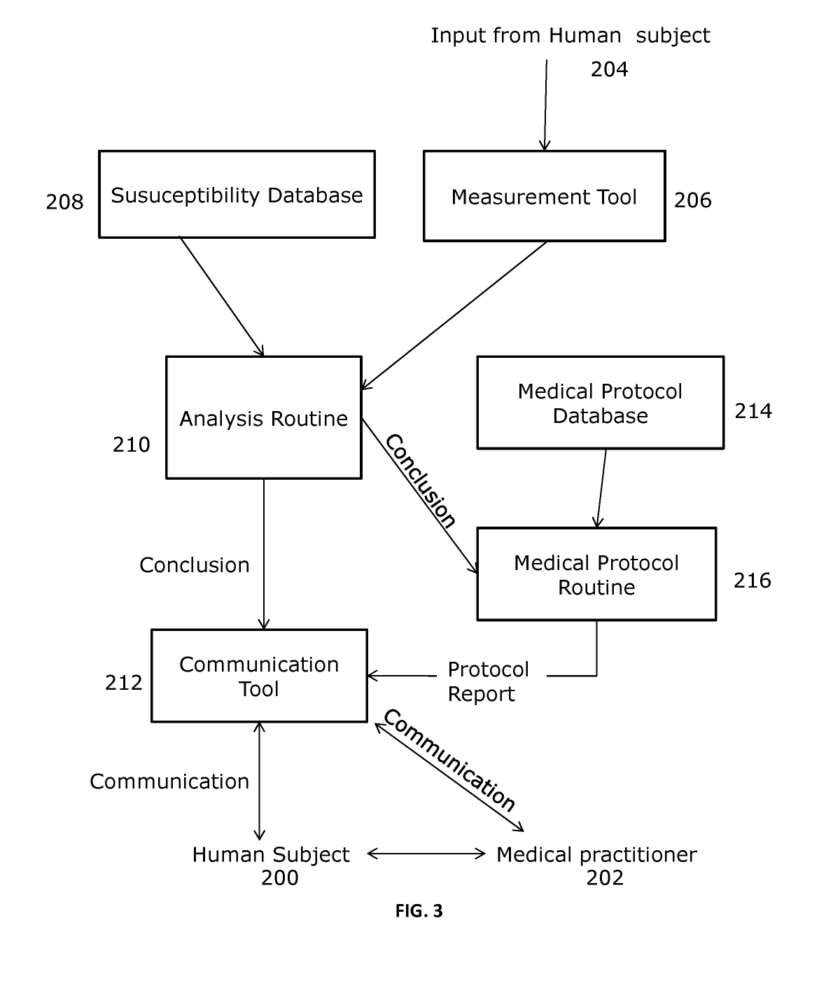

Referring to FIG. 3, an exemplary system includes a susceptibility database 208 that is operatively coupled to a computer-readable medium of the system and that contains population information correlating the presence or absence of one or more alleles of a polymorphic marker selected from rs1556283, rs7586009 and rs1983011, and markers in linkage disequilibrium therewith and susceptibility to breast cancer in a population of humans.

In certain embodiments, markers correlated with rs1556283 are selected from the group consisting of the markers set forth in Tables 3 (A and B). In certain embodiments, markers correlated with rs1983011 are selected from the group consisting of the markers set forth in Tables 5 (A and B). In certain embodiments, markers correlated with rs7586009 are selected from the group consisting of the markers set forth in Tables 4 (A and B). In some preferred embodiments, correlated markers with rs1983011 are selected from the group consisting of the markers set forth in Table 8. In some other preferred embodiments, correlated markers with rs1556283 are selected from the group consisting of the markers set forth in Table 6. In some other preferred embodiments, correlated markers with rs7586009 are selected from the group consisting of the markers set forth in Table 7. These correlated markers are thus particularly useful in the systems described herein.

In a simple variation, the susceptibility database contains 208 data relating to the frequency that a particular marker allele selected from the group has been observed in a population of humans with breast cancer and a population of humans free of breast cancer. Such data provides an indication as to the relative risk or odds ratio of developing breast cancer for a human subject that is identified as having the allele in question. In another variation, the susceptibility database includes similar data with respect to two or more markers, thereby providing a useful reference if the human subject has any of the two or more alleles of the two or more markers. In still another variation, the susceptibility database includes additional quantitative personal, medical, or genetic information about the individuals in the database diagnosed with breast cancer or free of breast cancer. Such information includes, but is not limited to, information about parameters such as age, sex, ethnicity, race, medical history, weight, blood pressure, family history of breast cancer, smoking history, and alcohol use in humans and impact of the at least one parameter on susceptibility to breast cancer. The information also can include information about other genetic risk factors for breast cancer besides the genetic variants described herein, for example high risk genetic risk factors, including mutations in the BRCA1 and BRCA2 genes. These more robust susceptibility databases can be used by an analysis routine 210 to calculate a combined score with respect to susceptibility or risk for developing breast cancer.

In addition to the susceptibility database 208, the system further includes a measurement tool 206 programmed to receive an input 204 from or about the human subject and generate an output that contains information about the presence or absence of the at least one marker allele of interest. (The input 204 is not part of the system per se but is illustrated in the schematic FIG. 3.) Thus, the input 204 will contain a specimen or contain data from which the presence or absence of the at least one marker allele can be directly read, or analytically determined. In a simple variation, the input contains annotated information about genotypes or allele counts for particular markers such as rs1556283, rs7586009 and rs1983011, and correlated markers therewith, in the genome of the human subject, in which case no further processing by the measurement tool 206 is required, except possibly transformation of the relevant information about the presence/absence of the at least one marker allele into a format compatible for use by the analysis routine 210 of the system.

In another variation, the input 204 from the human subject contains data that is unannotated or insufficiently annotated with respect to risk markers for breast cancer selected from rs1556283, rs7586009 and rs1983011, and correlated markers therewith, requiring analysis by the measurement tool 206. For example, the input can be genetic sequence of the chromosomal region or chromosome on which the markers reside, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one marker allele in a human subject from the data. For example, the measurement tool 206 contains instructions, preferably executable on a processor of the system, for analyzing the unannotated input data and determining the presence or absence of the marker allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant marker allele from the genomic sequence information.

In yet another variation, the input 204 from the human subject comprises a biological sample, such as a fluid (e.g., blood) or tissue sample that contains genetic material that can be analyzed to determine the presence or absence of particular marker allele(s) of interest. In this variation, an exemplary measurement tool 206 includes laboratory equipment for processing and analyzing the sample to determine the presence or absence (or identity) of the marker allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one marker allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one marker allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to breast cancer for the human subject. In simple terms, the analysis tool 210 looks at the marker alleles identified by the measurement tool 206 for the human subject, and compares this information to the susceptibility database 208, to determine a susceptibility to breast cancer for the subject. The susceptibility can be based on the single parameter (the identity of one or more marker alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to breast cancer. Such a conclusion is expressed in the conclusion in any statistically useful form, for example, as an odds ratio, a relative risk, or a lifetime risk for subject developing breast cancer.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 3, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to breast cancer for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to breast cancer when a medical practitioner can prescribe or administer a standard of care that can reduce susceptibility to breast cancer; and/or delay onset of breast cancer; and/or increase the likelihood of detecting the cancer at an early stage. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery.

For example, in some variations, the system further includes a medical protocol database 214 operatively connected to a computer-readable medium of the system and containing information correlating the presence or absence of the at least one marker allele of interest and medical protocols for human subjects at risk for the cancer. Such medical protocols include any variety of medicines, lifestyle changes, diagnostic tests, increased frequencies of diagnostic tests, and the like that are designed to achieve one of the aforementioned goals. The information correlating a marker allele with protocols could include, for example, information about the success with which the cancer is avoided or delayed, or success with which the cancer is detected early and treated, if a subject has a particular susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to breast cancer for the subject) and the medical protocol database 214, and (ii) generate a protocol report with respect to the probability that one or more medical protocols in the medical protocol database will achieve one or more of the goals of reducing susceptibility to the cancer; delaying onset of the cancer; and increasing the likelihood of detecting the cancer at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system include the communication tool 212. In some examples, the communication tool generates a communication that includes the protocol report in addition to, or instead of, the conclusion with respect to susceptibility.

Information about marker allele status not only can provide useful information about identifying or quantifying susceptibility to breast cancer; it can also provide useful information about possible causative factors for a human subject identified with breast cancer, and useful information about therapies for the patient. In some variations, systems of the invention are useful for these purposes.

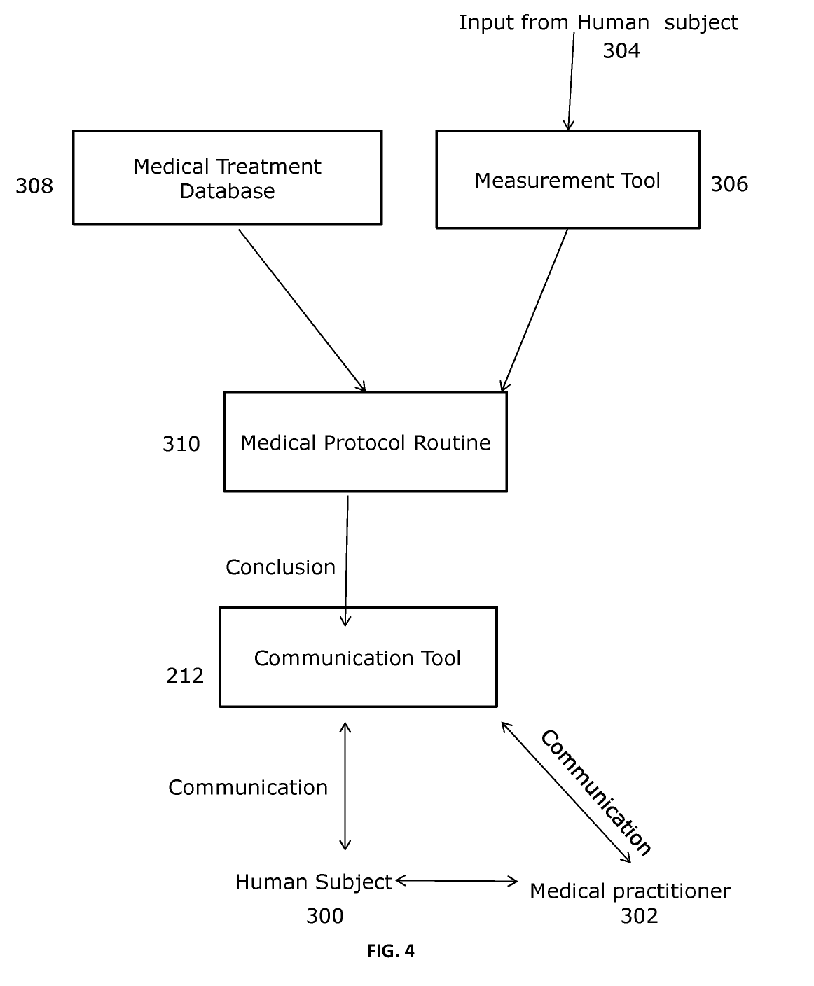

For instance, in some variations the invention is a system for assessing or selecting a treatment protocol for a subject diagnosed with breast cancer. An exemplary system, schematically depicted in FIG. 4, comprises:
(a) at least one processor;
(b) at least one computer-readable medium;
(c) a medical treatment database 308 operatively connected to a computer-readable medium of the system and containing information correlating the presence or absence of at least one allele of a marker selected from the group consisting of rs1556283, rs7586009 and rs1983011, and correlated markers in linkage disequilibrium therewith and efficacy of treatment regimens for breast cancer;
(d) a measurement tool 306 to receive an input (304, depicted in FIG. 4 but not part of the system per se) about the human subject and generate information from the input 304 about the presence or absence of the at least one marker allele in a human subject diagnosed with breast cancer; and
(e) a medical protocol routine or tool 310 operatively coupled to the medical treatment database 308 and the measurement tool 306, stored on a computer-readable medium of the system, and adapted to be executed on a processor of the system, to compare the information with respect to presence or absence of the at least one marker allele for the subject and the medical treatment database, and generate a conclusion with respect to at least one of:
   (i) the probability that one or more medical treatments will be efficacious for treatment of breast cancer for the patient; and
   (ii) which of two or more medical treatments for breast cancer will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 4, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In a further embodiment, the invention provides a computer-readable medium having computer executable instructions for determining susceptibility to breast cancer in a human individual, the computer readable medium comprising (i) sequence data identifying at least one allele of at least one polymorphic marker in the individual; and (ii) a routine stored on the computer readable medium and adapted to be executed by a processor to determine risk of developing breast cancer for the at least one polymorphic marker; wherein the at least one polymorphic marker is a marker selected from the group consisting of rs1556283, rs7586009 and rs1983011, and correlated markers in linkage disequilibrium therewith, that is predictive of susceptibility of breast cancer in humans.

In certain embodiments, markers correlated with rs1556283 are selected from the group consisting of the markers set forth in Tables 3 (A and B). In certain embodiments, markers correlated with rs1983011 are selected from the group consisting of the markers set forth in Tables 5 (A and B). In certain embodiments, markers correlated with rs7586009 are selected from the group consisting of the markers set forth in Tables 4 (A and B). In some preferred embodiments, correlated markers with rs1983011 are selected from the group consisting of the markers set forth in Table 8. In some other preferred embodiments, correlated markers with rs1556283 are selected from the group consisting of the markers set forth in Table 6. In some other preferred embodiments, correlated markers with rs7586009 are selected from the group consisting of the markers set forth in Table 7.

In certain embodiments, a report is prepared, which contains results of a determination of susceptibility of breast cancer. The report may suitably be written in any computer readable medium, printed on paper, or displayed on a visual display.

### Nucleic Acids and Polypeptides

The nucleic acids and polypeptides described herein can be used in methods and kits of the present invention, as described in the above. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the World Wide Web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, all or a portion of the nucleotide sequences as set forth in any one of SEQ ID NO:1-478; or a nucleotide sequence comprising, or consisting of, the complement of such sequences.

The nucleic acid fragments of the invention are suitably at least about 15, at least about 18, 20, 23 or 25 nucleotides, and can be 30, 40, 50, 100, 200, 500, 1000, 10,000 or more nucleotides in length. The fragments are suitably no more than 20,000 nucleotides in length, no more than 5000 nucleotides, no more than 1000 nucleotides, no more than 500 nucleotides, no more than 400 nucleotides, no more than 300 nucleotides, no more than 200 nucleotides, no more than 100 nucleotides, no more than 50 nucleotides or no more than 30 nucleotides in length.

The nucleic acid fragments of the invention may be used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

The present invention will now be exemplified by the following non-limiting examples.

### Example 1

In order to search widely for alleles of common SNPs associating with breast cancer susceptibility, we carried out a genome-wide SNP association study using Illumina HumanHap300 or Human CNV370-duo microarray. Genotyping was carried out on a population based sample of approximately 1,980 breast cancer cases and 36,219 controls from Iceland. In order to increase the power of the analysis we used a method that we refer to as “genealogy-based imputation” where known genotypes of relatives were used to infer the genotypes of untyped individuals [Rafnar et al., (2009) Nat Genet, 41, 221-227]. This resulted in an average addition of 621 breast cancer cases to the association analysis for each SNP that was represented on the Illumina chips. In order to increase the SNP coverage of the analysis, we imputed the genotypes for approximately 2.5 million autosomal SNPs in the HapMap European CEU sample using the program IMPUTE version 2 [Marchini et al., (2007) Nat Genet, 39, 906-913; Howie et al., (2009) PLoS Genetics 5(6), e1000529] and association values were determined by logistic regression using expected allele counts. Imputation of additional SNP genotypes was carried out only for individuals who had been directly typed on the Illumina chips. Association results were adjusted for relatedness among individuals using the method of genomic control [Devlin and Roeder, (1999) Biometrics, 55, 997-1004]

After excluding signals originating from previously known loci, we selected approximately 60 SNPs for further investigation, based on their association P values. We designed and generated single-track Centaurus genotyping assays for each of these SNPs. The assays were validated by typing them in the HapMap CEU European ancestry sample set. The SNPs were then genotyped in breast cancer case control foreign replication cohorts from the sets presented in Table 1. After replication genotyping, SNPs from three loci showed combined P values <5×10⁻⁷. Results from these SNPs are shown in Table 2.

The most significant association was for rs1556283 at chromosomal locus 21q21.1. The C allele of this SNP is common, with allele frequencies ranging from 0.72 to 0.85 in controls from different populations. This allele confers an estimated risk of breast cancer that is increased 1.14-fold for each allele carried. The closest gene to the SNP is NRIP1. NRIP1 is a nuclear protein that specifically interacts with the hormone-dependent activation domain AF2 of nuclear receptors. The protein modulates the transcriptional activity of the estrogen receptor in the presence of estrogen, mediating the contact between the estrogen receptor and the basal transcriptional machinery [Cavailles et al., (1995) EMBO J, 14, 3741-3751]. Estrogen exposure is a major risk factor for breast cancer and the expression of the estrogen receptor in breast tumours is of prime prognostic importance. Moreover, the estrogen response pathway is targeted by hormonal therapies such as Tamoxifen and Raloxifene which are used in primary and secondary prevention of breast cancer.

The second most significant association was with the C allele of rs7586009 at chromosomal locus 2p21. The C allele is present in frequencies ranging from 0.54 to 0.59 in controls from different populations. This allele confers an estimated risk of breast cancer that is increased 1.10-fold for each allele carried. The closest gene is LOC100134259, a gene of unknown function. Other genes in the locus which have potential as candidate breast cancer susceptibility genes are TTC7A, SOCS5, CRIPT and RHOQ.

The third most significant association was with the C allele of rs1983011 at chromosomal locus 3q26.32. The allele is present in frequencies ranging from 0.42 to 0.48 in controls from different populations. This allele confers an estimated risk of breast cancer that is increased 1.09-fold for each allele carried. The SNP occurs in a gene-poor region. The closest gene is TBL1XR1, a member of the WD40 repeat-containing protein family. These proteins may mediate protein-protein interactions and may be involved in signal transduction and other cellular regulatory functions.

TABLE 1
Overview of the sample sets used in the study

| Sample Set | Cases | Controls | Type | Reference |
|---|---|---|---|---|
| Iceland | 1980 | 36219 | Registry Ascertained Case: | a |
| | | | Population Based Control | |
| Hannover, | 1006 | 1010 | Clinic Ascertained Case: | b |
| Germany | | | Population Based Control | |
| (HaBCS) | | | | |
| Nijmegen, | 727 | 1830 | Registry Ascertained Case: | c |
| Netherlands | | | Population Based Control | |
| U.S.A. (Mayo | 1753 | 1487 | Clinic Ascertained Case: | d |
| Clinic Breast | | | Population Based Control | |
| Cancer Study | | | | |
| [MCBCS]) | | | | |
| Minsk, Belarus | 1934 | 1235 | Clinic Ascertained Case: | e |
| (HMBCS) | | | Population Based Control | |
| Zaragoza, | 1009 | 1719 | Clinic Ascertained Case: | c |
| Spain | | | Population Based Control | |
| Stockholm, | 818 | 1750 | Clinic Ascertained Case: | f |
| Sweden | | | Population Based Control | |
| U.S.A. | 1144 | 1141 | Prospective Study | g |
| (CGEMS) | | | Nested Case: Control | |
| Total | | 46391 | | |

References:
a Stacey, S.N et al., PLoS Med. (2006) 3, e217.
b Dork, T. et al., Cancer Res (2001) 61, 7608-7615
c Stacey, S.N. et al., Nat Genet. (2007) 39, 865-9.
d Olson, J.E. et al.. Breast Cancer Res Treat. (2007) 102, 237-47.
e Boqdanova, N. et al.. Breast Cancer Res Treat (2008) 118, 207-211
f Margolin, S. et al., Genet Test. (2004) 8(2), 127-32
g Hunter, D.J. et al., Nat Genet. (2007) 39, 870-874

TABLE 2
Association of rs1556283 (NRIP1), rs1983011,
and rs7586009 with Breast Cancer

| Sample Set | SNP | Allele | OR | 95% CI | P |
|---|---|---|---|---|---|
| ICELAND | rs1556283 | C | 1.19 | (1.06, 1.32) | 0.0024 |
| HANNOVER (HaBCS) | rs1556283 | C | 1.02 | (0.88, 1.18) | 0.83 |
| NIJMEGEN | rs1556283 | C | 1.19 | (1.02, 1.39) | 0.027 |
| U.S.A. (MCBCS) | rs1556283 | C | 1.18 | (1.04, 1.33) | 0.0082 |
| MINSK (HMBCS) | rs1556283 | C | 1.30 | (1.16, 1.47) | 7.40E−06 |
| ZARAGOZA | rs1556283 | C | 0.95 | (0.81, 1.11) | 0.52 |
| STOCKHOLM | rs1556283 | C | 1.06 | (0.93, 1.23) | 0.38 |
| CGEMS | rs1556283 | C | 1.09 | (0.94, 1.25) | 0.24 |
| COMBINED | rs1556283 | C | 1.14 | (1.09, 1.19) | 8.50E−08 |
| ICELAND | rs1983011 | C | 1.14 | (1.08, 1.20) | 7.90E−06 |
| HANNOVER (HaBCS) | rs1983011 | C | 1.00 | (0.88, 1.14) | 0.99 |
| NIJMEGEN | rs1983011 | C | 1.22 | (1.08, 1.37) | 0.0022 |
| U.S.A. (MCBCS) | rs1983011 | C | 1.03 | (0.93, 1.14) | 0.59 |
| MINSK (HMBCS) | rs1983011 | C | 1.12 | (1.01, 1.23) | 0.03 |
| ZARAGOZA | rs1983011 | C | 1.03 | (0.92, 1.15) | 0.65 |
| STOCKHOLM | rs1983011 | C | 1.11 | (0.98, 1.25) | 0.097 |
| CGEMS | rs1983011 | C | 0.99 | (0.88, 1.11) | 0.81 |
| COMBINED | rs1983011 | C | 1.09 | (1.05, 1.12) | 4.90E−07 |
| ICELAND | rs7586009 | C | 1.10 | (1.04, 1.16) | 0.001 |
| HANNOVER (HaBCS) | rs7586009 | C | 1.15 | (1.01, 1.30) | 0.031 |
| NIJMEGEN | rs7586009 | C | 1.09 | (0.96, 1.23) | 0.2 |
| U.S.A. (MCBCS) | rs7586009 | C | 1.08 | (0.97, 1.19) | 0.16 |
| MINSK (HMBCS) | rs7586009 | C | 1.15 | (1.03, 1.27) | 0.0099 |
| ZARAGOZA | rs7586009 | C | 1.06 | (0.93, 1.20) | 0.38 |
| STOCKHOLM | rs7586009 | C | 1.08 | (0.95, 1.22) | 0.22 |
| CGEMS | rs7586009 | C | 1.04 | (0.93, 1.16) | 0.54 |
| COMBINED | rs7586009 | C | 1.10 | (1.05, 1.14) | 3.50E−07 |

### Example 2

Surrogate markers of rs1556283, rs1983011 and rs7586009 were identified, using either sequence data from about 150 Icelanders (complete genome sequence data), or from the public 1000 genomes dataset (Caucasian CEU sample; see http://www.1000genomes.org). Results are shown in Tables 3, 4 and 5.

Furthermore, we determined association to breast cancer by imputation of genotypes for predicted surrogates of rs1556283, rs1983011 and rs7586009. Results of such imputation analysis are shown in Tables 6, 7 and 8. The data presented shows that surrogates are indeed useful for detecting risk of breast cancer, as illustrated by significant p-values of association.

TABLE 3A
Surrogate markers for marker rs1556283 on Chromosome 21 identified
in Icelandic samples. Shown are marker names (chromosome
followed by location in NCBI Build 36), position in
NCBI Build 36, r² values for the correlation with rs1556283,
the predicted risk alleles for the surrogate
markers, i.e. alleles that are correlated with the at-risk C allele of
rs1556283 and SEQ ID for flanking sequence of the marker.

| | Pos in | | Risk | Other | Seq |
|---|---|---|---|---|---|
| Marker | NCBI_B36 | r² | Allele | Allele | ID NO |
| rs2823070 | 15410486 | 0.290585 | G | C | 93 |
| rs2026852 | 15410557 | 0.251487 | G | A | 70 |
| rs4817662 | 15412114 | 0.294936 | G | T | 143 |
| rs4817666 | 15412462 | 0.294747 | A | G | 144 |
| rs2823074 | 15421527 | 0.238481 | A | C | 94 |
| rs2823075 | 15426902 | 0.338196 | G | A | 95 |
| rs2823080 | 15431719 | 0.428459 | G | A | 97 |
| rs2823081 | 15431939 | 0.28893 | T | A | 98 |
| rs2823082 | 15432624 | 0.428175 | T | G | 99 |
| rs2823083 | 15433269 | 0.303246 | A | G | 100 |
| rs926164 | 15435118 | 0.234996 | A | C | 174 |
| rs2823084 | 15435214 | 0.499984 | T | C | 101 |
| rs7281527 | 15436032 | 0.511343 | T | C | 163 |
| rs2823085 | 15437113 | 0.227641 | G | C | 102 |
| rs2823086 | 15437412 | 0.511728 | A | G | 103 |
| rs2823087 | 15437630 | 0.227641 | C | T | 104 |
| rs2823088 | 15437844 | 0.267456 | G | A | 105 |
| rs2823089 | 15437925 | 0.227641 | C | T | 106 |
| rs926166 | 15437935 | 0.22717 | G | A | 175 |
| rs55859143 | 15438022 | 0.511751 | A | G | 148 |
| rs12482834 | 15438153 | 0.267483 | T | C | 56 |
| rs741937 | 15439702 | 0.272328 | T | G | 170 |
| rs2049880 | 15439762 | 0.226926 | G | C | 71 |
| rs2823091 | 15439876 | 0.51 | A | T | 107 |
| rs2007783 | 15440395 | 0.511343 | G | T | 66 |
| rs2823093 | 15442703 | 0.443427 | G | A | 108 |
| rs2896680 | 15442806 | 0.46097 | A | T | 140 |
| rs2256038 | 15443047 | 0.280181 | G | A | 75 |
| rs2823096 | 15443953 | 0.747468 | A | G | 109 |
| rs2823097 | 15445015 | 0.276826 | G | C | 110 |
| rs9974193 | 15450904 | 0.476328 | T | G | 179 |
| rs2823106 | 15454531 | 0.998797 | T | C | 111 |
| rs9975709 | 15455511 | 0.47995 | C | A | 180 |
| rs62218221 | 15455545 | 0.998875 | T | C | 150 |
| rs7275529 | 15456069 | 0.478607 | T | C | 159 |
| rs2008843 | 15456839 | 0.997394 | T | C | 67 |
| rs2823107 | 15457405 | 0.479969 | A | G | 112 |
| rs55792425 | 15457964 | 0.99955 | A | T | 146 |
| rs62218223 | 15458191 | 0.986101 | G | A | 151 |
| rs11088297 | 15458583 | 0.479984 | C | T | 46 |
| rs11088298 | 15458584 | 0.479984 | T | A | 47 |
| rs11088299 | 15458627 | 0.479141 | T | C | 48 |
| rs12482761 | 15459318 | 0.986897 | T | C | 55 |
| rs990302 | 15460110 | 0.998187 | G | A | 178 |
| rs62218224 | 15460995 | 0.99955 | G | C | 152 |
| rs62218225 | 15461014 | 0.999591 | A | C | 153 |
| rs2896689 | 15462286 | 0.99955 | C | A | 141 |
| rs7282135 | 15463056 | 0.447482 | A | C | 164 |
| rs2223128 | 15464831 | 0.45138 | G | T | 74 |
| rs2823110 | 15464942 | 0.997175 | C | T | 113 |
| rs2823111 | 15465149 | 0.99955 | G | A | 114 |
| rs2823112 | 15465216 | 0.99955 | G | C | 115 |
| chr21:15465320 | 15465320 | 0.43368 | T | A | 26 |
| chr21:15465323 | 15465323 | 0.403346 | G | C | 27 |
| rs2142362 | 15465381 | 0.99955 | G | A | 72 |
| rs2823113 | 15465915 | 0.984949 | T | C | 116 |
| rs1882959 | 15466379 | 0.99955 | T | C | 61 |
| rs12482386 | 15466790 | 0.99955 | G | A | 54 |
| rs2823115 | 15466848 | 0.99957 | A | G | 117 |
| rs7279893 | 15467399 | 0.445226 | T | C | 162 |
| rs2823117 | 15467738 | 0.986897 | C | T | 118 |
| rs2823118 | 15467777 | 0.456441 | A | T | 119 |
| rs12483016 | 15467996 | 0.99955 | G | T | 57 |
| rs12483602 | 15468304 | 0.999555 | C | T | 58 |
| rs55797017 | 15468489 | 0.999602 | G | T | 147 |
| rs62218995 | 15469153 | 0.997394 | C | T | 154 |
| rs8128132 | 15469185 | 0.999417 | A | T | 173 |
| rs73176585 | 15469191 | 0.997257 | T | A | 167 |
| rs11909473 | 15469306 | 0.65325 | A | G | 51 |
| rs2823119 | 15469515 | 0.986897 | A | C | 120 |
| rs62218996 | 15469959 | 0.966295 | G | A | 155 |
| rs62218997 | 15470608 | 0.999976 | G | A | 156 |
| rs12482303 | 15471530 | 0.986897 | C | T | 53 |
| rs1014526 | 15471649 | 0.392633 | C | T | 45 |
| rs73176588 | 15472009 | 0.877975 | A | T | 168 |
| rs2823121 | 15473128 | 0.394011 | A | G | 121 |
| rs2823122 | 15473292 | 0.999549 | G | C | 122 |
| rs2823123 | 15474025 | 0.986897 | G | A | 123 |
| rs2823124 | 15474392 | 0.392104 | G | A | 124 |
| rs11088311 | 15474905 | 0.982094 | C | A | 49 |
| rs12373912 | 15475004 | 0.99775 | G | A | 52 |
| rs55778206 | 15476933 | 0.998158 | A | T | 145 |
| rs1882961 | 15478238 | 0.578269 | C | T | 62 |
| rs1556283 | 15478599 | 1 | C | T | 59 |
| rs965098 | 15479497 | 0.388807 | G | A | 177 |
| rs1882963 | 15481989 | 0.99931 | G | C | 63 |
| rs2823128 | 15485180 | 0.436716 | G | A | 125 |
| rs2823129 | 15485511 | 0.436716 | C | T | 126 |
| rs7282349 | 15486432 | 0.431963 | C | T | 165 |
| rs926167 | 15488394 | 0.406918 | T | C | 176 |
| rs2823131 | 15489599 | 0.402664 | G | A | 127 |
| rs7275438 | 15490199 | 0.406936 | C | T | 158 |
| rs7275695 | 15490395 | 0.40658 | C | T | 160 |
| rs2823132 | 15490727 | 0.405969 | T | C | 128 |
| rs2823133 | 15490812 | 0.402664 | A | C | 129 |
| rs2823134 | 15490934 | 0.406821 | A | G | 130 |
| rs2823135 | 15492355 | 0.402664 | A | G | 131 |
| rs2823136 | 15493822 | 0.407063 | G | C | 132 |
| rs7276622 | 15494147 | 0.407065 | G | A | 161 |
| rs11088317 | 15495993 | 0.442682 | C | T | 50 |
| rs2403907 | 15496326 | 0.398502 | C | A | 77 |
| rs2823137 | 15496913 | 0.375583 | T | A | 133 |
| rs2823138 | 15498364 | 0.507276 | G | A | 134 |
| rs2823139 | 15498654 | 0.296533 | G | A | 135 |
| rs73184544 | 15499420 | 0.267466 | G | T | 169 |
| rs1997595 | 15500030 | 0.286092 | A | C | 64 |
| rs1997596 | 15500319 | 0.269925 | C | T | 65 |
| rs2823140 | 15500671 | 0.282516 | G | A | 136 |
| rs56038390 | 15504581 | 0.268552 | A | G | 149 |

TABLE 3B
Surrogate markers of anchor marker rs1556283 on Chromosome 21. Markers
were selected using data from the publically available 1000 Genomes
project (http://www.1000genomes.org). Markers that have not been assigned
rs names are identified by their position in NCBI Build 36 of the human genome
assembly. Shown are risk alleles for the surrogate markers, i.e. alleles that
are correlated with the at-risk C allele of rs1556283. Linkage disequilibrium
measures D′ and R², and corresponding p-value, are also shown.
The last column refers to the sequence listing number, identifying
the particular SNP.

| | Pos in | Risk | Other | | | | Seq ID |
|---|---|---|---|---|---|---|---|
| Marker | NCBI_B36 | Allele | Allele | D′ | r² | p-value | NO |
| chr21: 15013475 | 15013475 | C | T | 0.56 | 0.24 | 8.40E−05 | 1 |
| rs2823040 | 15379229 | A | C | 0.89 | 0.22 | 4.90E−05 | 78 |
| rs2823041 | 15379788 | C | T | 0.66 | 0.41 | 5.20E−07 | 79 |
| rs2823042 | 15380069 | C | T | 0.89 | 0.22 | 4.90E−05 | 80 |
| rs2823045 | 15382630 | G | A | 0.76 | 0.47 | 2.20E−08 | 81 |
| chr21: 15383585 | 15383585 | T | A | 0.76 | 0.47 | 2.20E−08 | 2 |
| rs2823046 | 15383654 | G | A | 0.76 | 0.47 | 2.20E−08 | 82 |
| chr21: 15383729 | 15383729 | T | C | 0.75 | 0.42 | 2.00E−07 | 3 |
| chr21: 15384412 | 15384412 | C | G | 0.89 | 0.21 | 1.70E−05 | 4 |
| chr21: 15384640 | 15384640 | G | A | 0.76 | 0.47 | 2.20E−08 | 5 |
| rs2823052 | 15385989 | C | T | 0.75 | 0.42 | 1.00E−07 | 83 |
| chr21: 15386526 | 15386526 | C | T | 1 | 0.26 | 5.00E−10 | 6 |
| rs719112 | 15387005 | A | T | 1 | 0.28 | 1.60E−10 | 157 |
| chr21: 15387074 | 15387074 | T | C | 0.84 | 0.23 | 0.00012 | 7 |
| rs2823054 | 15387459 | G | C | 1 | 0.27 | 2.90E−10 | 84 |
| rs1005526 | 15388481 | G | A | 1 | 0.27 | 2.90E−10 | 44 |
| rs760404 | 15388760 | C | T | 1 | 0.27 | 2.90E−10 | 171 |
| chr21: 15389243 | 15389243 | T | A | 0.69 | 0.37 | 1.20E−06 | 8 |
| chr21: 15389951 | 15389951 | C | T | 1 | 0.25 | 8.60E−10 | 9 |
| rs2150369 | 15392405 | C | T | 1 | 0.27 | 2.90E−10 | 73 |
| chr21: 15392529 | 15392529 | G | A | 0.74 | 0.37 | 7.20E−07 | 10 |
| rs1003428 | 15395478 | T | A | 0.72 | 0.3 | 9.90E−06 | 43 |
| rs1003427 | 15395832 | G | T | 1 | 0.26 | 5.00E−10 | 42 |
| rs1556286 | 15396029 | T | G | 1 | 0.26 | 5.00E−10 | 60 |
| rs2823056 | 15396245 | G | A | 1 | 0.26 | 5.00E−10 | 85 |
| chr21: 15396612 | 15396612 | C | T | 1 | 0.21 | 7.70E−06 | 11 |
| rs2823058 | 15398520 | A | T | 0.73 | 0.35 | 1.80E−06 | 86 |
| rs9977308 | 15398972 | T | C | 1 | 0.25 | 8.60E−10 | 181 |
| rs2823059 | 15400165 | A | G | 0.52 | 0.24 | 0.00017 | 87 |
| chr21: 15402303 | 15402303 | A | C | 0.74 | 0.35 | 8.90E−07 | 12 |
| rs2823061 | 15403536 | G | A | 1 | 0.25 | 8.60E−10 | 88 |
| rs4816486 | 15404491 | A | G | 1 | 0.25 | 8.60E−10 | 142 |
| rs2823062 | 15404514 | T | G | 1 | 0.25 | 8.60E−10 | 89 |
| rs9978683 | 15405325 | G | A | 1 | 0.25 | 8.60E−10 | 183 |
| chr21: 15406415 | 15406415 | T | C | 1 | 0.53 | 2.30E−16 | 13 |
| rs7282412 | 15406522 | T | A | 1 | 0.25 | 8.60E−10 | 166 |
| rs2823065 | 15407768 | C | T | 1 | 0.25 | 8.60E−10 | 90 |
| rs2823066 | 15408186 | G | A | 1 | 0.25 | 8.60E−10 | 91 |
| rs2026849 | 15408497 | G | A | 1 | 0.28 | 1.60E−10 | 69 |
| rs2823069 | 15409657 | T | C | 0.68 | 0.21 | 6.80E−05 | 92 |
| rs2823070 | 15410486 | G | C | 0.8 | 0.51 | 2.30E−09 | 93 |
| chr21: 15410780 | 15410780 | C | T | 0.78 | 0.33 | 9.40E−07 | 14 |
| rs8127658 | 15410898 | T | C | 1 | 0.49 | 1.60E−15 | 172 |
| rs4817662 | 15412114 | G | T | 0.93 | 0.6 | 2.80E−11 | 143 |
| rs2403878 | 15412417 | C | T | 1 | 0.47 | 4.00E−15 | 76 |
| rs4817666 | 15412462 | A | G | 0.86 | 0.55 | 3.20E−10 | 144 |
| rs9977355 | 15417178 | G | A | 1 | 0.49 | 1.60E−15 | 182 |
| rs2016155 | 15420114 | G | A | 1 | 0.49 | 1.60E−15 | 68 |
| rs9985165 | 15420650 | T | C | 1 | 0.44 | 2.20E−14 | 184 |
| rs2823074 | 15421527 | A | C | 0.81 | 0.56 | 3.70E−10 | 94 |
| rs2823075 | 15426902 | G | A | 1 | 0.48 | 4.70E−12 | 95 |
| rs2823076 | 15428407 | G | A | 0.93 | 0.48 | 2.80E−10 | 96 |
| rs2823080 | 15431719 | G | A | 0.93 | 0.65 | 3.80E−12 | 97 |
| rs2823081 | 15431939 | T | A | 0.93 | 0.48 | 1.20E−09 | 98 |
| rs2823082 | 15432624 | T | G | 0.93 | 0.65 | 3.80E−12 | 99 |
| rs2823083 | 15433269 | A | G | 0.94 | 0.6 | 4.50E−12 | 100 |
| rs926164 | 15435118 | A | C | 0.77 | 0.51 | 1.60E−09 | 174 |
| rs2823084 | 15435214 | T | C | 1 | 0.66 | 2.60E−16 | 101 |
| rs7281527 | 15436032 | T | C | 1 | 0.75 | 9.50E−19 | 163 |
| rs2823085 | 15437113 | G | C | 0.76 | 0.48 | 5.50E−09 | 102 |
| rs2823086 | 15437412 | A | G | 1 | 0.75 | 9.50E−19 | 103 |
| rs2823087 | 15437630 | C | T | 0.76 | 0.48 | 5.50E−09 | 104 |
| rs2823088 | 15437844 | G | A | 0.76 | 0.48 | 5.50E−09 | 105 |
| rs2823089 | 15437925 | C | T | 0.75 | 0.42 | 5.20E−08 | 106 |
| rs926166 | 15437935 | G | A | 0.76 | 0.48 | 5.50E−09 | 175 |
| chr21: 15438022 | 15438022 | A | G | 1 | 0.75 | 9.50E−19 | 15 |
| rs12482834 | 15438153 | T | C | 0.76 | 0.48 | 5.50E−09 | 56 |
| rs741937 | 15439702 | T | G | 0.76 | 0.48 | 5.50E−09 | 170 |
| rs2049880 | 15439762 | G | C | 0.75 | 0.42 | 5.20E−08 | 71 |
| rs2823091 | 15439876 | A | T | 1 | 0.75 | 9.50E−19 | 107 |
| chr21: 15440395 | 15440395 | G | T | 1 | 0.61 | 3.60E−15 | 16 |
| rs2823093 | 15442703 | G | A | 1 | 0.65 | 7.30E−19 | 108 |
| rs2896680 | 15442806 | A | T | 1 | 0.65 | 7.30E−19 | 140 |
| rs2256038 | 15443047 | G | A | 1 | 0.49 | 1.60E−15 | 75 |
| rs2823096 | 15443953 | A | G | 1 | 0.8 | 4.30E−20 | 109 |
| rs2823097 | 15445015 | G | C | 1 | 0.47 | 4.00E−15 | 110 |
| rs9974193 | 15450904 | T | G | 1 | 0.47 | 4.00E−15 | 179 |
| rs2823106 | 15454531 | T | C | 1 | 1 | 2.40E−27 | 111 |
| rs9975709 | 15455511 | C | A | 1 | 0.39 | 2.40E−13 | 180 |
| chr21: 15455545 | 15455545 | T | C | 1 | 1 | 2.40E−27 | 17 |
| rs7275529 | 15456069 | T | C | 1 | 0.49 | 1.60E−15 | 159 |
| rs2008843 | 15456839 | T | C | 1 | 0.95 | 6.20E−25 | 67 |
| rs2823107 | 15457405 | A | G | 1 | 0.49 | 1.60E−15 | 112 |
| chr21: 15457964 | 15457964 | A | T | 1 | 1 | 2.40E−27 | 18 |
| chr21: 15458191 | 15458191 | G | A | 1 | 1 | 2.40E−27 | 19 |
| chr21: 15458583 | 15458583 | C | T | 1 | 0.49 | 1.60E−15 | 20 |
| chr21: 15458584 | 15458584 | T | A | 1 | 0.49 | 1.60E−15 | 21 |
| chr21: 15458627 | 15458627 | T | C | 1 | 0.49 | 1.60E−15 | 22 |
| rs12482761 | 15459318 | T | C | 1 | 1 | 2.40E−27 | 55 |
| rs990302 | 15460110 | G | A | 1 | 1 | 2.40E−27 | 178 |
| chr21: 15460995 | 15460995 | G | C | 1 | 1 | 2.40E−27 | 23 |
| chr21: 15461014 | 15461014 | A | C | 1 | 1 | 2.40E−27 | 24 |
| rs2896689 | 15462286 | C | A | 1 | 0.95 | 6.20E−25 | 141 |
| rs7282135 | 15463056 | A | C | 1 | 0.49 | 1.60E−15 | 164 |
| chr21: 15464779 | 15464779 | T | A | 1 | 0.44 | 4.30E−11 | 25 |
| rs2223128 | 15464831 | G | T | 1 | 0.49 | 1.60E−15 | 74 |
| rs2823110 | 15464942 | C | T | 1 | 1 | 2.40E−27 | 113 |
| rs2823111 | 15465149 | G | A | 1 | 0.7 | 1.70E−17 | 114 |
| rs2823112 | 15465216 | G | C | 1 | 1 | 2.40E−27 | 115 |
| rs2142362 | 15465381 | G | A | 1 | 1 | 2.40E−27 | 72 |
| rs2823113 | 15465915 | T | C | 1 | 1 | 2.40E−27 | 116 |
| rs1882959 | 15466379 | T | C | 1 | 1 | 2.40E−27 | 61 |
| rs12482386 | 15466790 | G | A | 1 | 1 | 2.40E−27 | 54 |
| rs2823115 | 15466848 | A | G | 1 | 1 | 2.40E−27 | 117 |
| rs7279893 | 15467399 | T | C | 1 | 0.47 | 4.00E−15 | 162 |
| rs2823117 | 15467738 | C | T | 1 | 1 | 2.40E−27 | 118 |
| rs2823118 | 15467777 | A | T | 1 | 0.47 | 4.00E−15 | 119 |
| rs12483016 | 15467996 | G | T | 1 | 1 | 2.40E−27 | 57 |
| chr21: 15468304 | 15468304 | C | T | 1 | 1 | 2.40E−27 | 28 |
| chr21: 15468489 | 15468489 | G | T | 1 | 1 | 2.40E−27 | 29 |
| chr21: 15469153 | 15469153 | C | T | 1 | 1 | 2.40E−27 | 30 |
| rs8128132 | 15469185 | A | T | 1 | 1 | 2.40E−27 | 173 |
| chr21: 15469191 | 15469191 | T | A | 1 | 1 | 2.40E−27 | 31 |
| rs11909473 | 15469306 | A | G | 1 | 0.6 | 8.80E−18 | 51 |
| rs2823119 | 15469515 | A | C | 1 | 1 | 2.40E−27 | 120 |
| chr21: 15469959 | 15469959 | G | A | 1 | 1 | 2.40E−27 | 32 |
| chr21: 15470608 | 15470608 | G | A | 1 | 1 | 2.40E−27 | 33 |
| rs12482303 | 15471530 | C | T | 1 | 1 | 2.40E−27 | 53 |
| rs1014526 | 15471649 | C | T | 1 | 0.42 | 5.10E−14 | 45 |
| chr21: 15472009 | 15472009 | A | T | 1 | 0.66 | 2.60E−16 | 34 |
| rs2823121 | 15473128 | A | G | 1 | 0.42 | 5.10E−14 | 121 |
| rs2823122 | 15473292 | G | C | 1 | 1 | 2.40E−27 | 122 |
| rs2823123 | 15474025 | G | A | 1 | 0.95 | 6.20E−25 | 123 |
| rs2823124 | 15474392 | G | A | 1 | 0.42 | 5.10E−14 | 124 |
| chr21: 15476933 | 15476933 | A | T | 1 | 0.85 | 1.60E−21 | 35 |
| rs1882961 | 15478238 | C | T | 1 | 0.65 | 7.30E−19 | 62 |
| rs965098 | 15479497 | G | A | 1 | 0.42 | 5.10E−14 | 177 |
| chr21: 15481989 | 15481989 | G | C | 0.95 | 0.9 | 1.20E−17 | 36 |
| rs2823128 | 15485180 | G | A | 0.86 | 0.41 | 2.20E−08 | 125 |
| rs2823129 | 15485511 | C | T | 0.86 | 0.41 | 2.20E−08 | 126 |
| rs7282349 | 15486432 | C | T | 0.86 | 0.41 | 2.20E−08 | 165 |
| rs926167 | 15488394 | T | C | 0.79 | 0.38 | 3.20E−07 | 176 |
| rs2823131 | 15489599 | G | A | 0.73 | 0.33 | 2.10E−06 | 127 |
| rs7275438 | 15490199 | C | T | 0.73 | 0.33 | 2.10E−06 | 158 |
| rs7275695 | 15490395 | C | T | 0.73 | 0.33 | 2.10E−06 | 160 |
| rs2823132 | 15490727 | T | C | 0.73 | 0.33 | 2.10E−06 | 128 |
| rs2823133 | 15490812 | A | C | 0.73 | 0.33 | 2.10E−06 | 129 |
| rs2823134 | 15490934 | A | G | 0.73 | 0.33 | 2.10E−06 | 130 |
| chr21: 15491897 | 15491897 | G | A | 0.71 | 0.28 | 2.00E−05 | 37 |
| chr21: 15491996 | 15491996 | G | A | 0.73 | 0.33 | 2.10E−06 | 38 |
| rs2823135 | 15492355 | A | G | 0.73 | 0.33 | 2.10E−06 | 131 |
| rs2823136 | 15493822 | G | C | 0.73 | 0.33 | 2.10E−06 | 132 |
| rs7276622 | 15494147 | G | A | 0.73 | 0.33 | 2.10E−06 | 161 |
| rs11088317 | 15495993 | C | T | 0.8 | 0.4 | 2.60E−07 | 50 |
| rs2403907 | 15496326 | C | A | 0.8 | 0.4 | 2.60E−07 | 77 |
| rs2823137 | 15496913 | T | A | 0.8 | 0.4 | 2.60E−07 | 133 |
| rs2823138 | 15498364 | G | A | 0.67 | 0.44 | 3.80E−08 | 134 |
| rs2823139 | 15498654 | G | A | 0.6 | 0.24 | 4.50E−05 | 135 |
| chr21: 15499530 | 15499530 | T | C | 1 | 0.25 | 1.20E−06 | 39 |
| rs1997595 | 15500030 | A | C | 0.59 | 0.22 | 8.70E−05 | 64 |
| rs1997596 | 15500319 | C | T | 0.59 | 0.22 | 8.70E−05 | 65 |
| rs2823140 | 15500671 | G | A | 0.59 | 0.22 | 8.70E−05 | 136 |
| rs2823141 | 15501676 | G | A | 0.64 | 0.23 | 8.70E−05 | 137 |
| rs2823142 | 15501823 | A | G | 0.68 | 0.22 | 0.00014 | 138 |
| rs56038390 | 15504581 | A | G | 0.59 | 0.22 | 8.70E−05 | 149 |
| rs2823144 | 15504804 | C | G | 0.68 | 0.22 | 0.00014 | 139 |
| chr21: 15522607 | 15522607 | A | G | 1 | 0.24 | 1.50E−09 | 40 |
| chr21: 15566068 | 15566068 | C | T | 1 | 0.21 | 7.70E−06 | 41 |

TABLE 4A
Surrogate markers for rs7586009 on Chromosome 2. Shown are
marker names (chromosome followed by location in NCBI
Build 36), position in NCBI Build 36, r² values for the
correlation with rs7586009, the predicted risk alleles for
the surrogate markers, i.e. alleles that are
correlated with the at-risk C allele of rs7586000 and
SEQ ID for flanking sequence of the marker.

| | Pos in | | Risk | Other | Seq |
|---|---|---|---|---|---|
| Marker | NCBI_B36 | r² | Allele | Allele | ID NO |
| rs6544932 | 46930495 | 0.233648 | G | C | 229 |
| rs72804479 | 46931429 | 0.356476 | C | T | 232 |
| rs13006048 | 46931823 | 0.224081 | T | C | 212 |
| rs12712978 | 46931824 | 0.221837 | T | G | 209 |
| chr2:46932275 | 46932275 | 0.391116 | C | T | 189 |
| rs9789584 | 46933453 | 0.530038 | G | A | 237 |
| rs61542554 | 46933517 | 0.357946 | C | G | 226 |
| rs61507430 | 46934700 | 0.548775 | C | G | 225 |
| rs13415138 | 46935055 | 0.593169 | C | T | 213 |
| rs6734058 | 46935877 | 0.236694 | G | C | 230 |
| rs11691225 | 46936756 | 0.241328 | T | G | 207 |
| rs10171029 | 46937895 | 0.241328 | C | G | 200 |
| chr2:46938159 | 46938159 | 0.386169 | G | A | 191 |
| rs2278712 | 46939452 | 0.554896 | C | G | 219 |
| rs2278713 | 46939458 | 0.554896 | C | T | 220 |
| rs8622 | 46939510 | 0.241328 | T | C | 234 |
| rs8622 | 46939510 | 0.241328 | C | T | 234 |
| rs875343 | 46940236 | 0.393601 | C | T | 235 |
| rs935380 | 46940486 | 0.391116 | C | T | 236 |
| rs58086358 | 46941703 | 0.557972 | G | A | 224 |
| rs4953431 | 46942424 | 0.396092 | G | T | 222 |
| rs11125097 | 46944448 | 0.243544 | T | C | 201 |
| rs11687608 | 46944845 | 0.24773 | T | C | 206 |
| rs13422107 | 46947398 | 0.576272 | G | C | 214 |
| rs11125098 | 46948511 | 0.576272 | G | T | 202 |
| rs12990097 | 46948709 | 0.25836 | C | G | 210 |
| rs4953434 | 46948993 | 0.372584 | G | A | 223 |
| rs11125099 | 46949951 | 0.265682 | A | G | 203 |
| rs11687528 | 46950457 | 0.264968 | C | G | 205 |
| rs11676288 | 46950462 | 0.289218 | G | A | 204 |
| rs35076028 | 46950695 | 0.266191 | C | T | 221 |
| rs13003608 | 46950761 | 0.262043 | G | A | 211 |
| rs7586009 | 46952472 | 1 | C | T | 233 |
| rs13427181 | 46953597 | 0.591753 | C | G | 215 |

TABLE 4B
Surrogate markers of rs7586009 on Chromosome 2. Markers were
selected using data from the publically available 1000 Genomes
project (http://www.1000genomes.org). Markers that have not
been assigned rs names are identified by their position in
NCBI Build 36 of the human genome assembly. Shown are predicted risk
alleles for the surrogate markers, i.e. alleles that are correlated
with at-risk C allele of rs7586009. Linkage disequilibrium measures
D′ and r², and corresponding p-value, are also shown.
The last column refers to the sequence listing number, identifying
the particular SNP.

| | Pos in | Risk | Other | | | | Seq ID |
|---|---|---|---|---|---|---|---|
| Marker | NCBI_B36 | Allele | Allele | D′ | r² | p-value | NO |
| chr2: 46919668 | 46919668 | A | G | 0.49 | 0.21 | 0.00017 | 185 |
| rs12463795 | 46920688 | C | T | 0.62 | 0.27 | 4.30E−05 | 208 |
| rs17774013 | 46920718 | C | T | 0.65 | 0.28 | 4.20E−05 | 216 |
| chr2: 46921044 | 46921044 | G | C | 0.75 | 0.34 | 3.80E−06 | 186 |
| rs6544930 | 46921405 | G | T | 0.62 | 0.27 | 4.30E−05 | 227 |
| rs6544931 | 46921545 | C | T | 0.62 | 0.27 | 4.30E−05 | 228 |
| chr2: 46924870 | 46924870 | G | T | 0.69 | 0.29 | 1.90E−05 | 187 |
| rs1867821 | 46925934 | T | A | 0.53 | 0.21 | 0.00012 | 218 |
| rs17774133 | 46926764 | C | T | 0.53 | 0.21 | 0.00012 | 217 |
| rs6544932 | 46930495 | G | C | 1 | 0.24 | 6.80E−10 | 229 |
| rs6742192 | 46931070 | G | A | 1 | 0.29 | 2.30E−11 | 231 |
| chr2: 46931429 | 46931429 | C | T | 0.93 | 0.46 | 1.10E−09 | 188 |
| chr2: 46932275 | 46932275 | C | T | 0.93 | 0.46 | 1.10E−09 | 189 |
| rs9789584 | 46933453 | G | A | 0.89 | 0.52 | 4.70E−11 | 237 |
| chr2: 46933517 | 46933517 | C | G | 0.93 | 0.46 | 1.10E−09 | 190 |
| rs13415138 | 46935055 | C | T | 0.89 | 0.52 | 4.70E−11 | 213 |
| rs11691225 | 46936756 | T | G | 1 | 0.31 | 3.90E−12 | 207 |
| rs10171029 | 46937895 | C | G | 1 | 0.33 | 1.60E−12 | 200 |
| chr2: 46938159 | 46938159 | G | A | 0.93 | 0.44 | 2.60E−09 | 191 |
| rs2278712 | 46939452 | C | G | 0.89 | 0.52 | 4.70E−11 | 219 |
| chr2: 46939458 | 46939458 | C | T | 0.89 | 0.52 | 4.70E−11 | 192 |
| rs8622 | 46939510 | T | C | 1 | 0.33 | 1.60E−12 | 234 |
| chr2: 46940236 | 46940236 | C | T | 0.93 | 0.44 | 2.60E−09 | 193 |
| chr2: 46940486 | 46940486 | C | T | 0.93 | 0.46 | 1.10E−09 | 194 |
| rs58086358 | 46941703 | G | A | 0.84 | 0.48 | 5.30E−10 | 224 |
| rs4953431 | 46942424 | G | T | 0.93 | 0.46 | 1.10E−09 | 222 |
| chr2: 46943776 | 46943776 | G | T | 1 | 0.21 | 9.90E−08 | 195 |
| chr2: 46944845 | 46944845 | T | C | 1 | 0.29 | 2.30E−11 | 196 |
| rs13422107 | 46947398 | G | C | 0.84 | 0.48 | 5.30E−10 | 214 |
| rs1 1125098 | 46948511 | G | T | 0.89 | 0.52 | 4.70E−11 | 202 |
| rs12990097 | 46948709 | C | G | 1 | 0.34 | 6.10E−13 | 210 |
| rs4953434 | 46948993 | G | A | 1 | 0.5 | 1.10E−16 | 223 |
| chr2: 46949951 | 46949951 | A | G | 1 | 0.34 | 6.10E−13 | 197 |
| chr2: 46950457 | 46950457 | C | G | 1 | 0.34 | 6.10E−13 | 198 |
| chr2: 46950695 | 46950695 | C | T | 1 | 0.34 | 6.10E−13 | 199 |
| rs13003608 | 46950761 | G | A | 1 | 0.34 | 6.10E−13 | 211 |
| rs13427181 | 46953597 | C | G | 0.91 | 0.66 | 2.20E−14 | 215 |

TABLE 5A
Surrogate markers for rs1983011 on Chromosome 3, determined
in the Icelandic population. Shown are marker names or ID's
(chromosome followed by location in NCBI Build 36),
position in NCBI Build 36, r² values, predicted risk alleles
for the surrogate markers, i.e. alleles that
are correlated with the at-risk C allele of rs1983011 and
SEQ ID for flanking sequence of the marker.

| | Pos in | | Risk | Other | Seq ID |
|---|---|---|---|---|---|
| Marker | NCBI_B36 | r² | Allele | Allele | NO |
| rs12630655 | 178820619 | 0.204946 | G | A | 238 |
| rs10936950 | 178820747 | 0.203454 | T | C | 298 |
| rs2242401 | 178820974 | 0.204837 | G | T | 339 |
| rs4857718 | 178821974 | 0.204856 | C | T | 361 |
| rs4857615 | 178821999 | 0.204818 | C | A | 356 |
| rs4857719 | 178822100 | 0.205233 | T | C | 362 |
| rs12496456 | 178823812 | 0.203773 | A | T | 309 |
| rs13091612 | 178824460 | 0.20479 | C | T | 322 |
| rs9875373 | 178824532 | 0.204321 | A | G | 400 |
| rs1875101 | 178825311 | 0.204903 | A | G | 334 |
| rs1875100 | 178825414 | 0.20545 | T | C | 333 |
| rs4989456 | 178825558 | 0.204783 | A | G | 366 |
| rs7613181 | 178827081 | 0.272359 | A | G | 392 |
| rs11714105 | 178827571 | 0.274129 | T | C | 303 |
| rs55686626 | 178827820 | 0.27312 | G | A | 367 |
| rs62298379 | 178833663 | 0.379359 | C | T | 378 |
| rs10936951 | 178836633 | 0.610231 | T | C | 299 |
| rs10936952 | 178836727 | 0.573393 | C | G | 300 |
| rs1827163 | 178837695 | 0.610231 | A | G | 330 |
| rs7633211 | 178838235 | 0.573393 | C | A | 394 |
| rs6767442 | 178839370 | 0.824391 | T | C | 381 |
| rs35313839 | 178839506 | 0.608535 | G | A | 350 |
| rs6768417 | 178840175 | 0.573393 | C | G | 382 |
| rs11922790 | 178844447 | 0.708729 | A | G | 306 |
| rs11922798 | 178844526 | 0.708678 | A | C | 307 |
| rs13090366 | 178844776 | 0.991177 | T | A | 321 |
| chr3:178846076 | 178846076 | 0.255917 | A | G | 264 |
| rs35874033 | 178846171 | 0.774587 | A | G | 352 |
| rs35227975 | 178846203 | 0.602508 | A | G | 348 |
| rs13074870 | 178846360 | 0.991177 | C | T | 314 |
| rs4857724 | 178847439 | 0.991177 | T | C | 363 |
| rs6780417 | 178848458 | 0.708729 | G | A | 383 |
| rs12485451 | 178848757 | 0.991177 | C | T | 308 |
| rs13060498 | 178849085 | 0.991177 | A | G | 312 |
| rs17571915 | 178849272 | 0.991177 | G | T | 329 |
| rs1499808 | 178850658 | 0.991177 | G | T | 324 |
| rs4857616 | 178851183 | 0.903468 | A | G | 357 |
| rs1967405 | 178851706 | 0.708729 | T | C | 335 |
| rs11922112 | 178852395 | 0.708729 | T | C | 305 |
| rs13090345 | 178854010 | 0.597651 | A | T | 320 |
| rs13076650 | 178855119 | 0.682085 | T | G | 317 |
| rs6789946 | 178856725 | 0.698651 | C | T | 387 |
| rs6789946 | rs6789946 | 0.698651 | T | C | 387 |
| rs6790341 | 178856921 | 0.697326 | G | A | 388 |
| rs6765691 | 178856991 | 0.995576 | A | G | 379 |
| rs12632573 | 178858618 | 0.615716 | C | G | 310 |
| rs1983011 | 178859580 | 1 | C | T | 336 |
| rs59222369 | 178861856 | 0.547673 | T | C | 370 |
| rs13322739 | 178862651 | 0.547673 | A | G | 323 |
| rs13069944 | 178862911 | 0.582499 | C | T | 239 |
| rs35276344 | 178863116 | 0.596562 | A | G | 349 |
| rs4857617 | 178864065 | 0.524157 | C | G | 358 |
| rs4337678 | 178864178 | 0.773456 | T | C | 353 |
| rs4857619 | 178864608 | 0.732796 | A | C | 359 |
| rs61652756 | 178865156 | 0.476486 | C | T | 375 |
| rs13088555 | 178865967 | 0.481483 | G | A | 319 |
| rs35105354 | 178866301 | 0.467668 | T | A | 347 |
| rs4481193 | 178868633 | 0.353977 | C | T | 354 |
| rs59600376 | 178868939 | 0.547673 | T | C | 371 |
| rs2863043 | 178868975 | 0.440354 | A | G | 340 |
| rs2902395 | 178868983 | 0.440354 | G | T | 344 |
| rs9860448 | 178869018 | 0.238618 | T | C | 399 |
| rs59817488 | 178869052 | 0.492239 | C | T | 373 |
| rs4632587 | 178869161 | 0.483485 | G | A | 355 |
| rs35088084 | 178869219 | 0.430099 | A | G | 346 |
| rs2902393 | 178869221 | 0.547673 | A | T | 343 |
| rs2902392 | 178869225 | 0.430099 | A | G | 342 |
| chr3:178869298 | 178869298 | 0.430099 | T | C | 272 |
| chr3:178869299 | 178869299 | 0.430099 | G | A | 273 |
| rs13075547 | 178869446 | 0.508611 | G | C | 316 |
| rs13075140 | 178869449 | 0.281051 | A | G | 315 |
| rs62283309 | 178869609 | 0.488878 | A | G | 376 |
| rs59735843 | 178869701 | 0.400919 | G | A | 372 |
| chr3:178869713 | 178869713 | 0.762175 | T | C | 276 |
| rs57296541 | 178869714 | 0.262217 | G | A | 368 |
| rs60668642 | 178869740 | 0.480791 | A | G | 374 |
| rs62283310 | 178869872 | 0.502744 | A | G | 377 |
| rs35689172 | 178870283 | 0.250704 | T | C | 351 |
| rs57405296 | 178884995 | 0.224989 | A | G | 369 |
| rs937507 | 178887925 | 0.228081 | G | A | 398 |
| rs10513748 | 178895268 | 0.233016 | C | T | 295 |
| rs10513748 | rs10513748 | 0.233016 | G | A | 295 |
| rs2133590 | 178910344 | 0.32699 | A | G | 337 |

TABLE 5B
Surrogate markers of rs1983011 on Chromosome 3. Markers were
selected using data from the publically available 1000 Genomes
project (http://www.1000genomes.org). Markers that have not
been assigned rs names are identified by their position in
NCBI Build 36 of the human genome assembly. Shown are risk alleles
for the surrogate markers, i.e. alleles that are correlated with
the at-risk C allele of rs1983011. Linkage disequilibrium measures
D′ and r², and corresponding p-value, are also shown.
The last column refers to the sequence listing number, identifying
the particular SNP.

| | Pos in | Risk | Other | | | | Seq ID |
|---|---|---|---|---|---|---|---|
| Marker | NCBI_B36 | Allele | Allele | D′ | r² | p-value | NO |
| chr3: 178662539 | 178662539 | C | T | 0.62 | 0.24 | 0.0015 | 240 |
| chr3: 178750434 | 178750434 | C | T | 1 | 0.24 | 8.40E−10 | 241 |
| chr3: 178783659 | 178783659 | C | T | 1 | 0.21 | 1.10E−08 | 242 |
| rs7616679 | 178791180 | C | T | 0.87 | 0.22 | 0.00078 | 393 |
| rs6802551 | 178805515 | C | G | 0.81 | 0.24 | 0.00023 | 390 |
| rs10936949 | 178809219 | A | G | 0.81 | 0.24 | 0.00023 | 297 |
| rs12696446 | 178809968 | T | C | 0.81 | 0.24 | 0.00023 | 311 |
| chr3: 178810822 | 178810822 | G | A | 0.9 | 0.28 | 2.00E−05 | 243 |
| rs4857717 | 178811016 | T | C | 0.81 | 0.24 | 0.00023 | 360 |
| rs6804861 | 178811523 | C | T | 0.81 | 0.24 | 0.00023 | 391 |
| rs937506 | 178811663 | G | C | 0.81 | 0.24 | 0.00023 | 397 |
| chr3: 178812795 | 178812795 | A | G | 0.82 | 0.25 | 9.20E−05 | 244 |
| chr3: 178813968 | 178813968 | T | C | 0.81 | 0.24 | 0.00023 | 245 |
| rs11714247 | 178814430 | A | G | 0.81 | 0.24 | 0.00023 | 304 |
| rs2201991 | 178815002 | A | G | 0.9 | 0.27 | 2.60E−05 | 338 |
| chr3: 178815311 | 178815311 | C | T | 0.81 | 0.24 | 0.00023 | 246 |
| chr3: 178816882 | 178816882 | G | A | 0.91 | 0.3 | 1.60E−05 | 247 |
| rs1566408 | 178816894 | A | G | 0.88 | 0.24 | 0.00032 | 326 |
| chr3: 178818228 | 178818228 | A | G | 0.89 | 0.23 | 9.60E−05 | 248 |
| chr3: 178818316 | 178818316 | C | T | 0.9 | 0.28 | 2.00E−05 | 249 |
| chr3: 178818596 | 178818596 | T | C | 0.88 | 0.22 | 0.00039 | 250 |
| chr3: 178818719 | 178818719 | C | G | 0.88 | 0.22 | 0.00039 | 251 |
| rs13083260 | 178819096 | T | G | 0.9 | 0.27 | 5.20E−05 | 318 |
| rs13060978 | 178819194 | C | G | 0.9 | 0.28 | 2.00E−05 | 313 |
| rs16828300 | 178820228 | G | A | 0.9 | 0.27 | 2.60E−05 | 327 |
| chr3: 178820662 | 178820662 | T | C | 1 | 0.25 | 3.50E−10 | 252 |
| rs10936950 | 178820747 | T | C | 0.9 | 0.27 | 2.60E−05 | 298 |
| rs2242401 | 178820974 | G | T | 0.89 | 0.25 | 6.50E−05 | 339 |
| chr3: 178821974 | 178821974 | C | T | 0.9 | 0.27 | 2.60E−05 | 253 |
| rs4857615 | 178821999 | C | A | 0.9 | 0.27 | 2.60E−05 | 356 |
| rs4857719 | 178822100 | T | C | 0.9 | 0.27 | 2.60E−05 | 362 |
| rs12496456 | 178823812 | A | T | 0.9 | 0.27 | 2.60E−05 | 309 |
| chr3: 178824460 | 178824460 | C | T | 0.89 | 0.25 | 6.50E−05 | 254 |
| chr3: 178824532 | 178824532 | A | G | 0.9 | 0.27 | 2.60E−05 | 255 |
| rs1875101 | 178825311 | A | G | 0.9 | 0.27 | 2.60E−05 | 334 |
| rs1875100 | 178825414 | T | C | 0.9 | 0.27 | 2.60E−05 | 333 |
| rs4989456 | 178825558 | A | G | 0.9 | 0.27 | 2.60E−05 | 366 |
| rs7613181 | 178827081 | A | G | 0.91 | 0.3 | 7.70E−06 | 392 |
| rs11714105 | 178827571 | T | C | 0.91 | 0.3 | 7.70E−06 | 303 |
| chr3: 178827820 | 178827820 | G | A | 0.91 | 0.3 | 7.70E−06 | 256 |
| chr3: 178833663 | 178833663 | C | T | 1 | 0.27 | 5.20E−10 | 257 |
| chr3: 178836633 | 178836633 | T | C | 0.95 | 0.59 | 1.40E−11 | 258 |
| chr3: 178836727 | 178836727 | C | G | 1 | 0.4 | 8.00E−14 | 259 |
| rs1827163 | 178837695 | A | G | 0.86 | 0.53 | 1.30E−09 | 330 |
| chr3: 178838235 | 178838235 | C | A | 0.93 | 0.48 | 1.80E−08 | 260 |
| rs6767442 | 178839370 | T | C | 0.96 | 0.8 | 7.70E−17 | 381 |
| chr3: 178839506 | 178839506 | G | A | 0.95 | 0.61 | 5.90E−12 | 261 |
| chr3: 178840175 | 178840175 | C | G | 0.93 | 0.48 | 1.80E−08 | 262 |
| chr3: 178840192 | 178840192 | A | G | 1 | 0.21 | 1.10E−08 | 263 |
| rs11922790 | 178844447 | A | G | 1 | 0.62 | 1.50E−20 | 306 |
| rs11922798 | 178844526 | A | C | 1 | 0.62 | 1.50E−20 | 307 |
| rs13090366 | 178844776 | T | A | 1 | 0.96 | 1.30E−33 | 321 |
| rs13074870 | 178846360 | C | T | 1 | 0.96 | 1.30E−33 | 314 |
| rs4857724 | 178847439 | T | C | 1 | 0.96 | 1.30E−33 | 363 |
| rs6780417 | 178848458 | G | A | 1 | 0.62 | 1.50E−20 | 383 |
| rs12485451 | 178848757 | C | T | 1 | 0.96 | 1.30E−33 | 308 |
| rs13060498 | 178849085 | A | G | 1 | 0.96 | 1.30E−33 | 312 |
| rs17571915 | 178849272 | G | T | 1 | 0.96 | 1.30E−33 | 329 |
| rs1499808 | 178850658 | G | T | 0.96 | 0.93 | 1.90E−21 | 324 |
| rs4857616 | 178851183 | A | G | 1 | 1 | 7.20E−36 | 357 |
| rs1967405 | 178851706 | T | C | 1 | 0.59 | 8.60E−20 | 335 |
| rs11922112 | 178852395 | T | C | 1 | 0.64 | 2.40E−21 | 305 |
| chr3: 178854010 | 178854010 | A | T | 1 | 0.68 | 2.90E−23 | 265 |
| rs13076650 | 178855119 | T | G | 1 | 0.64 | 2.40E−21 | 317 |
| rs6789946 | 178856725 | C | T | 1 | 0.64 | 2.40E−21 | 387 |
| rs6790341 | 178856921 | G | A | 1 | 0.64 | 2.40E−21 | 388 |
| rs6765691 | 178856991 | A | G | 1 | 1 | 7.20E−36 | 379 |
| rs12632573 | 178858618 | C | G | 1 | 0.68 | 2.90E−23 | 310 |
| chr3: 178862651 | 178862651 | A | G | 1 | 0.59 | 8.60E−20 | 266 |
| chr3: 178864608 | 178864608 | A | C | 1 | 1 | 7.20E−36 | 267 |
| rs13088555 | 178865967 | G | A | 1 | 0.7 | 4.90E−24 | 319 |
| chr3: 178866301 | 178866301 | T | A | 1 | 0.68 | 2.90E−23 | 268 |
| chr3: 178868633 | 178868633 | C | T | 1 | 0.42 | 2.00E−15 | 269 |
| chr3: 178869161 | 178869161 | G | A | 1 | 0.64 | 2.40E−21 | 270 |
| rs35088084 | 178869219 | A | G | 0.95 | 0.66 | 4.00E−13 | 346 |
| chr3: 178869221 | 178869221 | A | T | 0.95 | 0.66 | 4.00E−13 | 271 |
| chr3: 178869298 | 178869298 | T | C | 1 | 0.96 | 1.30E−33 | 272 |
| chr3: 178869299 | 178869299 | G | A | 1 | 0.96 | 1.30E−33 | 273 |
| chr3: 178869446 | 178869446 | G | C | 1 | 0.96 | 1.30E−33 | 274 |
| chr3: 178869449 | 178869449 | A | G | 1 | 0.96 | 1.30E−33 | 275 |
| chr3: 178871089 | 178871089 | T | C | 1 | 0.48 | 2.70E−16 | 277 |
| chr3: 178871686 | 178871686 | T | C | 0.72 | 0.22 | 0.001 | 278 |
| rs10513751 | 178872539 | G | A | 0.73 | 0.22 | 0.00053 | 296 |
| chr3: 178873245 | 178873245 | G | T | 0.73 | 0.22 | 0.00053 | 279 |
| rs7651286 | 178873811 | C | T | 0.73 | 0.22 | 0.00053 | 396 |
| rs16828483 | 178874534 | A | G | 0.73 | 0.22 | 0.00053 | 328 |
| chr3: 178874895 | 178874895 | T | C | 1 | 0.31 | 4.80E−11 | 280 |
| chr3: 178875966 | 178875966 | A | G | 0.73 | 0.22 | 0.00053 | 281 |
| chr3: 178876840 | 178876840 | T | C | 0.74 | 0.24 | 0.00034 | 282 |
| chr3: 178878643 | 178878643 | A | G | 0.72 | 0.21 | 0.0008 | 283 |
| chr3: 178879032 | 178879032 | A | G | 0.73 | 0.22 | 0.00053 | 284 |
| chr3: 178879377 | 178879377 | C | T | 0.73 | 0.22 | 0.00053 | 285 |
| rs57405296 | 178884995 | A | G | 0.73 | 0.35 | 2.60E−06 | 369 |
| chr3: 178885112 | 178885112 | T | C | 0.73 | 0.22 | 0.00053 | 286 |
| rs34585605 | 178885486 | C | T | 0.81 | 0.26 | 0.00017 | 345 |
| chr3: 178885728 | 178885728 | T | C | 0.73 | 0.22 | 0.00053 | 287 |
| chr3: 178886301 | 178886301 | G | A | 0.73 | 0.22 | 0.00053 | 288 |
| chr3: 178886371 | 178886371 | G | C | 0.73 | 0.22 | 0.00053 | 289 |
| rs937507 | 178887925 | G | A | 0.72 | 0.33 | 4.20E−06 | 398 |
| chr3: 178888847 | 178888847 | G | A | 0.78 | 0.21 | 0.0013 | 290 |
| rs10513748 | 178895268 | C | T | 0.72 | 0.33 | 4.20E−06 | 295 |
| chr3: 178906662 | 178906662 | C | G | 1 | 0.22 | 1.60E−08 | 291 |
| rs1499810 | 178908229 | G | A | 0.72 | 0.31 | 6.40E−06 | 325 |
| chr3: 178910344 | 178910344 | A | G | 0.86 | 0.37 | 6.20E−07 | 292 |
| rs6783540 | 178913108 | T | A | 0.65 | 0.22 | 0.00021 | 385 |
| chr3: 178913354 | 178913354 | G | A | 0.58 | 0.24 | 0.00032 | 293 |
| chr3: 178913462 | 178913462 | C | T | 0.65 | 0.22 | 0.00021 | 294 |
| rs67889675 | 178913614 | T | C | 0.65 | 0.22 | 0.00021 | 386 |
| rs1846586 | 178913853 | T | C | 0.65 | 0.22 | 0.00021 | 332 |
| rs1846585 | 178913998 | T | C | 0.65 | 0.22 | 0.00021 | 331 |
| rs4857725 | 178914377 | C | T | 0.58 | 0.24 | 0.00032 | 364 |
| rs4857726 | 178914424 | A | T | 0.7 | 0.29 | 1.80E−05 | 365 |
| rs6765836 | 178914773 | G | C | 0.58 | 0.24 | 0.00032 | 380 |
| rs6793647 | 178915813 | C | A | 0.7 | 0.29 | 1.80E−05 | 389 |
| rs6782388 | 178915993 | C | T | 0.65 | 0.22 | 0.00021 | 384 |
| rs28711160 | 178916822 | A | G | 0.7 | 0.29 | 1.80E−05 | 341 |
| rs7651130 | 178920202 | A | T | 0.56 | 0.21 | 0.00061 | 395 |
| rs1106387 | 178972962 | G | A | 0.64 | 0.21 | 0.0022 | 301 |
| rs1106388 | 178973005 | T | C | 0.64 | 0.21 | 0.0022 | 302 |

TABLE 6
Association for correlated markers of rs1556283 on chromosome 21 with Breast Cancer based on imputed genotypes.
Shown are: Marker rs-names or ID's (chromosome followed by location in NCBI Build 36), P-value of the association
to breast cancer, odds ratio for the minor allele, frequency of minor allele in Icelandic individuals, frequency
of minor allele in European individuals (obtained from 1000genome project), information content of the imputation,
location of marker on chromosome in NCBI Build 36, Minor allele, Major allele, and lastly, a reference to Seq
ID No for flanking sequence of the marker. It should be noted that when indicated OR values are greater than unity,
the Minor allele is the predicted at-risk allele; when indicated OR values are less than unity, other allele (Major
allele) is the predicted at-risk allele (Minor allele is in this case protective).

| | | OR for | Minor All | Minor All | | | | | |
|---|---|---|---|---|---|---|---|---|---|
| | | Minor | freq in | freq in | | Pos in | Minor | Major | Seq ID |
| Marker | P-value | Allele | Iceland (%) | Europe (%) | Info | B36 | Allele | Allele | NO: |
| rs2823040 | 0.225703 | 0.967 | 44.084 | 48.43 | 0.99075 | 15379229 | C | A | 78 |
| rs2823041 | 0.0735269 | 0.944 | 25.087 | 22.7 | 0.98421 | 15379788 | T | C | 79 |
| rs2823042 | 0.243566 | 0.969 | 43.934 | 48.56 | 0.99769 | 15380069 | T | C | 80 |
| rs2823045 | 0.0374598 | 0.937 | 25.658 | 22.7 | 0.99538 | 15382630 | A | G | 81 |
| rs35426920 | 0.0439194 | 0.938 | 25.459 | 22.7 | 0.99533 | 15383585 | A | T | 2 |
| rs2823046 | 0.0392585 | 0.936 | 24.978 | 24.15 | 0.98808 | 15383654 | A | G | 82 |
| rs2823047 | 0.0457753 | 0.939 | 25.722 | 22.83 | 0.99769 | 15383729 | C | T | 3 |
| rs2823049 | 0.323265 | 0.973 | 44.095 | 47.77 | 0.9971 | 15384412 | G | C | 4 |
| rs2823051 | 0.0370752 | 0.936 | 25.53 | 22.7 | 0.99727 | 15384640 | A | G | 5 |
| rs2823052 | 0.0239632 | 0.932 | 26.49 | 24.54 | 0.99375 | 15385989 | T | C | 83 |
| rs1022450 | 0.501248 | 0.982 | 43.095 | 49.48 | 0.9981 | 15386526 | T | C | 6 |
| rs719112 | 0.329695 | 0.974 | 44.858 | 49.87 | 0.99762 | 15387005 | T | A | 157 |
| rs2823054 | 0.297305 | 0.972 | 44.674 | 48.69 | 0.9968 | 15387459 | C | G | 84 |
| rs1005526 | 0.585922 | 0.985 | 43.054 | 48.16 | 0.99596 | 15388481 | A | G | 44 |
| rs760404 | 0.52958 | 0.983 | 42.984 | 48.16 | 0.99789 | 15388760 | T | C | 171 |
| rs57801259 | 0.0253298 | 0.933 | 26.396 | 24.8 | 0.9965 | 15389243 | A | T | 8 |
| rs2403877 | 0.450995 | 0.98 | 43.002 | 49.61 | 0.9974 | 15389951 | T | C | 9 |
| rs2150369 | 0.570514 | 1.02 | 27.338 | 49.87 | 0.76469 | 15392405 | C | — | 73 |
| rs2150369 | 0.660417 | 1.015 | 43.99 | 49.87 | 0.67521 | 15392405 | — | A | 73 |
| rs2150369 | 1 | 1 | 13.716 | 49.87 | 0.74513 | 15392405 | T | — | 73 |
| rs2150370 | 0.0217469 | 0.931 | 26.106 | 24.8 | 0.99517 | 15392529 | A | G | 10 |
| rs1003428 | 0.0234287 | 0.931 | 26.072 | 24.67 | 0.99342 | 15395478 | A | T | 43 |
| rs1003427 | 0.26618 | 0.97 | 44.443 | 49.87 | 0.99576 | 15395832 | T | G | 42 |
| rs1556286 | 0.286035 | 0.971 | 44.288 | 49.08 | 0.99398 | 15396029 | G | T | 60 |
| rs2823056 | 0.23873 | 0.968 | 44.294 | 48.69 | 0.99582 | 15396245 | A | G | 85 |
| rs2823058 | 0.00805408 | 0.922 | 27.768 | 25.07 | 0.99304 | 15398520 | T | A | 86 |
| rs9977308 | 0.291173 | 0.972 | 44.498 | 49.48 | 0.99681 | 15398972 | C | T | 181 |
| rs2823059 | 0.193403 | 0.955 | 18.853 | 19.82 | 0.98979 | 15400165 | G | A | 87 |
| rs55666326 | 0.0178767 | 0.928 | 26 | 24.41 | 0.99302 | 15402303 | C | A | 12 |
| rs2823061 | 0.594313 | 0.985 | 40.358 | 48.56 | 0.99389 | 15403536 | A | G | 88 |
| rs4816486 | 0.945373 | 1.002 | 40.547 | 48.43 | 0.99143 | 15404491 | G | A | 142 |
| rs2823062 | 0.979173 | 1.001 | 40.55 | 48.29 | 0.99131 | 15404514 | G | T | 89 |
| rs9978683 | 0.856802 | 0.995 | 40.538 | 48.43 | 0.99342 | 15405325 | A | G | 183 |
| rs7282276 | 0.213296 | 0.959 | 22.19 | 21.39 | 0.98878 | 15406415 | C | T | 13 |
| rs7282412 | 0.872709 | 0.996 | 40.388 | 48.82 | 0.99381 | 15406522 | A | T | 166 |
| rs2823065 | 0.893208 | 0.996 | 40.342 | 48.69 | 0.99405 | 15407768 | T | C | 90 |
| rs2823066 | 0.843754 | 0.995 | 40.388 | 48.56 | 0.99452 | 15408186 | A | G | 91 |
| rs2026849 | 0.812257 | 0.993 | 40.368 | 48.43 | 0.99426 | 15408497 | A | G | 69 |
| rs2823069 | 0.647913 | 0.987 | 34.247 | 42.91 | 0.97975 | 15409657 | C | T | 92 |
| rs2823070 | 0.640447 | 0.981 | 12.623 | 15.88 | 0.98733 | 15410486 | C | G | 93 |
| rs2026852 | 0.710994 | 0.988 | 25.751 | 28.74 | 0.98911 | 15410557 | A | G | 70 |
| rs7280817 | 0.267634 | 0.968 | 31.815 | 36.22 | 0.99086 | 15410780 | T | C | 14 |
| rs8127658 | 0.213106 | 0.964 | 33.161 | 37.93 | 0.99169 | 15410898 | C | T | 172 |
| rs4817662 | 0.625986 | 0.98 | 12.564 | 16.01 | 0.98493 | 15412114 | T | G | 143 |
| rs2403878 | 0.205129 | 0.964 | 32.858 | 37.93 | 0.99257 | 15412417 | T | C | 76 |
| rs4817666 | 0.661292 | 0.982 | 12.519 | 16.14 | 0.98901 | 15412462 | G | A | 144 |
| rs9977355 | 0.263995 | 0.967 | 30.422 | 35.56 | 0.99354 | 15417178 | A | G | 182 |
| rs2016155 | 0.223833 | 0.965 | 31.976 | 37.01 | 0.99479 | 15420114 | A | G | 68 |
| rs9985165 | 0.236147 | 0.966 | 31.878 | 38.06 | 0.99463 | 15420650 | C | T | 184 |
| rs2823074 | 0.727294 | 0.986 | 14.013 | 19.82 | 0.98317 | 15421527 | C | A | 94 |
| rs2823075 | 0.234448 | 0.943 | 8.506 | 12.99 | 0.98856 | 15426902 | A | G | 95 |
| rs2823076 | 0.117422 | 0.954 | 30.312 | 35.17 | 0.99715 | 15428407 | A | G | 96 |
| rs2823080 | 0.711452 | 0.983 | 10.277 | 15.49 | 0.98897 | 15431719 | A | G | 97 |
| rs2823081 | 0.537129 | 0.98 | 24.757 | 29.66 | 0.98966 | 15431939 | A | T | 98 |
| rs2823082 | 0.58664 | 0.976 | 10.387 | 15.49 | 0.98639 | 15432624 | G | T | 99 |
| rs2823083 | 0.308355 | 0.968 | 24.565 | 29.92 | 0.98877 | 15433269 | G | A | 100 |
| rs926164 | 0.0486325 | 0.93 | 17.18 | 22.57 | 0.98983 | 15435118 | C | A | 174 |
| rs2823084 | 0.361398 | 0.958 | 9.851 | 13.78 | 0.98472 | 15435214 | C | T | 101 |
| rs7281527 | 0.400711 | 0.962 | 9.786 | 14.04 | 0.98499 | 15436032 | C | T | 163 |
| rs2823085 | 0.044797 | 0.929 | 17.656 | 22.97 | 0.98981 | 15437113 | C | G | 102 |
| rs2823086 | 0.371747 | 0.959 | 9.815 | 14.04 | 0.98477 | 15437412 | G | A | 103 |
| rs2823087 | 0.0396279 | 0.928 | 17.693 | 22.97 | 0.99096 | 15437630 | T | C | 104 |
| rs2823088 | 0.0625169 | 0.931 | 15.786 | 21.13 | 0.99032 | 15437844 | A | G | 105 |
| rs2823089 | 0.0395961 | 0.928 | 17.686 | 22.97 | 0.99076 | 15437925 | T | C | 106 |
| rs926166 | 0.0395096 | 0.928 | 17.688 | 22.97 | 0.99071 | 15437935 | A | G | 175 |
| rs55859143 | 0.382113 | 0.96 | 9.821 | 14.04 | 0.98437 | 15438022 | G | A | 148 |
| rs12482834 | 0.0546906 | 0.929 | 15.777 | 21.13 | 0.99146 | 15438153 | C | T | 56 |
| rs741937 | 0.0744798 | 0.934 | 15.874 | 21.13 | 0.98971 | 15439702 | G | T | 170 |
| rs2049880 | 0.0392777 | 0.928 | 17.695 | 22.97 | 0.99068 | 15439762 | C | G | 71 |
| rs2823091 | 0.372292 | 0.959 | 9.816 | 14.04 | 0.9847 | 15439876 | T | A | 107 |
| rs2007783 | 0.365911 | 0.959 | 9.856 | 14.04 | 0.98435 | 15440395 | T | G | 66 |
| rs2823093 | 0.00864101 | 0.914 | 20.745 | 25.85 | 0.99134 | 15442703 | A | G | 108 |
| rs2896680 | 0.0042561 | 0.906 | 20.318 | 25.2 | 0.99016 | 15442806 | T | A | 140 |
| rs2256038 | 0.0338891 | 0.937 | 27.825 | 33.33 | 0.99178 | 15443047 | A | G | 75 |
| rs2823096 | 0.044339 | 0.923 | 13.935 | 18.24 | 0.99218 | 15443953 | G | A | 109 |
| rs2823097 | 0.0334035 | 0.937 | 27.85 | 33.6 | 0.99203 | 15445015 | C | G | 110 |
| rs9974193 | 0.00132356 | 0.909 | 31.957 | 38.06 | 0.99159 | 15450904 | G | T | 179 |
| rs2823106 | 0.00204578 | 0.895 | 18.188 | 22.7 | 0.98975 | 15454531 | C | T | 111 |
| rs9975709 | 0.00143865 | 0.91 | 31.784 | 37.66 | 0.99195 | 15455511 | A | C | 180 |
| rs62218221 | 0.00212663 | 0.895 | 18.211 | 22.7 | 0.98934 | 15455545 | C | T | 150 |
| rs7275529 | 0.00130282 | 0.909 | 31.814 | 37.66 | 0.99417 | 15456069 | C | T | 159 |
| rs2008843 | 0.00212795 | 0.895 | 18.213 | 22.7 | 0.98932 | 15456839 | C | T | 67 |
| rs2823107 | 0.00142506 | 0.91 | 31.86 | 37.66 | 0.99239 | 15457405 | G | A | 112 |
| rs55792425 | 0.00208155 | 0.895 | 18.171 | 22.7 | 0.99076 | 15457964 | T | A | 146 |
| rs62218223 | 0.00225612 | 0.896 | 18.178 | 22.7 | 0.99055 | 15458191 | A | G | 151 |
| rs11088297 | 0.00128282 | 0.909 | 31.925 | 37.93 | 0.99085 | 15458583 | T | C | 46 |
| rs77446902 | 0.00127224 | 0.909 | 31.903 | 37.93 | 0.99136 | 15458584 | A | T | 47 |
| rs11088299 | 0.00116678 | 0.909 | 31.987 | 37.93 | 0.99099 | 15458627 | C | T | 48 |
| rs12482761 | 0.00215759 | 0.896 | 18.286 | 22.57 | 0.98893 | 15459318 | C | T | 55 |
| rs990302 | 0.00220115 | 0.896 | 18.301 | 22.57 | 0.99125 | 15460110 | A | G | 178 |
| rs62218224 | 0.00224663 | 0.896 | 18.297 | 22.57 | 0.99187 | 15460995 | C | G | 152 |
| rs62218225 | 0.0022497 | 0.896 | 18.297 | 22.57 | 0.99187 | 15461014 | C | A | 153 |
| rs2896689 | 0.00222511 | 0.896 | 18.284 | 22.57 | 0.99165 | 15462286 | A | C | 141 |
| rs7282135 | 0.00257723 | 0.915 | 32.584 | 38.32 | 0.99382 | 15463056 | C | A | 164 |
| chr21: 15464779 | 0.0021008 | 0.895 | 18.272 | NA | 0.99189 | 15464779 | TA | T | 25 |
| rs2223128 | 0.00260179 | 0.915 | 32.584 | 38.32 | 0.99367 | 15464831 | T | G | 74 |
| rs2823110 | 0.00212067 | 0.896 | 18.28 | 22.57 | 0.99239 | 15464942 | T | C | 113 |
| rs2823111 | 0.00209751 | 0.895 | 18.276 | 22.57 | 0.99248 | 15465149 | A | G | 114 |
| rs2823112 | 0.002119 | 0.896 | 18.289 | 22.57 | 0.9927 | 15465216 | C | G | 115 |
| chr21: 15465320 | 0.502181 | 0.796 | 0.357 | NA | 0.54 | 15465320 | A | T | 26 |
| chr21: 15465323 | 0.652178 | 0.856 | 0.335 | NA | 0.54549 | 15465323 | C | G | 27 |
| rs2142362 | 0.00224663 | 0.896 | 18.297 | 22.57 | 0.99187 | 15465381 | A | G | 72 |
| rs2823113 | 0.00233284 | 0.896 | 18.232 | 22.57 | 0.99395 | 15465915 | C | T | 116 |
| rs1882959 | 0.00213455 | 0.896 | 18.262 | 22.57 | 0.99236 | 15466379 | C | T | 61 |
| rs12482386 | 0.002178 | 0.896 | 18.24 | 22.57 | 0.99327 | 15466790 | A | G | 54 |
| rs2823115 | 0.00215688 | 0.896 | 18.297 | 22.57 | 0.99165 | 15466848 | G | A | 117 |
| rs7279893 | 0.00222584 | 0.914 | 32.612 | 38.45 | 0.99397 | 15467399 | C | T | 162 |
| rs2823117 | 0.00239263 | 0.897 | 18.3 | 22.57 | 0.99128 | 15467738 | T | C | 118 |
| rs2823118 | 0.00301332 | 0.916 | 32.358 | 38.71 | 0.99425 | 15467777 | T | A | 119 |
| rs12483016 | 0.00211062 | 0.895 | 18.295 | 22.57 | 0.99152 | 15467996 | T | G | 57 |
| rs12483602 | 0.00205916 | 0.895 | 18.322 | 22.57 | 0.99184 | 15468304 | T | C | 58 |
| rs55797017 | 0.00217584 | 0.896 | 18.32 | 22.57 | 0.99116 | 15468489 | T | G | 147 |
| rs62218995 | 0.00211761 | 0.896 | 18.307 | 22.57 | 0.99191 | 15469153 | T | C | 154 |
| rs8128132 | 0.00208938 | 0.895 | 18.32 | 22.57 | 0.99207 | 15469185 | T | A | 173 |
| rs73176585 | 0.00236382 | 0.896 | 18.29 | NA | 0.98982 | 15469191 | A | T | 167 |
| rs11909473 | 0.000323018 | 0.891 | 25.012 | 30.71 | 0.9912 | 15469306 | G | A | 51 |
| rs2823119 | 0.00218485 | 0.896 | 18.324 | 22.57 | 0.991 | 15469515 | C | A | 120 |
| rs62218996 | 0.00191678 | 0.894 | 18.233 | 22.44 | 0.98982 | 15469959 | A | G | 155 |
| rs62218997 | 0.00213702 | 0.896 | 18.316 | 22.44 | 0.99101 | 15470608 | A | G | 156 |
| rs12482303 | 0.00199279 | 0.895 | 18.334 | 22.44 | 0.99081 | 15471530 | T | C | 53 |
| rs1014526 | 0.00279768 | 0.919 | 36.67 | 41.99 | 0.99461 | 15471649 | T | C | 45 |
| rs2823121 | 0.00391884 | 0.921 | 36.567 | 41.99 | 0.9954 | 15473128 | G | A | 121 |
| rs2823122 | 0.00236479 | 0.897 | 18.313 | 22.44 | 0.99193 | 15473292 | C | G | 122 |
| rs2823123 | 0.0023428 | 0.897 | 18.337 | 22.44 | 0.99141 | 15474025 | A | G | 123 |
| rs2823124 | 0.00431227 | 0.922 | 36.51 | 41.99 | 0.99509 | 15474392 | A | G | 124 |
| rs11088311 | 0.00195825 | 0.895 | 18.302 | 21.78 | 0.98923 | 15474905 | A | C | 49 |
| rs12373912 | 0.0028583 | 0.898 | 18.242 | 22.44 | 0.99195 | 15475004 | A | G | 52 |
| rs55778206 | 0.00239927 | 0.897 | 18.306 | 22.44 | 0.99203 | 15476933 | T | A | 145 |
| rs1882961 | 0.00976844 | 0.925 | 28.54 | 32.15 | 0.99472 | 15478238 | T | C | 62 |
| rs1556283 | 0.00175963 | 0.894 | 18.338 | 22.57 | 0.99161 | 15478599 | T | C | 59 |
| rs965098 | 0.00273757 | 0.918 | 36.51 | 41.99 | 0.99322 | 15479497 | A | G | 177 |
| rs1882963 | 0.00233786 | 0.896 | 18.261 | 22.44 | 0.99233 | 15481989 | C | G | 63 |
| rs2823128 | 0.00631833 | 0.921 | 29.205 | 31.23 | 0.99111 | 15485180 | A | G | 125 |
| rs2823129 | 0.0068503 | 0.921 | 29.217 | 31.23 | 0.99166 | 15485511 | T | C | 126 |
| rs7282349 | 0.00586284 | 0.92 | 29.198 | 31.23 | 0.9921 | 15486432 | T | C | 165 |
| rs926167 | 0.00379603 | 0.916 | 28.824 | 30.71 | 0.99605 | 15488394 | C | T | 176 |
| rs2823131 | 0.00172662 | 0.909 | 28.754 | 30.31 | 0.99562 | 15489599 | A | G | 127 |
| rs7275438 | 0.00357732 | 0.915 | 28.833 | 30.31 | 0.99593 | 15490199 | T | C | 158 |
| rs7275695 | 0.00615986 | 0.92 | 28.77 | 30.58 | 0.99427 | 15490395 | T | C | 160 |
| rs2823132 | 0.00496228 | 0.918 | 28.831 | 30.31 | 0.9951 | 15490727 | C | T | 128 |
| rs2823133 | 0.00498185 | 0.918 | 28.823 | 30.31 | 0.99517 | 15490812 | C | A | 129 |
| rs2823134 | 0.00488663 | 0.918 | 28.817 | 30.31 | 0.99429 | 15490934 | G | A | 130 |
| rs115193155 | 0.838728 | 1.014 | 5.086 | NA | 0.77393 | 15491897 | A | G | 37 |
| rs2823135 | 0.00495004 | 0.918 | 28.812 | 30.31 | 0.99467 | 15492355 | G | A | 131 |
| rs2823136 | 0.00557153 | 0.919 | 28.829 | 30.45 | 0.99473 | 15493822 | C | G | 132 |
| rs7276622 | 0.00414807 | 0.917 | 28.855 | 30.45 | 0.99582 | 15494147 | A | G | 161 |
| rs11088317 | 0.0138994 | 0.926 | 26.567 | 27.82 | 0.99502 | 15495993 | T | C | 50 |
| rs2403907 | 0.011126 | 0.926 | 28.513 | 29 | 0.99483 | 15496326 | A | C | 77 |
| rs2823137 | 0.00775244 | 0.922 | 27.961 | 27.56 | 0.99416 | 15496913 | A | T | 133 |
| rs2823138 | 0.0231726 | 0.928 | 22.831 | 21.39 | 0.99134 | 15498364 | A | G | 134 |
| rs2823139 | 0.0683286 | 0.949 | 33.351 | 32.28 | 0.99285 | 15498654 | A | G | 135 |
| rs73184544 | 0.000145947 | 0.835 | 9.971 | 9.32 | 0.98925 | 15499420 | T | G | 169 |
| rs1997595 | 0.122858 | 0.957 | 34.271 | 32.55 | 0.99185 | 15500030 | C | A | 64 |
| rs1997596 | 0.139171 | 0.958 | 34.359 | 32.55 | 0.99159 | 15500319 | T | C | 65 |
| rs2823140 | 0.0813776 | 0.951 | 34.821 | 32.81 | 0.99207 | 15500671 | A | G | 136 |
| rs2823141 | 0.0397022 | 0.944 | 40.321 | 38.98 | 0.99027 | 15501676 | A | G | 137 |
| rs2823142 | 0.0393338 | 0.944 | 40.338 | 38.98 | 0.99084 | 15501823 | G | A | 138 |
| rs56038390 | 0.340647 | 0.973 | 33.343 | 32.41 | 0.99099 | 15504581 | G | A | 149 |
| rs2823144 | 0.548009 | 0.983 | 37.299 | 37.93 | 0.98994 | 15504804 | G | C | 139 |
| rs8128542 | 0.736948 | 0.982 | 6.887 | 8.01 | 0.98973 | 15566068 | T | C | 41 |

TABLE 7
Association for correlated markers of rs7586009 on chromosome 2 with Breast Cancer based on imputed genotypes.
Shown are: Marker names, P-value of the association to breast cancer in Icelandic breast cancer patients,
odds ratio for the minor allele, frequency of minor allele in Icelandic patients, frequency of minor allele
in European patients (obtained from 1000genome project), information content of the imputation, location
of marker on chromosome in NCBI Build 36, Minor allele, Major allele, and lastly, a reference to Seq ID No
for flanking sequence of the marker. It should be noted that when indicated OR values are greater than unity,
the Minor allele is the predicted at-risk allele; when indicated OR values are less than unity, other allele
(Major allele) is the predicted at-risk allele (Minor allele is in this case protective).

| | | OR for | Minor All | Minor All | | | | | |
|---|---|---|---|---|---|---|---|---|---|
| | | Minor | freq in | freq in | | Pos in | Minor | Major | Seq ID |
| Marker | P-value | Allele | Iceland (%) | Europe (%) | Info | B36 | Allele | Allele | NO: |
| rs1947109 | 0.0955149 | 0.951 | 29.413 | 35.96 | 0.99254 | 46919668 | G | A | 185 |
| rs12463795 | 0.0466417 | 0.941 | 29.563 | 33.6 | 0.99418 | 46920688 | T | C | 208 |
| rs17774013 | 0.0472119 | 0.941 | 27.747 | 31.76 | 0.99656 | 46920718 | T | C | 216 |
| rs35772556 | 0.0400206 | 0.937 | 26.425 | 27.82 | 0.99441 | 46921044 | C | G | 186 |
| rs6544930 | 0.0453007 | 0.941 | 29.58 | 33.6 | 0.99391 | 46921405 | T | G | 227 |
| rs6544931 | 0.0410139 | 0.94 | 29.503 | 33.6 | 0.99404 | 46921545 | T | C | 228 |
| rs55642514 | 0.0505552 | 0.942 | 28.78 | 29.4 | 0.99499 | 46924870 | T | G | 187 |
| rs1867821 | 0.0587292 | 0.945 | 30.733 | 32.41 | 0.99667 | 46925934 | A | T | 218 |
| rs17774133 | 0.059942 | 0.945 | 30.282 | 32.41 | 0.99669 | 46926764 | T | C | 217 |
| rs6544932 | 0.477908 | 1.023 | 24.669 | 26.12 | 0.99476 | 46930495 | G | C | 229 |
| rs6742192 | 0.690202 | 1.013 | 24.302 | 26.64 | 0.99006 | 46931070 | G | A | 231 |
| rs72804479 | 0.0892292 | 0.948 | 26.388 | 26.51 | 0.98816 | 46931429 | T | C | 232 |
| rs13006048 | 0.0055134 | 1.085 | 31.691 | 29.27 | 0.99264 | 46931823 | T | C | 212 |
| rs12712978 | 0.0056166 | 1.084 | 31.857 | 29.27 | 0.99281 | 46931824 | T | G | 209 |
| rs113862557 | 0.0696961 | 0.944 | 26.42 | 26.51 | 0.98787 | 46932275 | T | C | 189 |
| rs9789584 | 0.358059 | 0.974 | 34.688 | 33.33 | 0.9915 | 46933453 | A | G | 237 |
| rs61542554 | 0.0945895 | 0.949 | 26.343 | 26.38 | 0.98803 | 46933517 | G | C | 226 |
| rs61507430 | 0.265449 | 0.968 | 34.404 | 33.2 | 0.99111 | 46934700 | G | C | 225 |
| rs13415138 | 0.254128 | 0.967 | 32.714 | 31.36 | 0.9915 | 46935055 | T | C | 213 |
| rs11691225 | 0.660167 | 1.014 | 24.186 | 28.22 | 0.99149 | 46936756 | T | G | 207 |
| rs10171029 | 0.633122 | 1.015 | 24.143 | 28.22 | 0.99768 | 46937895 | C | G | 200 |
| rs113628100 | 0.0633847 | 0.943 | 26.471 | 26.25 | 0.99034 | 46938159 | A | G | 191 |
| rs2278712 | 0.178644 | 0.962 | 34.515 | 33.33 | 0.99359 | 46939452 | G | C | 219 |
| rs2278713 | 0.177286 | 0.962 | 34.494 | 33.33 | 0.99375 | 46939458 | T | C | 220 |
| rs8622 | 0.656389 | 1.014 | 24.255 | 28.22 | 0.99635 | 46939510 | T | C | 234 |
| rs875343 | 0.0810215 | 0.947 | 26.514 | 26.25 | 0.99424 | 46940236 | T | C | 235 |
| rs935380 | 0.0703292 | 0.945 | 26.429 | 26.25 | 0.99386 | 46940486 | T | C | 236 |
| rs58086358 | 0.167583 | 0.961 | 34.485 | 33.33 | 0.99607 | 46941703 | A | G | 224 |
| rs4953431 | 0.0708931 | 0.945 | 26.542 | 26.38 | 0.99509 | 46942424 | T | G | 222 |
| rs11125097 | 0.0093536 | 1.079 | 32.504 | 29.79 | 0.99464 | 46944448 | T | C | 201 |
| rs11687608 | 0.630201 | 1.015 | 24.573 | 27.43 | 0.99838 | 46944845 | T | C | 206 |
| rs13422107 | 0.199775 | 0.963 | 33.243 | 31.5 | 0.99511 | 46947398 | C | G | 214 |
| rs11125098 | 0.215956 | 0.964 | 33.376 | 31.5 | 0.99443 | 46948511 | T | G | 202 |
| rs12990097 | 0.517562 | 1.021 | 24.141 | 27.3 | 0.99697 | 46948709 | C | G | 210 |
| rs4953434 | 0.0846233 | 0.947 | 26.625 | 26.12 | 0.99455 | 46948993 | A | G | 223 |
| rs11125099 | 0.392207 | 1.028 | 23.887 | 27.56 | 0.997 | 46949951 | A | G | 203 |
| rs11687528 | 0.397922 | 1.027 | 23.978 | 27.43 | 0.99693 | 46950457 | C | G | 205 |
| rs11676288 | 0.105629 | 1.052 | 25.013 | 29.27 | 0.9967 | 46950462 | G | A | 204 |
| rs35076028 | 0.460408 | 1.024 | 23.859 | 27.43 | 0.99675 | 46950695 | C | T | 221 |
| rs13003608 | 0.394469 | 1.028 | 23.847 | 27.43 | 0.99721 | 46950761 | G | A | 211 |
| rs13427181 | 0.00826496 | 0.927 | 37.002 | 36.09 | 0.99251 | 46953597 | G | C | 215 |

TABLE 8
Association for correlated markers of rs1983011 on chromosome 3 with Breast Cancer based on imputed genotypes.
Shown are: Marker rs-names or ID'S (chromosome followed by location in NCBI Build 36), P-value of the association,
odds ratio for the minor allele, frequency of minor allele in Icelandic individuals, frequency of minor allele
in European individuals (obtained from 1000genome project), information content of the imputation, location
of marker on chromosome in NCBI Build 36, Minor allele, Major allele, and lastly, a reference to Seq ID No for
flanking sequence of the marker. It should be noted that when indicated OR values are greater than unity, the
Minor allele is the predicted at-risk allele; when indicated OR values are less than unity, other allele (Major
allele) is the predicted at-risk allele (Minor allele is in this case protective).

| | | OR for | Minor All | Minor All | | | | | |
|---|---|---|---|---|---|---|---|---|---|
| | | Minor | freq in | freq in | | Pos in | Minor | Major | Seq ID |
| Marker | P-value | Allele | Iceland (%) | Europe (%) | Info | B36 | Allele | Allele | NO: |
| rs11716149 | 0.789736 | 1.008 | 34.122 | 36.35 | 0.98921 | 178662539 | C | T | 240 |
| rs73174803 | 0.0545221 | 0.943 | 27.884 | 22.7 | 0.99003 | 178750434 | T | C | 241 |
| rs4857710 | 0.00688415 | 0.927 | 40.492 | 38.58 | 0.99176 | 178783659 | T | C | 242 |
| rs7616679 | 0.00407143 | 0.923 | 41.31 | 38.71 | 0.98895 | 178791180 | T | C | 393 |
| rs6802551 | 0.215401 | 0.965 | 33.724 | 30.58 | 0.98872 | 178805515 | G | C | 390 |
| rs10936949 | 0.322048 | 0.972 | 33.624 | 30.71 | 0.99048 | 178809219 | G | A | 297 |
| rs12696446 | 0.225942 | 0.965 | 33.731 | 30.71 | 0.99072 | 178809968 | C | T | 311 |
| rs9871317 | 0.24589 | 0.967 | 33.823 | 30.71 | 0.99001 | 178810822 | A | G | 243 |
| rs4857717 | 0.223999 | 0.965 | 33.717 | 30.71 | 0.99095 | 178811016 | C | T | 360 |
| rs6804861 | 0.196564 | 0.963 | 33.858 | 30.71 | 0.98914 | 178811523 | T | C | 391 |
| rs937506 | 0.251791 | 0.967 | 33.755 | 30.71 | 0.98983 | 178811663 | C | G | 397 |
| rs2014126 | 0.279745 | 0.969 | 33.762 | 30.71 | 0.98929 | 178812795 | G | A | 244 |
| rs11714247 | 0.200491 | 0.963 | 33.748 | 30.58 | 0.98919 | 178814430 | G | A | 304 |
| rs2201991 | 0.22407 | 0.965 | 33.815 | 30.58 | 0.99021 | 178815002 | G | A | 338 |
| rs1123129 | 0.257249 | 0.968 | 33.747 | 30.58 | 0.99048 | 178815311 | T | C | 246 |
| rs1566409 | 0.169342 | 0.961 | 34.116 | 31.36 | 0.99019 | 178816882 | A | G | 247 |
| rs1566408 | 0.16843 | 0.961 | 34.136 | 31.36 | 0.9894 | 178816894 | G | A | 326 |
| rs35592194 | 0.170484 | 0.961 | 34.118 | 31.23 | 0.98997 | 178818228 | G | A | 248 |
| rs12497597 | 0.165895 | 0.961 | 34.124 | 31.23 | 0.98988 | 178818316 | T | C | 249 |
| rs9829760 | 0.153609 | 0.959 | 34.16 | 31.23 | 0.9899 | 178818596 | C | T | 250 |
| rs71304074 | 0.13889 | 0.958 | 34.156 | 31.1 | 0.98974 | 178818719 | G | C | 251 |
| rs13083260 | 0.170208 | 0.961 | 34.065 | 31.23 | 0.98818 | 178819096 | G | T | 318 |
| rs13060978 | 0.150113 | 0.959 | 34.145 | 31.23 | 0.9893 | 178819194 | G | C | 313 |
| rs16828300 | 0.151334 | 0.959 | 34.115 | 31.23 | 0.99005 | 178820228 | A | G | 327 |
| rs10936950 | 0.0913834 | 0.952 | 33.55 | 31.36 | 0.98536 | 178820747 | C | T | 298 |
| rs2242401 | 0.117773 | 0.955 | 33.814 | 31.36 | 0.98864 | 178820974 | T | G | 339 |
| rs4857718 | 0.100193 | 0.953 | 33.761 | 31.36 | 0.9894 | 178821974 | T | C | 361 |
| rs4857615 | 0.115497 | 0.955 | 33.617 | 31.36 | 0.98641 | 178821999 | A | C | 356 |
| rs4857719 | 0.0994391 | 0.953 | 33.781 | 31.36 | 0.98829 | 178822100 | C | T | 362 |
| rs12496456 | 0.0978997 | 0.953 | 33.798 | 31.36 | 0.98812 | 178823812 | T | A | 309 |
| rs13091612 | 0.100364 | 0.953 | 33.793 | 31.36 | 0.98854 | 178824460 | T | C | 322 |
| rs9875373 | 0.121509 | 0.956 | 33.814 | 31.36 | 0.98911 | 178824532 | G | A | 400 |
| rs1875101 | 0.116575 | 0.955 | 33.839 | 31.36 | 0.98973 | 178825311 | G | A | 334 |
| rs1875100 | 0.12155 | 0.956 | 33.832 | 31.36 | 0.98932 | 178825414 | C | T | 333 |
| rs4989456 | 0.0989684 | 0.953 | 33.79 | 31.36 | 0.98893 | 178825558 | G | A | 366 |
| rs7613181 | 0.0209606 | 0.936 | 37.461 | 37.4 | 0.98869 | 178827081 | G | A | 392 |
| rs11714105 | 0.0239068 | 0.938 | 37.396 | 37.4 | 0.98845 | 178827571 | C | T | 303 |
| rs55686626 | 0.0245009 | 0.938 | 37.281 | 37.4 | 0.98727 | 178827820 | A | G | 367 |
| rs62298379 | 0.153946 | 1.047 | 22.831 | 19.82 | 0.99037 | 178833663 | C | T | 378 |
| rs10936951 | 0.00458465 | 0.924 | 43.878 | 45.67 | 0.98821 | 178836633 | C | T | 299 |
| rs10936952 | 0.00603682 | 1.084 | 31.086 | 28.48 | 0.99311 | 178836727 | C | G | 300 |
| rs1827163 | 0.00384234 | 0.923 | 44.011 | 45.67 | 0.99145 | 178837695 | G | A | 330 |
| rs7633211 | 0.00646762 | 1.083 | 31.058 | 28.48 | 0.99255 | 178838235 | C | A | 394 |
| rs6767442 | 0.00130068 | 1.094 | 39.871 | 38.98 | 0.99243 | 178839370 | T | C | 381 |
| rs35313839 | 0.00263639 | 0.92 | 44.161 | 45.8 | 0.99134 | 178839506 | A | G | 350 |
| rs6768417 | 0.00676511 | 1.083 | 31.063 | 28.48 | 0.9925 | 178840175 | C | G | 382 |
| rs11922790 | 0.00173246 | 1.092 | 36.438 | 34.25 | 0.99039 | 178844447 | A | G | 306 |
| rs11922798 | 0.00208018 | 1.091 | 36.411 | 34.25 | 0.99336 | 178844526 | A | C | 307 |
| rs13090366 | 0.000139202 | 1.11 | 45.092 | 44.75 | 0.99113 | 178844776 | T | A | 321 |
| rs112232680 | 0.0123715 | 0.928 | 39.445 | NA | 0.90263 | 178846076 | G | A | 264 |
| rs35874033 | 0.00901432 | 0.927 | 43.699 | 44.88 | 0.92012 | 178846171 | G | A | 352 |
| rs35227975 | 0.00601934 | 0.926 | 43.726 | 41.73 | 0.98363 | 178846203 | G | A | 348 |
| rs13074870 | 0.000153157 | 1.11 | 45.065 | 44.75 | 0.9914 | 178846360 | C | T | 314 |
| rs4857724 | 0.000256713 | 1.106 | 45.034 | 44.75 | 0.98923 | 178847439 | T | C | 363 |
| rs6780417 | 0.00185897 | 1.092 | 36.475 | 34.25 | 0.98862 | 178848458 | G | A | 383 |
| rs12485451 | 0.000172968 | 1.109 | 45.025 | 44.75 | 0.9896 | 178848757 | C | T | 308 |
| rs13060498 | 0.000164998 | 1.109 | 45.098 | 44.75 | 0.991 | 178849085 | A | G | 312 |
| rs17571915 | 0.000168555 | 1.109 | 45.08 | 44.75 | 0.99145 | 178849272 | G | T | 329 |
| rs1499808 | 0.00017868 | 1.109 | 45.025 | 44.75 | 0.9902 | 178850658 | G | T | 324 |
| rs1967405 | 0.00233893 | 1.09 | 36.404 | 34.25 | 0.98968 | 178851706 | T | C | 335 |
| rs11922112 | 0.00257414 | 1.089 | 36.403 | 34.25 | 0.99016 | 178852395 | T | C | 305 |
| rs13090345 | 0.00432507 | 0.924 | 44.575 | 45.67 | 0.99046 | 178854010 | T | A | 320 |
| rs13076650 | 0.00171331 | 1.093 | 35.759 | 34.38 | 0.98939 | 178855119 | T | G | 317 |
| rs6789946 | 0.00163337 | 1.093 | 36.318 | 34.38 | 0.98995 | 178856725 | C | T | 387 |
| rs6790341 | 0.00111451 | 1.097 | 36.17 | 34.38 | 0.98986 | 178856921 | G | A | 388 |
| rs6765691 | 0.000124931 | 1.111 | 45.189 | 44.88 | 0.99065 | 178856991 | A | G | 379 |
| rs12632573 | 0.00248875 | 0.92 | 44.193 | 45.67 | 0.99194 | 178858618 | G | C | 310 |
| rs1983011 | 0.000124061 | 1.111 | 45.002 | 45.28 | 0.98884 | 178859580 | G | A | 336 |
| rs74344233 | 0.0116908 | 1.077 | 30.416 | 25.98 | 0.98922 | 178861856 | T | C | 370 |
| rs115233048 | 0.00935837 | 1.08 | 30.462 | 27.69 | 0.99221 | 178862651 | A | G | 323 |
| rs13069944 | 0.00381124 | 0.923 | 44.015 | 4.33 | 0.98877 | 178862911 | T | C | 239 |
| chr3: 178863116 | 0.00545611 | 0.926 | 44.107 | 43.04 | 0.98978 | 178863116 | G | A | 349 |
| rs4857617 | 0.0150809 | 0.935 | 47.535 | 49.87 | 0.97329 | 178864065 | G | C | 358 |
| rs4337678 | 0.00245246 | 1.089 | 39.637 | 42.91 | 0.9739 | 178864178 | T | C | 353 |
| rs4857619 | 0.00305526 | 1.087 | 38.777 | 41.6 | 0.97407 | 178864608 | A | C | 359 |
| rs61652756 | 0.00641746 | 0.927 | 47.794 | 47.64 | 0.97717 | 178865156 | T | C | 375 |
| rs13088555 | 0.00798692 | 0.929 | 48.037 | 49.87 | 0.97716 | 178865967 | A | G | 319 |
| rs35105354 | 0.00653934 | 0.928 | 47.769 | 49.87 | 0.97823 | 178866301 | A | T | 347 |
| rs4481193 | 0.0319632 | 1.06 | 47.283 | 47.38 | 0.99227 | 178868633 | C | T | 354 |
| rs147893886 | 0.0100861 | 1.079 | 30.471 | 27.43 | 0.99189 | 178868939 | T | C | 371 |
| rs140333310 | 0.028677 | 1.062 | 49.335 | 41.34 | 0.98889 | 178868975 | A | G | 340 |
| rs149895579 | 0.0296978 | 1.062 | 49.448 | 40.55 | 0.98657 | 178868983 | G | T | 344 |
| rs150372219 | 0.189882 | 1.037 | 40.834 | 29 | 0.9886 | 178869018 | T | C | 399 |
| rs141632341 | 0.0125908 | 1.076 | 31.112 | 28.08 | 0.99039 | 178869052 | C | T | 373 |
| rs4632587 | 0.0224911 | 0.939 | 47.258 | 47.51 | 0.98822 | 178869161 | A | G | 355 |
| rs149599358 | 0.0230637 | 0.94 | 49.907 | 43.83 | 0.98913 | 178869219 | G | A | 346 |
| rs147244465 | 0.00876527 | 1.08 | 30.463 | 25.46 | 0.99195 | 178869221 | A | T | 343 |
| rs140740493 | 0.0213532 | 0.939 | 49.932 | 43.7 | 0.9894 | 178869225 | G | A | 342 |
| rs150007940 | 0.0202565 | 0.938 | 49.979 | 45.28 | 0.99107 | 178869298 | C | T | 272 |
| rs141011169 | 0.0201924 | 0.938 | 49.978 | 45.67 | 0.9911 | 178869299 | A | G | 273 |
| rs146661873 | 0.0147539 | 0.935 | 46.861 | 31.23 | 0.98918 | 178869446 | C | G | 316 |
| rs140244555 | 0.0940726 | 1.047 | 44.633 | 42.78 | 0.9908 | 178869449 | A | G | 315 |
| rs137924870 | 0.0193239 | 1.073 | 28.452 | 25.59 | 0.98774 | 178869609 | A | G | 376 |
| rs147521015 | 0.0140819 | 1.07 | 49.026 | 38.58 | 0.97605 | 178869701 | G | A | 372 |
| rs149761592 | 0.00147243 | 1.093 | 39.28 | 36.88 | 0.98534 | 178869713 | T | C | 276 |
| rs143022468 | 0.0391973 | 0.936 | 24.858 | 9.97 | 0.98101 | 178869714 | A | G | 368 |
| rs141631802 | 0.0165276 | 0.936 | 47.605 | 43.57 | 0.9837 | 178869740 | G | A | 374 |
| rs143351816 | 0.109974 | 1.049 | 29.28 | 25.2 | 0.98784 | 178869872 | A | G | 377 |
| rs114631238 | 0.150084 | 1.04 | 43.221 | 39.37 | 0.99014 | 178870283 | T | C | 351 |
| rs56673356 | 0.70049 | 1.012 | 29.108 | 21.13 | 0.98929 | 178871089 | T | C | 277 |
| rs10513751 | 0.659109 | 1.013 | 28.738 | 22.57 | 0.98879 | 178872539 | G | A | 296 |
| rs62283317 | 0.662863 | 1.013 | 28.746 | 22.7 | 0.98797 | 178873245 | G | T | 279 |
| rs7651286 | 0.646368 | 1.014 | 28.779 | 22.7 | 0.9863 | 178873811 | C | T | 396 |
| rs16828483 | 0.659357 | 1.013 | 28.705 | 22.7 | 0.98962 | 178874534 | A | G | 328 |
| rs12486651 | 0.646427 | 1.014 | 28.786 | 22.7 | 0.99072 | 178874895 | T | C | 280 |
| rs12487647 | 0.654001 | 1.014 | 28.73 | 22.7 | 0.99052 | 178875966 | A | G | 281 |
| rs57793263 | 0.708571 | 1.011 | 28.718 | 22.7 | 0.9903 | 178876840 | T | C | 282 |
| rs62283321 | 0.670534 | 1.013 | 28.717 | 22.7 | 0.9898 | 178878643 | A | G | 283 |
| rs62283322 | 0.656758 | 1.013 | 28.993 | 22.7 | 0.99007 | 178879032 | A | G | 284 |
| rs62283323 | 0.654245 | 1.014 | 28.729 | 22.7 | 0.99036 | 178879377 | C | T | 285 |
| rs57405296 | 0.155892 | 0.962 | 45.614 | 49.61 | 0.99112 | 178884995 | G | A | 369 |
| rs73174869 | 0.726642 | 1.011 | 28.794 | 22.7 | 0.98857 | 178885112 | T | C | 286 |
| rs34585605 | 0.660167 | 1.013 | 28.76 | 22.7 | 0.99006 | 178885486 | C | T | 345 |
| rs62283343 | 0.657313 | 1.013 | 28.734 | 22.7 | 0.98919 | 178885728 | T | C | 287 |
| rs62283344 | 0.639926 | 1.014 | 28.715 | 22.83 | 0.98971 | 178886301 | G | A | 288 |
| rs62283346 | 0.630929 | 1.015 | 28.679 | 22.83 | 0.99012 | 178886371 | G | C | 289 |
| rs937507 | 0.112423 | 0.957 | 45.399 | 49.34 | 0.99217 | 178887925 | A | G | 398 |
| rs62283348 | 0.87726 | 1.005 | 27.651 | 21.78 | 0.98952 | 178888847 | G | A | 290 |
| rs10513748 | 0.124236 | 0.958 | 45.09 | 49.21 | 0.9935 | 178895268 | T | C | 295 |
| rs78945679 | 0.133187 | 1.07 | 10.18 | 12.34 | 0.98447 | 178906662 | C | G | 291 |
| rs1499810 | 0.753618 | 0.991 | 41.675 | 46.19 | 0.98778 | 178908229 | A | G | 325 |
| rs2133590 | 0.334109 | 0.972 | 31.523 | 37.27 | 0.9903 | 178910344 | G | A | 337 |
| rs6783540 | 0.39277 | 1.026 | 31.235 | 26.38 | 0.98772 | 178913108 | T | A | 385 |
| rs13097723 | 0.973115 | 1.001 | 42.626 | 48.95 | 0.99032 | 178913354 | A | G | 293 |
| rs73174897 | 0.328043 | 1.029 | 31.379 | 26.38 | 0.98685 | 178913462 | C | T | 294 |
| rs67889675 | 0.374806 | 1.027 | 31.245 | 26.38 | 0.99101 | 178913614 | T | C | 386 |
| rs1846586 | 0.341212 | 1.028 | 31.453 | 26.38 | 0.98931 | 178913853 | T | C | 332 |
| rs1846585 | 0.366689 | 1.027 | 31.239 | 26.38 | 0.99021 | 178913998 | T | C | 331 |
| rs4857725 | 0.982994 | 1.001 | 42.831 | 48.95 | 0.98928 | 178914377 | T | C | 364 |
| rs4857726 | 0.874019 | 0.996 | 39.514 | 45.14 | 0.98933 | 178914424 | T | A | 365 |
| rs6765836 | 0.987975 | 1 | 42.783 | 48.95 | 0.99041 | 178914773 | C | G | 380 |
| rs6793647 | 0.930311 | 0.998 | 39.601 | 45.28 | 0.98939 | 178915813 | A | C | 389 |
| rs6782388 | 0.411185 | 1.025 | 31.368 | 26.38 | 0.98915 | 178915993 | C | T | 384 |
| rs28711160 | 0.930311 | 0.998 | 39.596 | 45.41 | 0.9861 | 178916822 | G | A | 341 |
| rs7651130 | 0.772079 | 1.008 | 39.379 | 39.5 | 0.98843 | 178920202 | T | A | 395 |
| rs1106387 | 0.56251 | 1.018 | 28.025 | 29.66 | 0.99474 | 178972962 | A | G | 301 |
| rs1106388 | 0.618224 | 1.015 | 27.893 | 29.66 | 0.99509 | 178973005 | C | T | 302 |

### Example 3

Recently updated analysis using 2-way imputation techniques (described in Stacey et al., 2010) showed a stronger association between rs7586009 and breast cancer in Iceland (P value=2.55×10⁻⁵, odds ratio 1.12 for the C allele). This analysis was based on 4976 directly typed or imputed breast cancer patients and over 24,000 controls. Combined with the non-Icelandic replication samples, the combined association result is P value 1.2×10⁻⁸, OR 1.11 (95% CI 1.07-1.14).

Estrogen receptor (ER) negative breast cancer is a subtype of breast cancer arising in approximately 25% of cases in individuals of European ancestry. ER negative breast cancer presents challenges in terms of both prevention and treatment, primarily because it is not usually responsive to hormonal therapies. We therefore assessed whether the variants were significantly associated with an increased risk of ER negative breast cancer. We found that rs1983011 (C) is significantly associated with ER negative breast cancer (P value=0.037; OR=1.156 based on Icelandic two-way imputation). We also found that rs7856009 (C) is significantly associated with ER negative breast cancer (P value=3.6×10⁻⁴; OR=1.29).

1-34. (canceled)

35. A computer-readable medium having computer executable instructions for determining susceptibility to breast cancer in humans, the computer readable medium comprising:
data indicative of at least one polymorphic marker;
a routine stored on the computer readable medium and adapted to be executed by a processor to determine risk of developing breast cancer for the at least one polymorphic marker;
wherein the at least one polymorphic marker is selected from the group consisting of rs1556283, rs7586009 and rs1983011, and markers in linkage disequilibrium therewith.

36. The computer-readable medium of claim 35, wherein the medium contains data indicative of at least two polymorphic markers.

37. The computer-readable medium of claim 36, wherein the data indicative of the at least one polymorphic marker comprises sequence data identifying the presence or absence of at least one at-risk allele for breast cancer of the at least one polymorphic marker.

38. A system for identifying susceptibility to breast cancer in a human subject, the system comprising:
at least one processor;
at least one computer-readable medium;
a susceptibility database operatively coupled to a computer-readable medium of the system and containing population information correlating the presence or absence of at least one allele of at least one marker selected from the group consisting of rs1556283, rs7586009 and rs1983011, and markers correlated therewith, and susceptibility to breast cancer in a population of humans;
a measurement tool that receives an input about the human subject and generates information from the input about the presence or absence of the at least one allele in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to breast cancer for the human subject.

39. The system according to claim 38, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to breast cancer for the subject.

40. The system according to claim 39, wherein
markers correlated with rs1556283 are selected from the group consisting of the markers listed in Table 3;
markers correlated with rs7586009 are selected from the group consisting of the markers listed in Table 4; and
markers correlated with rs 1983011 are selected from the group consisting of the markers listed in Table 5.

41. The system according to claim 40, wherein the measurement tool comprises a tool stored on a computer-readable medium of the system and adapted to be executed by a processor of the system to receive a data input about a subject and determine information about the presence or absence of the at least one allele in a human subject from the data.

42. The system according to claim 41, wherein the data is genomic sequence information, and the measurement tool comprises a sequence analysis tool stored on a computer readable medium of the system and adapted to be executed by a processor of the system to determine the presence or absence of the at least allele from the genomic sequence information.

43. The system according to claim 42, wherein the input about the human subject is a biological sample from the human subject, and wherein the measurement tool comprises a tool to identify the presence or absence of the at least allele in the biological sample, thereby generating information about the presence or absence of the at least one allele in the human subject.

44. The system according to claim 43, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one allele based on the detection data.

45. The system according to claim 43, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one allele based on the nucleotide sequence information.

46. The system according to claim 45, further comprising:
a medical protocol database operatively connected to a computer-readable medium of the system and containing information correlating the presence or absence of the at least one allele and medical protocols for human subjects at risk for breast cancer; and
a medical protocol routine, operatively connected to the medical protocol database and the analysis routine, stored on a computer-readable medium of the system, and adapted to be executed on a processor of the system, to compare the conclusion from the analysis routine with respect to susceptibility to breast cancer for the subject and the medical protocol database, and generate a protocol report with respect to the probability that one or more medical protocols in the database will:
reduce susceptibility to breast cancer; or
delay onset of breast cancer; or
increase the likelihood of detecting breast cancer at an early stage to facilitate early treatment.

47. The system according to claim 46, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

48. The system according to claim 47, wherein the communication expresses the susceptibility to breast cancer in terms of odds ratio or relative risk or lifetime risk.

49. The system according to claim 48, wherein the communication further includes the protocol report.

50. The system according to claim 49, wherein the susceptibility database further includes information about at least one parameter selected from the group consisting of age, sex, ethnicity, race, medical history, weight, blood pressure, family history of breast cancer, and smoking history in humans and impact of the at least one parameter on susceptibility to breast cancer.

51-56. (canceled)
